SEQUENCE LISTING (101 sequences)

SEQ ID NO: 1 - protein, 5 aa, Artificial Sequence
  Synthetic
GGGGS

SEQ ID NO: 2 - protein, 45 aa, Artificial Sequence
  Synthetic
TTTPAPRPPTPAPTIASQPLSLRPEACRPAAGGAVHTRGLDFACD

SEQ ID NO: 3 - protein, 42 aa, Artificial Sequence
  Synthetic
AAAIEVMYPPPYLDNEKSNGTIIHVKGKHLCPSPLFPGPSKP

SEQ ID NO: 4 - protein, 24 aa, Artificial Sequence
  Synthetic
IYIWAPLAGTCGVLLLSLVITLYC

SEQ ID NO: 5 - protein, 68 aa, Artificial Sequence
  Synthetic
FWVLVVVGGVLACYSLLVTVAFIIFWVRSKRSRLLHSDYMNMTPRRPGPTRKHYQPYAPPRDFAAYRS

SEQ ID NO: 6 - protein, 112 aa, Artificial Sequence
  Synthetic
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 42 aa, Artificial Sequence
  Synthetic
KRGRKKLLYIFKQPFMRPVQTTQEEDGCSCRFPEEEEGGCEL

SEQ ID NO: 8 - DNA, 402 bp, Artificial Sequence
  Synthetic
gtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcg

SEQ ID NO: 9 - DNA, 1509 bp, Artificial Sequence
  Synthetic
atgtggctgcagtctctgctgctgctgggcaccgtggcctgtagcatcagcgagatcgtgctgacccagagccctggctctctggctgtgtctcctggcgagcgcgtgaccatgagctgcaagagcagccagagcgtgttcttcagcagctcccagaagaactacctggcctggtatcagcagatccccggccagagccccagactgctgatctactgggccagcaccagagaaagcggcgtgcccgatagattcaccggcagcggctctggcaccgacttcaccctgacaatcagcagcgtgcagcccgaggacctggccatctactactgccaccagtacctgagcagccggacctttggccagggcaccaagctggaaatcaagcggggcagcacaagcggcagcggaaagcctggatctggcgagggctctaccaagggccaggtgcagctgcagcagcctggcgccgaagtcgtgaaacctggcgcctccgtgaagatgtcctgcaaggccagcggctacaccttcaccagctactacatccactggatcaagcagacccctggacagggcctggaatgggtgggagtgatctaccccggcaacgacgacatcagctacaaccagaagttccagggcaaggccaccctgaccgccgacaagtctagcaccaccgcctacatgcagctgtccagcctgaccagcgaggacagcgccgtgtactactgcgccagagaagtgcggctgcggtacttcgatgtgtggggccagggaaccaccgtgaccgtgtctagcgccctgagcaacagcatcatgtacttcagccacttcgtgcccgtgtttctgcccgccaagcctaccacaacccctgcccctagacctcctaccccagcccctacaatcgccagccagcctctgtctctgaggcccgaggcttctagaccagctgctggcggagccgtgcacaccagaggcctggatatctacatctgggccccactggccggcacctgtggcgtgctgctgctgtctctcgtgatcaccaagagaggccggaagaagctgctgtacatcttcaagcagcccttcatgcggcccgtgcagaccacccaggaagaggacggctgtagctgccggttccccgaggaagaagaagggggctgcgagctgagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagacggaagaaccctcaggaaggcctgtataacgaactgcagaaagacaagatggccgaggcctactccgagatcggaatgaagggcgagcggagaagaggcaagggccacgatggactgtaccagggcctgagcaccgccaccaaggacacctatgacgccctgcacatgcaggccctgccccccaga

SEQ ID NO: 10 - protein, 503 aa, Artificial Sequence
  Synthetic
MWLQSLLLLGTVACSISEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRGSTSGSGKPGSGEGSTKGQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSALSNSIMYFSHFVPVFLPAKPTTTPAPRPPTPAPTIASQPLSLRPEASRPAAGGAVHTRGLDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - DNA, 10408 bp, Artificial Sequence
  Synthetic
tgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagaattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtggatctgcgatcgcgtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgtctagagctagcggatccccggaattcgacgccaccatgtggctgcagtctctgctgctgctgggcaccgtggcctgtagcatcagcgagatcgtgctgacccagagccctggctctctggctgtgtctcctggcgagcgcgtgaccatgagctgcaagagcagccagagcgtgttcttcagcagctcccagaagaactacctggcctggtatcagcagatccccggccagagccccagactgctgatctactgggccagcaccagagaaagcggcgtgcccgatagattcaccggcagcggctctggcaccgacttcaccctgacaatcagcagcgtgcagcccgaggacctggccatctactactgccaccagtacctgagcagccggacctttggccagggcaccaagctggaaatcaagcggggcagcacaagcggcagcggaaagcctggatctggcgagggctctaccaagggccaggtgcagctgcagcagcctggcgccgaagtcgtgaaacctggcgcctccgtgaagatgtcctgcaaggccagcggctacaccttcaccagctactacatccactggatcaagcagacccctggacagggcctggaatgggtgggagtgatctaccccggcaacgacgacatcagctacaaccagaagttccagggcaaggccaccctgaccgccgacaagtctagcaccaccgcctacatgcagctgtccagcctgaccagcgaggacagcgccgtgtactactgcgccagagaagtgcggctgcggtacttcgatgtgtggggccagggaaccaccgtgaccgtgtctagcgccctgagcaacagcatcatgtacttcagccacttcgtgcccgtgtttctgcccgccaagcctaccacaacccctgcccctagacctcctaccccagcccctacaatcgccagccagcctctgtctctgaggcccgaggcttctagaccagctgctggcggagccgtgcacaccagaggcctggatatctacatctgggccccactggccggcacctgtggcgtgctgctgctgtctctcgtgatcaccaagagaggccggaagaagctgctgtacatcttcaagcagcccttcatgcggcccgtgcagaccacccaggaagaggacggctgtagctgccggttccccgaggaagaagaagggggctgcgagctgagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagacggaagaaccctcaggaaggcctgtataacgaactgcagaaagacaagatggccgaggcctactccgagatcggaatgaagggcgagcggagaagaggcaagggccacgatggactgtaccagggcctgagcaccgccaccaaggacacctatgacgccctgcacatgcaggccctgccccccagagagggcagaggaagtcttctaacatgcggtgacgtggaggagaatcccggcccttccgggatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaaaggttaattaaggcgcgcccaattgaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtag

SEQ ID NO: 12 - DNA, 1476 bp, Artificial Sequence
  Synthetic
atggccctgcctgtgacagccctgctgctgcccctggctctgctgctgcatgccgccagacctgaggtgcagctggtgcagtctggcgccgaagtgaagaaacccggcagcagcgtgaaggtgtcctgcaaggccagcggctacaccatcaccgacagcaacatccactgggtgcgccaggcccctggccagagcctggaatggatcggctacatctacccctacaacggcggcaccgactacaaccagaagttcaagaaccgggccaccctgaccgtggacaaccccaccaacaccgcctacatggaactgagcagcctgcggagcgaggacaccgccttctactactgcgtgaacggcaacccctggctggcctactggggccagggaaccctggtgacagtgtctagcggcggaggcggatctggagggggaggatctggcggcggaggaagcgacatccagctgacccagagccccagcaccctgagcgccagcgtgggcgacagagtgaccatcacctgtcgggccagcgagagcctggacaactacggcatccggtttctgacctggttccagcagaagcccggcaaggcccccaagctgctgatgtacgccgccagcaatcagggcagcggcgtgcccagcagattcagcggctctggcagcggaaccgagttcaccctgaccatcagcagcctgcagcccgacgacttcgccacctactactgccagcagaccaaagaggtgccctggtccttcggccagggcaccaaggtggaagtgaagcggactagttccggagccgccgccatcgaagtgatgtacccccctccctacctggataacgagaagagcaacggcaccatcatccacgtgaagggaaagcacctgtgtcccagccccctgtttcccggccctagcaagcccttctgggtgctggtggtggtcggcggagtgctggcctgctacagcctcctggtgaccgtggccttcatcatcttctgggtgaggagcaagaggtccaggctgctgcacagcgactacatgaatatgacccccagaaggcccggccccaccagaaagcactatcagccctacgccccccccagggactttgccgcctacaggagcagggtgaagttcagcagatccgccgatgcccctgcttaccagcagggccagaaccagctgtataacgagctgaacctgggcaggagggaggaatacgacgtgctggataagaggaggggaagggaccccgagatgggcggaaagcccaggaggaagaacccccaggagggcctgtacaatgagctgcagaaagacaagatggccgaggcctacagcgagatcggcatgaagggcgagaggaggaggggcaagggccatgacggcctgtaccaaggcctgtccaccgccaccaaggatacctacgacgccctgcacatgcaggccctgcctcccaggggatcc

SEQ ID NO: 13 - protein, 492 aa, Artificial Sequence
  Synthetic
MALPVTALLLPLALLLHAARPEVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTSSGAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGS

SEQ ID NO: 14 - DNA, 10365 bp, Artificial Sequence
  Synthetic
tgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagaattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtggatctgcgatcgcgtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgtctagagctagcggatccccggaattcgacgccaccatggccctgcctgtgacagccctgctgctgcccctggctctgctgctgcatgccgccagacctgaggtgcagctggtgcagtctggcgccgaagtgaagaaacccggcagcagcgtgaaggtgtcctgcaaggccagcggctacaccatcaccgacagcaacatccactgggtgcgccaggcccctggccagagcctggaatggatcggctacatctacccctacaacggcggcaccgactacaaccagaagttcaagaaccgggccaccctgaccgtggacaaccccaccaacaccgcctacatggaactgagcagcctgcggagcgaggacaccgccttctactactgcgtgaacggcaacccctggctggcctactggggccagggaaccctggtgacagtgtctagcggcggaggcggatctggagggggaggatctggcggcggaggaagcgacatccagctgacccagagccccagcaccctgagcgccagcgtgggcgacagagtgaccatcacctgtcgggccagcgagagcctggacaactacggcatccggtttctgacctggttccagcagaagcccggcaaggcccccaagctgctgatgtacgccgccagcaatcagggcagcggcgtgcccagcagattcagcggctctggcagcggaaccgagttcaccctgaccatcagcagcctgcagcccgacgacttcgccacctactactgccagcagaccaaagaggtgccctggtccttcggccagggcaccaaggtggaagtgaagcggactagttccggagccgccgccatcgaagtgatgtacccccctccctacctggataacgagaagagcaacggcaccatcatccacgtgaagggaaagcacctgtgtcccagccccctgtttcccggccctagcaagcccttctgggtgctggtggtggtcggcggagtgctggcctgctacagcctcctggtgaccgtggccttcatcatcttctgggtgaggagcaagaggtccaggctgctgcacagcgactacatgaatatgacccccagaaggcccggccccaccagaaagcactatcagccctacgccccccccagggactttgccgcctacaggagcagggtgaagttcagcagatccgccgatgcccctgcttaccagcagggccagaaccagctgtataacgagctgaacctgggcaggagggaggaatacgacgtgctggataagaggaggggaagggaccccgagatgggcggaaagcccaggaggaagaacccccaggagggcctgtacaatgagctgcagaaagacaagatggccgaggcctacagcgagatcggcatgaagggcgagaggaggaggggcaagggccatgacggcctgtaccaaggcctgtccaccgccaccaaggatacctacgacgccctgcacatgcaggccctgcctcccaggggatccgagggcagaggaagtcttctaacatgcggtgacgtggaggagaatcccggcccttccgggatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaattaattaaccaattgaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtag

SEQ ID NO: 15 - DNA, 1473 bp, Artificial Sequence
  Synthetic
atggccctgcctgtgacagccctgctgctgcccctggctctgctgctgcatgccgccagacctgaggtgcagctggtgcagtctggcgccgaagtgaagaaacccggcagcagcgtgaaggtgtcctgcaaggccagcggctacaccatcaccgacagcaacatccactgggtgcgccaggcccctggccagagcctggaatggatcggctacatctacccctacaacggcggcaccgactacaaccagaagttcaagaaccgggccaccctgaccgtggacaaccccaccaacaccgcctacatggaactgagcagcctgcggagcgaggacaccgccttctactactgcgtgaacggcaacccctggctggcctactggggccagggaaccctggtgacagtgtctagcggcggaggcggatctggagggggaggatctggcggcggaggaagcgacatccagctgacccagagccccagcaccctgagcgccagcgtgggcgacagagtgaccatcacctgtcgggccagcgagagcctggacaactacggcatccggtttctgacctggttccagcagaagcccggcaaggcccccaagctgctgatgtacgccgccagcaatcagggcagcggcgtgcccagcagattcagcggctctggcagcggaaccgagttcaccctgaccatcagcagcctgcagcccgacgacttcgccacctactactgccagcagaccaaagaggtgccctggtccttcggccagggcaccaaggtggaagtgaagcggactagttccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 16 - protein, 491 aa, Artificial Sequence
  Synthetic
MALPVTALLLPLALLLHAARPEVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - DNA, 10362 bp, Artificial Sequence
  Synthetic
tgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagaattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtggatctgcgatcgcgtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgtctagagctagcggatccccggaattcgacgccaccatggccctgcctgtgacagccctgctgctgcccctggctctgctgctgcatgccgccagacctgaggtgcagctggtgcagtctggcgccgaagtgaagaaacccggcagcagcgtgaaggtgtcctgcaaggccagcggctacaccatcaccgacagcaacatccactgggtgcgccaggcccctggccagagcctggaatggatcggctacatctacccctacaacggcggcaccgactacaaccagaagttcaagaaccgggccaccctgaccgtggacaaccccaccaacaccgcctacatggaactgagcagcctgcggagcgaggacaccgccttctactactgcgtgaacggcaacccctggctggcctactggggccagggaaccctggtgacagtgtctagcggcggaggcggatctggagggggaggatctggcggcggaggaagcgacatccagctgacccagagccccagcaccctgagcgccagcgtgggcgacagagtgaccatcacctgtcgggccagcgagagcctggacaactacggcatccggtttctgacctggttccagcagaagcccggcaaggcccccaagctgctgatgtacgccgccagcaatcagggcagcggcgtgcccagcagattcagcggctctggcagcggaaccgagttcaccctgaccatcagcagcctgcagcccgacgacttcgccacctactactgccagcagaccaaagaggtgccctggtccttcggccagggcaccaaggtggaagtgaagcggactagttccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgagggcagaggaagtcttctaacatgcggtgacgtggaggagaatcccggcccttccgggatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaattaattaaccaattgaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtag

SEQ ID NO: 18 - DNA, 1467 bp, Artificial Sequence
  Synthetic
atggctctgcccgtcacagctctgctgctgcctctggccctgctgctgcacgccgccagacctcaggtgcagctcgtgcagagcggcgctgaggtgaagaaacctggcagcagcgtgaaggtgagctgcaaggcctccggctacaccttcaccgactacaacatgcactgggtgaggcaagcccctggccagggactggagtggatcggctacatctacccttacaacggcggcacaggctacaaccagaagttcaagtccaaggccaccatcaccgccgatgagtccaccaataccgcctacatggagctcagcagcctgaggtccgaggacacagccgtctactactgcgccaggggcaggcccgctatggactactggggccagggcaccctggtgacagtgagctctggtggcggcggatccggcggcggcggcagcggcggcggcggctccgacattcagatgacccagagccctagcagcctgagcgcttccgtgggagacagggtgaccatcacatgcagggcctccgagagcgtggacaattacggcatcagcttcatgaactggttccagcagaagcccggcaaggcccccaaactgctgatctatgccgccagcaatcagggctccggcgtgcctagcaggttttccggcagcggcagcggcaccgactttaccctgaccatctccagcctgcagcctgacgatttcgccacctactactgccagcagagcaaggaggtgccttggacctttggacagggcacaaaggtggagatcaagtccggagccgccgccatcgaagtgatgtacccccctccctacctggataacgagaagagcaacggcaccatcatccacgtgaagggaaagcacctgtgtcccagccccctgtttcccggccctagcaagcccttctgggtgctggtggtggtcggcggagtgctggcctgctacagcctcctggtgaccgtggccttcatcatcttctgggtgaggagcaagaggtccaggctgctgcacagcgactacatgaatatgacccccagaaggcccggccccaccagaaagcactatcagccctacgccccccccagggactttgccgcctacaggagcagggtgaagttcagcagatccgccgatgcccctgcttaccagcagggccagaaccagctgtataacgagctgaacctgggcaggagggaggaatacgacgtgctggataagaggaggggaagggaccccgagatgggcggaaagcccaggaggaagaacccccaggagggcctgtacaatgagctgcagaaagacaagatggccgaggcctacagcgagatcggcatgaagggcgagaggaggaggggcaagggccatgacggcctgtaccaaggcctgtccaccgccaccaaggatacctacgacgccctgcacatgcaggccctgcctcccaggggatcc

SEQ ID NO: 19 - protein, 489 aa, Artificial Sequence
  Synthetic
MALPVTALLLPLALLLHAARPQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKSGAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGS

SEQ ID NO: 20 - DNA, 10356 bp, Artificial Sequence
  Synthetic
tgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagaattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtggatctgcgatcgcgtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgtctagagctagcggatccccggaattcgacgccaccatggctctgcccgtcacagctctgctgctgcctctggccctgctgctgcacgccgccagacctcaggtgcagctcgtgcagagcggcgctgaggtgaagaaacctggcagcagcgtgaaggtgagctgcaaggcctccggctacaccttcaccgactacaacatgcactgggtgaggcaagcccctggccagggactggagtggatcggctacatctacccttacaacggcggcacaggctacaaccagaagttcaagtccaaggccaccatcaccgccgatgagtccaccaataccgcctacatggagctcagcagcctgaggtccgaggacacagccgtctactactgcgccaggggcaggcccgctatggactactggggccagggcaccctggtgacagtgagctctggtggcggcggatccggcggcggcggcagcggcggcggcggctccgacattcagatgacccagagccctagcagcctgagcgcttccgtgggagacagggtgaccatcacatgcagggcctccgagagcgtggacaattacggcatcagcttcatgaactggttccagcagaagcccggcaaggcccccaaactgctgatctatgccgccagcaatcagggctccggcgtgcctagcaggttttccggcagcggcagcggcaccgactttaccctgaccatctccagcctgcagcctgacgatttcgccacctactactgccagcagagcaaggaggtgccttggacctttggacagggcacaaaggtggagatcaagtccggagccgccgccatcgaagtgatgtacccccctccctacctggataacgagaagagcaacggcaccatcatccacgtgaagggaaagcacctgtgtcccagccccctgtttcccggccctagcaagcccttctgggtgctggtggtggtcggcggagtgctggcctgctacagcctcctggtgaccgtggccttcatcatcttctgggtgaggagcaagaggtccaggctgctgcacagcgactacatgaatatgacccccagaaggcccggccccaccagaaagcactatcagccctacgccccccccagggactttgccgcctacaggagcagggtgaagttcagcagatccgccgatgcccctgcttaccagcagggccagaaccagctgtataacgagctgaacctgggcaggagggaggaatacgacgtgctggataagaggaggggaagggaccccgagatgggcggaaagcccaggaggaagaacccccaggagggcctgtacaatgagctgcagaaagacaagatggccgaggcctacagcgagatcggcatgaagggcgagaggaggaggggcaagggccatgacggcctgtaccaaggcctgtccaccgccaccaaggatacctacgacgccctgcacatgcaggccctgcctcccaggggatccgagggcagaggaagtcttctaacatgcggtgacgtggaggagaatcccggcccttccgggatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaattaattaaccaattgaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtag

SEQ ID NO: 21 - DNA, 1464 bp, Artificial Sequence
  Synthetic
atggctctgcccgtcacagctctgctgctgcctctggccctgctgctgcacgccgccagacctcaggtgcagctcgtgcagagcggcgctgaggtgaagaaacctggcagcagcgtgaaggtgagctgcaaggcctccggctacaccttcaccgactacaacatgcactgggtgaggcaagcccctggccagggactggagtggatcggctacatctacccttacaacggcggcacaggctacaaccagaagttcaagtccaaggccaccatcaccgccgatgagtccaccaataccgcctacatggagctcagcagcctgaggtccgaggacacagccgtctactactgcgccaggggcaggcccgctatggactactggggccagggcaccctggtgacagtgagctctggtggcggcggatccggcggcggcggcagcggcggcggcggctccgacattcagatgacccagagccctagcagcctgagcgcttccgtgggagacagggtgaccatcacatgcagggcctccgagagcgtggacaattacggcatcagcttcatgaactggttccagcagaagcccggcaaggcccccaaactgctgatctatgccgccagcaatcagggctccggcgtgcctagcaggttttccggcagcggcagcggcaccgactttaccctgaccatctccagcctgcagcctgacgatttcgccacctactactgccagcagagcaaggaggtgccttggacctttggacagggcacaaaggtggagatcaagtccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 22 - protein, 488 aa, Artificial Sequence
  Synthetic
MALPVTALLLPLALLLHAARPQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - DNA, 10353 bp, Artificial Sequence
  Synthetic
tgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagaattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtggatctgcgatcgcgtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgtctagagctagcggatccccggaattcgacgccaccatggctctgcccgtcacagctctgctgctgcctctggccctgctgctgcacgccgccagacctcaggtgcagctcgtgcagagcggcgctgaggtgaagaaacctggcagcagcgtgaaggtgagctgcaaggcctccggctacaccttcaccgactacaacatgcactgggtgaggcaagcccctggccagggactggagtggatcggctacatctacccttacaacggcggcacaggctacaaccagaagttcaagtccaaggccaccatcaccgccgatgagtccaccaataccgcctacatggagctcagcagcctgaggtccgaggacacagccgtctactactgcgccaggggcaggcccgctatggactactggggccagggcaccctggtgacagtgagctctggtggcggcggatccggcggcggcggcagcggcggcggcggctccgacattcagatgacccagagccctagcagcctgagcgcttccgtgggagacagggtgaccatcacatgcagggcctccgagagcgtggacaattacggcatcagcttcatgaactggttccagcagaagcccggcaaggcccccaaactgctgatctatgccgccagcaatcagggctccggcgtgcctagcaggttttccggcagcggcagcggcaccgactttaccctgaccatctccagcctgcagcctgacgatttcgccacctactactgccagcagagcaaggaggtgccttggacctttggacagggcacaaaggtggagatcaagtccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgagggcagaggaagtcttctaacatgcggtgacgtggaggagaatcccggcccttccgggatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaattaattaaccaattgaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtag

SEQ ID NO: 24 - DNA, 1536 bp, Artificial Sequence
  Synthetic
atggccctgcctgtgacagccctgctgctgcccctggctctgctgctgcatgccgccagacctatggctctgcccgtgaccgctctcctcctgccactggcactgctcctccacgctgctagaccccaggtgcagctggtgcagtctggcgccgaagtgaagaaacccggcagcagcgtgaaggtgtcctgcaaggccagcggctacaccttcaccgactacaacatgcactgggtgcgccaggctccaggccagggactggaatggatcggctacatctacccctacaacggcggcaccggctacaaccagaagttcaagagcaaggccaccatcaccgccgacgagagcaccaacaccgcctacatggaactgagcagcctgcggagcgaggacaccgccgtgtactactgcgccagaggcagacccgccatggactactggggccagggcaccctggtgacagtgtctagcggaggcggaggctctggcggcggaggaagtggcggaggcggcagcgatatccagatgacccagagccccagcagcctgagcgccagcgtgggcgacagagtgaccatcacctgtcgggccagcgagagcgtggacaactacggcatcagcttcatgaactggttccagcagaagcccggcaaggcccccaagctgctgatctacgccgccagcaatcagggcagcggcgtgcccagcagattcagcggctctggcagcggcaccgacttcaccctgaacatcagcagcctgcagcccgacgacttcgccacctactactgccagcagagcaaagaggtgccctggaccttcggacagggcaccaaggtggaaatcaagactagttccggagccgccgccatcgaagtgatgtacccccctccctacctggataacgagaagagcaacggcaccatcatccacgtgaagggaaagcacctgtgtcccagccccctgtttcccggccctagcaagcccttctgggtgctggtggtggtcggcggagtgctggcctgctacagcctcctggtgaccgtggccttcatcatcttctgggtgaggagcaagaggtccaggctgctgcacagcgactacatgaatatgacccccagaaggcccggccccaccagaaagcactatcagccctacgccccccccagggactttgccgcctacaggagcagggtgaagttcagcagatccgccgatgcccctgcttaccagcagggccagaaccagctgtataacgagctgaacctgggcaggagggaggaatacgacgtgctggataagaggaggggaagggaccccgagatgggcggaaagcccaggaggaagaacccccaggagggcctgtacaatgagctgcagaaagacaagatggccgaggcctacagcgagatcggcatgaagggcgagaggaggaggggcaagggccatgacggcctgtaccaaggcctgtccaccgccaccaaggatacctacgacgccctgcacatgcaggccctgcctcccaggggatcc

SEQ ID NO: 25 - protein, 512 aa, Artificial Sequence
  Synthetic
MALPVTALLLPLALLLHAARPMALPVTALLLPLALLLHAARPQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLNISSLQPDDFATYYCQQSKEVPWTFGQGTKVEIKTSSGAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGS

SEQ ID NO: 26 - DNA, 10425 bp, Artificial Sequence
  Synthetic
tgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagaattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtggatctgcgatcgcgtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgtctagagctagcggatccccggaattcgacgccaccatggccctgcctgtgacagccctgctgctgcccctggctctgctgctgcatgccgccagacctatggctctgcccgtgaccgctctcctcctgccactggcactgctcctccacgctgctagaccccaggtgcagctggtgcagtctggcgccgaagtgaagaaacccggcagcagcgtgaaggtgtcctgcaaggccagcggctacaccttcaccgactacaacatgcactgggtgcgccaggctccaggccagggactggaatggatcggctacatctacccctacaacggcggcaccggctacaaccagaagttcaagagcaaggccaccatcaccgccgacgagagcaccaacaccgcctacatggaactgagcagcctgcggagcgaggacaccgccgtgtactactgcgccagaggcagacccgccatggactactggggccagggcaccctggtgacagtgtctagcggaggcggaggctctggcggcggaggaagtggcggaggcggcagcgatatccagatgacccagagccccagcagcctgagcgccagcgtgggcgacagagtgaccatcacctgtcgggccagcgagagcgtggacaactacggcatcagcttcatgaactggttccagcagaagcccggcaaggcccccaagctgctgatctacgccgccagcaatcagggcagcggcgtgcccagcagattcagcggctctggcagcggcaccgacttcaccctgaacatcagcagcctgcagcccgacgacttcgccacctactactgccagcagagcaaagaggtgccctggaccttcggacagggcaccaaggtggaaatcaagactagttccggagccgccgccatcgaagtgatgtacccccctccctacctggataacgagaagagcaacggcaccatcatccacgtgaagggaaagcacctgtgtcccagccccctgtttcccggccctagcaagcccttctgggtgctggtggtggtcggcggagtgctggcctgctacagcctcctggtgaccgtggccttcatcatcttctgggtgaggagcaagaggtccaggctgctgcacagcgactacatgaatatgacccccagaaggcccggccccaccagaaagcactatcagccctacgccccccccagggactttgccgcctacaggagcagggtgaagttcagcagatccgccgatgcccctgcttaccagcagggccagaaccagctgtataacgagctgaacctgggcaggagggaggaatacgacgtgctggataagaggaggggaagggaccccgagatgggcggaaagcccaggaggaagaacccccaggagggcctgtacaatgagctgcagaaagacaagatggccgaggcctacagcgagatcggcatgaagggcgagaggaggaggggcaagggccatgacggcctgtaccaaggcctgtccaccgccaccaaggatacctacgacgccctgcacatgcaggccctgcctcccaggggatccgagggcagaggaagtcttctaacatgcggtgacgtggaggagaatcccggcccttccgggatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaattaattaaccaattgaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtag

SEQ ID NO: 27 - protein, 12 aa, Artificial Sequence
  Synthetic
LGGSLPDYGMDV

SEQ ID NO: 28 - DNA, 1482 bp, Artificial Sequence
  Synthetic
atggccctccctgtcaccgccctgctgcttccgctggctcttctgctccacgccgctcggccccaagtgcaactcgtccagtccggtgcagaagtcaagaagccaggagaatcactcaagattagctgcaaaggcagcggctactccttcacttcctactggatcggctgggtgcgccagatgcccggaaagggactggagtggatgggaatcatctaccctggcgatagcgacaccagatactccccgagctttcaaggccaagtgaccatttcggccgacaagtcgatctccaccgcgtatctgcagtggagctcactgaaggcttcggacaccgccatgtactactgtgcccggctggggggaagcctgcccgattacggaatggacgtgtggggccagggaaccatggtcactgtgtcctccgcctccgggggtggaggctccggtggaggggggtccggtggtggaggatcagaaattgtgctgacccagtctccgctgtccttgcctgtgaccccgggcgaacccgcaagcatctcctgccggtcgtcgcagtccctgcttcactccaacggctacaactacctcgattggtacctccagaagcctggacagagcccacagctgttgatctacctgggctcgaaccgggcctcaggagtgccggacaggttctccggctccgggtcgggaaccgacttcacgctgaagatctcccgcgtggaggccgaggacgtgggcgtgtactattgcatgcaggcgctgcagacccttattacattcggacaggggactaaggtcgatatcaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 29 - protein, 494 aa, Artificial Sequence
  Synthetic
MALPVTALLLPLALLLHAARPQVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLGGSLPDYGMDVWGQGTMVTVSSASGGGGSGGGGSGGGGSEIVLTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTLITFGQGTKVDIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - DNA, 10371 bp, Artificial Sequence
  Synthetic
tgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagaattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtggatctgcgatcgcgtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgtctagagctagcggatccccggaattcgacgccaccatggccctccctgtcaccgccctgctgcttccgctggctcttctgctccacgccgctcggccccaagtgcaactcgtccagtccggtgcagaagtcaagaagccaggagaatcactcaagattagctgcaaaggcagcggctactccttcacttcctactggatcggctgggtgcgccagatgcccggaaagggactggagtggatgggaatcatctaccctggcgatagcgacaccagatactccccgagctttcaaggccaagtgaccatttcggccgacaagtcgatctccaccgcgtatctgcagtggagctcactgaaggcttcggacaccgccatgtactactgtgcccggctggggggaagcctgcccgattacggaatggacgtgtggggccagggaaccatggtcactgtgtcctccgcctccgggggtggaggctccggtggaggggggtccggtggtggaggatcagaaattgtgctgacccagtctccgctgtccttgcctgtgaccccgggcgaacccgcaagcatctcctgccggtcgtcgcagtccctgcttcactccaacggctacaactacctcgattggtacctccagaagcctggacagagcccacagctgttgatctacctgggctcgaaccgggcctcaggagtgccggacaggttctccggctccgggtcgggaaccgacttcacgctgaagatctcccgcgtggaggccgaggacgtgggcgtgtactattgcatgcaggcgctgcagacccttattacattcggacaggggactaaggtcgatatcaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgggagggcagaggaagtcttctaacatgcggtgacgtggaggagaatcccggcccttccgggatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaattaattaaccaattgaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtag

SEQ ID NO: 31 - protein, 13 aa, Artificial Sequence
  Synthetic
RGGYSDYDYYFDF

SEQ ID NO: 32 - DNA, 1482 bp, Artificial Sequence
  Synthetic
atggccctccctgtcaccgccctgctgcttccgctggctcttctgctccacgccgctcggccccaagtgcagctcgtccaatccggtgcagaagtgaagaagcctggcgaatccctgaagatctcatgcaaaggctcgggatacagcttcacctcatattggattggatgggtcagacagatgccaggaaagggtctggagtggatgggaatcatctacccgggagacagcgatacccggtactccccgagcttccagggacaggtcaccatctcggccgacaagtccattactactgcctacttgcaatggtcctcgctgcgcgcctccgatagcgccatgtactactgcgcgagaggcggctactccgactacgactactacttcgatttctggggacaggggacactcgtgactgtgtcctccgcgtcgggtggcggcggctcgggtggaggaggaagcggagggggaggctccgaaattgtgatgacccagtcacccctgtcgctccctgtgactcctggggaaccggcctccatctcctgccggagctcacagagcctgctgcactccaacggatacaactacctcgattggtaccttcagaagcccggccagtcgccccagctgctgatctacctggggtccaaccgggctagcggcgtgccggaccgcttctccggttccgggtctggaaccgacttcacgctgaaaatctccagggtggaggccgaggacgtgggagtgtattactgtatgcaggccctgcaaacccccttcacctttggcgggggcaccaaggtcgagattaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 33 - protein, 494 aa, Artificial Sequence
  Synthetic
MALPVTALLLPLALLLHAARPQVQLVQSGAEVKKPGESLKISCKGSGYSFTSYWIGWVRQMPGKGLEWMGIIYPGDSDTRYSPSFQGQVTISADKSITTAYLQWSSLRASDSAMYYCARGGYSDYDYYFDFWGQGTLVTVSSASGGGGSGGGGSGGGGSEIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPFTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - DNA, 10371 bp, Artificial Sequence
  Synthetic
tgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagaattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtggatctgcgatcgcgtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgtctagagctagcggatccccggaattcgacgccaccatggccctccctgtcaccgccctgctgcttccgctggctcttctgctccacgccgctcggccccaagtgcagctcgtccaatccggtgcagaagtgaagaagcctggcgaatccctgaagatctcatgcaaaggctcgggatacagcttcacctcatattggattggatgggtcagacagatgccaggaaagggtctggagtggatgggaatcatctacccgggagacagcgatacccggtactccccgagcttccagggacaggtcaccatctcggccgacaagtccattactactgcctacttgcaatggtcctcgctgcgcgcctccgatagcgccatgtactactgcgcgagaggcggctactccgactacgactactacttcgatttctggggacaggggacactcgtgactgtgtcctccgcgtcgggtggcggcggctcgggtggaggaggaagcggagggggaggctccgaaattgtgatgacccagtcacccctgtcgctccctgtgactcctggggaaccggcctccatctcctgccggagctcacagagcctgctgcactccaacggatacaactacctcgattggtaccttcagaagcccggccagtcgccccagctgctgatctacctggggtccaaccgggctagcggcgtgccggaccgcttctccggttccgggtctggaaccgacttcacgctgaaaatctccagggtggaggccgaggacgtgggagtgtattactgtatgcaggccctgcaaacccccttcacctttggcgggggcaccaaggtcgagattaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgggagggcagaggaagtcttctaacatgcggtgacgtggaggagaatcccggcccttccgggatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaattaattaaccaattgaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtag

SEQ ID NO: 35 - DNA, 1476 bp, Artificial Sequence
  Synthetic
atggccctgcctgtgacagccctgctgctgcctctggctctgctgctgcatgccgctagacccggatccgagatcgtgctgacacagagccctggaagcctggccgtgtctcctggcgagcgcgtgacaatgagctgcaagagcagccagagcgtgttcttcagcagctcccagaagaactacctggcctggtatcagcagatccccggccagagccccagactgctgatctactgggccagcaccagagaaagcggcgtgcccgatagattcaccggcagcggctctggcaccgacttcaccctgacaatcagcagcgtgcagcccgaggacctggccatctactactgccaccagtacctgagcagccggacctttggccagggcaccaagctggaaatcaagagaggcggcggaggctctggcggaggcggatctagtggcggaggatctcaggtgcagctgcagcagcctggcgccgaggtcgtgaaacctggcgcctctgtgaagatgtcctgcaaggccagcggctacaccttcaccagctactacatccactggatcaagcagacccctggacagggcctggaatgggtgggagtgatctaccccggcaacgacgacatcagctacaaccagaagttccagggcaaggccaccctgaccgccgacaagtctagcaccaccgcctacatgcagctgtccagcctgaccagcgaggacagcgccgtgtactactgcgccagagaagtgcggctgcggtacttcgatgtgtggggccagggaaccaccgtgaccgtgtcatctaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 36 - protein, 492 aa, Artificial Sequence
  Synthetic
MALPVTALLLPLALLLHAARPGSEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRGGGGSGGGGSSGGGSQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - DNA, 10365 bp, Artificial Sequence
  Synthetic
tgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagaattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtggatctgcgatcgcgtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgtctagagctagcggatccccggaattcgacgccaccatggccctgcctgtgacagccctgctgctgcctctggctctgctgctgcatgccgctagacccggatccgagatcgtgctgacacagagccctggaagcctggccgtgtctcctggcgagcgcgtgacaatgagctgcaagagcagccagagcgtgttcttcagcagctcccagaagaactacctggcctggtatcagcagatccccggccagagccccagactgctgatctactgggccagcaccagagaaagcggcgtgcccgatagattcaccggcagcggctctggcaccgacttcaccctgacaatcagcagcgtgcagcccgaggacctggccatctactactgccaccagtacctgagcagccggacctttggccagggcaccaagctggaaatcaagagaggcggcggaggctctggcggaggcggatctagtggcggaggatctcaggtgcagctgcagcagcctggcgccgaggtcgtgaaacctggcgcctctgtgaagatgtcctgcaaggccagcggctacaccttcaccagctactacatccactggatcaagcagacccctggacagggcctggaatgggtgggagtgatctaccccggcaacgacgacatcagctacaaccagaagttccagggcaaggccaccctgaccgccgacaagtctagcaccaccgcctacatgcagctgtccagcctgaccagcgaggacagcgccgtgtactactgcgccagagaagtgcggctgcggtacttcgatgtgtggggccagggaaccaccgtgaccgtgtcatctaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgggagggcagaggaagtcttctaacatgcggtgacgtggaggagaatcccggcccttccgggatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaattaattaaccaattgaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtag

SEQ ID NO: 38 - DNA, 2037 bp, Artificial Sequence
  Synthetic
atggccctgcctgtgacagccctgctgctgcctctggctctgctgctgcatgccgctagacccggatccgagatcgtgctgacacagagccctggaagcctggccgtgtctcctggcgagcgcgtgacaatgagctgcaagagcagccagagcgtgttcttcagcagctcccagaagaactacctggcctggtatcagcagatccccggccagagccccagactgctgatctactgggccagcaccagagaaagcggcgtgcccgatagattcaccggcagcggctctggcaccgacttcaccctgacaatcagcagcgtgcagcccgaggacctggccatctactactgccaccagtacctgagcagccggacctttggccagggcaccaagctggaaatcaagagaggcggcggaggctctggcggaggcggatctagtggcggaggatctcaggtgcagctgcagcagcctggcgccgaggtcgtgaaacctggcgcctctgtgaagatgtcctgcaaggccagcggctacaccttcaccagctactacatccactggatcaagcagacccctggacagggcctggaatgggtgggagtgatctaccccggcaacgacgacatcagctacaaccagaagttccagggcaaggccaccctgaccgccgacaagtctagcaccaccgcctacatgcagctgtccagcctgaccagcgaggacagcgccgtgtactactgcgccagagaagtgcggctgcggtacttcgatgtgtggggccagggaaccaccgtgaccgtgtcatcttccggagagagcaagtacggccctccctgccccccttgccctgcccccgagttcctgggcggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttcaatagcacctaccgggtggtgtccgtgctgaccgtgctgcaccaggactggctgaacggcaaggaatacaagtgtaaggtgtccaacaagggcctgcccagcagcatcgagaaaaccatcagcaaggccaagggccagcctcgggagccccaggtgtacaccctgccccctagccaagaggagatgaccaagaaccaggtgtccctgacctgcctggtgaagggcttctaccccagcgacatcgccgtggagtgggagagcaacggccagcccgagaacaactacaagaccaccccccctgtgctggacagcgacggcagcttcttcctgtacagccggctgaccgtggacaagagccggtggcaggagggcaacgtctttagctgctccgtgatgcacgaggccctgcacaaccactacacccagaagagcctgagcctgtccctgggcaagatgatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 39 - protein, 679 aa, Artificial Sequence
  Synthetic
MALPVTALLLPLALLLHAARPGSEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRGGGGSGGGGSSGGGSQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSSGESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - DNA, 10926 bp, Artificial Sequence
  Synthetic
tgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagaattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtggatctgcgatcgcgtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgtctagagctagcggatccccggaattcgacgccaccatggccctgcctgtgacagccctgctgctgcctctggctctgctgctgcatgccgctagacccggatccgagatcgtgctgacacagagccctggaagcctggccgtgtctcctggcgagcgcgtgacaatgagctgcaagagcagccagagcgtgttcttcagcagctcccagaagaactacctggcctggtatcagcagatccccggccagagccccagactgctgatctactgggccagcaccagagaaagcggcgtgcccgatagattcaccggcagcggctctggcaccgacttcaccctgacaatcagcagcgtgcagcccgaggacctggccatctactactgccaccagtacctgagcagccggacctttggccagggcaccaagctggaaatcaagagaggcggcggaggctctggcggaggcggatctagtggcggaggatctcaggtgcagctgcagcagcctggcgccgaggtcgtgaaacctggcgcctctgtgaagatgtcctgcaaggccagcggctacaccttcaccagctactacatccactggatcaagcagacccctggacagggcctggaatgggtgggagtgatctaccccggcaacgacgacatcagctacaaccagaagttccagggcaaggccaccctgaccgccgacaagtctagcaccaccgcctacatgcagctgtccagcctgaccagcgaggacagcgccgtgtactactgcgccagagaagtgcggctgcggtacttcgatgtgtggggccagggaaccaccgtgaccgtgtcatcttccggagagagcaagtacggccctccctgccccccttgccctgcccccgagttcctgggcggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttcaatagcacctaccgggtggtgtccgtgctgaccgtgctgcaccaggactggctgaacggcaaggaatacaagtgtaaggtgtccaacaagggcctgcccagcagcatcgagaaaaccatcagcaaggccaagggccagcctcgggagccccaggtgtacaccctgccccctagccaagaggagatgaccaagaaccaggtgtccctgacctgcctggtgaagggcttctaccccagcgacatcgccgtggagtgggagagcaacggccagcccgagaacaactacaagaccaccccccctgtgctggacagcgacggcagcttcttcctgtacagccggctgaccgtggacaagagccggtggcaggagggcaacgtctttagctgctccgtgatgcacgaggccctgcacaaccactacacccagaagagcctgagcctgtccctgggcaagatgatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgagggcagaggaagtcttctaacatgcggtgacgtggaggagaatcccggcccttccgggatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaattaattaaccaattgaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtag

SEQ ID NO: 41 - DNA, 1371 bp, Artificial Sequence
  Synthetic
atggccctgcccgtgaccgccctgctgctgcccctggccctgctgctgcacgccgccaggccccaggtgcagctggtgcagagcggcgccgaggtgaagaagcccggcagcagcgtgaaggtgagctgcaaggccagcggctacaccttcaccgactacaacatgcactgggtgaggcaggcccccggccagggcctggagtggatcggctacatctacccctacaacggcggcaccggctacaaccagaagttcaagagcaaggccaccatcaccgccgacgagagcaccaacaccgcctacatggagctgagcagcctgaggagcgaggacaccgccgtgtactactgcgccaggggcaggcccgccatggacgtgtggggccagggcaccctggtgaccgtgagcagcggcggcggcggcagcggcggcggcggcagcggcggcggcggcagcgacatccagatgacccagagccccagcagcctgagcgccagcgtgggcgacagggtgaccatcacctgcagggccagcgagagcgtggacaactacggcatcagcttcatgaactggttccagcagaagcccggcaaggcccccaagctgctgatctacgccgccagcaaccagggcagcggcgtgcccagcaggttcagcggcagcggcagcggcaccgacttcaccctgaccatcagcagcctgcagcccgacgacttcgccacctactactgccagcagagcaaggaggtgccctggaccttcggccagggcaccaaggtggagatcaagggcctggccgtgagcaccatcagcagcttcttcccccccggctaccagatctacatctgggcccccctggccggcacctgcggcgtgctgctgctgagcctggtgatcaccctgtactgcaagaggggcaggaagaagctgctgtacatcttcaagcagcccttcatgaggcccgtgcagaccacccaggaggaggacggctgcagctgcaggttccccgaggaggaggagggcggctgcgagctgagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 42 - protein, 457 aa, Artificial Sequence
  Synthetic
MALPVTALLLPLALLLHAARPQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDVWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - DNA, 10260 bp, Artificial Sequence
  Synthetic
tgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagaattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtggatctgcgatcgcgtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgtctagagctagcggatccccggaattcgacgccaccatggccctgcccgtgaccgccctgctgctgcccctggccctgctgctgcacgccgccaggccccaggtgcagctggtgcagagcggcgccgaggtgaagaagcccggcagcagcgtgaaggtgagctgcaaggccagcggctacaccttcaccgactacaacatgcactgggtgaggcaggcccccggccagggcctggagtggatcggctacatctacccctacaacggcggcaccggctacaaccagaagttcaagagcaaggccaccatcaccgccgacgagagcaccaacaccgcctacatggagctgagcagcctgaggagcgaggacaccgccgtgtactactgcgccaggggcaggcccgccatggacgtgtggggccagggcaccctggtgaccgtgagcagcggcggcggcggcagcggcggcggcggcagcggcggcggcggcagcgacatccagatgacccagagccccagcagcctgagcgccagcgtgggcgacagggtgaccatcacctgcagggccagcgagagcgtggacaactacggcatcagcttcatgaactggttccagcagaagcccggcaaggcccccaagctgctgatctacgccgccagcaaccagggcagcggcgtgcccagcaggttcagcggcagcggcagcggcaccgacttcaccctgaccatcagcagcctgcagcccgacgacttcgccacctactactgccagcagagcaaggaggtgccctggaccttcggccagggcaccaaggtggagatcaagggcctggccgtgagcaccatcagcagcttcttcccccccggctaccagatctacatctgggcccccctggccggcacctgcggcgtgctgctgctgagcctggtgatcaccctgtactgcaagaggggcaggaagaagctgctgtacatcttcaagcagcccttcatgaggcccgtgcagaccacccaggaggaggacggctgcagctgcaggttccccgaggaggaggagggcggctgcgagctgagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagggagggcagaggaagtcttctaacatgcggtgacgtggaggagaatcccggcccttccgggatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaattaattaaccaattgaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtag

SEQ ID NO: 44 - DNA, 1371 bp, Artificial Sequence
  Synthetic
atggccctgcccgtgaccgccctgctgctgcccctggccctgctgctgcacgccgccaggcccgaggtgcagctgcagcagagcggccccgagctggtgaagcccggcgccagcgtgaagatcagctgcaaggccagcggctacaccttcaccgactacaacatgcactgggtgaagcagagccacggcaagagcctggagtggatcggctacatctacccctacaacggcggcaccggctacaaccagaagttcaagagcaaggccaccctgaccgtggacaacagcagcagcaccgcctacatggacgtgaggagcctgaccagcgaggacagcgccgtgtactactgcgccaggggcaggcccgccatggactactggggccagggcaccagcgtgaccgtgagcagcggcggcggcggcagcggcggcggcggcagcggcggcggcggcagcgacatcgtgctgacccagagccccgccagcctggccgtgagcctgggccagagggccaccatcagctgcagggccagcgagagcgtggacaactacggcatcagcttcatgaactggttccagcagaagcccggccagccccccaagctgctgatctacgccgccagcaaccagggcagcggcgtgcccgccaggttcagcggcagcggcagcggcaccgacttcagcctgaacatccaccccatggaggaggacgacaccgccatgtacttctgccagcagagcaaggaggtgccctggaccttcggcggcggcaccaagctggagatcaagggcctggccgtgagcaccatcagcagcttcttcccccccggctaccagatctacatctgggcccccctggccggcacctgcggcgtgctgctgctgagcctggtgatcaccctgtactgcaagaggggcaggaagaagctgctgtacatcttcaagcagcccttcatgaggcccgtgcagaccacccaggaggaggacggctgcagctgcaggttccccgaggaggaggagggcggctgcgagctgagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 45 - protein, 457 aa, Artificial Sequence
  Synthetic
MALPVTALLLPLALLLHAARPEVQLQQSGPELVKPGASVKISCKASGYTFTDYNMHWVKQSHGKSLEWIGYIYPYNGGTGYNQKFKSKATLTVDNSSSTAYMDVRSLTSEDSAVYYCARGRPAMDYWGQGTSVTVSSGGGGSGGGGSGGGGSDIVLTQSPASLAVSLGQRATISCRASESVDNYGISFMNWFQQKPGQPPKLLIYAASNQGSGVPARFSGSGSGTDFSLNIHPMEEDDTAMYFCQQSKEVPWTFGGGTKLEIKGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - DNA, 10260 bp, Artificial Sequence
  Synthetic
tgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagaattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtggatctgcgatcgcgtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgtctagagctagcggatccccggaattcgacgccaccatggccctgcccgtgaccgccctgctgctgcccctggccctgctgctgcacgccgccaggcccgaggtgcagctgcagcagagcggccccgagctggtgaagcccggcgccagcgtgaagatcagctgcaaggccagcggctacaccttcaccgactacaacatgcactgggtgaagcagagccacggcaagagcctggagtggatcggctacatctacccctacaacggcggcaccggctacaaccagaagttcaagagcaaggccaccctgaccgtggacaacagcagcagcaccgcctacatggacgtgaggagcctgaccagcgaggacagcgccgtgtactactgcgccaggggcaggcccgccatggactactggggccagggcaccagcgtgaccgtgagcagcggcggcggcggcagcggcggcggcggcagcggcggcggcggcagcgacatcgtgctgacccagagccccgccagcctggccgtgagcctgggccagagggccaccatcagctgcagggccagcgagagcgtggacaactacggcatcagcttcatgaactggttccagcagaagcccggccagccccccaagctgctgatctacgccgccagcaaccagggcagcggcgtgcccgccaggttcagcggcagcggcagcggcaccgacttcagcctgaacatccaccccatggaggaggacgacaccgccatgtacttctgccagcagagcaaggaggtgccctggaccttcggcggcggcaccaagctggagatcaagggcctggccgtgagcaccatcagcagcttcttcccccccggctaccagatctacatctgggcccccctggccggcacctgcggcgtgctgctgctgagcctggtgatcaccctgtactgcaagaggggcaggaagaagctgctgtacatcttcaagcagcccttcatgaggcccgtgcagaccacccaggaggaggacggctgcagctgcaggttccccgaggaggaggagggcggctgcgagctgagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagggagggcagaggaagtcttctaacatgcggtgacgtggaggagaatcccggcccttccgggatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaattaattaaccaattgaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtag

SEQ ID NO: 47 - DNA, 1383 bp, Artificial Sequence
  Synthetic
atggccctgcccgtgaccgccctgctgctgcccctggccctgctgctgcacgccgccaggccccaggtgcagctgcagcagcccggcgccgaggtggtgaagcccggcgccagcgtgaagatgagctgcaaggccagcggctacaccttcaccagctactacatccactggatcaagcagacccccggccagggcctggagtgggtgggcgtgatctaccccggcaacgacgacatcagctacaaccagaagttcaagggcaaggccaccctgaccgccgacaagagcagcaccaccgcctacatgcagctgagcagcctgaccagcgaggacagcgccgtgtactactgcgccagggaggtgaggctgaggtacttcgacgtgtggggcgccggcaccaccgtgaccgtgagcagcggcggcggcggcagcggcggcggcggcagcggcggcggcggcagcaacatcatgctgacccagagccccagcagcctggccgtgagcgccggcgagaaggtgaccatgagctgcaagagcagccagagcgtgttcttcagcagcagccagaagaactacctggcctggtaccagcagatccccggccagagccccaagctgctgatctactgggccagcaccagggagagcggcgtgcccgacaggttcaccggcagcggcagcggcaccgacttcaccctgaccatcagcagcgtgcagagcgaggacctggccatctactactgccaccagtacctgagcagcaggaccttcggcggcggcaccaagctggagatcaagaggggcctggccgtgagcaccatcagcagcttcttcccccccggctaccagatctacatctgggcccccctggccggcacctgcggcgtgctgctgctgagcctggtgatcaccctgtactgcaagaggggcaggaagaagctgctgtacatcttcaagcagcccttcatgaggcccgtgcagaccacccaggaggaggacggctgcagctgcaggttccccgaggaggaggagggcggctgcgagctgagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 48 - protein, 461 aa, Artificial Sequence
  Synthetic
MALPVTALLLPLALLLHAARPQVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSSGGGGSGGGGSGGGGSNIMLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKRGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - DNA, 10272 bp, Artificial Sequence
  Synthetic
tgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagaattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtggatctgcgatcgcgtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgtctagagctagcggatccccggaattcgacgccaccatggccctgcccgtgaccgccctgctgctgcccctggccctgctgctgcacgccgccaggccccaggtgcagctgcagcagcccggcgccgaggtggtgaagcccggcgccagcgtgaagatgagctgcaaggccagcggctacaccttcaccagctactacatccactggatcaagcagacccccggccagggcctggagtgggtgggcgtgatctaccccggcaacgacgacatcagctacaaccagaagttcaagggcaaggccaccctgaccgccgacaagagcagcaccaccgcctacatgcagctgagcagcctgaccagcgaggacagcgccgtgtactactgcgccagggaggtgaggctgaggtacttcgacgtgtggggcgccggcaccaccgtgaccgtgagcagcggcggcggcggcagcggcggcggcggcagcggcggcggcggcagcaacatcatgctgacccagagccccagcagcctggccgtgagcgccggcgagaaggtgaccatgagctgcaagagcagccagagcgtgttcttcagcagcagccagaagaactacctggcctggtaccagcagatccccggccagagccccaagctgctgatctactgggccagcaccagggagagcggcgtgcccgacaggttcaccggcagcggcagcggcaccgacttcaccctgaccatcagcagcgtgcagagcgaggacctggccatctactactgccaccagtacctgagcagcaggaccttcggcggcggcaccaagctggagatcaagaggggcctggccgtgagcaccatcagcagcttcttcccccccggctaccagatctacatctgggcccccctggccggcacctgcggcgtgctgctgctgagcctggtgatcaccctgtactgcaagaggggcaggaagaagctgctgtacatcttcaagcagcccttcatgaggcccgtgcagaccacccaggaggaggacggctgcagctgcaggttccccgaggaggaggagggcggctgcgagctgagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagggagggcagaggaagtcttctaacatgcggtgacgtggaggagaatcccggcccttccgggatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaattaattaaccaattgaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtag

SEQ ID NO: 50 - DNA, 1365 bp, Artificial Sequence
  Synthetic
atggccctgcccgtgaccgccctgctgctgcccctggccctgctgctgcacgccgccaggccccaggtgcagctgcagcagagcggccccgagctggtgaggcccggcaccttcgtgaagatcagctgcaaggccagcggctacaccttcaccaactacgacatcaactgggtgaaccagaggcccggccagggcctggagtggatcggctggatctaccccggcgacggcagcaccaagtacaacgagaagttcaaggccaaggccaccctgaccgccgacaagagcagcagcaccgcctacctgcagctgaacaacctgaccagcgagaacagcgccgtgtacttctgcgccagcggctacgaggacgccatggactactggggccagggcaccagcgtgaccgtgagcagcggcggcggcggcagcggcggcggcggcagcggcggcggcggcagcgacatcaagatgacccagagccccagcagcatgtacgccagcctgggcgagagggtgatcatcaactgcaaggccagccaggacatcaacagctacctgagctggttccagcagaagcccggcaagagccccaagaccctgatctacagggccaacaggctggtggacggcgtgcccagcaggttcagcggcagcggcagcggccaggactacagcctgaccatcagcagcctggagtacgaggacatgggcatctactactgcctgcagtacgacgagttccccctgaccttcggcgccggcaccaagctggagctgaagaggggcctggccgtgagcaccatcagcagcttcttcccccccggctaccagatctacatctgggcccccctggccggcacctgcggcgtgctgctgctgagcctggtgatcaccctgtactgcaagaggggcaggaagaagctgctgtacatcttcaagcagcccttcatgaggcccgtgcagaccacccaggaggaggacggctgcagctgcaggttccccgaggaggaggagggcggctgcgagctgagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 51 - protein, 455 aa, Artificial Sequence
  Synthetic
MALPVTALLLPLALLLHAARPQVQLQQSGPELVRPGTFVKISCKASGYTFTNYDINWVNQRPGQGLEWIGWIYPGDGSTKYNEKFKAKATLTADKSSSTAYLQLNNLTSENSAVYFCASGYEDAMDYWGQGTSVTVSSGGGGSGGGGSGGGGSDIKMTQSPSSMYASLGERVIINCKASQDINSYLSWFQQKPGKSPKTLIYRANRLVDGVPSRFSGSGSGQDYSLTISSLEYEDMGIYYCLQYDEFPLTFGAGTKLELKRGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - DNA, 10254 bp, Artificial Sequence
  Synthetic
tgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagaattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtggatctgcgatcgcgtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgtctagagctagcggatccccggaattcgacgccaccatggccctgcccgtgaccgccctgctgctgcccctggccctgctgctgcacgccgccaggccccaggtgcagctgcagcagagcggccccgagctggtgaggcccggcaccttcgtgaagatcagctgcaaggccagcggctacaccttcaccaactacgacatcaactgggtgaaccagaggcccggccagggcctggagtggatcggctggatctaccccggcgacggcagcaccaagtacaacgagaagttcaaggccaaggccaccctgaccgccgacaagagcagcagcaccgcctacctgcagctgaacaacctgaccagcgagaacagcgccgtgtacttctgcgccagcggctacgaggacgccatggactactggggccagggcaccagcgtgaccgtgagcagcggcggcggcggcagcggcggcggcggcagcggcggcggcggcagcgacatcaagatgacccagagccccagcagcatgtacgccagcctgggcgagagggtgatcatcaactgcaaggccagccaggacatcaacagctacctgagctggttccagcagaagcccggcaagagccccaagaccctgatctacagggccaacaggctggtggacggcgtgcccagcaggttcagcggcagcggcagcggccaggactacagcctgaccatcagcagcctggagtacgaggacatgggcatctactactgcctgcagtacgacgagttccccctgaccttcggcgccggcaccaagctggagctgaagaggggcctggccgtgagcaccatcagcagcttcttcccccccggctaccagatctacatctgggcccccctggccggcacctgcggcgtgctgctgctgagcctggtgatcaccctgtactgcaagaggggcaggaagaagctgctgtacatcttcaagcagcccttcatgaggcccgtgcagaccacccaggaggaggacggctgcagctgcaggttccccgaggaggaggagggcggctgcgagctgagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagggagggcagaggaagtcttctaacatgcggtgacgtggaggagaatcccggcccttccgggatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaattaattaaccaattgaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtag

SEQ ID NO: 53 - DNA, 1395 bp, Artificial Sequence
  Synthetic
atggccctgcccgtgaccgccctgctgctgcccctggccctgctgctgcacgccgccaggcccgaggtgaagctgcaggagagcggccccgagctggtgaagcccggcgccagcgtgaagatgagctgcaaggccagcggctacaagttcaccgactacgtggtgcactggctgaagcagaagcccggccagggcctggagtggatcggctacatcaacccctacaacgacggcaccaagtacaacgagaagttcaagggcaaggccaccctgaccagcgacaagagcagcagcaccgcctacatggaggtgagcagcctgaccagcgaggacagcgccgtgtactactgcgccagggactacaggtacgaggtgtacggcatggactactggggccagggcaccagcgtgaccgtgagcagcggcggcggcggcagcggcggcggcggcagcggcggcggcggcagcgacatcgtgctgacccagagccccaccatcatgagcgccagccccggcgagagggtgaccatgacctgcaccgccagcagcagcgtgaactacatccactggtaccagcagaagagcggcgacagccccctgaggtggatcttcgacaccagcaaggtggccagcggcgtgcccgccaggttcagcggcagcggcagcggcaccagctacagcctgaccatcagcaccatggaggccgaggacgccgccacctactactgccagcagtggaggagctaccccctgaccttcggcgacggcaccaggctggagctgaagagggccgacgccgcccccaccgtgagcggcctggccgtgagcaccatcagcagcttcttcccccccggctaccagatctacatctgggcccccctggccggcacctgcggcgtgctgctgctgagcctggtgatcaccctgtactgcaagaggggcaggaagaagctgctgtacatcttcaagcagcccttcatgaggcccgtgcagaccacccaggaggaggacggctgcagctgcaggttccccgaggaggaggagggcggctgcgagctgagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 54 - protein, 465 aa, Artificial Sequence
  Synthetic
MALPVTALLLPLALLLHAARPEVKLQESGPELVKPGASVKMSCKASGYKFTDYVVHWLKQKPGQGLEWIGYINPYNDGTKYNEKFKGKATLTSDKSSSTAYMEVSSLTSEDSAVYYCARDYRYEVYGMDYWGQGTSVTVSSGGGGSGGGGSGGGGSDIVLTQSPTIMSASPGERVTMTCTASSSVNYIHWYQQKSGDSPLRWIFDTSKVASGVPARFSGSGSGTSYSLTISTMEAEDAATYYCQQWRSYPLTFGDGTRLELKRADAAPTVSGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - DNA, 10284 bp, Artificial Sequence
  Synthetic
tgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagaattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtggatctgcgatcgcgtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgtctagagctagcggatccccggaattcgacgccaccatggccctgcccgtgaccgccctgctgctgcccctggccctgctgctgcacgccgccaggcccgaggtgaagctgcaggagagcggccccgagctggtgaagcccggcgccagcgtgaagatgagctgcaaggccagcggctacaagttcaccgactacgtggtgcactggctgaagcagaagcccggccagggcctggagtggatcggctacatcaacccctacaacgacggcaccaagtacaacgagaagttcaagggcaaggccaccctgaccagcgacaagagcagcagcaccgcctacatggaggtgagcagcctgaccagcgaggacagcgccgtgtactactgcgccagggactacaggtacgaggtgtacggcatggactactggggccagggcaccagcgtgaccgtgagcagcggcggcggcggcagcggcggcggcggcagcggcggcggcggcagcgacatcgtgctgacccagagccccaccatcatgagcgccagccccggcgagagggtgaccatgacctgcaccgccagcagcagcgtgaactacatccactggtaccagcagaagagcggcgacagccccctgaggtggatcttcgacaccagcaaggtggccagcggcgtgcccgccaggttcagcggcagcggcagcggcaccagctacagcctgaccatcagcaccatggaggccgaggacgccgccacctactactgccagcagtggaggagctaccccctgaccttcggcgacggcaccaggctggagctgaagagggccgacgccgcccccaccgtgagcggcctggccgtgagcaccatcagcagcttcttcccccccggctaccagatctacatctgggcccccctggccggcacctgcggcgtgctgctgctgagcctggtgatcaccctgtactgcaagaggggcaggaagaagctgctgtacatcttcaagcagcccttcatgaggcccgtgcagaccacccaggaggaggacggctgcagctgcaggttccccgaggaggaggagggcggctgcgagctgagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagggagggcagaggaagtcttctaacatgcggtgacgtggaggagaatcccggcccttccgggatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaattaattaaccaattgaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtag

SEQ ID NO: 56 - DNA, 1083 bp, Artificial Sequence
  Synthetic
atgctgctgctggtgaccagcctgctgctgtgcgaactgccgcatccggcgtttctgctgattccggaggtgcagctggtggagtctgggggaggcttggtacagcctggagggtccctgagactctcctgtgcagcctctggattcaccttcagtagctatggcatgagctgggtccgccaggctccaagacaagggcttgagtgggtggccaacataaagcaagatggaagtgagaaatactatgcggactcagtgaagggccgattcaccatctccagagacaattccaagaacacgctgtatctgcaaatgaacagcctgagagccgaggacacagccacgtattactgtgcgaaagaaaatgtggactggggccagggcaccctggtcaccgtctcctcagcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggccacgacggcctgtaccagggcctgagcaccgccaccaaggacacctacgacgccctgcacatgcaggccctgccccccagg

SEQ ID NO: 57 - protein, 361 aa, Artificial Sequence
  Synthetic
MLLLVTSLLLCELPHPAFLLIPEVQLVESGGGLVQPGGSLRLSCAASGFTFSSYGMSWVRQAPRQGLEWVANIKQDGSEKYYADSVKGRFTISRDNSKNTLYLQMNSLRAEDTATYYCAKENVDWGQGTLVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - DNA, 9972 bp, Artificial Sequence
  Synthetic
tgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagaattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtggatctgcgatcgcgtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgtctagagctagcggatccccggaattcgacgccaccatgctgctgctggtgaccagcctgctgctgtgcgaactgccgcatccggcgtttctgctgattccggaggtgcagctggtggagtctgggggaggcttggtacagcctggagggtccctgagactctcctgtgcagcctctggattcaccttcagtagctatggcatgagctgggtccgccaggctccaagacaagggcttgagtgggtggccaacataaagcaagatggaagtgagaaatactatgcggactcagtgaagggccgattcaccatctccagagacaattccaagaacacgctgtatctgcaaatgaacagcctgagagccgaggacacagccacgtattactgtgcgaaagaaaatgtggactggggccagggcaccctggtcaccgtctcctcagcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggccacgacggcctgtaccagggcctgagcaccgccaccaaggacacctacgacgccctgcacatgcaggccctgccccccagggagggcagaggaagtcttctaacatgcggtgacgtggaggagaatcccggcccttccgggatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaattaattaaccaattgaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtag

SEQ ID NO: 59 - DNA, 708 bp, Artificial Sequence
  Synthetic
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaag

SEQ ID NO: 60 - protein, 356 aa, Artificial Sequence
  Synthetic
MELGLSWVVLAALLQGVQAQVKLEESGGGSVQAGESLRLSCTASGITFRDYDIDWYRQAPGKEREWLATITPSGTTHYPDSVKGRATISRDSAKNTVYLQMNSLKPEDTARYECNTLAYWGSGTQVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 120 aa, Artificial Sequence
  Synthetic
DVQLVESGGGLVQPGGSLRLSCSVSGNIDRFYAMGWYRQAPGKQRELVAQLTNNEITTYGDSVEGQFSISGDFDANTVYLQMDSLKPEDTAVYYCHAHVTTTRWSQDYYWGQGTRVTVSS

SEQ ID NO: 62 - protein, 8 aa, Artificial Sequence
  Synthetic
GRTADIYN

SEQ ID NO: 63 - protein, 8 aa, Artificial Sequence
  Synthetic
ITWIGRTP

SEQ ID NO: 64 - protein, 13 aa, Artificial Sequence
  Synthetic
NAAHYLEGNTDYY

SEQ ID NO: 65 - protein, 25 aa, Artificial Sequence
  Synthetic
QVQLVETGGGLVRAGGSLRLSCAAS

SEQ ID NO: 66 - protein, 17 aa, Artificial Sequence
  Synthetic
IGWFRQAPGKEREFVAA

SEQ ID NO: 67 - protein, 38 aa, Artificial Sequence
  Synthetic
YYADAVKGRFAFSTDSAKNTVSLQMDNLKPEDTGVYYC

SEQ ID NO: 68 - protein, 11 aa, Artificial Sequence
  Synthetic
WGQGTQVTVSS

SEQ ID NO: 69 - protein, 103 aa, Artificial Sequence
  Synthetic
QVQLVQPGGSLRLFCVASEEFFSIYAMGWYRQAPGKQHEMVARFTRDGKITYADSAKGRFTITRDAKNTLNLQMNGLIPEDTAVYYCNINHYWGQGTQVTVSS

SEQ ID NO: 70 - protein, 8 aa, Artificial Sequence
  Synthetic
EEFFSIYA

SEQ ID NO: 71 - protein, 7 aa, Artificial Sequence
  Synthetic
FTRDGKI

SEQ ID NO: 72 - protein, 8 aa, Artificial Sequence
  Synthetic
GNIDRFYA

SEQ ID NO: 73 - protein, 5 aa, Artificial Sequence
  Synthetic
NINHY

SEQ ID NO: 74 - protein, 18 aa, Artificial Sequence
  Synthetic
QVQLVQPGGSLRLFCVAS

SEQ ID NO: 75 - protein, 17 aa, Artificial Sequence
  Synthetic
MGWYRQAPGKQHEMVAR

SEQ ID NO: 76 - protein, 37 aa, Artificial Sequence
  Synthetic
TYADSAKGRFTITRDAKNTLNLQMNGLIPEDTAVYYC

SEQ ID NO: 77 - protein, 7 aa, Artificial Sequence
  Synthetic
LTNNEIT

SEQ ID NO: 78 - protein, 14 aa, Artificial Sequence
  Synthetic
HAHVTTTRWSQDYY

SEQ ID NO: 79 - protein, 25 aa, Artificial Sequence
  Synthetic
DVQLVESGGGLVQPGGSLRLSCSVS

SEQ ID NO: 80 - protein, 17 aa, Artificial Sequence
  Synthetic
MGWYRQAPGKQRELVAQ

SEQ ID NO: 81 - protein, 38 aa, Artificial Sequence
  Synthetic
TYGDSVEGQFSISGDFDANTVYLQMDSLKPEDTAVYYC

SEQ ID NO: 82 - protein, 11 aa, Artificial Sequence
  Synthetic
WGQGTRVTVSS

SEQ ID NO: 83 - protein, 120 aa, Artificial Sequence
  Synthetic
QVQLVETGGGLVRAGGSLRLSCAASGRTADIYNIGWFRQAPGKEREFVAAITWIGRTPYYADAVKGRFAFSTDSAKNTVSLQMDNLKPEDTGVYYCNAAHYLEGNTDYYWGQGTQVTVSS

SEQ ID NO: 84 - DNA, 30 bp, Artificial Sequence
  Synthetic
ggaggaaaaactgtttcatacagaaggcgt

SEQ ID NO: 85 - DNA, 31 bp, Artificial Sequence
  Synthetic
tagagggtatataatggaagctcgaattcca

SEQ ID NO: 86 - DNA, 221 bp, Artificial Sequence
  Synthetic
ggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtgatctagacttagagggtatataatggaagctcgaattcca

SEQ ID NO: 87 - DNA, 1179 bp, Artificial Sequence
  Synthetic
ggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 88 - DNA, 717 bp, Artificial Sequence
  Synthetic
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgtcctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactactttagcgacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaag

SEQ ID NO: 89 - DNA, 708 bp, Artificial Sequence
  Synthetic
atggtgagcaagggcgaggagaataacatggccatcatcaaggagttcatgcgcttcaaggtgcgcatggagggctccgtgaacggccacgagttcgagatcgagggcgagggcgagggccgcccctacgagggctttcagaccgctaagctgaaggtgaccaagggtggccccctgcccttcgcctgggacatcctgtcccctcatttcacctacggctccaaggcctacgtgaagcaccccgccgacatccccgactacttcaagctgtccttccccgagggcttcaagtgggagcgcgtgatgaactacgaggacggcggcgtggtgaccgtgacccaggactcctccctgcaggacggcgagttcatctacaaggtgaagctgcgcggcaccaacttcccctccgacggccccgtgatgcagaagaagaccatgggctgggaggcctcctccgagcggatgtaccccgaggacggtgccctgaagggcaagatcaagatgaggctgaagctgaaggacggcggccactacacctccgaggtcaagaccacctacaaggccaagaagcccgtgcagctgcccggcgcctacatcgtcgacatcaagttggacatcacctcccacaacgaggactacaccatcgtggaacagtacgaacgcgccgagggccgccactccaccggcggcatggacgagctgtacaag

SEQ ID NO: 90 - protein, 365 aa, Artificial Sequence
  Synthetic
MELGLSWVVLAALLQGVQAQVQLVETGGGLVRAGGSLRLSCAASGRTADIYNIGWFRQAPGKEREFVAAITWIGRTPYYADAVKGRFAFSTDSAKNTVSLQMDNLKPEDTGVYYCNAAHYLEGNTDYYWGQGTQVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - protein, 348 aa, Artificial Sequence
  Synthetic
MELGLSWVVLAALLQGVQAQVQLVQPGGSLRLFCVASEEFFSIYAMGWYRQAPGKQHEMVARFTRDGKITYADSAKGRFTITRDAKNTLNLQMNGLIPEDTAVYYCNINHYWGQGTQVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 92 - protein, 365 aa, Artificial Sequence
  Synthetic
MELGLSWVVLAALLQGVQADVQLVESGGGLVQPGGSLRLSCSVSGNIDRFYAMGWYRQAPGKQRELVAQLTNNEITTYGDSVEGQFSISGDFDANTVYLQMDSLKPEDTAVYYCHAHVTTTRWSQDYYWGQGTRVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 93 - protein, 10 aa, Artificial Sequence
  Synthetic
GGGGSGGGGS

SEQ ID NO: 94 - protein, 488 aa, Artificial Sequence
  Synthetic
MALPVTALLLPLALLLHAARPQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKSGAAATTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGS

SEQ ID NO: 95 - protein, 485 aa, Artificial Sequence
  Synthetic
MALPVTALLLPLALLLHAARPQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKSGAAATTTPAPRPPTPAPTIASQPLSLRPEACRPAAGGAVHTRGLDFACDIYIWAPLAGTCGVLLLSLVITLYCKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGS

SEQ ID NO: 96 - protein, 528 aa, Artificial Sequence
  Synthetic
MALPVTALLLPLALLLHAARPQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKSGAAATTTPAPRPPTPAPTIASQPLSLRPEACRPAAGGAVHTRGLDFACDIYIWAPLAGTCGVLLLSLVITLYCKWYSHSKEKIQNLSLISLANLPPSGLANAVAEGIRSEENIYTIEENVYEVEEPNEYYCYVSSRQQPSQPLGCRFAMPRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGS

SEQ ID NO: 97 - protein, 489 aa, Artificial Sequence
  Synthetic
MALPVTALLLPLALLLHAARPDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKGGGGSGGGGSGGGGSQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSSGAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGS

SEQ ID NO: 98 - protein, 112 aa, Artificial Sequence
  Synthetic
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 99 - protein, 15 aa, Artificial Sequence
  Synthetic
GGGGSGGGGSGGGGS

SEQ ID NO: 100 - protein, 116 aa, Artificial Sequence
  Synthetic
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSS

SEQ ID NO: 101 - protein, 111 aa, Artificial Sequence
  Synthetic
DIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIK

(19) United States
(12) Patent Application Publication
(10) Pub. No.: US 2023/0372484 A1
(43) Pub. Date: Nov. 23, 2023

(54) CHIMERIC ANTIGEN RECEPTORS FOR TREATMENT OF CANCER

(71) Applicant: VOR BIOPHARMA INC., Cambridge, MA (US)

(72) Inventors: Sadik Kassim, Cambridge, MA (US); Julian Scherer, Cambridge, MA (US)

(73) Assignee: VOR BIOPHARMA INC., Cambridge, MA (US)

(21) Appl. No.: 18/026,092

(22) PCT Filed: Sep. 14, 2021
(86) PCT No.: PCT/US2021/050331

§ 371 Date: Mar. 13, 2023

Related U.S. Application Data
(60) Provisional application No. 63/078,237, filed on Sep. 14, 2020.

(51) Int. Cl. A61K 39/00; C07K 16/28; A61P 35/00

(52) U.S. Cl. CPC A61K 39/4631; C07K 16/2896; A61P 35/00; C07K 2319/03; C07K 2319/02; C07K 2317/53

(57) ABSTRACT

Provided are chimeric antigen receptors (CARs) with binding specificity for CD33. Nucleic acids, vectors, host cells, populations of cells expressing the CARs, and pharmaceutical compositions relating to the CARs are also disclosed, and methods including the treatment of CD33-related diseases, in particular, leukemias such as acute myeloid leukemia (AML).

### RELATED APPLICATIONS

This application claims the benefit under 35 U.S.C. 119(e) of U.S. provisional application No. 63/078,237, filed Sep. 14, 2020, which is incorporated by reference herein in its entirety.

### BACKGROUND

Acute myelogenous leukemia is a highly aggressive acute leukemia, representing the second most common leukemia occurring in children and adolescents and young adults (AYAs). Despite current treatment regimens, which include intensive cycles of multi-agent chemotherapy, and frequently consolidation with allogeneic donor stem cell transplantation to achieve cure, only 60% of children and AYAs with AML will be achieve long-term remission. New therapeutic strategies are needed to increase remission rates, decrease relapse and to improve overall survival.

### SUMMARY

Aspects of the present disclosure provide chimeric antigen receptors (CARs) comprising an antigen binding domain specific for CD33, a transmembrane domain, and an intracellular T cell signaling domain. In some embodiments, the CARs comprise one or more additional domains, such as a linker region, a hinge region, and one or more costimulatory signaling domain. Aspects of the present disclosure provide CAR constructs comprising any of the amino acid sequences as described herein.

Further aspects of the disclosure provide related nucleic acids, recombinant expression vectors, host cells, populations of cells, and pharmaceutical compositions relating to the CAR constructs of the present disclosure or cells expressing such CAR constructs.

Additional embodiments of the invention provide methods of treating a hematopoietic malignancy (e.g., acute myeloid leukemia (AML), myelodysplastic syndrome (MDS)) in a subject by administering to the subject a population of immune cells comprising a CAR specific for CD33 alone or in combination with a population of hematopoietic cells, wherein the hematopoietic cells are genetically-engineered such that the gene encoding CD33 engineered to reduce or eliminate the expression of CD33.

Aspects of the present disclosure provide isolated nucleic acids molecule encoding a chimeric antigen receptor (CAR). In some embodiments, the CAR comprises a CD33 binding domain, a transmembrane domain, and an intracellular signaling domain. In some embodiments, the encoded CD33 binding domain comprises a heavy chain variable region and/or a light chain variable region. In some embodiments, the encoded transmembrane domain comprises a transmembrane domain of a protein selected from CD8a or CD28. In some embodiments, the encoded intracellular signaling domain comprises a functional signaling domain of CD3ζ. In some embodiments, the CARs comprise one or more additional domains, such as a linker region, a hinge region, and one or more costimulatory signaling domain.

In some embodiments, the heavy chain variable region and the light chain variable region are joined by a linker. In some embodiments, the encoded CD33 binding domain comprises a single-chain variable fragment (scFv), an Fab, an F(ab′)2, a dsFv, a diabody, Nanobody® (single domain antibody, also referred to as VHH), or a tiabody.

In some embodiments, the encoded CD33 binding domain is connected to the transmembrane domain by a hinge region. In some embodiments, the encoded hinge region comprises a hinge region of a protein selected from CD8a, IgG4, or CD28.

In some embodiments, the encoded CAR further comprises one or more co-stimulatory domains. In some embodiments, the one more co-stimulatory domains comprises a functional signaling domain of 4-1BB and/or CD28.

In some embodiments, the isolated nucleic acid sequence further comprises a promoter sequence. In some embodiments, the promoter sequence is a SFFV (silencing-prone spleen focus forming virus) promoter sequence or an EF1α promoter sequence.

In some embodiments, the encoded CAR comprises (i) an amino acid sequence of any one of SEQ ID NOs: 10, 13, 16, 19, 22, 25, 29, 33, 36, 39, 42, 45, 48, 51, 54, 57, and 60-92; or (ii) an amino acid sequence having 95-99% identity to any one of SEQ ID NOs: 10, 13, 16, 19, 22, 25, 29, 33, 36, 39, 42, 45, 48, 51, 54, 57, and 60-92. In some embodiments, the nucleic acid molecule comprises (i) a nucleotide sequence selected from any one of SEQ ID NOs: 9, 12, 15, 18, 21, 24, 28, 32, 35, 38, 41, 44, 47, 50, 53, and 56; or (ii) a nucleotide sequence with 95-99% identity to any one of SEQ ID NOs: 9, 12, 15, 18, 21, 24, 28, 32, 35, 38, 41, 44, 47, 50, 53, and 56.

In some embodiments, an expression vector comprising any of the nucleic acid molecules encoding any of the CARs as described herein. In some embodiments, the vector is a DNA vector, an RNA vector, a plasmid, a lentivirus vector, an adenoviral vector, or a retrovirus vector. In some embodiments, the expression vector comprises (i) a nucleotide sequence selected from any one of SEQ ID NOs: 11, 14, 17, 20, 23, 26, 30, 34, 37, 40, 43, 46, 49, 52, 55, and 58; or (ii) a nucleotide sequence with 95-99% identity to any one of SEQ ID NOs: 11, 14, 17, 20, 23, 26, 30, 34, 37, 40, 43, 46, 49, 52, 55, and 58.

In another aspect, the disclosure provides immune effector cells comprising any of the nucleic acid molecules as described herein. In some aspects, the disclosure provides immune effectors comprising any of the CARs as described herein. In some embodiments, the cell is selected from the group consisting of a T cell, a Natural Killer (NK) cell, a cytotoxic T lymphocyte (CTL), a regulatory T cell, a human embryonic stem cell, and a pluripotent stem cell from which lymphoid cells may be differentiated. The present disclosure also provides populations of cells comprising at least one immune effector cell comprising any of the nucleic acid molecules as described herein. The present disclosure also provides populations of cells comprising at least one immune effector cell comprising any of the CARs as described herein.

A further aspect of the disclosure provides pharmaceutical composition comprising any of the populations of immune effector cells comprising the nucleic acids and/or any of the CARs described herein and a pharmaceutically acceptable carrier.

In another aspect, the disclosure features a method of treating a hematopoietic malignancy. In some embodiments, the method comprises administering to a subject in need thereof an effective amount of an agent targeting CD33. In some embodiments, the agent is an immune cell expressing a chimeric receptor (CAR). In some embodiments, the CAR comprises: an antigen-binding domain that binds CD33 comprising a heavy chain variable region and/or a light chain variable region; a transmembrane domain comprising a transmembrane domain of a protein selected from CD8a or CD28; and an intracellular signaling domain comprising a functional signaling domain of CD3. In some embodiments, the CARs comprise one or more additional domains, such as a linker region, a hinge region, and one or more costimulatory signaling domain.

In some embodiments, the method further comprises administering a population of hematopoietic cells, wherein the hematopoietic cells are genetically-engineered such that the gene encoding CD33 that is targeted by the antigen-binding domain is engineered to reduce or eliminate the expression of CD33. In some embodiments, the immune cells, the hematopoietic cells, or both, are allogeneic or autologous.

In some embodiments, the hematopoietic cells are hematopoietic stem cells. In some embodiments, the hematopoietic cells are hematopoietic progenitor cells. In some embodiments, the hematopoietic cells are hematopoietic stem and progenitor cells. In some embodiments, the hematopoietic stem cells are from bone marrow cells or peripheral blood mononuclear cells (PBMCs). In some embodiments, the hematopoietic stem cells are CD34+/CD33−.

In some embodiments, the hematopoietic cells are prepared by editing the endogenous gene coding for CD33 to reduce or eliminate the expression of CD33. In some embodiments, the endogenous gene is edited using a CRISPR system (e.g., by an RNA-guided nuclease, e.g., CRISPR-Cas9, CRISPR-Cas12a).

In some embodiments, the subject has or has been diagnosed with a hematopoietic malignancy or pre-malignancy characterized by the expression of CD33 on malignant cells or pre-malignant cells. In some embodiments, the subject has Hodgkin's lymphoma, non-Hodgkin's lymphoma, myelodysplastic syndrome, leukemia, or multiple myeloma. In some embodiments, the leukemia is acute myeloid leukemia (AML), chronic myelogenous leukemia, acute lymphoblastic leukemia, chronic lymphoblastic leukemia, or myelodysplastic syndrome (MDS).

In some embodiments, the immune cells comprise one or more cell types selected from the group consisting of a T cell, a Natural Killer (NK) cell, a cytotoxic T lymphocyte (CTL), a regulatory T cell, a human embryonic stem cell, and a pluripotent stem cell from which lymphoid cells may be differentiated.

In some embodiments, the antigen-binding domain of the CAR is a single-chain variable fragment (scFv), an Fab, an F(ab′)2, a dsFv, a diabody, a Nanobody® (single domain antibody), or a triabody that specifically binds CD33. In some embodiments, the heavy chain variable region and the light chain variable region of the antigen-binding domain are joined by a linker. In some embodiments, the antigen-binding domain is connected to the transmembrane domain by a hinge region. In some embodiments, the hinge region comprises a hinge region of a protein selected from CD8a, IgG4, or CD28. In some embodiments, the CAR further comprises one or more co-stimulatory domains. In some embodiments, the one more co-stimulatory domains comprises a functional signaling domain of 4-1BB and/or CD28.

In some embodiments, the encoded CAR comprises (i) an amino acid sequence of any one of SEQ ID NOs: 10, 13, 16, 19, 22, 25, 29, 33, 36, 39, 42, 45, 48, 51, 54, 57, and 60-92; or (ii) an amino acid sequence having 95-99% identity to any one of SEQ ID NOs: 10, 13, 16, 19, 22, 25, 29, 33, 36, 39, 42, 45, 48, 51, 54, 57, and 60-92. In some embodiments, the CAR is encoded by a nucleotide sequence that is (i) selected from any one of SEQ ID NOs: 9, 12, 15, 18, 21, 24, 28, 32, 35, 38, 41, 44, 47, 50, 53, and 56; or (ii) 95-99% identical to any one of SEQ ID NOs: 9, 12, 15, 18, 21, 24, 28, 32, 35, 38, 41, 44, 47, 50, 53, and 56.

### Definitions

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

The articles “a” and “an” are used herein to refer to one or to more than one (i.e., to at least one) of the grammatical object of the article. By way of example, “an element” means one element or more than one element.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Agent: As used herein, the term “agent” (or “biological agent” or “therapeutic agent”), refers to a molecule that may be expressed, released, secreted or delivered to a target by a modified cell (e.g., an immune cell comprising a chimeric antigen receptor) described herein. An agent includes, but is not limited to, a nucleic acid, an antibiotic, an anti-inflammatory agent, an antibody or fragments thereof, a chimeric antigen receptor, an antibody agent or fragments thereof, a growth factor, a cytokine, an enzyme, a protein (e.g., an RNAse inhibitor), a peptide, a fusion protein, a synthetic molecule, an organic molecule (e.g., a small molecule), a carbohydrate, a lipid, a hormone, a microsome, a derivative or a variation thereof, and any combinations thereof. An agent may bind any cell moiety, such as a receptor, an antigenic determinant, or other binding site present on a target or target cell. An agent may diffuse or be transported into a cell, where it may act intracellularly.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprising two identical heavy chain polypeptides (about 50 kD each) and two identical light chain polypeptides (about 25 kD each) that associate with each other into what is commonly referred to as a “Y-shaped” structure. Each heavy chain comprises at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain comprises two domains—an amino-terminal variable (VL) domain, followed by a carboxy-terminal constant (CL) domain, separated from one another by another “switch”. Intact antibody tetramers comprise two heavy chain-light chain dimers in which the heavy and light chains are linked to one another by a single disulfide bond; two other disulfide bonds connect the heavy chain hinge regions to one another, so that the dimers are connected to one another and a tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complementarity determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including, for example, effector cells that mediate cytotoxicity. Affinity and/or other binding attributes of Fc regions for Fc receptors can be modulated through glycosylation or other modification. In some embodiments, antibodies produced and/or utilized in accordance with the present invention (e.g., as a component of a CAR) include glycosylated Fc domains, including Fc domains with modified or engineered glycosylation. In some embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal. In some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc, as is known in the art. Moreover, the term “antibody”, as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present invention is in a format selected from, but not limited to, intact IgA, IgG, IgE or IgM antibodies; bi- or multi-specific antibodies (e.g., Zybodies®, etc); antibody fragments such as is used herein in the broadest sense and encompasses various antibody structures, including but not limited to monoclonal antibodies, polyclonal antibodies, multispecific antibodies (e.g., bispecific antibodies), and/or antibody fragments (preferably those fragments that exhibit the desired antigen-binding activity). An antibody described herein can be an immunoglobulin, heavy chain antibody, light chain antibody, LRR-based antibody, or other protein scaffold with antibody-like properties, as well as other immunological binding moiety known in the art, including, e.g., a Fab, Fab′, Fab′2, Fab2, Fab3, F(ab′)2, Fd, Fv, Feb, scFv, SMIP, antibody, diabody, triabody, tetrabody, minibody, nanobody (single domain antibody, VHH), maxibody, tandab, DVD, BiTe, TandAb, or the like, or any combination thereof. The subunit structures and three-dimensional configurations of different classes of antibodies are known in the art. In some embodiments, an antibody may lack a covalent modification (e.g., attachment of a glycan) that it would have if produced naturally. In some embodiments, an antibody may contain a covalent modification (e.g., attachment of a glycan, a payload (e.g., a detectable moiety, a therapeutic moiety, a catalytic moiety, etc), or other pendant group (e.g., poly-ethylene glycol, etc.).

Antigen-binding fragment: An “antigen-binding fragment” refers to a portion of an intact antibody that binds the antigen to which the intact antibody binds. An antigen-binding fragment of an antibody includes any naturally occurring, enzymatically obtainable, synthetic, or genetically engineered polypeptide or glycoprotein that specifically binds an antigen to form a complex. Exemplary antibody fragments include, but are not limited to, Fv, Fab, Fab′, Fab′-SH, F(ab′)2; diabodies; linear antibodies; single-chain antibody molecules (e.g. scFv or VHH or VH or VL domains only); and multispecific antibodies formed from antibody fragments. In some embodiments, the antigen-binding fragments of the antibodies described herein are scFvs. In some embodiments, the antigen-binding fragments of the antibodies described herein are VHH domains only. As with full antibody molecules, antigen-binding fragments may be mono-specific or multispecific (e.g., bispecific). A multispecific antigen-binding fragment of an antibody may comprise at least two different variable domains, wherein each variable domain is capable of specifically binding to a separate antigen or to a different epitope of the same antigen.

Antibody heavy chain: As used herein, the term “antibody heavy chain” refers to the larger of the two types of polypeptide chains present in all antibody molecules in their naturally occurring conformations.

Antibody light chain: As used herein, the term “antibody light chain” refers to the smaller of the two types of polypeptide chains present in all antibody molecules in their naturally occurring conformations.

Synthetic antibody: As used herein, the term “synthetic antibody” refers to an antibody that is generated using recombinant DNA technology, such as, for example, an antibody expressed by a bacteriophage or yeast as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

Antigen: As used herein, the term “antigen” or “Ag” refers to a molecule that is capable of provoking an immune response. This immune response may involve either antibody production, the activation of specific immunologically-competent cells, or both. A skilled artisan will understand that any macromolecule, including virtually all proteins or peptides, can serve as an antigen. Furthermore, antigens can be derived from recombinant or genomic DNA. A skilled artisan will understand that any DNA that comprises a nucleotide sequences or a partial nucleotide sequence encoding a protein that elicits an immune response encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

Autologous: As used herein, the term “autologous” refers to any material (e.g., a population of cells) derived from an individual to which it is later to be re-introduced into the same individual.

Allogeneic: As used herein, the term “allogeneic” refers to any material (e.g., a population of cells) derived from a different animal of the same species.

Xenogeneic: As used herein, the term “xenogeneic” refers to any material (e.g., a population of cells) derived from an animal of a different species.

Cancer: As used herein, the term “cancer” refers to a disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In certain embodiments, the cancer is medullary thyroid carcinoma.

Conservative sequence modifications: As used herein, the term “conservative sequence modifications” refers to amino acid modifications that do not significantly affect or alter the binding characteristics of an antibody containing the amino acid sequence. Such conservative modifications include amino acid substitutions, additions and deletions. Modifications can be introduced into an antibody compatible with various embodiments by standard techniques known in the art, such as site-directed mutagenesis and PCR-mediated mutagenesis. Conservative amino acid substitutions are ones in which an amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

Co-stimulatory ligand. As used herein, the term “co-stimulatory ligand” refers to a molecule on an antigen presenting cell (e.g., an APC, dendritic cell, B cell, and the like) that specifically binds a cognate co-stimulatory molecule on an immune cell (e.g., a T lymphocyte), thereby providing a signal which mediates an immune cell response, including, but not limited to, proliferation, activation, differentiation, and the like. A co-stimulatory ligand can include, but is not limited to, CD7, B7-1 (CD80), B7-2 (CD86), CD28, PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on an immune cell (e.g., a T lymphocyte), such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

Cytotoxic: As used herein, the term “cytotoxic” or “cytotoxicity” refers to killing or damaging cells. In one embodiment, cytotoxicity of the metabolically enhanced cells is improved, e.g. increased cytolytic activity of immune cells (e.g., T lymphocytes). In some embodiments, cytotoxicity of the cells described herein (i.e., cells expressing the CARs described herein) is improved, e.g. increased cytolytic activity of immune cells (e.g., T lymphocytes). In some embodiments, cytotoxicity of the cells described herein (i.e., cells expressing the CARs described herein) for a target cell expressing an CD33) is improved, e.g increased cytolytic activity of immune cells (e.g., T lymphocytes).

Effective amount: As used herein, an “effective amount” as described herein refers to a dose that is adequate to prevent or treat cancer in an individual. Amounts effective for a therapeutic or prophylactic use will depend on, for example, the stage and severity of the disease or disorder being treated, the age, weight, and general state of health of the patient, and the judgment of the prescribing physician. The size of the dose will also be determined by the active selected, method of administration, timing and frequency of administration, the existence, nature, and extent of any adverse side-effects that might accompany the administration of a particular active, and the desired physiological effect. It will be appreciated by one of skill in the art that various diseases or disorders could require prolonged treatment involving multiple administrations, for example using the CARs described herein in each or various rounds of administration. By way of example and not intending to limit the invention, when the CARs described herein are provided in a host cell expressing the CAR, an exemplary dose of host cells may be a minimum of one million cells (1×10⁶ cells/dose).

For purposes of the invention, the amount or dose of an agent comprising an immune cell containing a CAR construct described herein administered should be sufficient to effect a therapeutic or prophylactic response in the subject or animal over a reasonable time frame. For example, the dose should be sufficient to bind to antigen, or detect, treat or prevent cancer, a hematopoietic malignancy or premalignancy, in a period of from about 2 hours or longer, e.g., about 12 to about 24 or more hours, from the time of administration. In certain embodiments, the time period could be even longer. The dose will be determined by the efficacy of the particular CARs described herein and the condition of the animal (e.g., human), as well as the body weight of the animal (e.g., human) to be treated.

Effector function: As used herein, “effector function” or “effector activity” refers to a specific activity carried out by an immune cell in response to stimulation of the immune cell. For example, an effector function of a T lymphocyte includes, recognizing an antigen and killing a cell that expresses the antigen.

Encoding: As used herein, “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Endogenous: As used herein “endogenous” refers to any material from or produced inside a particular organism, cell, tissue or system.

Exogenous: As used herein, the term “exogenous” refers to any material introduced from or produced outside a particular organism, cell, tissue or system.

Expand: As used herein, the term “expand” refers to increasing in number, as in an increase in the number of cells, for example, immune cells, e.g., T lymphocytes, and/or hematopoietic cells. In one embodiment, immune cells, e.g., T lymphocytes, NK cells, and/or hematopoietic cells that are expanded ex vivo increase in number relative to the number originally present in a culture. In some embodiments, immune cells, e.g., T lymphocytes, NK cells, and/or hematopoietic cells that are expanded ex vivo increase in number relative to other cell types in a culture. In some embodiments, expansion may occur in vivo. The term “ex vivo,” as used herein, refers to cells that have been removed from a living organism, (e.g., a human) and propagated outside the organism (e.g., in a culture dish, test tube, or bioreactor).

Expression: As used herein, the term “expression” of a nucleic acid sequence refers to generation of any gene product from a nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Expression vector: As used herein, the term “expression vector” or “recombinant expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses).

Fragment. As used herein, the terms “fragment” or “portion” refers to a structure that includes a discrete portion of the whole, but lacks one or more moieties found in the whole structure. In some embodiments, a fragment consists of such a discrete portion. In some embodiments, a fragment consists of or comprises a characteristic structural element or moiety found in the whole. In some embodiments, a nucleotide fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500, or more monomeric units (e.g., nucleic acids) as found in the whole nucleotide. In some embodiments, a nucleotide fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99%, or more of the monomeric units (e.g., residues) found in the whole nucleotide. The whole material or entity may in some embodiments be referred to as the “parent” of the whole.

Functional Portion: As used herein, the term “functional portion” when used in reference to a CAR refers to any part or fragment of the CAR constructs of the invention, which part or fragment retains the biological activity of the CAR construct of which it is a part (the parent CAR construct). Functional portions encompass, for example, those parts of a CAR construct that retain the ability to recognize target cells, or detect, treat, or prevent cancer, such as a hematopoietic malignancy or pre-malignancy, to a similar extent, the same extent, or to a higher extent, as the parent CAR construct. In reference to the parent CAR construct, the functional portion can comprise, for instance, about 10%, about 25%, about 30%, about 50%, about 68%, about 80%, about 90%, about 95%, or more, of the parent CAR.

The functional portion can comprise additional amino acids at the amino or carboxy terminus of the portion, or at both termini, which additional amino acids are not found in the amino acid sequence of the parent CAR construct. Desirably, the additional amino acids do not interfere with the biological function of the functional portion, e.g., recognize target cells, detect cancer, treat or prevent a cancer, such as hematopoietic malignancy or pre-malignancy, etc. More desirably, the additional amino acids enhance the biological activity as compared to the biological activity of the parent CAR construct.

Functional Variant: As used herein, the term “functional variant,” as used herein, refers to a CAR construct, polypeptide, or protein having substantial or significant sequence identity or similarity to a parent CAR construct, which functional variant retains the biological activity of the CAR of which it is a variant. Functional variants encompass, for example, those variants of the CAR construct described herein (the parent CAR construct) that retain the ability to recognize target cells to a similar extent, the same extent, or to a. higher extent, as the parent CAR construct. In reference to the parent CAR construct, the functional variant can, for instance, be at least about 30%, about 50%, about 75%, about 80%, about 90%, about 91%, about 92%, about 93%, about 94%, about 95%, about 96%, about 97%, about 98%, about 99% or more identical in amino acid sequence to the parent CAR construct.

A functional variant can, for example, comprise the amino acid sequence of the parent CAR with at least one conservative amino acid substitution. Alternatively or additionally, the functional variants can comprise the amino acid sequence of the parent CAR construct with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent CAR construct.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). As will be understood by those skilled in the art, a variety of algorithms are available that permit comparison of sequences in order to determine their degree of homology, including by permitting gaps of designated length in one sequence relative to another when considering which residues “correspond” to one another in different sequences. Calculation of the percent homology between two nucleic acid sequences, for example, can be performed by aligning the two sequences for optimal comparison purposes (e.g., gaps can be introduced in one or both of a first and a second nucleic acid sequences for optimal alignment and non-corresponding sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position; when a position in the first sequence is occupied by a similar nucleotide as the corresponding position in the second sequence, then the molecules are similar at that position. The percent homology between the two sequences is a function of the number of identical and similar positions shared by the sequences, taking into account the number of gaps, and the length of each gap, which needs to be introduced for optimal alignment of the two sequences. As will be evident to one of ordinary skill in the art, the percent homology may be assessed across the full length of the amino acid or nucleic acid sequences, or a portion thereof (e.g., one or more domains or regions).

Identity: As used herein, the term “identity” refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical. As will be evident to one of ordinary skill in the art, the percent identity may be assessed across the full length of the amino acid or nucleic acid sequences, or a portion thereof (e.g., one or more domains or regions).

Substantial identity: As used herein, the term “substantial identity” refers to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues. In the context of a CDR, reference to “substantial identity” typically refers to a CDR having an amino acid sequence at least 80%, preferably at least 85%, at least 90%, at least 95%, at least 98% or at least 99% identical to that of a reference CDR.

Immune cell: As used herein, the term “immune cell,” refers to a cell that is involved in an immune response, e.g., promotion of an immune response. Examples of immune cells include, but are not limited to, T-lymphocytes, natural killer (NK) cells, macrophages, monocytes, dendritic cells, neutrophils, eosinophils, mast cells, platelets, large granular lymphocytes, Langerhans' cells, or B-lymphocytes. A source of immune cells (e.g., T lymphocytes) can be obtained from a subject, such as a healthy donor subject or a subject that has been diagnosed with a hematopoietic malignancy or pre-malignancy.

Immune response: As used herein the term “immune response” refers to a cellular and/or systemic response to an antigen that occurs when lymphocytes identify antigenic molecules as foreign and induce the formation of antibodies and/or activate lymphocytes to remove the antigen.

Immunoglobulin: As used herein, the term “immunoglobulin” or “Ig,” refers to a class of proteins that function as antibodies. Antibodies expressed by B cells are sometimes referred to as a BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is an immunoglobulin that has no known antibody function, but may serve as an antigen receptor. IgE is an immunoglobulin that mediates immediate hypersensitivity by causing release of mediators from mast cells and basophils upon exposure to allergen.

Isolated: As used herein, the term “isolated” refers to something altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Modified: As used herein, the term “modified” refers to a changed state or structure of a molecule or cell of the invention. Molecules may be modified in many ways, including chemically, structurally, and functionally. Cells may be modified through the introduction of nucleic acids.

Modulating: As used herein the term “modulating,” refers to mediating a detectable increase or decrease in the level of a response and/or a change in the nature of a response in a subject compared with the level and/or nature of a response in the subject in the absence of a treatment or compound, and/or compared with the level and/or nature of a response in an otherwise identical but untreated subject. The term encompasses perturbing and/or affecting a native signal or response thereby mediating a beneficial therapeutic response in a subject, preferably, a human.

Monoclonal Antibody: A “monoclonal antibody” or “mAb” refers to an antibody obtained from a population of substantially homogeneous antibodies, i.e., the individual antibodies comprising the population are identical and/or bind the same epitope, except for possible variant antibodies (e.g., containing naturally occurring mutations or arising during production of a monoclonal antibody preparation), such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

Nucleic acid: As used herein, the term “nucleic acid” refers to a polymer of at least three nucleotides. In some embodiments, a nucleic acid comprises DNA. In some embodiments, a nucleic acid comprises RNA. In some embodiments, a nucleic acid is single stranded. In some embodiments, a nucleic acid is double stranded. In some embodiments, a nucleic acid comprises both single and double stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises one or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long.

Operably linked: As used herein, the term “operably linked” refers to functional linkage between, for example, a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

Polynucleotide: As used herein, the term “polynucleotide” refers to a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and polymerase chain reaction (PCR) methods, and the like, and by synthetic means.

Polypeptide: As used herein, the term “polypeptide” refers to any polymeric chain of residues (e.g., amino acids) that are typically linked by peptide bonds. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Signal transduction pathway: As used herein, the term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell.

Single chain antibodies: As used herein, the term “single chain antibodies” refers to antibodies formed by recombinant DNA techniques in which immunoglobulin heavy and light chain fragments are linked to the Fv region via an engineered span of amino acids. Various methods of generating single chain antibodies are known, including those described in U.S. Pat. No. 4,694,778; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041.

Specifically binds: As used herein, the term “specifically binds,” with respect to an antigen binding domain, such as an antibody agent, refers to an antigen binding domain or antibody agent which recognizes a specific antigen, but does not substantially recognize or bind other molecules in a sample. For example, an antigen binding domain or antibody agent that specifically binds to an antigen from one species may also bind to that antigen from one or more species. But, such cross-species reactivity does not itself alter the classification of an antigen binding domain or antibody agent as specific. In another example, an antigen binding domain or antibody agent that specifically binds to an antigen may also bind to different allelic forms of the antigen. However, such cross reactivity does not itself alter the classification of an antigen-binding domain or antibody agent as specific. In some instances, the terms “specific binding” or “specifically binding,” can be used in reference to the interaction of an antigen binding domain or antibody agent, a protein, or a peptide with a second chemical species, to mean that the interaction is dependent upon the presence of a particular structure (e.g., an antigenic determinant or epitope) on the chemical species; for example, an antigen binding domain or antibody agent recognizes and binds to a specific protein structure rather than to proteins generally. If an antigen binding domain or antibody agent is specific for epitope “A”, the presence of a molecule containing epitope A (or free, unlabeled A), in a reaction containing labeled “A” and the antigen binding domain or antibody agent, will reduce the amount of labeled A bound to the antibody. “Specifically binds,” with respect to ligand such as CD33-binding fragment thereof, and its respective receptor (e.g., a specific target antigen), refers to an antigen binding domain that does not substantially recognize or bind other molecules in a sample, such as other antigens.

Subject: As used herein, the term “subject” refers to an organism, for example, a mammal (e.g., a human, a non-human mammal, a non-human primate, a primate, a laboratory animal, a mouse, a rat, a hamster, a gerbil, a cat, or a dog). In some embodiments a human subject is an adult, adolescent, or pediatric subject. In some embodiments, a subject is suffering from a disease, disorder or condition, e.g., a disease, disorder, or condition that can be treated as provided herein, e.g., a cancer, such ss a hematopoietic malignancy or pre-malignancy. In some embodiments, a subject is susceptible to a disease, disorder, or condition; in some embodiments, a susceptible subject is predisposed to and/or shows an increased risk (as compared to the average risk observed in a reference subject or population) of developing the disease, disorder, or condition. In some embodiments, a subject displays one or more symptoms of a disease, disorder, or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered. In some embodiments, the subject has been diagnosed with the disease, disorder, or condition.

Substantially purified: As used herein, the term “substantially purified,” for example as applied to a cell, refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

Target. As used herein, the term “target” refers to a cell, tissue, organ, or site within the body that is the subject of provided methods, systems, and/or compositions, for example, a cell, tissue, organ or site within a body that is in need of treatment or is preferentially bound by, for example, an antibody (or fragment thereof) or a CAR.

Target site: As used herein, the term “target site” or “target sequence” refers to a genomic nucleic acid sequence that defines a portion of a nucleic acid to which a binding molecule (e.g., an antigen-binding domain of a CAR, e.g., a CD33 binding fragment of any of the CARs described herein) may specifically bind under conditions sufficient for binding to occur.

T cell receptor: As used herein, the term “T cell receptor” or “TCR” refers to a complex of membrane proteins that participate in the activation of T cells in response to the presentation of antigen. A TCR is responsible for recognizing antigens bound to major histocompatibility complex molecules. A TCR comprises a heterodimer of an alpha (a) and beta (β) chain, although in some cells the TCR comprises gamma and delta (γ/δ) chains. TCRs may exist in alpha/beta and gamma/delta forms, which are structurally similar but have distinct anatomical locations and functions. Each chain comprises two extracellular domains, a variable and constant domain. In some embodiments, a TCR may be modified on any cell comprising a TCR, including, for example, a helper T cell, a cytotoxic T cell, a memory T cell, regulatory T cell, natural killer T cell, and gamma delta T cell.

Therapeutic: As used herein, the term “therapeutic” refers to a treatment. A therapeutic effect is obtained by suppression, remission, or eradication of a disease state. A therapeutic effect may be obtained by prevention (prophylaxis).

Transfected. As used herein, the term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

Treat: As used herein, the term “treat,” “treatment,” or “treating” refers to partial or complete alleviation, amelioration, delay of onset of, inhibition, relief, and/or reduction in incidence and/or severity of one or more symptoms or features of a disease, disorder, and/or condition. In some embodiments, treatment may be administered to a subject who does not exhibit signs or features of a disease, disorder, and/or condition (e.g., may be prophylactic). In some embodiments, treatment may be administered to a subject who exhibits only early or mild signs or features of the disease, disorder, and/or condition, for example for the purpose of decreasing the risk of developing pathology associated with the disease, disorder, and/or condition. In some embodiments, treatment may be administered to a subject who exhibits established, severe, and/or late-stage signs of the disease, disorder, or condition. In some embodiments, treating may comprise administering to an immune cell (e.g., a T lymphocyte, NK cell) or contacting an immune cell with a modulator of a pathway activated by in vitro transcribed mRNA. In some embodiments, the methods described herein are for prevention of a disease, disorder, and/or condition or one or more symptoms or features of a disease, disorder, and/or condition.

Tumor: As used herein, the term “tumor” refers to an abnormal growth of cells or tissue. In some embodiments, a tumor may comprise cells that are precancerous (e.g., benign), malignant, pre-metastatic, metastatic, and/or non-metastatic. In some embodiments, a tumor is associated with, or is a manifestation of, a cancer. In some embodiments, a tumor may be a disperse tumor or a liquid tumor. In some embodiments, a tumor may be a solid tumor.

Vector: As used herein, the term “vector” refers to a composition of matter that comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### BRIEF DESCRIPTION OF THE DRAWINGS

FIGS. 1A-1C show flow cytometry analysis plots of exemplary reporter cells as described herein. FIG. 1A shows Jurkat cells containing the mOrange reporter molecule under control of the constitutively active ElFalpha promoter and mTurquoise reporter molecule (mTurq) under control of an IL-2 reporter system described herein. Cells were either not activated (“−PMA/Ion,” top row) or activated using phorbol myristate acetate (PMA) and ionomycin (“+PMA/Ion,” bottom row). The left column of plots show cells expressing the mOrange reporter molecule; the middle column shows cells expressing the mTurquoise reporter molecule; and the right column shows cells expressing CD69, an indicator of T cell activation. FIG. 1B show Jurkat cells containing the mTurquoise reporter molecule (mTurq) under control of the constitutively active ElFalpha promoter and mOrange reporter molecule under control of an IL-2 reporter system described herein. Cells were either not activated (“−PMA/Ion,” top row) or activated using phorbol myristate acetate (PMA) and ionomycin (“+PMA/Ion,” bottom row). The left column of plots show cells expressing the mTurquoise reporter molecule; the middle column shows cells expressing the mOrange reporter molecule; and the right column shows cells expressing CD69, an indicator of T cell activation. FIG. 1C shows a plot of quantification flow cytometric analysis of FIGS. 1A and 1B. The y-axis shows the percentage of cells expressing the second reporter molecule (FP2), which was under control of an IL-2 reporter system described herein, based on the cells expressing the first reporter molecule (FP1), which was under control of the constitutively active promoter EF1a. Cells were either not activated (“−PMA/Ion”) or activated using phorbol myristate acetate (PMA) and ionomycin (“+PMA/Ion”). “EF1a_mOrange_IL-2_mTurq” refers to Jurkat cells containing the mOrange reporter molecule under control of the constitutively active ElFalpha promoter (FP1) and mTurquoise reporter molecule (mTurq) under control of an IL-2 reporter system described herein (FP2). “EF1a_mTurq_IL-2_mOrange” refers to Jurkat cells containing the mTurquoise reporter molecule under control of the constitutively active ElFalpha promoter (FP1) and mOrange reporter molecule under control of an IL-2 reporter system described herein (FP2).

FIG. 2 shows a graph of the fold increase in IL-2 inducible fluorescent protein (FP2; either mTurq in black or mOrange in light gray) upon exposure of Jurkat cells to MOLM13 CD33-expressing cells, where the Jurkat cells express the indicated CAR or co-stimulatory protein.

FIG. 3 shows a graph of the absolute change in IL-2 inducible fluorescence (ΔFP2) (either mTurq in black or mOrange in light gray) upon exposure of Jurkat cells to MOLM13 CD33-expressing cells, where the Jurkat cells express the indicated CAR or co-stimulatory protein.

FIGS. 4A and 4B show schematics of exemplary genetic constructs containing reporter molecules under control of the constitutive activate EF-1a promote FIG. 4A shows mOrange under control of the constitutive activate EF-1a promoter. FIG. 4A shows mTurquoise under control of the constitutive activate EF-1a promoter. These constructs provide constitutive expression of the relevant fluorescent protein in transfected cells.

FIGS. 5A and 5B show schematics of exemplary genetic constructs of the IL-2 reporter systems described herein. FIG. 5A shows the mOrange reporter molecule under control of a minimal NFAT-responsive promoter containing 6 NFAT binding sites and a minimal IL-2 promoter (“minP”) and the mTurquoise reporter molecule (mTurq) under control of the constitutively active ElFalpha promoter. FIG. 5B shows the mTurquoise reporter molecule under control of a minimal NFAT-responsive promoter containing 6 NFAT binding sites and a minimal IL-2 promoter (“minP”) and the mOrange reporter molecule under control of the constitutively active ElFalpha promoter. These constructs provide for expression of the reporter molecule under control of the IL-2 reporter system upon CAR activation, which may be assessed relative to expression of the reporter molecule under control of the constitutive promoter.

### DETAILED DESCRIPTION

Provided herein are chimeric antigen receptors (e.g., also referred to herein as CARs) comprising an anti-CD33 binding domain, a transmembrane region, and a signaling domain. In some embodiments, the CARs described herein further comprise any one or more of a hinge domain, linker region, and a costimulatory signaling domain. Also provided herein are nucleic acid constructs and vectors encoding any of the CARs described herein. Also provided herein are cells (e.g., immune cells such as T lymphocytes or NK cells) expressing the CARs and/or comprising any of the nucleic acids encoding the CARs described herein. Additionally, the present disclosure provides, in some embodiments, administration of a CAR, a nucleic acid or vector encoding the CAR, or a population of cells that express the CAR to treat a disease or disorder, such as a hematopoietic malignancy or pre-malignancy.

In some aspects, the present disclosure provides methods for treating a disease, disorder, or condition that is characterized by the expression of CD33 on malignant or pre-malignant cells. In some embodiments, the methods involve administering any of the CARs described herein, which target and bind CD33 through a CD33 binding domain.

Acute Myeloid Leukemia (AML)

Acute Myeloid Leukemia (AML) is an aggressive malignancy that is normally treated using intensive cytotoxic chemotherapeutic regimens with limited alternative therapeutic options when the disease becomes refractory to cytotoxic chemotherapy. Acute myeloid leukemia (AML) is a cancer of the bone marrow that needs more effective therapies. According to the National Cancer Institute, more than 60,000 people in the U.S. have AML, and less than 30% of patients survive five years following diagnosis.

CD33 and AML

CD33, also known as Siglec (Sialic-acid-binding immunoglobulin-like lectin) plays a role in mediating cell-cell interactions and in maintaining immune cells in a resting state. CD33 preferentially recognizes and binds alpha-2,3- and more avidly alpha-2,6-linked sialic acid-bearing glycans and upon engagement of ligands such as C1q or syalylated glycoproteins, two immunoreceptor tyrosine-based inhibitory motifs (ITIMs) located in the cytoplasmic tail of CD33 are phosphorylated by Src-like kinases such as LCK. These phosphorylations provide docking sites for the recruitment and activation of protein-tyrosine phosphatases PTPN6/SHP-1 and PTPN11/SHP-2. In turn, these phosphatases regulate downstream pathways through dephosphorylation of signaling molecules. One of the repressive effect of CD33 on monocyte activation requires phosphoinositide 3-kinase/PI3K.

CD33 is expressed on the surface of the vast majority of AML blasts and chronic myeloid leukemia in blast crisis. It is also aberrantly expressed on a subset of T cell acute lymphoblastic leukemias. Normal tissue expression is restricted to normal myeloid cells. Currently, treating AML with a therapy that targets CD33 can be effective, but the therapy may be limited in utility due to toxicity to the normal blood and bone marrow.

Clinical trials using anti CD33 monoclonal antibody based therapy have shown improved survival in a subset of AML patients when combined with standard chemotherapy, these effects were also accompanied by safety and efficacy concerns. Other efforts aimed at targeting AML cells have involved the generation of T cells expressing chimeric antigen receptors (CARs) that selectively target CD33 in AML. Buckley et al., Curr. Hematol. Malig. Rep. (2015) (2):65. However, the data is limited and there are uncertainties about how effective (whether all targeted cells are eliminated) this approach may be in treating the patient. Additionally, since myeloid lineage cells are indispensable for life, depleting a subject of myeloid lineage cells could have detrimental effects on survival of the patient.

Aspects of the present disclosure provide a CAR comprising an anti-CD33 antigen-binding domain. The antigen-binding domain specifically binds to CD33. In this regard, a CAR of the present disclosure comprises an anti-CD33 antigen-binding domain comprising, consisting of, or consisting essentially of, a single chain variable fragment (scFv) of an antigen-binding domain. Additionally, a CD33 CAR as described herein, may comprise any one or more additional domains, such as a hinge domain, a transmembrane domain, and one or more intracellular signaling domains (including one or more co-stimulatory domains). Additionally, the present disclosure provides, in some embodiments, administration of a population of immune cells modified to comprise any of the CD33 CARs described herein to treat a hematopoietic malignancy or pre-malignancy, e.g., AML or MDS.

Additionally, the present disclosure provides, in some embodiments, administration of a population of hematopoietic cells that are deficient in the lineage-specific cell-surface antigen (CD33). The combination of treatment is based, at least in part, on the discovery that agents comprising an antigen-binding fragment that binds a lineage-specific cell-surface antigen (e.g., immune cells expression a chimeric receptor that targets CD33) selectively cause cell death of cells expressing the lineage-specific cell-surface antigen, whereas cells that are deficient for the antigen (e.g., genetically engineered hematopoietic cells) evade cell death caused thereby.

As such, in some embodiments, the present disclosure provides methods of administering a combination of therapies including agents comprising an antigen-binding fragment that binds a lineage-specific cell-surface antigen (e.g., immune cells expressing a chimeric receptor that targets CD33) and a population of hematopoietic cells that are deficient in the lineage-specific cell-surface antigen (CD33).

### Chimeric Antigen Receptors

In general, a CAR is an artificially constructed hybrid protein or polypeptide containing the antigen-binding domain of one or more antibodies (e.g., single chain variable fragment (scFv)) linked to T-cell signaling domains. Characteristics of CARs include their ability to redirect T-cell specificity and reactivity toward a selected target in a non-MHC-restricted manner, exploiting the antigen-binding properties of monoclonal antibodies. The non-MHC-restricted antigen recognition gives T cells expressing CARs the ability to recognize antigen independent of antigen processing, thus bypassing a major mechanism of tumor escape. Moreover, when expressed in T-cells, CARs advantageously do not dimerize with endogenous T cell receptor (TCR) alpha and beta chains. The phrases “antigen(ic) specificity” and “elicit antigen-specific response,” as used herein, means that the CAR can specifically bind to and immunologically recognize antigen, such that binding of the CAR to the antigen elicits an immune response.

Of the conventional CARs containing an antigen-binding domain of an antibody, there are three generations of CARs. “First generation” CARs are typically composed of an extracellular antigen-binding domain (e.g., a scFv), which is fused to a transmembrane domain, which is fused to cytoplasmic/intracellular signaling domain. First generation CARs can provide de novo antigen recognition and cause activation of both CD4+ and CD8+ T cells through their CD3ζ chain signaling domain in a single fusion molecule, independent of HLA-mediated antigen presentation. “Second generation” CARs add an intracellular signaling domain from various co-stimulatory signaling molecules (e.g., CD28, 4-1BB, ICOS, OX40, CD27, CD40/My88 and NKGD2) to the cytoplasmic tail of the CAR to provide additional signals to the T cell. Second generation CARs comprise those that provide both co-stimulation (e.g., CD28 or 4-1BB) and activation (CD3). “Third generation” CARs comprise those that provide multiple co-stimulatory domains (e.g., CD28 and 4-1BB) and a signaling domain providing activation (e.g., CD3).

In some embodiments, a CAR described herein comprises an extracellular portion of the CAR containing anti-CD33 binding domain, a transmembrane domain, and a signaling domain. In some embodiments, the CAR further comprises one or more of a linker region, hinge region, and co-stimulatory signaling domains. In some embodiments, the CAR further comprises a signal peptide/signal sequence.

A CAR can consist of or consist essentially of the specified amino acid sequence or sequences described herein, such that other components, e.g., other amino acids, do not materially change the biological activity of the functional variant.

CARs of the present disclosure (including functional portions and functional variants) can be of any length, i.e., can comprise any number of amino acids, provided that the CARs (or functional portions or functional variants thereof) retain their biological activity, e.g., the ability to specifically bind to the target antigen (e.g., CD33), detect diseased cells in a mammal, or treat or prevent disease in a mammal, etc. For example, the CAR can be about 50 to about 5000 amino acids long, such as 50, 70, 75, 100, 125, 150, 175, 200, 300, 400, 500, 600, 700, 800, 900, 1000 or more amino acids in length.

In some embodiments, CAR constructs (including functional portions and functional variants of the invention) can comprise synthetic amino acids in place of one or more naturally-occurring amino acids. Such synthetic amino acids are known in the art, and include, for example, aminocyclohexane carboxylic acid, norleucine, a-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, b-phenylserine b-hydroxyphenylalanine, phenylglycine, a-naphthylalanine, cyclohexylalanine, cyclohexylglycine, indoline-2-carboxylic acid, 1,2, 3, 4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, a-aminocycloheptane carboxylic acid, a-(2-amino-2-norbornane)-carboxylic acid, a,g-diaminobutyric acid, a,b-diaminopropionic acid, homophenylalanine, and a-tert-butylglycine.

In some embodiments, CAR constructs (including functional portions and functional variants) can be glycosylated, amidated, carboxylated, phosphorylated, esterified, N-acylated, cyclized via, e.g. a disulfide bridge, or converted into an acid addition salt and/or optionally dimerized or polymerised, or conjugated.

In some embodiments, CAR constructs (including functional portions and functional variants thereof) can be obtained by methods known in the art. In some embodiments, CAR constructs may be made by any suitable method of making polypeptides or proteins, including de novo synthesis. CAR constructs can be recombinantly produced using the nucleic acids described herein using standard recombinant methods. See, for instance, Green et al., Molecular Cloning: A Laboratory Manual, 4th ed., Cold Spring Harbor Press, Cold Spring Harbor, N Y 2012. Further, portions of some of the CAR constructs described herein (including functional portions and functional variants thereof) can be isolated and/or purified from a source, such as a plant, a bacterium, an insect, a mammal, e.g., a rat, a human, etc. Methods of isolation and purification are well known in the art. Alternatively, the CAR constructs described herein (including functional portions and functional variants thereof) can be commercially synthesized by companies, such as Synpep (Dublin, CA), Peptide Technologies Corp. (Gaithersburg, MD), and Multiple Peptide Systems (San Diego, CA). In this respect, the CAR constructs can be synthetic, recombinant, isolated, and/or purified.

Further provided herein are nucleic acids comprising a nucleotide sequence encoding any of the CAR constructs described herein (including functional portions and functional variants thereof). The nucleic acids described herein may comprise a nucleotide sequence encoding any of the leader sequences (e.g., signal peptides), antigen binding domains, transmembrane domains, linker regions, costimulatory signaling domains, and/or intracellular T cell signaling domains described herein.

Antigen Binding Domain

In some embodiments, any of the CARs described herein comprises an antigen-binding domain that binds to an antigen (e.g., a lineage-specific cell surface antigen) on a target cell. As used herein, the terms “lineage-specific cell-surface antigen” and “cell-surface lineage-specific antigen” may be used interchangeably and refer to any antigen that is sufficiently present on the surface of a cell and is associated with one or more populations of cell lineage(s). For example, the antigen may be present on one or more populations of cell lineage(s) and absent (or at reduced levels) on the cell-surface of other cell populations.

In general, lineage-specific cell-surface antigens can be classified based on a number of factors such as whether the antigen and/or the populations of cells that present the antigen are required for survival and/or development of the host organism.

In some embodiments, the cell-surface lineage-specific antigen may be a cancer antigen, for example a cell-surface lineage-specific antigen that is differentially present on cancer cells. In some embodiments, the cancer antigen is an antigen that is specific to a tissue or cell lineage. Examples of cell-surface lineage-specific antigen that are associated with a specific type of cancer include, without limitation, CD20, CD22 (Non-Hodgkin's) lymphoma, B-cell lymphoma, chronic lymphocytic leukemia (CLL)), CD52 (B-cell CLL), CD33 (Acute myelogenous leukemia (AML)), CD10 (gp100) (Common (pre-B) acute lymphocytic leukemia and malignant melanoma), CD3/T-cell receptor (TCR) (T-cell lymphoma and leukemia), CD79/B-cell receptor (BCR) (B-cell lymphoma and leukemia), CD26 (epithelial and lymphoid malignancies), RCAS1 (gynecological carcinomas, biliary adenocarcinomas and ductal adenocarcinomas of the pancreas) as well as prostate specific membrane antigen. In some embodiments, the cell-surface antigen is CD33 and is associated with AML cells.

Any antibody or antigen-binding fragment thereof know in the art can be used for constructing a CAR as described herein. The antigen-binding domain may comprise any antigen-binding portion of an antibody. The antigen-binding portion can be any portion that has at least one antigen binding site, such as Fab, F(ab′)2, dsFv, scFv, diabodies, Nanobody®, and triabodies. In some embodiments, the antigen-binding portion is a single-chain variable region fragment (scFv) antigen-binding fragment. An scFv is a truncated Fab fragment including the variable (V) domain of an antibody heavy chain linked to a V domain of a light antibody chain via a synthetic peptide linker, which can be generated using routine recombinant DNA technology techniques. Similarly, disulfide-stabilized variable region fragments (dsFv) can be prepared by recombinant DNA technology.

The antigen-binding domain can include, but is not limited to, a monoclonal antibody, a polyclonal antibody, a synthetic antibody, a human antibody, a humanized antibody, a non-human antibody, and any fragment thereof. Thus, in one embodiment, the antigen binding domain portion comprises a mammalian antibody or a fragment thereof.

In some instances, the antigen-binding domain is derived from the same species in which the CAR will ultimately be used herein. For example, for use in humans, the antigen binding domain of the CAR comprises a human antibody, a humanized antibody, or a fragment thereof. Human antibodies can be made by a variety of methods known in the art including phage display methods using antibody libraries derived from human immunoglobulin sequences, including improvements to these techniques. See, also, U.S. Pat. Nos. 4,444,887 and 4,716,111; and PCT publications WO 98/46645, WO15 98/50433, WO 98/24893, WO 98/16654, WO 96/34096, WO 96/33735, and WO 91/10741; each of which is incorporated herein by reference in its entirety. In some embodiments, the antigen-binding domain is derived from a different species as the species in which the CAR will ultimately be used herein. For example, the antigen-binding domain may be derived from a camelid species but used in a human.

For example, antibodies specific to a lineage-specific antigen of interest can be made by the conventional hybridoma technology. The lineage-specific antigen, which may be coupled to a carrier protein such as KLH, can be used to immunize a host animal for generating antibodies binding to that complex. The route and schedule of immunization of the host animal are generally in keeping with established, and conventional techniques for antibody stimulation and production, as further described herein. General techniques for production of mouse, humanized, and human antibodies are known in the art and are described herein. It is contemplated that any mammalian subject including humans or antibody producing cells therefrom can be manipulated to serve as the basis for production of mammalian, including human hybridoma cell lines. Typically, the host animal is inoculated intraperitoneally, intramuscularly, orally, subcutaneously, intraplantar, and/or intradermally with an amount of immunogen including, as described herein.

Hybridomas can be prepared from the lymphocytes and immortalized myeloma cells using the general somatic cell hybridization technique of Kohler. B. and Milstein. C. Nature (1975) 256:495-497 or as modified by Buck, D. W., et al., In Vitro (1982), 18:377-381. Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce the TCR-like monoclonal antibodies described herein. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas that may be used as source of antibodies encompass all derivatives, progeny cells of the parent hybridomas that produce monoclonal antibodies capable of binding to a lineage-specific antigen. Hybridomas that produce such antibodies may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity if present, can be removed, for example, by running the preparation over adsorbents made of the immunogen attached to a solid phase and eluting or releasing the desired antibodies off the immunogen. Immunization of a host animal with a target antigen or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glutaraldehyde, succinic anhydride, SOCl, or R1N═C═NR, where R and R1 are different alkyl groups, can yield a population of antibodies (e.g., monoclonal antibodies).

If desired, an antibody of interest (e.g., produced by a hybridoma) may be sequenced and the polynucleotide sequence may then be cloned into a vector for expression or propagation. The sequence encoding the antibody of interest may be maintained in vector in a host cell and the host cell can then be expanded and frozen for future use. In an alternative, the polynucleotide sequence may be used for genetic manipulation to “humanize” the antibody or to improve the affinity (affinity maturation), or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity to the lineage-specific antigen. It will be apparent to one of skill in the art that one or more polynucleotide changes can be made to the antibody and still maintain its binding specificity to the target antigen.

In other embodiments, fully human antibodies can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse® from Amgen, Inc. (Fremont, Calif.) and HuMAb-Mouse® and TC Mouse™ from Medarex, Inc. (Princeton, N.J.). In another alternative, antibodies may be made recombinantly by phage display or yeast technology. See, for example, U.S. Pat. Nos. 5,565,332; 5,580,717; 5,733,743; and 6,265,150; and Winter et al., Annu. Rev. Immunol. (1994) 12:433-455. Alternatively, the phage display technology (McCafferty et al., Nature (1990) 348:552-553) can be used to produce human antibodies and antibody and antibody fragments in vitro, from immunoglobulin variable (V) domain gene repertoires from unimmunized donors.

Antigen-binding fragments of an intact antibody (full-length antibody) can be prepared via routine methods. For example, F(ab′)2 fragments can be produced by pepsin digestion of an antibody molecule, and Fab fragments that can be generated by reducing the disulfide bridges of F(ab′)2 fragments.

Genetically engineered antibodies, and bi-specific antibodies, can be produced via, e.g., conventional recombinant technology. In one example, DNA encoding a monoclonal antibodies specific to a target antigen can be readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into one or more expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., Proc. Nat. Acad. Sci. (1984) 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

Techniques developed for the production of “chimeric antibodies” are well known in the art. See, e.g., Morrison et al. Proc. Natl. Acad. Sci. USA (1984) 81, 6851; Neuberger et al. Nature (1984) 312, 604; and Takeda et al. Nature (1984) 314:452.

Methods for constructing humanized antibodies are also well known in the art. See, e.g., Queen et al., Proc. Natl. Acad. Sci. USA (1989) 86:10029-10033 (1989). In one example, variable regions of VH and VL of a parent non-human antibody are subjected to three-dimensional molecular modeling analysis following methods known in the art. Next, framework amino acid residues predicted to be important for the formation of the correct CDR structures are identified using the same molecular modeling analysis. In parallel, human VH and VL chains having amino acid sequences that are homologous to those of the parent non-human antibody are identified from any antibody gene database using the parent VH and VL sequences as search queries. Human VH and VL acceptor genes are then selected.

The CDR regions within the selected human acceptor genes can be replaced with the CDR regions from the parent non-human antibody or functional variants thereof. When necessary, residues within the framework regions of the parent chain that are predicted to be important in interacting with the CDR regions (see above description) can be used to substitute for the corresponding residues in the human acceptor genes.

A single-chain antibody can be prepared via recombinant technology by linking a nucleotide sequence coding for a heavy chain variable region and a nucleotide sequence coding for a light chain variable region. Preferably, a flexible linker is incorporated between the two variable regions. Alternatively, techniques described for the production single chain antibodies (U.S. Pat. Nos. 4,946,778 and 4,704,692) can be adapted to produce a phage or yeast scFv library and scFv clones specific to a lineage-specific antigen can be identified from the library following routine procedures. Positive clones can be subjected to further screening to identify those that bind lineage-specific antigen.

In some aspects, the antigen-binding domain is operably linked to another domain of the CAR, such as the transmembrane domain or the intracellular domain, for expression in the cell. In some embodiments, a nucleic acid encoding the antigen-binding domain is operably linked to a nucleic acid encoding a transmembrane domain and a nucleic acid encoding an intracellular domain.

Exemplary CD33 Antigen Binding Domains

In some embodiments, CARs described herein target CD33 and comprise an extracellular region comprising an anti-CD33 binding domain. In some embodiments, a lineage-specific antigen of interest is CD33 and the antigen-binding domain of a CAR specifically binds CD33, for example, human CD33. In some embodiments, the CAR comprises an anti-CD33 antigen-binding domain comprising, consisting of, or consisting essentially of, an antigen-binding fragment such as a single chain variable fragment (scFv) of the antigen-binding domain.

In some embodiments, the CAR comprises an anti-CD33 antigen binding domain of hP67.6 (Cowan et al., Front. Biosci. (2013) (Landmark Ed.), 18: 1311-1334 and U.S. Pat. No. 5,739,116, each incorporated by reference herein), M195 (Co et al., J. Immunol., (1992) 148: 1149-1154, incorporated by reference herein), or Hu195 (Co et al., supra). In some embodiments, a CAR comprises anti-CD33 antigen-binding domain comprising, consisting of, or consisting essentially of, a single chain variable fragment (scFv) of the antigen-binding domain of hP67.6, M195, or Hu195 or a portion thereof.

In some embodiments, the anti-CD33 antigen-binding domain includes at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98% 99% or more of an anti-CD33 antigen-binding domain, such that the fragment retains the ability to bind CD33. In some embodiments, the anti-CD33 antigen-binding domain includes at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98% 99% or more of hP67.6, M195, or Hu195, such that the fragment retains the ability to bind CD33.

In some embodiments, an anti-CD33 antigen-binding domain is a monoclonal antibody, or antigen-binding fragment thereof. In some embodiments, an anti-CD33 antigen-binding domain is a humanized antibody, or antigen-binding fragment thereof.

Exemplary anti-CD33 antibodies or antigen-binding fragments thereof can include, but are not limited to, SEQ ID NOs: 60-101. In some embodiments, an anti-CD33 antigen binding domain comprises an scFv of an antibody light chain. In some embodiments, an anti-CD33 antigen binding domain comprises an scFv of an antibody heavy chain. In some embodiments, an anti-CD33 antibody or antigen-binding fragment thereof comprises an amino acid sequence shown in any one of SEQ ID NOs: 60-101, or a sequence with at least 80, 85, 90, 91, 92, 93, 94, 95, 96, 97, 98, or 99% identity to any one of SEQ ID NOs: 60-101. In some embodiments, an anti-CD33 antigen binding domain comprises an scFv of an antibody heavy chain comprising an amino acid sequence of SEQ ID NO: 60 (or a sequence with at least 80, 85, 90, 91, 92, 93, 94, 95, 96, 97, 98, or 99% identity to any one of SEQ ID NO: 60), and an scFv of an antibody light chain comprising an amino acid sequence of SEQ ID NO: 90 (or a sequence with at least 80, 85, 90, 91, 92, 93, 94, 95, 96, 97, 98, or 99% identity to any one of SEQ ID NO: 90). In some embodiments, an anti-CD33 antigen binding domain comprises an scFv of an antibody heavy chain comprising an amino acid sequence of SEQ ID NO: 91 (or a sequence with at least 80, 85, 90, 91, 92, 93, 94, 95, 96, 97, 98, or 99% identity to any one of SEQ ID NO: 91), and an scFv of an antibody light chain comprising an amino acid sequence of SEQ ID NO: 92 (or a sequence with at least 80, 85, 90, 91, 92, 93, 94, 95, 96, 97, 98, or 99% identity to any one of SEQ ID NO: 92). In some embodiments, an anti-CD33 antigen binding domain comprises an scFv of an antibody heavy chain comprising an amino acid sequence of SEQ ID NO:100 (or a sequence with at least 80, 85, 90, 91, 92, 93, 94, 95, 96, 97, 98, or 99% identity to any one of SEQ ID NO:100), and an scFv of an antibody light chain comprising an amino acid sequence of SEQ ID NO: 101 (or a sequence with at least 80, 85, 90, 91, 92, 93, 94, 95, 96, 97, 98, or 99% identity to any one of SEQ ID NO: 101).

Exemplary Mylo Binding Sequences:

scFv heavy chain:
[SEQ ID NO: 60]
EVQLVQSGAEVKKPGSSVKVSCKASGYTITDSNIHWVRQAPGQSLEWIG
YIYPYNGGTDYNQKFKNRATLTVDNPTNTAYMELSSLRSEDTAFYYCVN
GNPWLAYWGQGTLVTVSS
scFv light chain:
[SEQ ID NO: 90
DIQLTQSPSTLSASVGDRVTITCRASESLDNYGIRFLTWFQQKPGKAPK
LLMYAASNQGSGVPSRFSGSGSGTEFTLTISSLQPDDFATYYCQQTKEV
PWSFGQGTKVEVKR

Exemplary M195 Binder Sequences:

scFv heavy chain:
[SEQ ID NO: 91]
QVQLVQSGAEVKKPGSSVKVSCKASGYTFTDYNMHWVRQAPGQGLEWIG
YIYPYNGGTGYNQKFKSKATITADESTNTAYMELSSLRSEDTAVYYCAR
GRPAMDYWGQGTLVTVSS
scFv light chain:
[SEQ ID NO: 92]
DIQMTQSPSSLSASVGDRVTITCRASESVDNYGISFMNWFQQKPGKAPK
LLIYAASNQGSGVPSRFSGSGSGTDFTLTISSLQPDDFATYYCQQSKEV
PWTFGQGTKVEIK

Exemplary h195 Binding Sequences:

scFv heavy chain:
[SEQ ID NO: 100]
QVQLVQSGAEVKKPGSSVKVSCKASGYTFTDYNMHWVRQAPGQGLEWIG
YIYPYNGGTGYNQKFKSKATITADESTNTAYMELSSLRSEDTAVYYCAR
GRPAMDYWGQGTLVTVSS
scFv light chain:
[SEQ ID NO: 101]
DIQMTQSPSSLSASVGDRVTITCRASESVDNYGISFMNWFQQKPGKAPK
LLIYAASNQGSGVPSRFSGSGSGTDFTLTISSLQPDDFATYYCQQSKEV
PWTFGQGTKVEIK

In some embodiments, a nucleic acid encoding the anti-CD33 antigen binding domain is operably linked to a nucleic acid encoding a linker region, a nucleic acid encoding a transmembrane domain, and/or a nucleic acid encoding an intracellular domain (e.g., a costimulatory signaling domain, a signaling domain).

In some embodiments, the CAR comprises a linker region. In some embodiments, the light chain variable region and the heavy chain variable region of the antigen-binding domain can be joined to each other by a linker. In some embodiments, the antigen-binding domain can be joined to another domain, such as a transmembrane domain, hinge, and/or intracellular domain with a linker region. The linker may comprise any suitable amino acid sequence. In some embodiments, the linker is a Gly/Ser linker from about 1 to about 100, from about 3 to about 20, from about 5 to about 30, from about 5 to about 18, or from about 3 to about 8 amino acids in length and consists of glycine and/or serine residues in sequence. Accordingly, the Gly/Ser linker may consist of glycine and/or serine residues. Preferably, the Gly/Ser linker comprises the amino acid sequence of GGGGS (SEQ ID NO: 1), and multiple SEQ ID NO: 1 may be present within the linker. Any linker sequence may be used as a spacer between the antigen-binding domain and any other domain of the CAR, such as the transmembrane domain. In some, embodiments, the region linker is ([G]x[S]y)z, for example wherein x can be 1-10, 7 can be 1-3, and z can be 1-5. In some embodiments, the linker region comprises the amino acid sequence GGGGSGGGGS (SEQ ID NO: 93). In some embodiments, the linker region comprises the amino acid sequence GGGGSGGGGSGGGGS (SEQ ID NO: 99).

In some embodiments, the antigen-binding domain comprises one or more leader sequences (signal peptides, signal sequence), such as those described herein. In some embodiments, the leader sequence may be positioned at the amino terminus of the CAR within the CAR construct. The leader sequence may comprise any suitable leader sequence, e.g., any CARs described herein may comprise any leader sequence, such as those described herein. In some embodiments, while the leader sequence may facilitate expression of the released CARs on the surface of the cell, the presence of the leader sequence in an expressed CAR is not necessary in order for the CAR to function. In some embodiments, upon expression of the CAR on the cell surface, the leader sequence may be cleaved off. Accordingly, in some embodiments, the released CARs (e.g., surface expressed) lack a leader sequence. In some embodiments, the CARs within the CAR construct lack a leader sequence.

Hinge

In some embodiments, the CAR comprises a hinge/spacer region that links the extracellular antigen-binding domain to another domain, such as a transmembrane domain. The hinge/spacer region can be flexible enough to allow the antigen-binding domain to orient in different directions to facilitate target antigen recognition. In some embodiments, the hinge domain is a portion of the hinge domain of CD8a or CD28, e.g., a fragment containing at least 15 (e.g., 20, 25, 30, 35, or 40) consecutive amino acids of the hinge domain of CD8a or CD28.

In some embodiments, the CAR comprises a hinge domain, such as a hinge domain from CD8, CD28, or IgG4. In some embodiments, the hinge domain is a CD8 (e.g., CD8a) hinge domain. In some embodiments, the CD8 hinge domain is human (e.g., obtained from/derived from a human protein sequence). In some embodiments, the CD8 hinge domain comprises, consists of, or consists essentially of SEQ ID NO: 2.

CD8 hinge region
[SEQ ID NO: 2]
TTTPAPRPPTPAPTIASQPLSLRPEACRPAAGGAVHTRGLDFACD

In some embodiments, the hinge domain is a CD28 hinge domain. In some embodiments, the CD28 hinge domain is human (e.g., obtained from/derived from a human protein sequence). In some embodiments, the CD28 hinge domain comprises, consists of, or consists essentially of SEQ ID NO: 3.

CD28 hinge region
[SEQ ID NO: 3]
AAATEVMYPPPYLDNEKSNGTTTHVKGKHLCPSPLFPGPSKP

Hinge domains of antibodies, such as an IgG, IgA, IgM, IgE, or IgD antibody, are also compatible for use in the chimeric receptors described herein. In some embodiments, the hinge domain is the hinge domain that joins the constant domains CH1 and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody. In some embodiments, the hinge domain is an IgG4 hinge domain.

Also within the scope of the present disclosure are CARs comprising a hinge domain that is a non-naturally occurring peptide. In some embodiments, the hinge domain between the C-terminus of the extracellular ligand-binding domain of an Fc receptor and the N-terminus of the transmembrane domain is a peptide linker, such as a (GlyxSer)n linker, wherein x and n, independently can be an integer between 3 and 12, including 3, 4, 5, 6, 7, 8, 9, 10, 11, 12, or more.

Additional peptide linkers that may be used in a hinge domain of the chimeric receptors described herein are known in the art. See, e.g., Wriggers et al. Current Trends in Peptide Science (2005) 80(6): 736-74 and PCT Publication No. WO 2012/088461.

In some embodiments, the hinge/spacer region of a presently disclosed CAR comprises a native or modified hinge region of a CD28 polypeptide as described herein. In certain embodiments, the hinge/spacer region of a presently disclosed CAR construct comprises a native or modified hinge region of a CD8a polypeptide as described herein. In certain embodiments, the hinge/spacer region of a presently disclosed CAR construct comprises a native or modified hinge region of a IgG4 polypeptide as described herein.

Transmembrane Domain

With respect to the transmembrane domain, a CAR can be designed to comprise a transmembrane domain that connects the antigen-binding domain of the CAR to an intracellular region of the CAR. In some embodiments, the transmembrane domain is naturally associated with one or more of the domains in the CAR. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of the alpha, beta or zeta chain of the T-cell receptor, CD28, CD3 epsilon, CD45, CD4, CD5, CD8, CD8a, CD9, CD 16, CD22, CD33, CD37, CD64, CD80, CD86, CD134, CD137, CD154, Toll-like receptor 1 (TLR1), TLR2, TLR3, TLR4, TLR5, TLR6, TLR7, TLR8, and TLR9.

In some embodiments, the transmembrane domain may be synthetic, in which case it will comprise predominantly hydrophobic residues such as leucine and valine. Preferably a triplet of phenylalanine, tryptophan and valine will be found at each end of a synthetic transmembrane domain.

In some embodiments, the transmembrane domain is a CD8 (e.g., CD8a) transmembrane domain. In some embodiments, the CD8 transmembrane domain is human (e.g., obtained from/derived from a human protein sequence). In some embodiments, a CD8 transmembrane domain comprises, consists of, or consists essentially of SEQ ID NO: 4.

CD8 transmembrane region
[SEQ ID NO: 4]
TYTWAPLAGTCGVLLLSLVTTLYC

In some embodiments, the transmembrane domain is a CD28 transmembrane domain. In some embodiments, the CD28 transmembrane domain is human (e.g., obtained from/derived from a human protein sequence). In some embodiments, the CD28 transmembrane domain comprises, consists of, or consists essentially of SEQ ID NO: 5.

CD28 transmembrane domain
[SEQ ID NO: 5]
FWVLVVVGGVLACYSLLVTVAFTTFWVR
SKRSRLLHSDYMNMTPRRPGPTRKHYQP
YAPPRDFAAYRS

Intracellular Signaling Domains

In some embodiments, the CAR construct comprises an intracellular signaling domain, which may be comprised of one or more signaling domains and costimulatory domains. The intracellular signaling domain of the CAR, is involved in activation of the cell in which the CAR is expressed. In some embodiments, the intracellular signaling domain of the CAR construct described herein is involved in activation of a T lymphocyte or NK cells. In some embodiments, the signaling domain of the CAR construct described herein includes a domain involved in signal activation and/or transduction.

Examples of an intracellular signaling domains for use in the CAR constructs described herein include, but are not limited to, the cytoplasmic portion of a surface receptor, co-stimulatory molecule, and any molecule that acts in concert to initiate signal transduction in a cell (e.g., an immune cell (e.g., a T lymphocyte), NK cell), as well as any derivative or variant of these elements and any synthetic sequence that has the same functional capability.

Examples of the signaling domains that may be used in the intracellular signaling domain of the CARs described herein include, without limitation, a fragment or domain from one or more molecules or receptors including, but are not limited to, TCR, CD3 zeta (CD3), CD3 gamma, CD3 delta, CD3 epsilon, CD86, common FcR gamma, FcR beta (Fc Epsilon Rib), CD79a, CD79b, Fcgamma RIIa, DAP10, DAP 12, T cell receptor (TCR), CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD 1 id, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD 162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof.

Any cytoplasmic signaling domain can be used in the CARs described herein. In general, a cytoplasmic signaling domain relays a signal, such as interaction of an extracellular ligand-binding domain with its ligand, to stimulate a cellular response, such as inducing an effector function of the cell (e.g., cytotoxicity).

As will be evident to one of ordinary skill in the art, a factor involved in T cell activation is the phosphorylation of immunoreceptor tyrosine-based activation motif (ITAM) of a cytoplasmic signaling domain. Any ITAM-containing domain known in the art may be used to construct the chimeric receptors described herein, and included as part of the cytoplasmic signaling domain. In general, an ITAM motif may comprise two repeats of the amino acid sequence YxxL/I separated by 6-8 amino acids, wherein each x is independently any amino acid, producing the conserved motif YxxL/Ix(6-8)YxxL/I. In some embodiments, the cytoplasmic signaling domain is from CD3ζ.

CD3ζ associates with TCRs to produce a signal and contains immunoreceptor tyrosine-based activation motifs (ITAMs). In some embodiments, a CD3 intracellular T cell signaling sequence is human (e.g., obtained from or derived from a human protein). In some embodiments, a CD3 intracellular T cell signaling sequence comprises, consists of, or consists essentially of the amino acid sequence of SEQ ID NO: 6 or 98, or a sequence that is at least 70%, at least 75%, at least 80%, at least 85%, at least 90%, at least 95%, or at least 99% identical the amino acid sequence of SEQ ID NO: 6 or 98. In some embodiments, an intracellular T cell signaling domain comprises a CD3 that contains on or more mutated and/or deleted ITAMs.

CD3ζ signaling domain (variant A)
[SEQ ID NO: 6]
RVKFSRSADAPAYKQGQNQLYNELNLGRREE
YDVLDKRRGRDPEMGGKPRRKNPQEGLYNEL
QKDKMAEAYSETGMKGERRRGKGHDGLYQGL
STATKDTYDALHMQALPPR
CD3ζ signaling domain (variant B)
[SEQ ID NO: 98]
RVKFSRSADAPAYQQGQNQLYNELNLGRREE
YDVLDKRRGRDPEMGGKPRRKNPQEGLYNEL
QKDKMAEAYSETGMKGERRRGKGHDGLYQGL
STATKDTYDALHMQALPPR

In certain non-limiting embodiments, an intracellular signaling domain of the CAR further comprises at least one (e.g., 1, 2, 3 or more) co-stimulatory signaling domain. In some embodiments, the co-stimulatory signaling domain comprises at least one co-stimulatory molecule, which can provide optimal lymphocyte activation. In general, many immune cells require co-stimulation, in addition to stimulation of an antigen-specific signal, to promote cell proliferation, differentiation and survival, and to activate effector functions of the cell. Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory protein may be compatible for use in the chimeric receptors described herein. The type(s) of co-stimulatory signaling domains may be selected based on factors such as the type of the cells in which the CARs would be expressed (e.g., primary T cells, T cell lines, NK cell lines) and the desired immune effector function (e.g., cytotoxicity).

Examples of such co-stimulatory signaling domains include a fragment or domain from one or more molecules or receptors including, without limitation, are not limited to 4-1BB, CD28, ICOS, TLR1, TLR2, TLR3, TLR4, TLR5, TLR6, TLR7, TLR8, TLR9, TLR10, TLR11, CD116 receptor beta chain, CSF1-R, LRP1/CD91, SR-A1, SR-A2, MARCO, SR-CL1, SR-CL2, SR-C, SR-E, CR1, CR3, CR4, dectin 1, DEC-205, DC-SIGN, CD14, CD36, LOX-1, CD11b, together with any of the signaling domains listed in the above paragraph in any combination. In some embodiments, the intracellular signaling domain of the CAR includes any portion of one or more co-stimulatory signaling molecules, such as at least one signaling domain from CD3, Fc epsilon RI gamma chain, any derivative or variant thereof, including any synthetic sequence thereof that has the same functional capability, and any combination thereof.

In some embodiments, one or more co-stimulatory signaling domains (e.g., 1, 2, 3, or more) are included in a CAR construct with a CD3 intracellular T cell signaling sequence. In some embodiments, the one or more co-stimulatory signaling domains are selected from CD137 (4-1BB) and CD28, or a combination thereof. In some embodiments, the CAR comprises a 4-1BB (CD137) costimulatory signaling domain. In some embodiments, the CAR comprises a CD28 costimulatory signaling domain. In some embodiments, the CAR comprises both a 4-1BB costimulatory signaling domain and a CD28 costimulatory signaling domain.

4-1BB, also known as CD137, transmits a potent costimulatory signal to T cells, promoting differentiation and enhancing long-term survival of T lymphocytes. In some embodiments, a 4-1BB intracellular signaling sequence is human (e.g., obtained from/derived from a human protein sequence). In some embodiments, the 4-1BB intracellular T cell signaling sequence comprises, consists of, or consists essentially of the amino acid sequence of SEQ ID NO: 7. In some embodiments, the 4-1BB costimulatory signaling domain comprises, consists of, or consists essentially of the amino acid sequence of SEQ ID NO: 7, or a sequence that is at least 70%, at least 75%, at least 80%, at least 85%, at least 90%, at least 95%, or at least 99% identical the amino acid sequence of SEQ ID NO: 7.

4-1BB costimulatory signaling domain
[SEQ ID NO: 7]
KRGRKKLLYTFKQPFMRPVQTTQEEDGCSCRFPEEEEGGCEL

Some suitable costimulatory domains are provided herein, and other suitable costimulatory domains and costimulatory domain sequences will be apparent to the skilled artisan based on the present disclosure in view of the knowledge in the art. Suitable costimulatory domains include, for example, those described in Weinkove et al., Selecting costimulatory domains for chimeric antigen receptors: functional and clinical considerations, Clin Transl Immunology. 2019; 8(5): e1049, the entire contents of which are incorporated herein by reference.

Between the antigen-binding domain and the transmembrane domain of the CAR, or between the intracellular signaling domain and the transmembrane domain of the CAR, a spacer domain may be incorporated. As used herein, the term “spacer domain” generally means any oligo- or polypeptide that functions to link the transmembrane domain to, either the antigen binding domain or, the intracellular domain in the polypeptide chain. In some embodiments, the spacer domain may comprise up to 300 amino acids, preferably 10 to 100 amino acids and most preferably 25 to 50 amino acids. In some embodiments, a short oligo- or polypeptide linker, preferably between 2 and 10 amino acids in length may form the linkage between the transmembrane domain and the intracellular domain of the CAR. An example of a linker includes a glycine-serine doublet.

### Signal Peptides

In some embodiments, any of the CARs described herein may further comprise a signal peptide (signal sequence). In general, signal peptides are short amino acid sequences that target a polypeptide to a site in a cell. In some embodiments, the signal peptide directs the CAR to the secretory pathway of the cell and will allow for integration and anchoring of the CAR into the lipid bilayer at the cell surface. Signal sequences including signal sequences of naturally occurring proteins or synthetic, non-naturally occurring signal sequences, that are compatible for use in the chimeric receptors described herein will be evident to one of skill in the art.

The CARs described herein may be prepared in constructs with, e.g., self-cleaving peptides, such that the CAR constructs containing anti-CD33 CAR components are bicistronic, tricistronic, etc.

Various CAR constructs and numerous elements of CAR constructs (for example, various CD33 binding domains, signal peptides, linkers, hinge sequences, transmembrane domains, costimulatory domains, and signaling domains) are disclosed herein, and those of skill in the art will be able to ascertain the sequences of these elements and of additional suitable elements known in the art based on the present disclosure in view of the knowledge in the art. Exemplary CAR element sequences, e.g., for CD33 binding domains, signal peptides, linkers, hinge sequences, transmembrane domains, costimulatory domains, and signaling domains, are disclosed in PCT/US2019/022309, published as WO/2019/178382, e.g., throughout the specification and in Tables 1-6, the entire contents of which are incorporated herein by reference.

### Vectors

Nucleic acids encoding any of the CAR constructs described herein can be incorporated into a vector, such as a recombinant expression vector. In this regard, an embodiment of the invention provides recombinant expression vectors comprising any of the nucleic acids of the invention. For purposes herein, the terms “recombinant expression vector” and “vector” may be used interchangeably and refer to a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell.

In some embodiments, vectors are not naturally-occurring as a whole. However, parts of the vectors can be naturally-occurring. The inventive recombinant expression vectors can comprise any type of nucleotides, including, but not limited to DNA and RNA, which can be single-stranded or double-stranded, synthesized or obtained in part from natural sources, and which can contain natural, non-natural or altered nucleotides. In some embodiments, the vector is a DNA vector. In some embodiments, the vector is an RNA vector. The vectors can comprise naturally-occurring or non-naturally-occurring internucleotide linkages, or both types of linkages. In some embodiments, a non-naturally occurring or altered nucleotides or internucleotide linkages do not hinder the transcription or replication of the vector.

The vector may be any suitable recombinant expression vector, and can be used to transform or transfect any suitable host cell. Suitable vectors include those designed for propagation and expansion or for expression or both, such as plasmids and viruses. A vector can be selected from the group consisting of the pUC series (Fermentas Life Sciences, Glen Burnie, MD), the pBluescript series (Stratagene, LaJolla, CA), the pET series (Novagen, Madison, WI), the pGEX series (Pharmacia Biotech, Uppsala, Sweden), and the pEX series (Clontech, Palo Alto, CA). Bacteriophage vectors, such as LGTlO, λGT11, LZapII (Stratagene), λEMBT4, and λNMI149, also can be used. Examples of plant expression vectors include pBIO1, pBI101.2, pBI101.3, pBH21 and pBIN19 (Clontech). Examples of animal expression vectors include pEUK-CI, pMAM, and pMAMneo (Clontech). The recombinant expression vector may be a viral vector, e.g., an adenoviral vector, a retroviral vector, or a lentiviral vector. In some embodiments, the vector is an adenoviral vector. In some embodiments, the vector is a retroviral vector. In some embodiments, the vector is a lentiviral vector.

In some embodiments, the vectors of the invention can be prepared using standard recombinant DNA techniques described in, for example, Green et al., supra. Constructs of expression vectors, which are circular or linear, can be prepared to contain a replication system functional in a prokaryotic or eukaryotic host cell. Replication systems can be derived, e.g., from ColEl, 2μ plasmid, λ, SV40, bovine papilloma virus, and the like.

A recombinant expression vector may comprise regulatory sequences, such as transcription and translation initiation and termination codons, which are specific to the type of host cell (e.g., bacterium, fungus, plant, or animal) into which the vector is to be introduced, as appropriate, and taking into consideration whether the vector is DNA- or RNA-based. A recombinant expression vector may also comprise restriction sites to facilitate cloning.

A vector can include one or more marker genes, which allow for selection of transformed or transfected host cells. Marker genes include biocide resistance, e.g., resistance to antibiotics, heavy metals, etc., complementation in an auxotrophic host to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

Further, the vectors can be made to include a suicide gene. As used herein, the term “suicide gene” refers to a gene that causes the cell expressing the suicide gene to die. A suicide gene can be a gene that confers sensitivity to an agent, e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine deaminase, purine nucleoside phosphorylase, and nitroreductase.

Promoters

In some embodiments, a recombinant expression vector can comprise a native or nonnative promoter operably linked to the nucleotide sequence encoding the CAR construct (including functional portions and functional variants thereof), or to the nucleotide sequence which is complementary to or which hybridizes to the nucleotide sequence encoding the CAR construct. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, a SFFV promoter, an EF1α promoter, an SV40 promoter, an RSV promoter, or a promoter found in the long-terminal repeat of the murine stem cell virus. In some embodiments the promoter is an SFFV promoter (e.g., as represented in SEQ ID NO: 8).

SFFV promoter
[SEQ ID NO: 8]
GTAACGCCATTTTGCAAGGCATGGAAAAATACCAAACCAA
GAATAGAGAAGTTCAGATCAAGGGCGGGTACATGAAAATA
GCTAACGTTGGGCCAAACAGGATATCTGCGGTGAGCAGTT
TCGGCCCCGGCCCGGGGCCAAGAACAGATGGTCACCGCAG
TTTCGGCCCCGGCCCGAGGCCAAGAACAGATGGTCCCCAG
ATATGGCCCAACCCTCAGCAGTTTCTTAAGACCCATCAGA
TGTTTCCAGGCTCCCCCAAGGACCTGAAATGACCCTGCGC
CTTATTTGAATTAACCAATCAGCCTGCTTCTCGCTTCTGT
TCGCGCGCTTCTGCTTCCCGAGCTCTATAAAAGAGCTCAC
AACCCCTCACTCGGCGCGCCAGTCCTCCGACAGACTGAGT
CG

The vectors described herein can be designed for transient expression, stable expression, or for both. Alternatively or in addition, the recombinant expression vectors can be made for constitutive expression or for inducible expression.

Included in the scope of the invention are conjugates, e.g., bioconjugates, comprising any of the CAR constructs (including any of the functional portions or variants thereof), nucleic acids, recombinant expression vectors, host cells, or populations of host cells described herein. Conjugates, as well as methods of synthesizing conjugates in general, are known in the art.

Production of Modified Cells

Aspects of the present disclosure provide methods for modifying a cell comprising introducing a chimeric antigen receptor (CAR) into cell, (e.g., an immune cell, such as a T lymphocyte or NK cell), wherein the CAR comprises an antigen binding domain, a transmembrane domain, and an intracellular signaling domain, e.g., of a co-stimulatory molecule, and wherein the immune cell expresses the CAR and possesses targeted effector activity. In some embodiments, the CAR further comprise a linker region, a hinge region, and/or at least one costimulatory domains. In some embodiments, introducing the CAR into the cell comprises introducing a nucleic acid sequence encoding the CAR. In some embodiments, introducing the nucleic acid sequence comprises electroporating a mRNA encoding the CAR.

In some embodiments, the cell may be an immune cell, such as T lymphocyte or an NK cell. A T lymphocyte can be any T cell, such as a cultured T cell, e.g., a primary T cell, or a T cell from a cultured T cell line, e.g., TIB-153™, Jurkat, SupT1, etc., or a T cell obtained from a mammal. If obtained from a mammal, a T cell can be obtained from numerous sources, including but not limited to blood, bone marrow, lymph node, the thymus, or other tissues or fluids. T cells can also be enriched for or purified. In some embodiments, the T cell is a human T cell. In some embodiments, the T cell may be a T cell isolated from a human. A T cell can be any type of T cell and can be of any developmental stage, including but not limited to, CD4+/CD8+ double positive T cells, CD4+ helper T cells, e.g., Th1 and Th2 cells, CD8+ T cells (e.g., cytotoxic T cells), tumor infiltrating cells, memory T cells, naïve T cells, and the like. A T cell may be a CD8+ T cell or a CD4+ T cell. In some embodiments, the T cell is an alpha/beta T cell. In some embodiments, the T cell is a gamma/delta T cell. In some embodiments, the immune cell is a natural killer T cell (NKT cell). In some embodiments, the immune cell is a natural killer cell (NK cell).

Methods of introducing and expressing genes, such as the CAR, into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, transduction (e.g., lentiviral transduction, retroviral transduction), electroporation (e.g., DNA or RNA electroporation), and the like. Methods for producing cells comprising vectors and/or exogenous nucleic acids are well known in the art. See, for example, Sambrook et al., 2012, MOLECULAR CLONING: A LABORATORY MANUAL, volumes 1-4, Cold Spring Harbor Press, NY). Nucleic acids can be introduced into target cells using commercially available methods which include electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany). Nucleic acids can also be introduced into cells using cationic liposome mediated transfection using lipofection, using polymer encapsulation, using peptide mediated transfection, or using biolistic particle delivery systems such as “gene guns” (see, for example, Nishikawa, et al. Hum Gene Ther., 12(8):861-70 (2001).

In one aspect, the DNA or RNA construct is introduced into the cells by electroporation. See, e.g., the formulations and methodology of electroporation of nucleic acid constructs into mammalian cells as taught in US Publication Nos. US 2004/0014645, US 2005/0052630A1, US 2005/0070841 A1, US 2004/0059285A1, and US 2004/0092907A1, which are incorporated herein by reference. The various parameters including electric field strength required for electroporation of any known cell type are generally known in the relevant research literature as well as numerous patents and applications in the field. See e.g., U.S. Pat. Nos. 6,678,556, 7,171,264, and 7,173,116. Apparatus for therapeutic application of electroporation are available commercially, e.g., the MedPulser™ DNA Electroporation Therapy System (Inovio/Genetronics, San Diego, Calif), and are described in patents such as U.S. Pat. Nos. 6,567,694; 6,516,223, 5,993,434, 6,181,964, 6,241,701, and 6,233,482; electroporation may also be used for transfection of cells in vitro as described e.g. in US Publication No. US 2007/0128708A1. Electroporation may also be utilized to deliver nucleic acids into cells in vitro. Accordingly, electroporation-mediated administration into cells of nucleic acids including expression constructs utilizing any of the many available devices and electroporation systems known to those of skill in the art present additional means for delivering DNA or RNA of interest to a target cell.

Biological methods for introducing a polynucleotide of interest into a host cell include the use of DNA and RNA vectors. RNA vectors include vectors having an RNA promoter and/other relevant domains for production of a RNA transcript. Viral vectors, and especially retroviral vectors, have become the most widely used method for inserting genes into mammalian, e.g., human cells. Other viral vectors may be derived from lentivirus, poxviruses, herpes simplex virus, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the molecules described herein, in order to confirm the presence of the nucleic acids in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (e.g., ELISAs and Western blots) or by assays described herein to identify agents falling within the scope of the invention. In some embodiments, the methods further involve selecting the cells in which the exogenous nucleic acids have been introduced (and expressed) from a population of cells, such as through use of a selectable marker.

### CAR Constructs

In some embodiments, the CAR construct includes particular components including an antigen-binding domain (e.g., CD33 binding domain), a transmembrane domain, a hinge domain, and one or more costimulatory/intracellular signaling domains. In some embodiments, the CAR further comprises one or more of a linker region, hinge domain region, and/or one or more costimulatory/intracellular signaling domains. A CAR construct may include any combinations of the exemplary elements described herein, for example, any of the antigen binding domains, transmembrane domains, hinge domains, and any one or more co-stimulatory/intracellular signaling domains described herein. In some embodiments, any of the CARs described herein may further comprise a signal peptide (signal sequence).

In some embodiments, the CAR comprises, from N-terminus to C-terminus: (a) the anti-CD33 antigen-binding domain; (b) the transmembrane region, and (c) the signaling domain. In some embodiments, the CAR does not comprise a costimulatory signaling domain. In some embodiments, the CAR further comprises a signal peptide/signal sequence at the N-terminus of the CAR, which may be removed from the protein upon surface presentation.

Additional embodiments of the invention provide full-length exemplary CAR constructs encoded by any one or more of the nucleic acid sequence sequences set forth below.

1. Mylo-CD8-41BB-CD3z

An exemplary CAR construct, as described herein, comprises a CD33 binding domain (MyloTarg (also referred to as “h67.6”)), a CD8a transmembrane domain, a CD8a hinge domain, a CD137 (4-1BB) co-stimulatory domain, and a CD3ζ intracellular signaling domain.

In some embodiments, a CAR in encoded by a nucleic acid sequence that comprises the sequence that is shown in SEQ ID NO: 9, or in a nucleic acid sequence that is at least 70%, at least 75%, at least 80%, at least 85%, at least 90%, at least 95%, or at least 99% identical to the nucleic acid sequence shown in SEQ ID NO: 9.

[SEQ ID NO: 9]
ATGTGGCTGCAGTCTCTGCTGCTGCTGGGCACCGTGGCCT
GTAGCATCAGCGAGATCGTGCTGACCCAGAGCCCTGGCTC
TCTGGCTGTGTCTCCTGGCGAGCGCGTGACCATGAGCTGC
AAGAGCAGCCAGAGCGTGTTCTTCAGCAGCTCCCAGAAGA
ACTACCTGGCCTGGTATCAGCAGATCCCCGGCCAGAGCCC
CAGACTGCTGATCTACTGGGCCAGCACCAGAGAAAGCGGC
GTGCCCGATAGATTCACCGGCAGCGGCTCTGGCACCGACT
TCACCCTGACAATCAGCAGCGTGCAGCCCGAGGACCTGGC
CATCTACTACTGCCACCAGTACCTGAGCAGCCGGACCTTT
GGCCAGGGCACCAAGCTGGAAATCAAGCGGGGCAGCACAA
GCGGCAGCGGAAAGCCTGGATCTGGCGAGGGCTCTACCAA
GGGCCAGGTGCAGCTGCAGCAGCCTGGCGCCGAAGTCGTG
AAACCTGGCGCCTCCGTGAAGATGTCCTGCAAGGCCAGCG
GCTACACCTTCACCAGCTACTACATCCACTGGATCAAGCA
GACCCCTGGACAGGGCCTGGAATGGGTGGGAGTGATCTAC
CCCGGCAACGACGACATCAGCTACAACCAGAAGTTCCAGG
GCAAGGCCACCCTGACCGCCGACAAGTCTAGCACCACCGC
CTACATGCAGCTGTCCAGCCTGACCAGCGAGGACAGCGCC
GTGTACTACTGCGCCAGAGAAGTGCGGCTGAGCCACTTCG
TGCCCGTGTTTCTGCCCGCCAAGCCTACCACAACCCCTGC
CCCTAGACCTCCTACCCCAGCCCCTACAATCGCCAGCCAG
CCTCTGTCTCTGAGGCCCGAGGCTTCTAGACCAGCTGCTG
GCGGAGCCGTGCACACCAGAGGCCTGGATATCTACATCTG
GGCCCCACTGGCCGGCACCTGTGGCGTGCTGCTGCTGTCT
CTCGTGATCACCAAGAGAGGCCGGAAGAAGCTGCTGTACA
TCTTCAAGCAGCCCTTCATGCGGCCCGTGCAGACCACCCA
GGAAGAGGACGGCTGTAGCTGCCGGTTCCCCGAGGAAGAA
GAAGGGGGCTGCGAGCTGAGAGTGAAGTTCAGCAGAAGCG
CCGACGCCCCTGCCTATCAGCAGGGCCAGAACCAGCTGTA
CAACGAGCTGAACCTGGGCAGACGGGAAGAGTACGACGTG
CTGGACAAGCGGAGAGGCAGGGACCCTGAGATGGGCGGCA
AGCCCAGACGGAAGAACCCTCAGGAAGGCCTGTATAACGA
ACTGCAGAAAGACAAGATGGCCGAGGCCTACTCCGAGATC
GGAATGAAGGGCGAGCGGAGAAGAGGCAAGCCCCCCAGA

In some embodiments, a CAR comprises an amino acid sequence shown in SEQ ID NO: 10, or an amino acid sequence that is at least 70%, at least 75%, at least 80%, at least 85%, at least 90%, at least 95%, or at least 99% identical to the amino acid sequence shown in SEQ ID NO: 10. In amino acid sequences recited in the CAR Constructs section, the following annotations may be used: underline denotes a leader sequence; bold denotes a heavy chain of an antibody or antigen-binding domain; italics denote a linker; dotted underline denotes a light chain of an antibody or antigen-binding domain; long dashed underline denotes a hinge domain; double underline denotes a transmembrane domain; italics with dotted underline denotes a costimulatory domain; and bold underline denotes an intracellular signaling domain.

[SEQ ID NO: 10]
MWLQSLLLLGTVACSISEIVLTQSPGSLAVSPGERVTMSCKSSQSVFFSSSQKNYLAWYQQIPGQSPRLLIYWAS
TRESGVPDRFTGSGSGTDFTLTISSVQPEDLAIYYCHQYLSSRTFGQGTKLEIKRGSTSGSGKPGSGEGSTKGQV
QLQQPGAEVVKPGASVKMSCKASGYTFTSYYIHWIKQTPGQGLEWVGVIYPGNDDISYNQKFQGKATLTADKSST

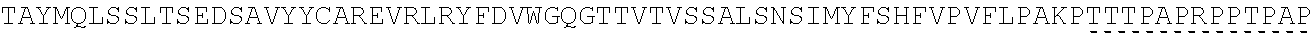

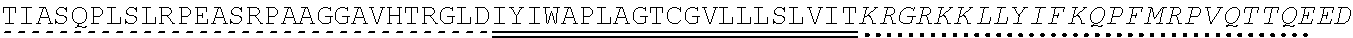

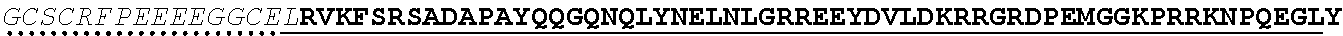

NELQKDKMAEAYSEIGMKGERRRGKGHDGLYQGLSTATKDTYDALHMQALPPR

In some embodiments, a CAR construct as shown in SEQ ID NO: 9 is included in a recombinant expression vector. In some embodiments the recombinant expression vector includes a promoter (e.g., an SFFV promoter or and EF1α promoter). In some embodiments, a recombinant expression vector including the CAR of SEQ ID NO: 9 comprises the sequence that is shown in SEQ ID NO: 11, or in a nucleic acid sequence that is at least 70%, at least 75%, at least 80%, at least 85%, at least 90%, at least 95%, or at least 99% identical to the nucleic acid sequence shown in SEQ ID NO: 11.

In some embodiments, a recombinant expression vector including the CAR of SEQ ID NO: 4 comprises the sequence that is shown in SEQ ID NO: 9, but does not include the SFFV promoter sequence (as shown in SEQ ID NO: 8). In some embodiments, a recombinant expression vector comprises the nucleic acid sequence shown in SEQ ID NO: 4, but does not include the SFFV sequence (as shown in SEQ ID NO: 8), and instead includes an EF1α promoter sequence.

[SEQ ID NO: 11]
Tgggtctctctggttagaccagatctgagcctgggagctc
tctggctaactagggaacccactgcttaagcctcaataaa
gcttgccttgagtgcttcaagtagtgtgtgcccgtctgtt
gtgtgactctggtaactagagatccctcagacccttttag
tcagtgtggaaaatctctagcagtggcgcccgaacaggga
cttgaaagcgaaagggaaaccagaggagctctctcgacgc
aggactcggcttgctgaagcgcgcacggcaagaggcgagg
ggcggcgactggtgagtacgccaaaaattttgactagcgg
aggctagaaggagagagatgggtgcgagagcgtcagtatt
aagcgggggagaattagatcgcgatgggaaaaaattcggt
taaggccagggggaaagaaaaaatataaattaaaacatat
agtatgggcaagcagggagctagaacgattcgcagttaat
cctggcctgttagaaacatcagaaggctgtagacaaatac
tgggacagctacaaccatcccttcagacaggatcagaaga
acttagatcattatataatacagtagcaaccctctattgt
gtgcatcaaaggatagagataaaagacaccaaggaagctt
tagacaagatagaggaagagcaaaacaaaagtaagaccac
cgcacagcaagcggccactgatcttcagacctggaggagg
agatatgagggacaattggagaagtgaattatataaatat
aaagtagtaaaaattgaaccattaggagtagcacccacca
aggcaaagagaagagtggtgcagagagaaaaaagagcagt
gggaataggagctttgttccttgggttcttgggagcagca
ggaagcactatgggcgcagcgtcaatgacgctgacggtac
aggccagacaattattgtctggtatagtgcagcagcagaa
caatttgctgagggctattgaggcgcaacagcatctgttg
caactcacagtctggggcatcaagcagctccaggcaagaa
tcctggctgtgccttggaatgctagttggagtaataaatc
tctggaacagaattggaatcacacgacctggatggagtgg
gacagagaaattaacaattacacaagcttaatacactcct
taattgaagaatcgcaaaaccagcaagaaaagaatgaaca
agaattattggaattagataaatgggcaagtttgtggaat
tggtttaacataacaaattggctgtggtatataaaattat
tcataatgatagtaggaggcttggtaggtttaagaatagt
ttttgctgtactttctatagtgaatagagttaggcaggga
tattcaccattatcgtttcagacccacctcccaaccccga
ggggacccgacaggcccgaaggaatagaagaagaaggtgg
agagagagacagagacagatccattcgattagtgaacgga
tctcgacggtatcggttaacttttaaaagaaaagggggga
ttggggggtacagtgcaggggaaagaatagtagacataat
agcaacagacatacaaactaaagaattacaaaaacaaatt
acaaaattcaaaattttatcgatactagtggatctGCGAT
CGCGTAACGCCATTTTGCAAGGCATGGAAAAATACCAAAC
CAAGAATAGAGAAGTTCAGATCAAGGGCGGGTACATGAAA
ATAGCTAACGTTGGGCCAAACAGGATATCTGCGGTGAGCA
GTTTCGGCCCCGGCCCGGGGCCAAGAACAGATGGTCACCG
CAGTTTCGGCCCCGGCCCGAGGCCAAGAACAGATGGTCCC
CAGATATGGCCCAACCCTCAGCAGTTTCTTAAGACCCATC
AGATGTTTCCAGGCTCCCCCAAGGACCTGAAATGACCCTG
CGCCTTATTTGAATTAACCAATCAGCCTGCTTCTCGCTTC
TGTTCGCGCGCTTCTGCTTCCCGAGCTCTATAAAAGAGCT
CACAACCCCTCACTCGGCGCGCCAGTCCTCCGACAGACTG
AGTCGTCTAGAgctagcGGATCCCCGGAATTCGACGCCAC
CATGTGGCTGCAGTCTCTGCTGCTGCTGGGCACCGTGGCC
TGTAGCATCAGCGAGATCGTGCTGACCCAGAGCCCTGGCT
CTCTGGCTGTGTCTCCTGGCGAGCGCGTGACCATGAGCTG
CAAGAGCAGCCAGAGCGTGTTCTTCAGCAGCTCCCAGAAG
AACTACCTGGCCTGGTATCAGCAGATCCCCGGCCAGAGCC
CCAGACTGCTGATCTACTGGGCCAGCACCAGAGAAAGCGG
CGTGCCCGATAGATTCACCGGCAGCGGCTCTGGCACCGAC
TTCACCCTGACAATCAGCAGCGTGCAGCCCGAGGACCTGG
CCATCTACTACTGCCACCAGTACCTGAGCAGCCGGACCTT
TGGCCAGGGCACCAAGCTGGAAATCAAGCGGGGCAGCACA
AGCGGCAGCGGAAAGCCTGGATCTGGCGAGGGCTCTACCA
AGGGCCAGGTGCAGCTGCAGCAGCCTGGCGCCGAAGTCGT
GAAACCTGGCGCCTCCGTGAAGATGTCCTGCAAGGCCAGC
GGCTACACCTTCACCAGCTACTACATCCACTGGATCAAGC
AGACCCCTGGACAGGGCCTGGAATGGGTGGGAGTGATCTA
CCCCGGCAACGACGACATCAGCTACAACCAGAAGTTCCAG
GGCAAGGCCACCCTGACCGCCGACAAGTCTAGCACCACCG
CCTACATGCAGCTGTGCCAGGGAACCACCGTGACCGTGTC
TAGCGCCCTGAGCAACAGCATCATGTACTTCAGCCACTTC
GTGCCCGTGTTTCTGCCCGCCAAGCCTACCACAACCCCTG
CCCCTAGACCTCCTACCCCAGCCCCTACAATCGCCAGCCA
GCCTCTGTCTCTGAGGCCCGAGGCTTCTAGACCAGCTGCT
GGCGGAGCCGTGCACACCAGAGGCCTGGATATCTACATCT
GGGCCCCACTGGCCGGCACCTGTGGCGTGCTGCTGCTGTC
TCTCGTGATCACCAAGAGAGGCCGGAAGAAGCTGCTGTAC
ATCTTCAAGCAGCCCTTCATGCGGCCCGTGCAGACCACCC
AGGAAGAGGACGGCTGTAGCTGCCGGTTCCCCGAGGAAGA
AGAAGGGGGCTGCGAGCTGAGAGTGAAGTTCAGCAGAAGC
GCCGACGCCCCTGCCTATCAGCAGGGCCAGAACCAGCTGT
ACAACGAGCTGAACCTGGGCAGACGGGAAGAGTACGACGT
GCTGGACAAGCGGAGAGGCAGGGACCCTGAGATGGGCGGC
AAGCCCAGACGGAAGAACCCTCAGGAAGGCCTGTATAACG
AACTGCAGAAAGACAAGATGGCCGAGGCCTACTCCGAGAT
CGGAATGAAGGGCGAGCGGAGAAGAGGCAAGGGCCACGAT
GGACTGTACCAGGGCCTGAGCACCGCCACCAAGGACACCT
ATGACGCCCTGCACATGCAGGCCCTGCCCCCCAGAgaggg
cagaggaagtcttctaacatgcggtgacgtggaggagaat
cccggcccttccgggatgaccgagtacaagcccacggtgc
gcctcgccacccgcgacgacgtccccagggccgtacgcac
cctcgccgccgcgttcgccgactaccccgccacgcgccac
accgtcgatccggaccgccacatcgagcgggtcaccgagc
tgcaagaactcttcctcacgcgcgtcgggctcctggagac
ctccgcgccccgcaacctccccttctacgagcggctcggc
ttcaccgtcaccgccgacgtcgaggtgcccgaaggaccgc
gcacctggtgcatgacccgcaagcccggtgcctgaAAGGT
TAATTAAGGCGCGCCCAATTGaatcaacctctggattaca
aaatttgtgaaagattgactggtattcttaactatgttgc
tccttttacgctatgtggatacgctgctttaatgcctttg
tatcatgctattgcttcccgtatggctttcattttctcct
ccttgtataaatcctggttgctgtctctttatgaggagtt
gtggcccgttgtcaggcaacgtggcgtggtgtgcactgtg
tttgctgacgcaacccccactggttggggcattgccacca
cctgtcagctcctttccgggactttcgctttccccctccc
tattgccacggcggaactcatcgccgcctgccttgcccgc
tgctggacaggggctcggctgttgggcactgacaattccg
tggtgttgtcggggaaatcatcgtcctttccttggctgct
cgcctgtgttgccacctggattctgcgcgggacgtccttc
tgctacgtcccttcggccctcaatccagcggaccttcctt
cccgcggcctgctgccggctctgcggcctcttccgcgtct
tcgccttcgccctcagacgagtcggatctccctttgggcc
gcctccccgcctggtacctttaagaccaatgacttacaag
gcagctgtagatcttagccactttttaaaagaaaaggggg
gactggaagggctaattcactcccaacgaaaataagatct
gctttttgcttgtactgggtctctctggttagaccagatc
tgagcctgggagctctctggctaactagggaacccactgc
ttaagcctcaataaagcttgccttgagtgcttcaagtagt
gtgtgcccgtctgttgtgtgactctggtaactagagatcc
ctcagacccttttagtcagtgtggaaaatctctagcaCGT
ATGTGTATGATACATAAGGTTATGTATTAATTGTAGCCGC
GTTCTAACGACAATATGTACAAGCCTAATTGTGTAGCATC
TGGCTTACTGAAGCAGACCCTATCATCTCTCTCGTAAACT
GCCGTCAGAGTCGGTTTGGTTGGACGAACCTTCTGAGTTT
CTGGTAACGCCGTCCCGCACCCGGAAATGGTCAGCGAACC
AATCAGCAGGGTCATCGCTAGCCAGATCCTCTACGCCGGA
CGCATCGTGGCCGGCATCACCGGCGCCACAGGTGCGGTTG
CTGGCGCCTATATCGCCGACATCACCGATGGGGAAGATCG
GGCTCGCCACTTCGGGCTCATGAGCGCTTGTTTCGGCGTG
GGTATGGTGGCAGGCCCCGTGGCCGGGGGACTGTTGGGCG
CCATCTCCTTGCATGCACCATTCCTTGCGGCGGCGGTGCT
CAACGGCCTCAACCTACTACTGGGCTGCTTCCTAATGCAG
GAGTCGCATAAGGGAGAGCGTCGAATGGTGCACTCTCAGT
ACAATCTGCTCTGATGCCGCATAGTTAAGCCAGCCCCGAC
ACCCGCCAACACCCGCTGACGCGCCCTGACGGGCTTGTCT
GCTCCCGGCATCCGCTTACAGACAAGCTGTGACCGTCTCC
GGGAGCTGCATGTGTCAGAGGTTTTCACCGTCATCACCGA
AACGCGCGAGACGAAAGGGCCTCGTGATACGCCTATTTTT
ATAGGTTAATGTCATGATAATAATGGTTTCTTAGACGTCA
GGTGGCACTTTTCGGGGAAATGTGCGCGGAACCCCTATTT
GTTTATTTTTCTAAATACATTCAAATATGTATCCGCTCAT
GAGACAATAACCCTGATAAATGCTTCAATAATATTGAAAA
AGGAAGAGTATGAGTATTCAACATTTCCGTGTCGCCCTTA
TTCCCTTTTTTGCGGCATTTTGCCTTCCTGTTTTTGCTCA
CCCAGAAACGCTGGTGAAAGTAAAAGATGCTGAAGATCAG
TTGGGTGCACGAGTGGGTTACATCGAACTGGATCTCAACA
GCGGTAAGATCCTTGAGAGTTTTCGCCCCGAAGAACGTTT
TCCAATGATGAGCACTTTTAAAGTTCTGCTATGTGGCGCG
GTATTATCCCGTATTGACGCCGGGCAAGAGCAACTCGGTC
GCCGCATACACTATTCTCAGAATGACTTGGTTGAGTACTC
ACCAGTCACAGAAAAGCATCTTACGGATGGCATGACAGTA
AGAGAATTATGCAGTGCTGCCATAACCATGAGTGATAACA
CTGCGGCCAACTTACTTCTGACAACGATCGGAGGACCGAA
GGAGCTAACCGCTTTTTTGCACAACATGGGGGATCATGTA
ACTCGCCTTGATCGTTGGGAACCGGAGCTGAATGAAGCCA
TACCAAACGACGAGCGTGACACCACGATGCCTGTAGCAAT
GGCAACAACGTTGCGCAAACTATTAACTGGCGAACTACTT
ACTCTAGCTTCCCGGCAACAATTAATAGACTGGATGGAGG
CGGATAAAGTTGCAGGACCACTTCTGCGCTCGGCCCTTCC
GGCTGGCTGGTTTATTGCTGATAAATCTGGAGCCGGTGAG
CGTGGGTCTCGCGGTATCATTGCAGCACTGGGGCCAGATG
GTAAGCCCTCCCGTATCGTAGTTATCTACACGACGGGGAG
TCAGGCAACTATGGATGAACGAAATAGACAGATCGCTGAG
ATAGGTGCCTCACTGATTAAGCATTGGTAACTGTCAGACC
AAGTTTACTCATATATACTTTAGATTGATTTAAAACTTCA
TTTTTAATTTAAAAGGATCTAGGTGAAGATCCTTTTTGAT
AATCTCATGACCAAAATCCCTTAACGTGAGTTTTCGTTCC
ACTGAGCGTCAGACCCCGTAGAAAAGATCAAAGGATCTTC
TTGAGATCCTTTTTTTCTGCGCGTAATCTGCTGCTTGCAA
ACAAAAAAACCACCGCTACCAGCGGTGGTTTGTTTGCCGG
ATCAAGAGCTACCAACTCTTTTTCCGAAGGTAACTGGCTT
CAGCAGAGCGCAGATACCAAATACTGTTCTTCTAGTGTAG
CCGTAGTTAGGCCACCACTTCAAGAACTCTGTAGCACCGC
CTACATACCTCGCTCTGCTAATCCTGTTACCAGTGGCTGC
TGCCAGTGGCGATAAGTCGTGTCTTACCGGGTTGGACTCA
AGACGATAGTTACCGGATAAGGCGCAGCGGTCGGGCTGAA
CGGGGGGTTCGTGCACACAGCCCAGCTTGGAGCGAACGAC
CTACACCGAACTGAGATACCTACAGCGTGAGCTATGAGAA
AGCGCCACGCTTCCCGAAGGGAGAAAGGCGGACAGGTATC
CGGTAAGCGGCAGGGTCGGAACAGGAGAGCGCACGAGGGA
GCTTCCAGGGGGAAACGCCTGGTATCTTTATAGTCCTGTC
GGGTTTCGCCACCTCTGACTTGAGCGTCGATTTTTGTGAT
GCTCGTCAGGGGGGCGGAGCCTATGGAAAAACGCCAGCAA
CGCGGCCTTTTTACGGTTCCTGGCCTTTTGCTGGCCTTTT
GCTCACATGTTCTTTCCTGCGTTATCCCCTGATTCTGTGG
ATAACCGTATTACCGCCTTTGAGTGAGCTGATACCGCTCG
CCGCAGCCGAACGACCGAGCGCAGCGAGTCAGTGAGCGAG
GAAGCGGAAGAGCGCCCAATACGCAAACCGCCTCTCCCCG
CGCGTTGGCCGATTCATTAATGCAGCTGTGGAATGTGTGT
CAGTTAGGGTGTGGAAAGTCCCCAGGCTCCCCAGCAGGCA
GAAGTATGCAAAGCATGCATCTCAATTAGTCAGCAACCAG
GTGTGGAAAGTCCCCAGGCTCCCCAGCAGGCAGAAGTATG
CAAAGCATGCATCTCAATTAGTCAGCAACCATAGTCCCGC
CCCTAACTCCGCCCATCCCGCCCCTAACTCCGCCCAGTTC
CGCCCATTCTCCGCCCCATGGCTGACTAATTTTTTTTATT
TATGCAGAGGCCGAGGCCGCCTCGGCCTCTGAGCTATTCC
AGAAGTAGTGAGGAGGCTTTTTTGGAGGCCTAGGCTTTTG
CAAAAAGCTTGGACACAAGACAGGCTTGCGAGATATGTTT
GAGAATACCACTTTATCCCGCGTCAGGGAGAGGCAGTGCG
TAAAAAGACGCGGACTCATGTGAAATACTGGTTTTTAGTG
CGCCAGATCTCTATAATCTCGCGCAACCTATTTTCCCCTC
GAACACTTTTTAAGCCGTAGATAAACAGGCTGGGACACTT
CACATGAGCGAAAAATACATCGTCACCTGGGACATGTTGC
AGATCCATGCACGTAAACTCGCAAGCCGACTGATGCCTTC
TGAACAATGGAAAGGCATTATTGCCGTAAGCCGTGGCGGT
CTGTACCGGGTGCGTTACTGGCGCGTGAACTGGGTATTCG
TCATGTCGATACCGTTTGTATTTCCAGCTACGATCACGAC
AACCAGCGCGAGCTTAAAGTGCTGAAACGCGCAGAAGGCG
ATGGCGAAGGCTTCATCGTTATTGATGACCTGGTGGATAC
CGGTGGTACTGCGGTTGCGATTCGTGAAATGTATCCAAAA
GCGCACTTTGTCACCATCTTCGCAAAACCGGCTGGTCGTC
CGCTGGTTGATGACTATGTTGTTGATATCCCGCAAGATAC
CTGGATTGAACAGCCGTGGGATATGGGCGTCGTATTCGTC
CCGCCAATCTCCGGTCGCTAATCTTTTCAACGCCTGGCAC
TGCCGGGCGTTGTTCTTTTTAACTTCAGGCGGGTTACAAT
AGTTTCCAGTAAGTATTCTGGAGGCTGCATCCATGACACA
GGCAAACCTGAGCGAAACCCTGTTCAAACCCCGCTTTAAA
CATCCTGAAACCTCGACGCTAGTCCGCCGCTTTAATCACG
GCGCACAACCGCCTGTGCAGTCGGCCCTTGATGGTAAAAC
CATCCCTCACTGGTATCGCATGATTAACCGTCTGATGTGG
ATCTGGCGCGGCATTGACCCACGCGAAATCCTCGACGTCC
AGGCACGTATTGTGATGAGCGATGCCGAACGTACCGACGA
TGATTTATACGATACGGTGATTGGCTACCGTGGCGGCAAC
TGGATTTATGAGTGGGCCCCGGATCTTTGTGAAGGAACCT
TACTTCTGTGGTGTGACATAATTGGACAAACTACCTACAG
AGATTTAAAGCTCTAAGGTAAATATAAAATTTTTAAGTGT
ATAATGTGTTAAACTACTGATTCTAATTGTTTGTGTATTT
TAGATTCCAACCTATGGAACTGATGAATGGGAGCAGTGGT
GGAATGCCTTTAATGAGGAAAACCTGTTTTGCTCAGAAGA
AATGCCATCTAGTGATGATGAGGCTACTGCTGACTCTCAA
CATTCTACTCCTCCAAAAAAGAAGAGAAAGGTAGAAGACC
CCAAGGACTTTCCTTCAGAATTGCTAAGTTTTTTGAGTCA
TGCTGTGTTTAGTAATAGAACTCTTGCTTGCTTTGCTATT
TACACCACAAAGGAAAAAGCTGCACTGCTATACAAGAAAA
TTATGGAAAAATATTCTGTAACCTTTATAAGTAGGCATAA
CAGTTATAATCATAACATACTGTTTTTTCTTACTCCACAC
AGGCATAGAGTGTCTGCTATTAATAACTATGCTCAAAAAT
TGTGTACCTTTAGCTTTTTAATTTGTAAAGGGGTTAATAA
GGAATATTTGATGTATAGTGCCTTGACTAGAGATCATAAT
CAGCCATACCACATTTGTAGAGGTTTTACTTGCTTTAAAA
AACCTCCCACACCTCCCCCTGAACCTGAAACATAAAATGA
ATGCAATTGTTGTTGTTAACTTGTTTATTGCAGCTGGTTT
GTCCAAACTCATCAATGTATCTTATCATGTCTGGATCAAC
TGGATAACTCAAGCTAACCAAAATCATCCCAAACTTCCCA
CCCCATACCCTATTACCACTGCCAATTACCTGTGGTTTCA
TTTACTCTAAACCTGTGATTCCTCTGAATTATTTTCATTT
TAAAGAAATTGTATTTGTTAAATATGTACTACAAACTTAG
TAG

2. Mylo-CD28-CD28-CD3z

An exemplary CAR construct, as described herein, comprises a CD33 binding domain (MyloTarg (h67.6)), a C28 transmembrane domain, a CD28 hinge domain, a CD28 co-stimulatory domain, and a CD3ζ intracellular signaling domain.

In some embodiments, a CAR in encoded by a nucleic acid sequence that comprises the sequence that is shown in SEQ ID NO: 12, or in a nucleic acid sequence that is at least 70%, at least 75%, at least 80%, at least 85%, at least 90%, at least 95%, or at least 99% identical to the nucleic acid sequence shown in SEQ ID NO: 12.

[SEQ ID NO: 12]
GTGCAGTCTGGCGCCGAAGTGAAGAAACCCGGCAGCAGCG
TGAAGGTGTCCTGCAAGGCCAGCGGCTACACCATCACCGA
CAGCAACATCCACTGGGTGCGCCAGGCCCCTGGCCAGAGC
CTGGAATGGATCGGCTACATCTACCCCTACAACGGCGGCA
CCGACTACAACCAGAAGTTCAAGAACCGGGCCACCCTGAC
CGTGGACAACCCCACCAACACCGCCTACATGGAACTGAGC
AGCCTGCGGAGCGAGGACACCGCCTTCTACTACTGCGTGA
ACGGCAACCCCTGGCTGGCCTACTGGGGCCAGGGAACCCT
GGTGACAGTGTCTAGCGGCGGAGGCGGATCTGGAGGGGGA
GGATCTGGCGGCGGAGGAAGCGACATCCAGCTGACCCAGA
GCCCCAGCACCCTGAGCGCCAGCGTGGGCGACAGAGTGAC
CATCACCTGTCGGGCCAGCGAGAGCCTGGACAACTACGGC
ATCCGGTTTCTGACCTGGTTCCAGCAGAAGCCCGGCAAGG
CCCCCAAGCTGCTGATGTACGCCGCCAGCAATCAGGGCAG
CGGCGTGCCCAGCAGATTCAGCGGCTCTGGCAGCGGAACC
GAGTTCACCCTGACCATCAGCAGCCTGCAGCCCGACGACT
TCGCCACCTACTACTGCCAGCAGACCAAAGAGGTGCCCTG
GTCCTTCGGCCAGGGCACCAAGGTGGAAGTGAAGCGGACT
AGTTCCGGAGCCGCCGCCATCGAAGTGATGTACCCCCCTC
CCTACCTGGATAACGAGAAGAGCAACGGCACCATCATCCA
CGTGAAGGGAAAGCACCTGTGTCCCAGCCCCCTGTTTCCC
GGCCCTAGCAAGCCCTTCTGGGTGCTGGTGGTGGTCGGCG
GAGTGCTGGCCTGCTACAGCCTCCTGGTGACCGTGGCCTT
CATCATCTTCTGGGTGAGGAGCAAGAGGTCCAGGCTGCTG
CACAGCGACTACATGAATATGACCCCCAGAAGGCCCGGCC
CCACCAGAAAGCACTATCAGCCCTACGCCCCCCCCAGGGA
CTTTGCCGCCTACAGGAGCAGGGTGAAGTTCAGCAGATCC
GCCGATGCCCCTGCTTACCAGCAGGGCCAGAACCAGCTGT
ATAACGAGCTGAACCTGGGCAGGAGGGAGGAATACGACGT
GCTGGATAAGAGGAGGGGAAGGGACCCCGAGATGGGCGGA
AAGCCCAGGAGGAAGAACCCCCAGGAGGGCCTGTACAATG
AGCTGCAGAAAGACAAGATGGCCGAGGCCTACAGCGAGAT
CGGCATGAAGGGCGAGAGGAGGAGGGGCAAGGGCCATGAC
GGCCTGTACCAAGGCCTGTCCACCGCCACCAAGGATACCT
ACGACGCCCTGCACATGCAGGCCCTGCCTCCCAGGGGATC
C

In some embodiments, a CAR comprises an amino acid sequence shown in SEQ ID NO: 13, or an amino acid sequence that is at least 70%, at least 75%, at least 80%, at least 85%, at least 90%, at least 95%, or at least 99% identical to the amino acid sequence shown in SEQ ID NO: 13.

[SEQ ID NO: 13]
MALPVTALLLPLALLLHAARPEVQLVQSGAEVKKPGSSVKVSCKASGYTITDSNIHWVRQAPGQSLEWIGYIYPY
NGGTDYNQKFKNRATLTVDNPTNTAYMELSSLRSEDTAFYYCVNGNPWLAYWGQGTLVTVSSGGGGSGGGGSGGG

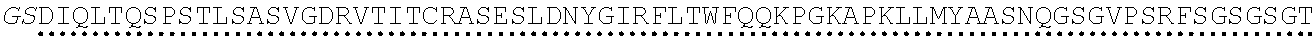

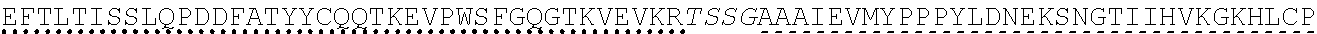

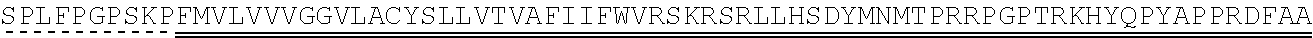

YRSRVKFSRSADAPAYQQGQNQLYNELNLGRREEYDVLDKRRGRDPEMGGKPRRKNPQEGLYNELQKDKMAEAYS
EIGMKGERRRGKGHDGLYQGLSTATKDTYDALHMQALPPRGS

In some embodiments, a CAR construct as shown in SEQ ID NO: 12 is included in a recombinant expression vector. In some embodiments the recombinant expression vector includes a promoter (e.g., an SFFV promoter or and EF1α promoter). In some embodiments, a recombinant expression vector including the CAR of SEQ ID NO: 12 comprises the sequence that is shown in SEQ ID NO: 14, or in a nucleic acid sequence that is at least 70%, at least 75%, at least 80%, at least 85%, at least 90%, at least 95%, or at least 99% identical to the nucleic acid sequence shown in SEQ ID NO: 14.

In some embodiments, a recombinant expression vector including the CAR of SEQ ID NO: 5 comprises the sequence that is shown in SEQ ID NO: 14, but does not include the SFFV promoter sequence (as shown in SEQ ID NO: 8). In some embodiments, a recombinant expression vector comprises the nucleic acid sequence shown in SEQ ID NO: 14, but does not include the SFFV sequence (as shown in SEQ ID NO: 8), and instead includes an EF1α promoter sequence.

[SEQ ID NO: 14]
Tgggtctctctggttagaccagatctgagcctgggagctc
tctggctaactagggaacccactgcttaagcctcaataaa
gcttgccttgagtgcttcaagtagtgtgtgcccgtctgtt
gtgtgactctggtaactagagatccctcagacccttttag
tcagtgtggaaaatctctagcagtggcgcccgaacaggga
cttgaaagcgaaagggaaaccagaggagctctctcgacgc
aggactcggcttgctgaagcgcgcacggcaagaggcgagg
ggcggcgactggtgagtacgccaaaaattttgactagcgg
aggctagaaggagagagatgggtgcgagagcgtcagtatt
aagcgggggagaattagatcgcgatgggaaaaaattcggt
taaggccagggggaaagaaaaaatataaattaaaacatat
agtatgggcaagcagggagctagaacgattcgcagttaat
cctggcctgttagaaacatcagaaggctgtagacaaatac
tgggacagctacaaccatcccttcagacaggatcagaaga
acttagatcattatataatacagtagcaaccctctattgt
gtgcatcaaaggatagagataaaagacaccaaggaagctt
tagacaagatagaggaagagcaaaacaaaagtaagaccac
cgcacagcaagcggccactgatcttcagacctggaggagg
agatatgagggacaattggagaagtgaattatataaatat
aaagtagtaaaaattgaaccattaggagtagcacccacca
aggcaaagagaagagtggtgcagagagaaaaaagagcagt
gggaataggagctttgttccttgggttcttgggagcagca
ggaagcactatgggcgcagcgtcaatgacgctgacggtac
aggccagacaattattgtctggtatagtgcagcagcagaa
caatttgctgagggctattgaggcgcaacagcatctgttg
caactcacagtctggggcatcaagcagctccaggcaagaa
tcctggctgtggaaagatacctaaaggatcaacagctcct
ggggatttggggttgctctggaaaactcatttgcaccact
gctgtgccttggaatgctagttggagtaataaatctctgg
aacagaattggaatcacacgacctggatggagtgggacag
agaaattaacaattacacaagcttaatacactccttaatt
gaagaatcgcaaaaccagcaagaaaagaatgaacaagaat
tattggaattagataaatgggcaagtttgtggaattggtt
taacataacaaattggctgtggtatataaaattattcata
atgatagtaggaggcttggtaggtttaagaatagtttttg
ctgtactttctatagtgaatagagttaggcagggatattc
accattatcgtttcagacccacctcccaaccccgagggga
cccgacaggcccgaaggaatagaagaagaaggtggagaga
gagacagagacagatccattcgattagtgaacggatctcg
acggtatcggttaacttttaaaagaaaaggggggattggg
gggtacagtgcaggggaaagaatagtagacataatagcaa
cagacatacaaactaaagaattacaaaaacaaattacaaa
attcaaaattttatcgatactagtggatctGCGATCGCGT
AACGCCATTTTGCAAGGCATGGAAAAATACCAAACCAAGA
ATAGAGAAGTTCAGATCAAGGGCGGGTACATGAAAATAGC
TAACGTTGGGCCAAACAGGATATCTGCGGTGAGCAGTTTC
GGCCCCGGCCCGGGGCCAAGAACAGATGGTCACCGCAGTT
TCGGCCCCGGCCCGAGGCCAAGAACAGATGGTCCCCAGAT
ATGGCCCAACCCTCAGCAGTTTCTTAAGACCCATCAGATG
TTTCCAGGCTCCCCCAAGGACCTGAAATGACCCTGCGCCT
TATTTGAATTAACCAATCAGCCTGCTTCTCGCTTCTGTTC
GCGCGCTTCTGCTTCCCGAGCTCTATAAAAGAGCTCACAA
CCCCTCACTCGGCGCGCCAGTCCTCCGACAGACTGAGTCG
TCTAGAgctagcGGATCCCCGGAATTCGACGCCACCATGG
CCCTGCCTGTGACAGCCCTGCTGCTGCCCCTGGCTCTGCT
GCTGCATGCCGCCAGACCTGAGGTGCAGCTGGTGCAGTCT
GGCGCCGAAGTGAAGAAACCCGGCAGCAGCGTGAAGGTGT
CCTGCAAGGCCAGCGGCTACACCATCACCGACAGCAACAT
CCACTGGGTGCGCCAGGCCCCTGGCCAGAGCCTGGAATGG
ATCGGCTACATCTACCCCTACAACGGCGGCACCGACTACA
ACCAGAAGTTCAAGAACCGGGCCACCCTGACCGTGGACAA
CCCCACCAACACCGCCTACATGGAACTGAGCAGCCTGCGG
AGCGAGGACACCGCCTTCTACTACTGCGTGAACGGCAACC
CCTGGCTGGCCTACTGGGGCCAGGGAACCCTGGTGACAGT
GTCTAGCGGCGGAGGCGGATCTGGAGGGGGAGGATCTGGC
GGCGGAGGAAGCGACATCCAGCTGACCCAGAGCCCCAGCA
CCCTGAGCGCCAGCGTGGGCGACAGAGTGACCATCACCTG
TCGGGCCAGCGAGAGCCTGGACAACTACGGCATCCGGTTT
CTGACCTGGTTCCAGCAGAAGCCCGGCAAGGCCCCCAAGC
TGCTGATGTACGCCGCCAGCAATCAGGGCAGCGGCGTGCC
CAGCAGATTCAGCGGCTCTGGCAGCGGAACCGAGTTCACC
CTGACCATCAGCAGCCTGCAGCCCGACGACTTCGCCACCT
ACTACTGCCAGCAGACCAAAGAGGTGCCCTGGTCCTTCGG
CCAGGGCACCAAGGTGGAAGTGAAGCGGACTAGTTCCGGA
GCCGCCGCCATCGAAGTGATGTACCCCCCTCCCTACCTGG
CTAGCAAGCCCTTCTGGGTGCTGGTGGTGGTCGGCGGAGT
GCTGGCCTGCTACAGCCTCCTGGTGACCGTGGCCTTCATC
ATCTTCTGGGTGAGGAGCAAGAGGTCCAGGCTGCTGCACA
GCGACTACATGAATATGACCCCCAGAAGGCCCGGCCCCAC
CAGAAAGCACTATCAGCCCTACGCCCCCCCCAGGGACTTT
GCCGCCTACAGGAGCAGGGTGAAGTTCAGCAGATCCGCCG
ATGCCCCTGCTTACCAGCAGGGCCAGAACCAGCTGTATAA
CGAGCTGAACCTGGGCAGGAGGGAGGAATACGACGTGCTG
GATAAGAGGAGGGGAAGGGACCCCGAGATGGGCGGAAAGC
CCAGGAGGAAGAACCCCCAGGAGGGCCTGTACAATGAGCT
GCAGAAAGACAAGATGGCCGAGGCCTACAGCGAGATCGGC
ATGAAGGGCGAGAGGAGGAGGGGCAAGGGCCATGACGGCC
TGTACCAAGGCCTGTCCACCGCCACCAAGGATACCTACGA
CGCCCTGCACATGCAGGCCCTGCCTCCCAGGGGATCCgag
ggcagaggaagtcttctaacatgcggtgacgtggaggaga
atcccggcccttccgggatgaccgagtacaagcccacggt
gcgcctcgccacccgcgacgacgtccccagggccgtacgc
accctcgccgccgcgttcgccgactaccccgccacgcgcc
acaccgtcgatccggaccgccacatcgagctctacgagcg
gctcggcttcaccgtcaccgccgacgtcgaggtgcccgaa
ggaccgcgcacctggtgcatgacccgcaagcccggtgcct
gaATTAATTAACCAATTGaatcaacctctggattacaaaa
tttgtgaaagattgactggtattcttaactatgttgctcc
ttttacgctatgtggatacgctgctttaatgcctttgtat
catgctattgcttcccgtatggctttcattttctcctcct
tgtataaatcctggttgctgtctctttatgaggagttgtg
gcccgttgtcctcctttccgggactttcgctttccccctc
cctattgccacggcggaactcatcgccgcctgccttgccc
gctgctccctttgggccgcctccccgcctggtacctttaa
gaccaatgacttacaaggcagctgtagatcttagccactt
tttaaaagaaaaggggggactggaagggctaattcactcc
caacgaaaataagatctgctttttgcttgtactgggtctc
tctggttagaccagatctgagcctgggagctctctggcta
actagggaacccactgcttaagcctcaataaagcttgcct
tgagtgcttcaagtagtgtgtgcccgtctgttgtgtgact
ctggtaactagagatccctcagacccttttagtcagtgtg
gaaaatctctagcaCGTATGTGTATGATACATAAGGTTAT
GTATTAATTGTAGCCGCGTTCTAACGACAATATGTACAAG
CCTAATTGTGTAGCATCTGGCTTACTGAAGCAGACCCTAT
CATCTCTCTCGTAAACTGCCGTCAGAGTCGGTTTGGTTGG
ACGAACCTTCTGAGTTTCTGGTAACGCCGTCCCGCACCCG
GAAATGGTCAGCGAACCAATCAGCAGGGTCATCGCTAGCC
AGATCCTCTACGCCGGACGCATCGTGGCCGGCATCACCGG
CGCCACAGGTGCGGTTGCTGGCGCCTATATCGCCGACATC
ACCGATGGGGAAGATCGGGCTCGCCACTTCGGGCTCATGA
GCGCTTGTTTCGGCGTGGGTATGGTGGCAGGCCCCGTGGC
CGGGGGACTGTTGGGCGCCATCTCCTTGCATGCACCATTC
CTTGCGGCGGCGGTGCTCAACGGCCTCAACCTACTACTGG
GCTGCTTCCTAATGCAGGAGTCGCATAAGGGAGAGCGTCG
AATGGTGCACTCTCAGTACAATCTGCTCTGATGCCGCATA
GTTAAGCCAGCCCCGACACCCGCCAACACCCGCTGACGCG
CCCTGACGGGCTTGTCTGCTCCCGGCATCCGCTTACAGAC
AAGCTGTGACCGTCTCCGGGAGCTGCATGTGTCAGAGGTT
TTCACCGTCATCACCGAAACGCGCGAGACGAAAGGGCCTC
GTGATACGCCTATTTTTATAGGTTAATGTCATGATAATAA
TGGTTTCTTAGACGTCAGGTGGCACTTTTCGGGGAAATGT
GCGCGGAACCCCTATTTGTTTATTTTTCTAAATACATTCA
AATATGTATCCGCTCATGAGACAATAACCCTGATAAATGC
TTCAATAATATTGAAAAAGGAAGAGTATGAGTATTCAACA
TTTCCGTGTCGCCCTTATTCCCTTTTTTGCGGCATTTTGC
CTTCCTGTTTTTGCTCACCCAGAAACGCTGGTGAAAGTAA
AAGATGCTGAAGATCAGTTGGGTGCACGAGTGGGTTACAT
CGAACTGGATCTCAACAGCGGTAAGATCCTTGAGAGTTTT
CGCCCCGAAGAACGTTTTCCAATGATGAGCACTTTTAAAG
TTCTGCTATGTGGCGCGGTATTATCCCGTATTGACGCCGG
GCAAGAGCAACTCGGTCGCCGCATACACTATTCTCAGAAT
GACTTGGTTGAGTACTCACCAGTCACAGAAAAGCATCTTA
CGGATGGCATGACAGTAAGAGAATTATGCAGTGCTGCCAT
AACCATGAGTGATAACACTGCGGCCAACTTACTTCTGACA
ACGATCGGAGGACCGAAGGAGCTAACCGCTTTTTTGCACA
ACATGGGGGATCATGTAACTCGCCTTGATCGTTGGGAACC
GGAGCTGAATGAAGCCATACCAAACGACGAGCGTGACACC
ACGATGCCTGTAGCAATGGCAACAACGTTGCGCAAACTAT
TAACTGGCGAACTACTTACTCTAGCTTCCCGGCAACAATT
AATAGACTGGATGGAGGCGGATAAAGTTGCAGGACCACTT
CTGCGCTCGGCCCTTCCGGCTGGCTGGTTTATTGCTGATA
AATCTGGAGCCGGTGAGCGTGGGTCTCGCGGTATCATTGC
AGCACTGGGGCCAGATGGTAAGCCCTCCCGTATCGTAGTT
ATCTACACGACGGGGAGTCAGGCAACTATGGATGAACGAA
ATAGACAGATCGCTGAGATAGGTGCCTCACTGATTAAGCA
TTGGTAACTGTCAGACCAAGTTTACCTCATGACCAAAATC
CCTTAACGTGAGTTTTCGTTCCACTGAGCGTCAGACCCCG
TAGAAAAGATCAAAGGATCTTCTTGAGATCCTTTTTTTCT
GCGCGTAATCTGCTGCTTGCAAACAAAAAAACCACCGCTA
CCAGCGGTGGTTTGTTTGCCGGATCAAGAGCTACCAACTC
TTTTTCCGAAGGTAACTGGCTTCAGCAGAGCGCAGATACC
AAATACTGTTCTTCTAGTGTAGCCGTAGTTAGGCCACCAC
TTCAAGAACTCTGTAGCACCGCCTACATACCTCGCTCTGC
TAATCCTGTTACCAGTGGCTGCTGCCAGTGGCGATAAGTC
GTGTCTTACCGGGTTGGACTCAAGACGATAGTTACCGGAT
AAGGCGCAGCGGTCGGGCTGAACGGGGGGTTCGTGCACAC
AGCCCAGCTTGGAGCGAACGACCTACACCGAACTGAGATA
CCTACAGCGTGAGCTATGAGAAAGCGCCACGCTTCCCGAA
GGGAGAAAGGCGGACAGGTATCCGGTAAGCGGCAGGGTCG
GAACAGGAGAGCGCACGAGGGAGCTTCCAGGGGGAAACGC
CTGGTATCTTTATAGTCCTGTCGGGTTTCGCCACCTCTGA
CTTGAGCGTCGATTTTTGTGATGCTCGTCAGGGGGGCGGA
GCCTATGGAAAAACGCCAGCAACGCGGCCTTTTTACGGTT
CCTGGCCTTTTGCTGGCCTTTTGCTCACATGTTCTTTCCT
GCGTTATCCCCTGATTCTGTGGATAACCGTATTACCGCCT
TTGAGTGAGCTGATACCGCTCGCCGCAGCCGAACGACCGA
GCGCAGCGAGTCAGTGAGCGAGGAAGCGGAAGAGCGCCCA
ATACGCAAACCGCCTCTCCCCGCGCGTTGGCCGATTCATT
AATGCAGCTGTGGAATGTGTGTCAGTTAGGGTGTGGAAAG
TCCCCAGGCTCCCCAGCAGGCAGAAGTATGCAAAGCATGC
ATCTCAATTAGTCAGCAACCAGGTGTGGAAAGTCCCCAGG
CTCCCCAGCAGGCAGAAGTATGCAAAGCATGCATCTCAAT
TAGTCAGCAACCATAGTCCCGCCCCTAACTCCGCCCATCC
CGCCCCTAACTCCGCCCAGTTCCGCCCATTCTCCGCCCCA
TGGCTGACTAATTTTTTTTATTTATGCAGAGGCCGAGGCC
GCCTCGGCCTCTGAGCTATTCCAGAAGTAGTGAGGAGGCT
TTTTTGGAGGCCTAGGCTTTTGCAAAAAGCTTGGACACAA
GACAGGCTTGCGAGATATGTTTGAGAATACCACTTTATCC
CGCGTCAGGGAGAGGCAGTGCGTAAAAAGACGCGGACTCA
TGTGAAATACTGGTTTTTAGTGCGCCAGATCTCTATAATC
TCGCGCAACCTATTTTCCCCTCGAACACTTTTTAAGCCGT
AGATAAACAGGCTGGGACACTTCACATGAGCGAAAAATAC
ATCGTCACCTGGGACATGTTGCAGATCCATGCACGTAAAC
TCGCAAGCCGACTGATGCCTTCTGAACAATGGAAAGGCAT
TATTGCCGTAAGCCGTGGCGGTCTGTACCGGGTGCGTTAC
TGGCGCGTGAACTGGGTATTCGTCATGTCGATACCGTTTG
TATTTCCAGCTACGATCACGACAACCAGCGCGAGCTTAAA
GTGCTGAAACGCGCAGAAGGCGATGGCGAAGGCTTCATCG
TTATTGATGACCTGGTGGATACCGGTGGTATGGTTGATGA
CTATGTTGTTGATATCCCGCAAGATACCTGGATTGAACAG
CCGTGGGATATGGGCGTCGTATTCGTCCCGCCAATCTCCG
GTCGCTAATCTTTTCAACGCCTGGCACTGCCGGGCGTTGT
TCTTTTTAACTTCAGGCGGGTTACAATAGTTTCCAGTAAG
TATTCTGGAGGCTGCATCCATGACACAGGCAAACCTGAGC
GAAACCCTGTTCAAACCCCGCTTTAAACATCCTGAAACCT
CGACGCTAGTCCGCCGCTTTAATCACGGCGCACAACCGCC
TGTGCAGTCGGCCCTTGATGGTAAAACCATCCCTCACTGG
TATCGCATGATTAACCGTCTGATGTGGATCTGGCGCGGCA
TTGACCCACGCGAAATCCTCGACGTCCAGGCACGTATTGT
GATGAGCGATGCCGAACGTACCGACGATGATTTATACGAT
ACGGTGATTGGCTACCGTGGCGGCAACTGGATTTATGAGT
GGGCCCCGGATCTTTGTGAAGGAACCTTACTTCTGTGGTG
TGACATAATTGGACAAACTACCTACAGAGATTTAAAGCTC
TAAGGTAAATATAAAATTTTTAAGTGTATAATGTGTTAAA
CTACTGATTCTAATTGTTTGTGTATTTTAGATTCCAACCT
ATGGAACTGATGAATGGGAGCAGTGGTGGAATGCCTTTAA
TGAGGAAAACCTGTTTTGCTCAGAAGAAATGCCATCTAGT
GATGATGAGGCTACTGCTGACTCTCAACATTCTACTCCTC
CAAAAAAGAAGAGAAAGGTAGAAGACCCCAAGGACTTTCC
TTCAGAATTGCTAAGTTTTTTGAGTCATGCTGTGTTTAGT
AATAGAACTCTTGCTTGCTTTGCTATTTACACCACAAAGG
AAAAAGCTGCACTGCTATACAAGAAAATTATGGAAAAATA
TTCTGTAACCTTTATAAGTAGGCATAACAGTTATAATCAT
AACATACTGTTTTTTCTTACTCCACACAGGCATAGAGTGT
CTGCTATTAATAACTATGCTCAAAAATTGTGTACCTTTAG
CTTTTTAATTTGTAAAGGGGTTAATAAGGAATATTTGATG
TATAGTGCCTTGACTAGAGATCATAATCAGCCATACCACA
TTTGTAGAGGTTTTACTTGCTTTAAAAAACCTCCCACACC
TCCCCCTGAACCTGAAACATAAAATGAATGCAATTGTTGT
TGTTAACTTGTTTATTGCAGCTTATAATGGTTACAAATAA
AGCAATAGCATCACAAATTTCACAAATAAAGCATTTTTTT
CACTGCATTCTAGTTGTGGTTTGTCCAAACTCATCAATGT
ATCTTATCATGTCTGGATCAACTGGATAACTCAAGCTAAC
CAAAATCATCCCAAACTTCCCACCCCATACCCTATTACCA
CTGCCAATTACCTGTGGTTTCATTTACTCTAAACCTGTGA
TTCCTCTGAATTATTTTCATTTTAAAGAAATTGTATTTGT
TAAATATGTACTACAAACTTAGTAG

3. Mylo-CD8-41BB-CD3z_2

An exemplary CAR construct, as described herein, comprises a CD33 binding domain (MyloTarg (h67.6)), a CD8a transmembrane domain, a CD8a hinge domain, a CD137 (4-1BB) co-stimulatory domain, and a CD3ζ intracellular signaling domain.

In some embodiments, a CAR in encoded by a nucleic acid sequence that comprises the sequence that is shown in SEQ ID NO: 15, or in a nucleic acid sequence that is at least 70%, at least 75%, at least 80%, at least 85%, at least 90%, at least 95%, or at least 99% identical to the nucleic acid sequence shown in SEQ ID NO: 15.

[SEQ ID NO: 15]
ATGGCCCTGCCTGTGACAGCCCTGCTGCTGCCCCTGGCTC
TGCTGCTGCATGCCGCCAGACCTGAGGTGCAGCTGGTGCA
GTCTGGCGCCGAAGTGAAGAAACCCGGCAGCAGCGTGAAG
GTGTCCTGCAAGGCCAGCGGCTACACCATCACCGACAGCA
ACATCCACTGGGTGCGCCAGGCCCCTGGCCAGAGCCTGGA
ATGGATCGGCTACATCTACCCCTACAACGGCGGCACCGAC
TACAACCAGAAGTTCAAGAACCGGGCCACCCTGACCGTGG
ACAACCCCACCAACACCGCCTACTGGGGCCAGGGAACCCT
GGTGACAGTGTCTAGCGGCGGAGGCGGATCTGGAGGGGGA
GGATCTGGCGGCGGAGGAAGCGACATCCAGCTGACCCAGA
GCCCCAGCACCCTGAGCGCCAGCGTGGGCGACAGAGTGAC
CATCACCTGTCGGGCCAGCGAGAGCCTGGACAACTACGGC
ATCCGGTTTCTGACCTGGTTCCAGCAGAAGCCCGGCAAGG
CCCCCAAGCTGCTGATGTACGCCGCCAGCAATCAGGGCAG
CGGCGTGCCCAGCAGATTCAGCGGCTCTGGCAGCGGAACC
GAGTTCACCCTGACCATCAGCAGCCTGCAGCCCGACGACT
TCGCCACCTACTACTGCCAGCAGACCAAAGAGGTGCCCTG
GTCCTTCGGCCAGGGCACCAAGGTGGAAGTGAAGCGGACT
AGTTCCGGAACCACGACGCCAGCGCCGCGACCACCAACAC
CGGCGCCCACCATCGCGTCGCAGCCCCTGTCCCTGCGCCC
AGAGGCGTGCCGGCCAGCGGCGGGGGGCGCAGTGCACACG
AGGGGGCTGGACTTCGCCTGTGATATCTACATCTGGGCGC
CCTTGGCCGGGACTTGTGGGGTCCTTCTCCTGTCACTGGT
TATCACCCTTTACTGCAAACGGGGCAGAAAGAAACTCCTG
TATATATTCAAACAACCATTTATGAGACCAGTACAAACTA
CTCAAGAGGAAGATGGCTGTAGCTGCCGATTTCCAGAAGA
AGAAGAAGGAGGATGTGAACTGAGAGTGAAGTTCAGCAGG
AGCGCAGACGCCCCCGCGTACAAGCAGGGCCAGAACCAGC
TCTATAACGAGCTCAATCTAGGACGAAGAGAGGAGTACGA
TGTTTTGGACAAGAGACGTGGCCGGGACCCTGAGATGGGG
GGAAAGCCGAGAAGGAAGAACCCTCAGGAAGGCCTGTACA
ATGAACTGCAGAAAGATAAGATGGCGGAGGCCTACAGTGA
GATTGGGATGAAAGGCGAGCGCCGGAGGGGCAAGGGGCAC
GATGGCCTTTACCAGGGTCTCAGTACAGCCACCAAGGACA
CCTACGACGCCCTTCACATGCAGGCCCTGCCCCCTCGC

In some embodiments, a CAR comprises an amino acid sequence shown in SEQ ID NO: 16, or an amino acid sequence that is at least 70%, at least 75%, at least 80%, at least 85%, at least 90%, at least 95%, or at least 99% identical to the amino acid sequence shown in SEQ ID NO: 16.

[SEQ ID NO: 16]
MALPVTALLLPLALLLHAARPEVQLVQSGAEVKKPGSSVKVSCKASGYTITDSNIHWVRQAPGQSLEWIGYTYPY
NGGTDYNQKFKNRATLTVDNPTNTAYMELSSLRSEDTAFYYCVNGNPWLAYWGQGTLVTVSSGGGGSGGGGSGGG

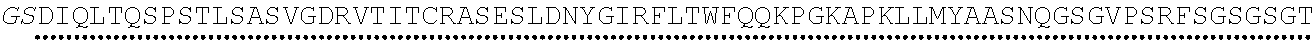

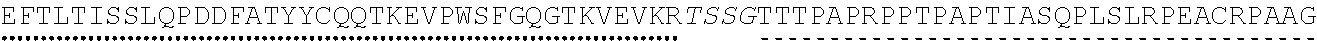

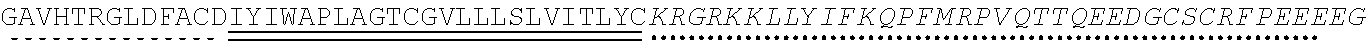

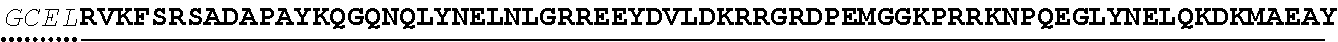

SEIGMKGERRRGKGHDGLYQGLSTATKDTYDALHMQALPPR

In some embodiments, a CAR construct as shown in SEQ ID NO: 15 is included in a recombinant expression vector. In some embodiments the recombinant expression vector includes a promoter (e.g., an SFFV promoter or and EF1α promoter). In some embodiments, a recombinant expression vector including the CAR of SEQ ID NO: 15 comprises the sequence that is shown in SEQ ID NO: 17, or in a nucleic acid sequence that is at least 70%, at least 75%, at least 80%, at least 85%, at least 90%, at least 95%, or at least 99% identical to the nucleic acid sequence shown in SEQ ID NO: 17.

In some embodiments, a recombinant expression vector including the CAR of SEQ ID NO: 15 comprises the sequence that is shown in SEQ ID NO: 17, but does not include the SFFV promoter sequence (as shown in SEQ ID NO: 8). In some embodiments, a recombinant expression vector comprises the nucleic acid sequence shown in SEQ ID NO: 17, but does not include the SFFV sequence (as shown in SEQ ID NO: 8), and instead includes an EF1α promoter sequence.

[SEQ ID NO: 17]
Tgggtctctctggttagaccagatctgagcctgggagctc
tctggctaactagggaacccactgcttaagcctcaataaa
gcttgccttgagtgcttcaagtagtgtgtgcccgtctgtt
gtgtgactctggtaactagagatccctcagacccttttag
tcagtgtggaaaatctctagcagtggcgcccgaacaggga
cttgaaagcgaaagggaaaccagaggagctctctcgacgc
aggactcggcttgctgaagcgcgcacggcaagaggcgagg
ggcggcgactggtgagtacgccaaaaattttgactagcgg
aggctagaaggagagagatgggtgcgagagcgtcagtatt
aagcgggggagaattagatcgcgatgggaaaaaattcggt
taaggccagggggaaagaaaaaatataaattaaaacatat
agtatgggcaagcagggagctagaacgattcgcagttaat
cctggcctgttagaaacatcagaaggctgtagacaaatac
tgggacagctacaaccatcccttcagacaggatcagaaga
acttagatcattatataatacagtagcaaccctctattgt
gtgcatcaaaggatagagataaaagacaccaaggaagctt
tagacaagatagaggaagagcaaaacaaaagtaagaccac
cgcacagcaagcggccactgatcttcagacctggaggagg
agatatgagggacaattggagaagtgaattatataaatat
aaagtagtaaaaattgaaccattaggagtagcacccacca
aggcaaagagaagagtggtgcagaggtcaatgacgctgac
ggtacaggccagacaattattgtctggtatagtgcagcag
cagaacaatttgctgagggctattgaggcgcaacagcatc
tgttgcaactcacagtctggggcatcaagcagctccaggc
aagaatcctggctgtgccttggaatgctagttggagtaat
aaatctctggaacagaattggaatcacacgacctggatgg
agtgggacagagaaattaacaattacacaagcttaataca
ctccttaattgaagaatcgcaaaaccagcaagaaaagaat
gaacaagaattattggaattagataaatgggcaagtttgt
ggaattggtttaacataacaaattggctgtggtatataaa
attattcataatgatagtaggaggcttggtaggtttaaga
atagtttttgctgtactttctatagtgaatagagttaggc
agggatattcaccattatcgtttcagacccacctcccaac
cccgaggggacccgacaggcccgaaggaatagaagaagaa
ggtggagagagagacagagacagatccattcgattagtga
acggatctcgacggtatcggttaacttttaaaagaaaagg
ggggattggggggtacagtgcaggggaaagaatagtagac
ataatagcaacagacatacaaactaaagaattacaaaaac
aaattacaaaattcaaaattttatcgatactagtggatct
GCGATCGCGTAACGCCATTTTGCAAGGCATGGAAAAATAC
CAAACCAAGAATAGAGAAGTTCAGATCAAGGGCGGGTACA
TGAAAATAGCTAACGTTGGGCCAAACAGGATATCTGCGGT
GAGCAGTTTCGGCCCCGGCCCGGGGCCAAGAACAGATGGT
CACCGCAGTTTCGGCCCCGGCCCGAGGCCAAGAACAGATG
GTCCCCAGATATGGCCCAACCCTCAGCAGTTTCTTAAGAC
CCATCAGATGTTTCCAGGCTCCCCCAAGGACCTGAAATGA
CCCTGCGCCTTATTTGAATTAACCAATCAGCCTGCTTCTC
GCTTCTGTTCGCGCGCTTCTGCTTCCCGAGCTCTATAAAA
GAGCTCACAACCCCTCACTCGGCGCGCCAGTCCTCCGACA
GACTGAGTCGTCTAGAgctagcGGATCCCCGGAATTCGAC
GCCACCATGGCCCTGCCTGTGACAGCCCTGCTGCTGCCCC
TGGCTCTGCTGCTGCATGCCGCCAGACCTGAGGTGCAGCT
GGTGCAGTCTGGCGCCGAAGTGAAGAAACCCGGCAGCAGC
GTGAAGGTGTCCTGCAAGGCCAGCGGCTACACCATCACCG
ACAGCAACATCCACTGGGTGCGCCAGGCCCCTGGCCAGAG
CCTGGAATGGATCGGCTACATCTACCCCTACAACGGCGGC
ACCGACTACAACCAGAAGTTCAAGAACCGGGCCACCCTGA
CCGTGGACAACCCCACCAACACCGCCTACATGGAACTGAG
CAGCCTGCGGAGCGAGGACACCGCCTTCTACTACTGCGTG
AACGGCAACCCCTGGCTGGCCTACTGGGGCCAGGGAACCC
TGGTGACAGTGTCTAGCGGCGGAGGCGGATCTGGAGGGGG
AGGATCTGGCGGCGGAGGAAGCGACATCCAGCTGACCCAG
AGCCCCAGCACCCTGAGCGCCAGCGTGGGCGACAGAGTGA
CCATCACCTGTCGGGCCAGCGAGAGCCTGGACAACTACGG
CATCCGGTTTCTGACCTGGTTCCAGCAGAAGCCCGGCAAG
GCCCCCAAGCTGCTGATGTACGCCGCCAGCAATCAGGGCA
GCGGCGTGCCCAGCAGATTCAGCGGCTCTGGCAGCGGAAC
CGAGTTCACCCTGACCATCAGCAGCCTGCAGCCCGACGAC
TTCGCCACCTACTACTGCCAGCAGACCAAAGAGGTGCCCT
GGTCCTTCGGCCAGGGCACCAAGGTGGAAGTGAAGCGGAC
TAGTTCCGGAACCACGACGCCAGCGCCGCGACCACCAACA
CCGGCGCCCACCATCGCGTCGCAGCCCCTGTCCCTGCGCC
CAGAGGCGTGCCGGCCAGCGGCGGGGGGCGCAGTGCACAC
GAGGGGGCTGGACTTCGCCTGTGATATCTACATCTGGGCG
CCCTTGGCCGGGACTTGTGGGGTCCTTCTCCTGTCACTGG
TTATCACCCTTTACTGCAAACGGGGCAGAAAGAAACTCCT
GTATATATTCAAACAACCATTTATGAGACCAGTACAAACT
ACTCAAGAGGAAGATGGCTGTAGCTGCCGATTTCCAGAAG
AAGAAGAAGGAGGATGTGAACTGAGAGTGAAGTTCAGCAG
GAGCGCAGACGCCCCCGCGTACAAGCAGGGCCAGAACCAG
CTCTATAACGAGCTCAATCTAGGACGAAGAGAGGAGTACG
ATGTTTTGGACAAGAGACGTGGCCGGGACCCTGAGATGGG
GGGAAAGCCGAGAAGGAAGAACCCTCAGGAAGGCCTGTAC
AATGAACTGCAGAAAGATAAGATGGCGGAGGCCTACAGTG
AGATTGGGATGAAAGGCGAGCGCCGGAGGGGCAAGGGGCA
CGATGGCCTTTACCAGGGTCTCAGTACAGCCACCAAGGAC
ACCTACGACGCCCTTCACATGCAGGCCCTGCCCCCTCGCg
agggcagaggaagtcttctaacatgcggtgacgtggagga
gaatcccggcccttccgggatgaccgagtacaagcccacg
gtgcgcctcgccacccgcgacgacgtccccagggccgtac
gcaccctcgccgccgcgttcgccgactaccccgccacgcg
ccacaccgtcgatccggaccgccacatcgagcgggtcacc
gagctgcaagaactcttcctcacgcgcgtcgggctcgaca
tcggcaaggtgtgggtcgcggacgacggcgccgcggtggc
ggtctggaccacgccggagagcgtcgaagcgggggcggtg
ttcgccgagatcggcccgcgcatggccgagttgagcggtt
cccggctggccgcgcagcaacagatggaaggcctcctggc
gccgcaccggcccaaggagcccgcgtggttcctggccacc
gtcggcgtctcgcccgaccaccagggcaagggtctgggca
gcgccgtcgtgctccccggagtggaggcggccgagcgcgc
cggggtgcccgccttcctggagacctccgcgccccgcaac
ctccccttctacgagcggctcggcttcaccgtcaccgccg
acgtcgaggtgcccgaaggaccgcgcacctggtgcatgac
ccgcaagcccggtgcctgaATTAATTAACCAATTGaatca
acctctggattacaaaatttgtgaaagattgactggtatt
cttaactatgttgctccttttacgctatgtggatacgctg
ctttaatgcctttgtatcatgctattgcttcccgtatggc
tttcattttctcctccttgtataaatcctggttgctgtct
ctttatgaggagttgtggcccgttgtcaggcaacgtggcg
tggtgtgcactgtgtttgctgacgcaacccccactggttg
gggcattgccaccacctgtcagctcctttccgggactttc
gctttccccctccctattgccacggcggaactcatcgccg
cctgccttgcccgctgctggaaaagaaaaggggggactgg
aagggctaattcactcccaacgaaaataagatctgctttt
tgcttgtactgggtctctctggttagaccagatctgagcc
tgggagctctctggctaactagggaacccactgcttaagc
ctcaataaagcttgccttgagtgcttcaagtagtgtgtgc
ccgtctgttgtgtgactctggtaactagagatccctcaga
cccttttagtcagtgtggaaaatctctagcaCGTATGTGT
ATGATACATAAGGTTATGTATTAATTGTAGCCGCGTTCTA
ACGACAATATGTACAAGCCTAATTGTGTAGCATCTGGCTT
ACTGAAGCAGACCCTATCATCTCTCTCGTAAACTGCCGTC
AGAGTCGGTTTGGTTGGACGAACCTTCTGAGTTTCTGGTA
ACGCCGTCCCGCACCCGGAAATGGTCAGCGAACCAATCAG
CAGGGTCATCGCTAGCCAGATCCTCTACGCCGGACGCATC
GTGGCCGGCATCACCGGCGCCACAGGTGCGGTTGCTGGCG
CCTATATCGCCGACATCACCGATGGGGAAGATCGGGCTCG
CCACTTCGGGCTCATGAGCGCTTGTTTCGGCGTGGGTATG
GTGGCAGGCCCCGTGGCCGGGGGACTGTTGGGCGCCATCT
CCTTGCATGCACCATTCCTTGCGGCGGCGGTGCTCAACGG
CCTCAACCTACTACTGGGCTGCTTCCTAATGCAGGAGTCG
CATAAGGGAGAGCGTCGAATGGTGCACTCTCAGTACAATC
TGCTCTGATGCCGCATAGTTAAGCCAGCCCCGACACCCGC
CAACACCCGCTGACGCGCCCTGACGGGCTTGTCTGCTCCC
GGCATCCGCTTACAGACAAGCTGTGACCGTCTCCGGGAGC
TGCATGTGTCAGAGGTTTTCACCGTCATCACCGAAACGCG
CGAGACGAAAGGGCCTCGTGATACGCCTATTTTTATAGGT
TAATGTCATGATAATAATGGTTTCTTAGACGTCAGGTGGC
ACTTTTCGGGGAAATGTGCGCGGAACCCCTATTTGTTTAT
TTTTCTAAATACATTCAAATATGTATCCGCTCATGAGACA
ATAACCCTGATAAATGCTTCAATAATATTGAAAAAGGAAG
AGTATGAGTATTCAACATTTCCGTGTCGCCCTTATTCCCT
TTTTTGCGGCATTTTGCCTTCCTGTTTTTGCTCACCCAGA
AACGCTGGTGAAAGTAAAAGATGCTGAAGATCAGTTGGGT
GCACGAGTGGGTTACATCGAACTGGATCTCAACAGCGGTA
AGATCCTTGAGAGTTTTCGCCCCGAAGAACGTTTTCCAAT
GATGAGCACTTTTAAAGTTCTGCTATGTGGCGCGGTATTA
TCCCGTATTGACGCCGGGCAAGAGCAACTCGGTCGCCGCA
TACACTATTCTCAGAATGACTTGGTTGAGTACTCACCAGT
CACAGAAAAGCATCTTACGGATGGCATGACAGTAAGAGAA
TTATGCAGTGCTGCCATAACCATGAGTGATAACACTGCGG
CCAACTTACTTCTGACAACGATCGGAGGACCGAAGGAGCT
AACCGCTTTTTTGCACAACATGGGGGATCATGTAACTCGC
CTTGATCGTTGGGAACCGGAGCTGAATGAAGCCATACCAA
ACGACGAGCGTGACACCACGATGCCTGTAGCAATGGCAAC
AACGTTGCGCAAACTATTAACTGGCGAACTACTTACTCTA
GCTTCCCGGCAACAATTAATAGACTGGATGGAGGCGGATA
AAGTTGCAGGACCACTTCTGCGCTCGGCCCTTCCGGCTGG
CTGGTTTATTGCTGATAAATCTGGAGCCGGTGAGCGTGGG
TCTCGCGGTATCATTGCAGCACTGGGGCCAGATGGTAAGC
CCTCCCGTATCGTAGTTATCTACACGACGGGGAGTCAGGC
AACTATGGATGAACGAAATAGACAGATCGCTGAGATAGGT
GCCTCACTGATTAAGCATTGGTAACTGTCAGACCAAGTTT
ACTCATATATACTTTAGATTGATTTAAAACTTCATTTTTA
ATTTAAAAGGATCTAGGTGAAGATCCTTTTTGATAATCTC
ATGACCAAAATCCCTTAACGTGAGTTTTCGTTCCACTGAG
CGTCAGACCCCGTAGAAAAGATCAAAGGATCTTCTTGAGA
TCCTTTTTTTCTGCGCGTAATCTGCTGCTTGCAAACAAAA
AAACCACCGCTACCAGCGGTGGTTTGTTTGCCGGATCAAG
AGCTACCAACTCTTTTTCCGAAGGTAACTGGCTTCAGCAG
AGCGCAGATACCAAATACTGTTCTTCTAGTGTAGCCGTAG
TTAGGCCACCACTTCAAGAACTCTGTAGCACCGCCTACAT
ACCTCGCTCTGCTAATCCTGTTACCAGTGGCTGCTGCCAG
TGGCGATAAGTCGTGTCTTACCGGGTTGGACTCAAGACGA
TAGTTACCGGATAAGGCGCAGCGGTCGGGCTGAACGGGGG
GTTCGTGCACACAGCCCAGCTTGGAGCGAACGACCTACAC
CGAACTGAGATACCTACAGCGTGAGCTATGAGAAAGCGCC
ACGCTTCCCGAAGGGAGAAAGGCGGACAGGTATCCGGTAA
GCGGCAGGGTCGGAACAGGAGAGCGCACGAGGGAGCTTCC
AGGGGGAAACGCCTGGTATCTTTATAGTCCTGTCGGGTTT
CGCCACCTCTGACTTGAGCGTCGATTTTTGTGATGCTCGT
CAGGGGGGCGGAGCCTATGGAAAAACGCCAGCAACGCGGC
CTTTTTACGGTTCCTGGCCTTTTGCTGGCCTTTTGCTCAC
ATGTTCTTTCCTGCGTTATCCCCTGATTCTGTGGATAACC
GTATTACCGCCTTTGAGTGAGCTGATACCGCTCGCCGCAG
CCGAACGACCGAGCGCAGCGAGTCAGTGAGCGAGGAAGCG
GAAGAGCGCCCAATACGCAAACCGCCTCTCCCCGCGCGTT
GGCCGATTCATTAATGCAGCTGTGGAATGTGTGTCAGTTA
GGGTGTGGAAAGTCCCCAGGCTCCCCAGCAGGCAGAAGTA
TGCAAAGCATGCATCTCAATTAGTCAGCAACCAGGTGTGG
AAAGTCCCCAGGCTCCCCAGCAGGCAGAAGTATGCAAAGC
ATGCATCTCAATTAGTCAGCAACCATAGTCCCGCCCCTAA
CTCCGCCCATCCCGCCCCTAACTCCGCCCAGTTCCGCCCA
TTCTCCGCCCCATGGCTGACTAATTTTTTTTATTTATGCA
GAGGCCGAGGCCGCCTCGGCCTCTGAGCTATTCCAGAAGT
AGTGAGGAGGCTTTTTTGGAGGCCTAGGCTTTTGCAAAAA
GCTTGGACACAAGACAGGCTTGCGAGATATGTTTGAGAAT
ACCACTTTATCCCGCGTCAGGGAGAGGCAGTGCGTAAAAA
GACGCGGACTCATGTGAAATACTGGTTTTTAGTGCGCCAG
ATCTCTATAATCTCGCGCAACCTATTTTCCCCTCGAACAC
TTTTTAAGCCGTAGATAAACAGGCTGGGACACTTCACATG
AGCGAAAAATACATCGTCACCTGGGACATGTTGCAGATCC
ATGCACGTAAACTCGCAAGCCGACTGATGCCTTCTGAACA
ATGGAAAGGCATTATTGCCGTAAGCCGTGGCGGTCTGTAC
CGGGTGCGTTACTGGCGCGTGAACTGGGTATTCGTCATGT
CGATACCGTTTGTATTTCCAGCTACGATCACGACAACCAG
CGCGAGCTTAAAGTGCTGAAACGCGCAGAAGGCGATGGCG
AAGGCTTCATCGTTATTGATGACCTGGTGGATACCGGTGG
TACTGCGGTTGCGATTCGTGAAATGTATCCAAAAGCGCAC
TTTGTCACCATCTTCGCAAAACCGGCTGGTCGTCCGCTGG
TTGATGACTATGTTGTTGATATCCCGCAAGATACCTGGAT
TGAACAGCCGTGGGATATGGGCGTCGTATTCGTCCCGCCA
ATCTCCGGTCGCTAATCTTTTCAACGCCTGGCACTGCCGG
GCGTTGTTCTTTTTAACTTCAGGCGGGTTACAATAGTTTC
CAGTAAGTATTCTGGAGGCTGCATCCATGACACAGGCAAA
CCTGAGCGAAACCCTGTTCAAACCCCGCTTTAAACATCCT
GAAACCTCGACGCTAGTCCGCCGCTTTAATCACGGCGCAC
AACCGCCTGTGCAGTCGGCCCGCGAAATCCTCGACGTCCA
GGCACGTATTGTGATGAGCGATGCCGAACGTACCGACGAT
GATTTATACGATACGGTGATTGGCTACCGTGGCGGCAACT
GGATTTATGAGTGGGCCCCGGATCTTTGTGAAGGAACCTT
ACTTCTGTGGTGTGACATAATTGGACAAACTACCTACAGA
GATTTAAAGCTCTAAGGTAAATATAAAATTTTTAAGTGTA
TAATGTGTTAAACTACTGATTCTAATTGTTTGTGTATTTT
AGATTCCAACCTATGGAACTGATGAATGGGAGCAGTGGTG
GAATGCCTTTAATGAGGAAAACCTGTTTTGCTCAGAAGAA
ATGCCATCTAGTGATGATGAGGCTACTGCTGACTCTCAAC
ATTCTACTCCTCCAAAAAAGAAGAGAAAGGTAGAAGACCC
CAAGGACTTTCCTTCAGAATTGCTAAGTTTTTTGAGTCAT
GCTGTGTTTAGTAATAGAACTCTTGCTTGCTTTGCTATTT
ACACCACAAAGGAAAAAGCTGCACTGCTATACAAGAAAAT
TATGGAAAAATATTCTGTAACCTTTATAAGTAGGCATAAC
AGTTATAATCATAACATACTGTTTTTTCTTACTCCACACA
GGCATAGAGTGTCTGCTATTAATAACTATGCTCAAAAATT
GTGTACCTTTAGCTTTTTAATTTGTAAAGGGGTTAATAAG
GAATATTTGATGTATAGTGCCTTGACTAGAGATCATAATC
AGCCATACCACATTTGTAGAGGTTTTACTTGCTTTAAAAA
ACCTCCCACACCTCCCCCTGAACCTGAAACATAAAATGAA
TGCAATTGTTGTTGTTAACTTGTTTATTGCAGCTTATAAT
GGTTACAAATAAAGCAATAGCATCACAAATTTCACAAATA
GGATAACTCAAGCTAACCAAAATCATCCCAAACTTCCCAC
CCCATACCCTATTACCACTGCCAATTACCTGTGGTTTCAT
TTACTCTAAACCTGTGATTCCTCTGAATTATTTTCATTTT
AAAGAAATTGTATTTGTTAAATATGTACTACAAACTTAGT
AG

4. hM195-CD28-CD28-CD3z

An exemplary CAR construct, as described herein, comprises a CD33 binding domain (hM195 (Lintuzumab)), a CD28 transmembrane domain, a CD28 hinge domain, a CD28 co-stimulatory domain, and a CD3ζ intracellular signaling domain.

In some embodiments, a CAR in encoded by a nucleic acid sequence that comprises the sequence that is shown in SEQ ID NO: 18, or in a nucleic acid sequence that is at least 70%, at least 75%, at least 80%, at least 85%, at least 90%, at least 95%, or at least 99% identical to the nucleic acid sequence shown in SEQ ID NO: 18.

[SEQ ID NO: 18]
ATGGCTCTGCCCGTCACAGCTCTGCTGCTGCCTCTGGCCCT
GCTGCTGCACGCCGCCAGACCTCAGGTGCAGCTCGTGCAG
AGCGGCGCTGAGGTGAAGAAACCTGGCAGCAGCGTGAAGG
TGAGCTGCAAGGCCTCCGGCTACACCTTCACCGACTACAA
CATGCACTGGGTGAGGCAAGCCCCTGGCCAGGGACTGGAG
TGGATCGGCTACATCTACCCTTACAACGGCGGCACAGGCT
ACAACCAGAAGTTCAAGTCCAAGGCCACCATCACCGCCGA
TGAGTCCACCAATACCGCCTACATGGAGCTCAGCAGCCTG
AGGTCCGAGGACACAGCCGTCTACTACTGCGCCAGGGGCA
GGCCCGCTATGGACTACTGGGGCCAGGGCACCCTGGTGAC
AGTGAGCTCTGGTGGCGGCGGATCCGGCGGCGGCGGCAGC
GGCGGCGGCGGCTCCGACATTCAGATGACCCAGAGCCCTA
GCAGCCTGAGCGCTTCCGTGGGAGACAGGGTGACCATCAC
ATGCAGGGCCTCCGAGAGCGTGGACAATTACGGCATCAGC
TTCATGAACTGGTTCCAGCAGAAGCCCGGCAAGGCCCCCA
AACTGCTGATCTATGCCGCCAGCAATCAGGGCTCCGGCGT
GCCTAGCAGGTTTTCCGGCAGCGGCAGCGGCACCGACTTT
ACCCTGACCATCTCCAGCCTGCAGCCTGACGATTTCGCCA
CCTACTACTGCCAGCAGAGCAAGGAGGTGCCTTGGACCTT
TGGACAGGGCACAAAGGTGGAGATCAAGTCCGGAGCCGCC
GCCATCGAAGTGATGTACCCCCCTTTTCCCGGCCCTAGCA
AGCCCTTCTGGGTGCTGGTGGTGGTCGGCGGAGTGCTGGC
CTGCTACAGCCTCCTGGTGACCGTGGCCTTCATCATCTTC
TGGGTGAGGAGCAAGAGGTCCAGGCTGCTGCACAGCGACT
ACATGAATATGACCCCCAGAAGGCCCGGCCCCACCAGAAA
GCACTATCAGCCCTACGCCCCCCCCAGGGACTTTGCCGCC
TACAGGAGCAGGGTGAAGTTCAGCAGATCCGCCGATGCCC
CTGCTTACCAGCAGGGCCAGAACCAGCTGTATAACGAGCT
GAACCTGGGCAGGAGGGAGGAATACGACGTGCTGGATAAG
AGGAGGGGAAGGGACCCCGAGATGGGCGGAAAGCCCAGGA
GGAAGAACCCCCAGGAGGGCCTGTACAATGAGCTGCAGAA
AGACAAGATGGCCGAGGCCTACAGCGAGATCGGCATGAAG
GGCGAGAGGAGGAGGGGCAAGGGCCATGACGGCCTGTACC
AAGGCCTGTCCACCGCCACCAAGGATACCTACGACGCCCT
GCACATGCAGGCCCTGCCTCCCAGGGGATCC
C

In some embodiments, a CAR comprises an amino acid sequence shown in SEQ ID NO: 19, or an amino acid sequence that is at least 70%, at least 75%, at least 80%, at least 85%, at least 90%, at least 95%, or at least 99% identical to the amino acid sequence shown in SEQ ID NO: 19.

[SEQ ID NO: 19]
MALPVTALLLPLALLLHAARPQVQLVQSGAEVKKPGSSVKVSCKASGYTFTDYNMHWVRQAPGQGLEWIGYIYPY
NGGTGYNQKFKSKATITADESTNTAYMELSSLRSEDTAVYYCARGRPAMDYWGQGTLVTVSSGGGGSGGGGSGGG

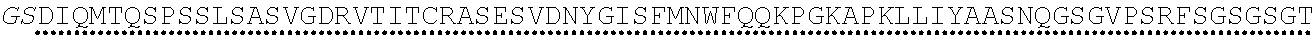

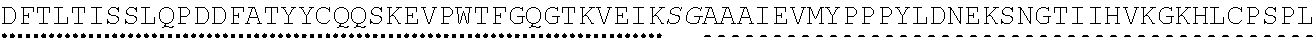

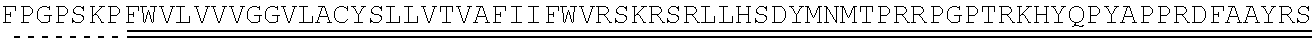

RVKFSRSADAPAYQQGQNQLYNELNLGRREEYDVLDKRRGRDPEMGGKPRRKNPQEGLYNELQKDKMAEAYSEIG
MKGERRRGKGHDGLYQGLSTATKDTYDALHMQALPPRGS

In some embodiments, a CAR construct as shown in SEQ ID NO: 18 is included in a recombinant expression vector. In some embodiments the recombinant expression vector includes a promoter (e.g., an SFFV promoter or and EF1α promoter). In some embodiments, a recombinant expression vector including the CAR of SEQ ID NO: 18 comprises the sequence that is shown in SEQ ID NO: 20, or in a nucleic acid sequence that is at least 70%, at least 75%, at least 80%, at least 85%, at least 90%, at least 95%, or at least 99% identical to the nucleic acid sequence shown in SEQ ID NO: 20.

In some embodiments, a recombinant expression vector including the CAR of SEQ ID NO: 18 comprises the sequence that is shown in SEQ ID NO: 20, but does not include the SFFV promoter sequence (as shown in SEQ ID NO: 8). In some embodiments, a recombinant expression vector comprises the nucleic acid sequence shown in SEQ ID NO: 20, but does not include the SFFV sequence (as shown in SEQ ID NO: 8), and instead includes an EF1α promoter sequence.

[SEQ ID NO: 20]
Tgggtctctctggttagaccagatctgagcctgggagctc
tctggctaactagggaacccactgcttaagcctcaataaa
gcttgccttgagtgcttcaagtagtgtgtgcccgtctgtt
gtgtgactctggtaactagagatccctcagacccttttag
tcagtgtggaaaatctctagcagtggcgcccgaacaggga
cttgaaagcgaaagggaaaccagaggagctctctcgacgc
aggactcggcttgctgaagcgcgcacggcaagaggcgagg
ggcggcgactggtgagtacgccaaaaattttgactagcgg
aggctagaaggagagagatgggtgcgagagcgtcagtatt
aagcgggggagaattagatcgcgatgggaaaaaattcggt
taaggccagggggaaagaaaaaatataaattaaaacatat
agtatgggcaagcagggagctagaacgattcgcagttaat
cctggcctgttagaaacatcagaaggctgtagacaaatac
tgggacagctacaaccatcccttcagacaggatcagaaga
acttagatcattatataatacagtagcaaccctctattgt
gtgcatcaaaggatagagataaaagacaccaaggaagctt
tagacaagatagaggaagagcaaaacaaaagtaagaccac
cgcacagcaagcggccactgatcttcagacctggaggagg
agatatgagggacaattggagaagtgaattatataaatat
aaagtagtaaaaattgaaccattaggagtagcacccacca
aggcaaagagaagagtggtgcagagagaaaaaagagcagt
gggaataggagctttgttccttgggttcttgggagcagca
ggaagcactatgggcgcagcgtcaatgacgctgacggtac
aggccagacaattattgtctggtatagtgcagcagcagaa
caatttgctgagggctattgaggcgcaacagcatctgttg
caactcacagtctggggcatcaagcagctccaggcaagaa
tcctggctgtggaaagatacctaaaggatcaacagctcct
ggggatttggggttgctctggaaaactcatttgcaccact
gctgtgccttggaatgctagttggagtaataaatctctgg
aacagaattggaatcacacgacctggatggagtgggacag
agaaattaacaattacacaagcttaatacactccttaatt
gaagaatcgcaaaaccagcaagaaaagaatgaacaagaat
tattggaattagataaatgggcaagtttgtggaattggtt
taacataacaaattggctgtggtatataaaattattcata
atgatagtaggaggcttggtaggtttaagaatagtttttg
ctgtactttctatagtgaatagagttaggcagggatattc
accattatcgtttcagacccacctcccaaccccgagggga
cccgacaggcccgaaggaatagaagaagaaggtggagaga
gagacagagacagatccattcgattagtgaacggatctcg
acggtatcggttaacttttaaaagaaaaggggggattggg
gggtacagtgcaggggaaagaatagtagacataatagcaa
cagacatacaaactaaagaattacaaaaacaaattacaaa
attcaaaattttatcgatactagtggatctGCGATCGCGT
AACGCCATTTTGCAAGGCATGGAAAAATACCAAACCAAGA
ATAGAGAAGTTCAGATCAAGGGCGGGTACATGAAAATAGC
TAACGTTGGGCCAAACAGGATATCTGCGGTGAGCAGTTTC
GGCCCCGGCCCGGGGCCAAGAACAGATGGTCACCGCAGTT
TCGGCCCCGGCCCGAGGCCAAGAACAGATGGTCCCCAGAT
ATGGCCCAACCCTCAGCAGTTTCTTAAGACCCATCAGATG
TTTCCAGGCTCCCCCAAGGACCTGAAATGACCCTGCGCCT
TATTTGAATTAACCAATCAGCCTGCTTCTCGCTTCTGTTC
GCGCGCTTCTGCTTCCCGAGCTCTATAAAAGAGCTCACAA
CCCCTCACTCGGCGCGCCAGTCCTCCGACAGACTGAGTCG
TCTAGAgctagcGGATCCCCGGAATTCGACGCCACCATGG
CTCTGCCCGTCACAGCTCTGCTGCTGCCTCTGGCCCTGCT
GCTGCACGCCGCCAGACCTCAGGTGCAGCTCGTGCAGAGC
GGCGCTGAGGTGAAGAAACCTGGCAGCAGCGTGAAGGTGA
GCTGCAAGGCCTCCGGCTACACCTTCACCGACTACAACAT
GCACTGGGTGAGGCAAGCCCCTGGCCAGGGACTGGAGTGG
ATCGGCTACATCTACCCTTACAACGGCGGCACAGGCTACA
ACCAGAAGTTCAAGTCCAAGGCCACCATCACCGCCGATGA
GTCCACCAATACCGCCTACATGGAGCTCAGCAGCCTGAGG
TCCGAGGACACAGCCGTCTACTACTGCGCCAGGGGCAGGC
CCGCTATGGACTACTGGGGCCAGGGCACCCTGGTGACAGT
GAGCTCTGGTGGCGGCGGATCCGGCGGCGGCGGCAGCGGC
GGCGGCGGCTCCGACATTCAGATGACCCAGAGCCCTAGCA
GCCTGAGCGCTTCCGTGGGAGACAGGGTGACCATCACATG
CAGGGCCTCCGAGAGCGTGGACAATTACGGCATCAGCTTC
ATGAACTGGTTCCAGCAGAAGCCCGGCAAGGCCCCCAAAC
TGCTGATCTATGCCGCCAGCAATCAGGGCTCCGGCGTGCC
TAGCAGGTTTTCCGGCAGCGGCAGCGGCACCGACTTTACC
CTGACCATCTCCAGCCTGCAGCCTGACGATTTCGCCACCT
ACTACTGCCAGCAGAGCAAGGAGGTGCCTTGGACCTTTGG
ACAGGGCACAAAGGTGGAGATCAAGTCCGGAGCCGCCGCC
ATCGAAGTGATGTACCCCCCTCCCTACCTGGATAACGAGA
AGAGCAACGGCACCATCATCCACGTGAAGGGAAAGCACCT
GTGTCCCAGCCCCCTGTTTCCCGGCCCTAGCAAGCCCTTC
TGGGTGCTGGTGGTGGTCGGCGGAGTGCTGGCCTGCTACA
GCCTCCTGGTGACCGTGGCCTTCATCATCTTCTGGGTGAG
GAGCAAGAGGTCCAGGCTGCTGCACAGCGACTACATGAAT
ATGACCCCCAGAAGGCCCGGCCCCACCAGAAAGCACTATC
AGCCCTACGCCCCCCCCAGGGACTTTGCCGCCTACAGGAG
CAGGGTGAAGTTCAGCAGATCCGCCGATGCCCCTGCTTAC
CAGCAGGGCCAGAACCAGCTGTATAACGAGCTGAACCTGG
GCAGGAGGGAGGAATACGACGTGCTGGATAAGAGGAGGGG
AAGGGACCCCGAGATGGGCGGAAAGCCCAGGAGGAAGAAC
CCCCAGGAGGGCCTGTACAATGAGCTGCAGAAAGACAAGA
TGGCCGAGGCCTACAGCGAGATCGGCATGAAGGGCGAGAG
GAGGAGGGGCAAGGGCCATGACGGCCTGTACCAAGGCCTG
TCCACCGCCACCAAGGATACCTACGACGCCCTGCACATGC
AGGCCCTGCCTCCCAGGGGATCCgagggcagaggaagtct
tctaacatgcggtgacgtggaggagaatcccggcccttcc
gggatgaccgagtacaagcccacggtgcgcctcgccaccc
gcgacgacgtccccagggccgtacgcaccctcgccgccgc
gttcgccgactaccccgccacgcgccacaccgtcgatccg
gaccgccacatcgagcgggtcaccgagctgcaagaactct
tcctcacgcgcgtcgggctcgacatcggcaaggtgtgggt
cgcggacgacggcgccgcggtggcggtctggaccacgccg
gagagcgtcgaagcgggggcggtgttcgccgagatcggcc
cgcgcatggccgagttgagcggttcccggctggccgcgca
gcaacagatggaaggcctcctggcgccgcaccggcccaag
gagcccgcgtggttcctggccaccgtcggcgtctcgcccg
accaccagggcaagggtctgggcagcgccgtcgtgctccc
cggagtggaggcggccgagcgcgccggggtgcccgccttc
ctggagacctccgcgccccgcaacctccccttctacgagc
ggctcggcttcaccgtcaccgccgacgtcgaggtgcccga
aggaccgcgcacctggtgcatgacccgcaagcccggtgcc
tgaATTAATTAACCAATTGaatcaacctctggattacaaa
atttgtgaaagattgactggtattcttaactatgttgctc
cttttacgctatgtggatacgctgctttaatgcctttgta
tcatgctattgcttcccgtatggctttcattttctcctcc
ttgtataaatcctggttgctgtctctttatgaggagttgt
ggcccgttgtcaggcaacgtggcgtggtgtgcactgtgtt
tgctgacgcaacccccactggttggggcattgccaccacc
tgtcagctcctttccgggactttcgctttccccctcccta
ttgccacggcggaactcatcgccgcctgccttgcccgctg
ctggacaggggctcggctgttgggcactgacaattccgtg
gtgttgtcggggaaatcatcgtcctttccttggctgctcg
cctgtgttgccacctggattctgcgcgggacgtccttctg
ctacgtcccttcggccctcaatccagcggaccttccttcc
cgcggcctgctgccggctctgcggcctcttccgcgtcttc
gccttcgccctcagacgagtcggatctccctttgggccgc
ctccccgcctggtacctttaagaccaatgacttacaaggc
agctgtagatcttagccactttttaaaagaaaagggggga
ctggaagggctaattcactcccaacgaaaataagatctgc
tttttgcttgtactgggtctctctggttagaccagatctg
agcctgggagctctctggctaactagggaacccactgctt
aagcctcaataaagcttgccttgagtgcttcaagtagtgt
gtgcccgtctgttgtgtgactctggtaactagagatccct
cagacccttttagtcagtgtggaaaatctctagcaCGTAT
GTGTATGATACATAAGGTTATGTATTAATTGTAGCCGCGT
TCTAACGACAATATGTACAAGCCTAATTGTGTAGCATCTG
GCTTACTGAAGCAGACCCTATCATCTCTCTCGTAAACTGC
CGTCAGAGTCGGTTTGGTTGGACGAACCTTCTGAGTTTCT
GGTAACGCCGTCCCGCACCCGGAAATGGTCAGCGAACCAA
TCAGCAGGGTCATCGCTAGCCAGATCCTCTACGCCGGACG
CATCGTGGCCGGCATCACCGGCGCCACAGGTGCGGTTGCT
GGCGCCTATATCGCCGACATCACCGATGGGGAAGATCGGG
CTCGCCACTTCGGGCTCATGAGCGCTTGTTTCGGCGTGGG
TATGGTGGCAGGCCCCGTGGCCGGGGGACTGTTGGGCGCC
ATCTCCTTGCATGCACCATTCCTTGCGGCGGCGGTGCTCA
ACGGCCTCAACCTACTACTGGGCTGCTTCCTAATGCAGGA
GTCGCATAAGGGAGAGCGTCGAATGGTGCACTCTCAGTAC
AATCTGCTCTGATGCCGCATAGTTAAGCCAGCCCCGACAC
CCGCCAACACCCGCTGACGCGCCCTGACGGGCTTGTCTGC
TCCCGGCATCCGCTTACAGACAAGCTGTGACCGTCTCCGG
GAGCTGCATGTGTCAGAGGTTTTCACCGTCATCACCGAAA
CGCGCGAGACGAAAGGGCCTCGTGATACGCCTATTTTTAT
AGGTTAATGTCATGATAATAATGGTTTCTTAGACGTCAGG
TGGCACTTTTCGGGGAAATGTGCGCGGAACCCCTATTTGT
TTATTTTTCTAAATACATTCAAATATGTATCCGCTCATGA
GACAATAACCCTGATAAATGCTTCAATAATATTGAAAAAG
GAAGAGTATGAGTATTCAACATTTCCGTGTCGCCCTTATT
CCCTTTTTTGCGGCATTTTGCCTTCCTGTTTTTGCTCACC
CAGAAACGCTGGTGAAAGTAAAAGATGCTGAAGATCAGTT
GGGTGCACGAGTGGGTTACATCGAACTGGATCTCAACAGC
GGTAAGATCCTTGAGAGTTTTCGCCCCGAAGAACGTTTTC
CAATGATGAGCACTTTTAAAGTTCTGCTATGTGGCGCGGT
ATTATCCCGTATTGACGCCGGGCAAGAGCAACTCGGTCGC
CGCATACACTATTCTCAGAATGACTTGGTTGAGTACTCAC
CAGTCACAGAAAAGCATCTTACGGATGGCATGACAGTAAG
AGAATTATGCAGTGCTGCCATAACCATGAGTGATAACACT
GCGGCCAACTTACTTCTGACAACGATCGGAGGACCGAAGG
AGCTAACCGCTTTTTTGCACAACATGGGGGATCATGTAAC
TCGCCTTGATCGTTGGGAACCGGAGCTGAATGAAGCCATA
CCAAACGACGAGCGTGACACCACGATGCCTGTAGCAATGG
CAACAACGTTGCGCAAACTATTAACTGGCGAACTACTTAC
TCTAGCTTCCCGGCAACAATTAATAGACTGGATGGAGGCG
GATAAAGTTGCAGGACCACTTCTGCGCTCGGCCCTTCCGG
CTGGCTGGTTTATTGCTGATAAATCTGGAGCCGGTGAGCG
TGGGTCTCGCGGTATCATTGCAGCACTGGGGCCAGATGGT
AAGCCCTCCCGTATCGTAGTTATCTACACGACGGGGAGTC
AGGCAACTATGGATGAACGAAATAGACAGATCGCTGAGAT
AGGTGCCTCACTGATTAAGCATTGGTAACTGTCAGACCAA
GTTTACTCATATATAAAAATCCCTTAACGTGAGTTTTCGT
TCCACTGAGCGTCAGACCCCGTAGAAAAGATCAAAGGATC
TTCTTGAGATCCTTTTTTTCTGCGCGTAATCTGCTGCTTG
CAAACAAAAAAACCACCGCTACCAGCGGTGGTTTGTTTGC
CGGATCAAGAGCTACCAACTCTTTTTCCGAAGGTAACTGG
CTTCAGCAGAGCGCAGATACCAAATACTGTTCTTCTAGTG
TAGCCGTAGTTAGGCCACCACTTCAAGAACTCTGTAGCAC
CGCCTACATACCTCGCTCTGCTAATCCTGTTACCAGTGGC
TGCTGCCAGTGGCGATAAGTCGTGTCTTACCGGGTTGGAC
TCAAGACGATAGTTACCGGATAAGGCGCAGCGGTCGGGCT
GAACGGGGGGTTCGTGCACACAGCCCAGCTTGGAGCGAAC
GACCTACACCGAACTGAGATACCTACAGCGTGAGCTATGA
GAAAGCGCCACGCTTCCCGAAGGGAGAAAGGCGGACAGGT
ATCCGGTAAGCGGCAGGGTCGGAACAGGAGAGCGCACGAG
GGAGCTTCCAGGGGGAAACGCCTGGTATCTTTATAGTCCT
GTCGGGTTTCGCCACCTCTGACTTGAGCGTCGATTTTTGT
GATGCTCGTCAGGGGGGCGGAGCCTATGGAAAAACGCCAG
CAACGCGGCCTTTTTACGGTTCCTGGCCTTTTGCTGGCCT
TTTGCTCACATGTTCTTTCCTGCGTTATCCCCTGATTCTG
TGGATAACCGTATTACCGCCTTTGAGTGAGCTGATACCGC
TCGCCGCAGCCGAACGACCGAGCGCAGCGAGTCAGTGAGC
GAGGAAGCGGAAGAGCGCCCAATACGCAAACCGCCTCTCC
CCGCGCGTTGGCCGATTCATTAATGCAGCTGTGGAATGTG
TGTCAGTTAGGGTGTGGAAAGTCCCCAGGCTCCCCAGCAG
GCAGAAGTATGCAAAGCATGCATCTCAATTAGTCAGCAAC
CAGGTGTGGAAAGTCCCCAGGCTCCCCAGCAGGCAGAAGT
ATGCAAAGCATGCATCTCAATTAGTCAGCAACCATAGTCC
CGCCCCTAACTCCGCCCATCCCGCCCCTAACTCCGCCCAG
TTCCGCCCATTCTCCGCCCCATGGCTGACTAATTTTTTTT
ATTTATGCAGAGGCCGAGGCCGCCTCGGCCTCTGAGCTAT
TCCAGAAGTAGTGAGGAGGCTTTTTTGGAGGCCTAGGCTT
TTGCAAAAAGCTTGGACACAAGACAGGCTTGCGAGATATG
TTTGAGAATACCACTTTATCCCGCGTCAGGGAGAGGCAGT
GCGTAAAAAGACGCGGACTCATGTGAAATACTGGTTTTTA
GTGCGCCAGATCTCTATAATCTCGCGCAACCTATTTTCCC
CTCGAACACTTTTTAAGCCGTAGATAAACAGGCTGGGACA
CTTCACATGAGCGAAAAATACATCGTCACCTGGGACATGT
TGCAGATCCATGCACGTAAACTCGCAAGCCGACTGATGCC
TTCTGAACAATGGAAAGGCATTATTGCCGTAAGCCGTGGC
GGTCTGTACCGGGTGCGTTACTGGCGCGTGAACTGGGTAT
TCGTCATGTCGATACCGTTTGTATTTCCAGCTACGATCAC
GACAACCAGCGCGAGCTTAAAGTGCTGAAACGCGCAGAAG
GCGATGGCGAAGGCTTCATCGTTATTGATGACCTGGTGGA
TACCGGTGGTACTGCGGTTGCGATTCGTGAAATGTATCCA
AAAGCGCACTTTGTCACCATCTTCGCAAAACCGGCTGGTC
GTCCGCTGGTTGATGACTATGTTGTTGATATCCCGCAAGA
TACCTGGATTGAACAGCCGTGGGATATGGGCGTCGTATTC
GTCCCGCCAATCTCCGGTCGCTAATCTTTTCAACGCCTGG
CACTGCCGGGCGTTGTTCTTTTTAACTTCAGGCGGGTTAC
AATAGTTTCCAGTAAGTATTCTGGAGGCTGCATCCATGAC
ACAGGCAAACCTGAGCGAAACCCTGTTCAAACCCCGCTTT
AAACATCCTGAAACCTCGACGCTAGTCCGCCGCTTTAATC
ACGGCGCACAACCGCCTGTGCAGTCGGCCCTTGATGGTAA
AACCATCCCTCACTGGTATCGCATGATTAACCGTCTGATG
TGGATCTGGCGCGGCATTGACCCACGCGAAATCCTCGACG
TCCAGGCACGTATTGTGATGAGCGATGCCGAACGTACCGA
CGATGATTTATACGATACGGTGATTGGCTACCGTGGCGGC
AACTGGATTTATGAGTGGGCCCCGGATCTTTGTGAAGGAA
CCTTACTTCTGTGGTGTGACATAATTGGACAAACTACCTA
CAGAGATTTAAAGCTCTAAGGTAAATATAAAATTTTTAAG
TGTATAATGTGTTAACTTTAATGAGGAAAACCTGTTTTGC
TCAGAAGAAATGCCATCTAGTGATGATGAGGCTACTGCTG
ACTCTCAACATTCTACTCCTCCAAAAAAGAAGAGAAAGGT
AGAAGACCCCAAGGACTTTCCTTCAGAATTGCTAAGTTTT
TTGAGTCATGCTGTGTTTAGTAATAGAACTCTTGCTTGCT
TTGCTATTTACACCACAAAGGAAAAAGCTGCACTGCTATA
CAAGAAAATTATGGAAAAATATTCTGTAACCTTTATAAGT
AGGCATAACAGTTATAATCATAACATACTGTTTTTTCTTA
CTCCACACAGGCATAGAGTGTCTGCTATTAATAACTATGC
TCAAAAATTGTGTACCTTTAGCTTTTTAATTTGTAAAGGG
GTTAATAAGGAATATTTGATGTATAGTGCCTTGACTAGAG
ATCATAATCAGCCATACCACATTTGTAGAGGTTTTACTTG
CTTTAAAAAACCTCCCACACCTCCCCCTGAACCTGAAACA
TAAAATGAATGCAATTGTTGTTGTTAACTTGTTTATTGCA
GCTTATAATGGTTACAAATAAAGCAATAGCATCACAAATT
TCACAAATAAAGCATTTTTTTCACTGCATTCTAGTTGTGG
TTTGTCCAAACTCATCAATGTATCTTATCATGTCTGGATC
AACTGGATAACTCAAGCTAACCAAAATCATCCCAAACTTC
CCACCCCATACCCTATTACCACTGCCAATTACCTGTGGTT
TCATTTACTCTAAACCTGTGATTCCTCTGAATTATTTTCA
TTTTAAAGAAATTGTATTTGTTAAATATGTACTACAAACT
TAGTAG

5. hM195-CD8-41BB-CD3z

An exemplary CAR construct, as described herein, comprises a CD33 binding domain (hM195 (Lintuzumab)), a CD8a transmembrane domain, a CD8a hinge domain, a CD137 (4-1BB) co-stimulatory domain, and a CD3ζ intracellular signaling domain.

In some embodiments, a CAR in encoded by a nucleic acid sequence that comprises the sequence that is shown in SEQ ID NO: 21, or in a nucleic acid sequence that is at least 70%, at least 75%, at least 80%, at least 85%, at least 90%, at least 95%, or at least 99% identical to the nucleic acid sequence shown in SEQ ID NO: 21.

[SEQ ID NO: 21]
ATGGCTCTGCCCGTCACAGCTCTGCTGCTGCCTCTGGCCC
TGCTGCTGCACGCCGCCAGACCTCAGGTGCAGCTCGTGCA
GAGCGGCGCTGAGGTGAAGAAACCTGGCAGCAGCGTGAAG
GTGAGCTGCAAGGCCTCCGGCTACACCTTCACCGACTACA
ACATGCACTGGGTGAGGCAAGCCCCTGGCCAGGGACTGGA
GTGGATCGGCTACATCTACCCTTACAACGGCGGCACAGGC
TACAACCAGAAGTTCAAGTCCAAGGCCACCATCACCGCCG
ATGAGTCCACCAATACCGCCTACATGGAGCTCAGCAGCCT
GAGGTCCGAGGACACAGCCGTCTACTACTGCGCCAGGGGC
AGGCCCGCTATGGACTACTGGGGCCAGGGCACCCTGGTGA
CAGTGAGCTCTGGTGGCGGCGGATCCGGCGGCGGCGGCAG
CGGCGGCGGCGGCTCCGACATTCAGATGACCCAGAGCCCT
AGCAGCCTGAGCGCTTCCGTGGGAGACAGGGTGACCATCA
CATGCAGGGCCTCCGAGAGCGTGGACAATTACGGCATCAG
CTTCATGAACTGGTTCCAGCAGAAGCCCGGCAAGGCCCCC
AAACTGCTGATCTATGCCGCCAGCAATCAGGGCTCCGGCG
TGCCTAGCAGGTTTTCCGGCAGCGGCAGCGGCACCGACTT
TACCCTGACCATCTCCAGCCTGCAGCCTGACGATTTCGCC
ACCTACTACTGCCAGCAGAGCAAGGAGGTGCCTTGGACCT
TTGGACAGGGCACAAAGGTGGAGATCAAGTCCGGAACCAC
GACGCCAGCGCCGCGACCACCAACACCGGCGCCCACCATC
GCGTCGCAGCCCCTGTCCCTGCGCCCAGAGGCGTGCCGGC
CAGCGGCGGGGGGCGCAGTGCACACGAGGGGGCTGGACTT
CGCCTGTGATATCTACATCTGGGCGCCCTTGGCCGGGACT
TGTGGGGTCCTTCTCCTGTCACTGGTTATCACCCTTTACT
GCAAACGGGGCAGAAAGAAACTCCTGTATATATTCAAACA
ACCATTTATGAGACCAGTACAAACTACTCAAGAGGAAGAT
GGCTGTAGCTGCCGATTTCCAGAAGAAGAAGAAGGAGGAT
GTGAACTGAGAGTGAAGTTCAGCAGGAGCGCAGACGCCCC
CGCGTACAAGCAGGGCCAGAACCAGCTCTATAACGAGCTC
AATCTAGGACGAAGAGAGGAGTACGATGTTTTGGACAAGA
GACGTGGCCGGGACCCTGAGATGGGGGGAAAGCCGAGAAG
GAAGAACCCTCAGGAAGGCCTGTACAATGAACTGCAGAAA
GATAAGATGGCGGAGGCCTACAGTGAGATTGGGATGAAAG
GCGAGCGCCGGAGGGGCAAGGGGCACGATGGCCTTTACCA
GGGTCTCAGTACAGCCACCAAGGACACCTACGACGCCCTT
CACATGCAGGCCCTGCCCCCTCGC

In some embodiments, a CAR comprises an amino acid sequence shown in SEQ ID NO: 22, or an amino acid sequence that is at least 70%, at least 75%, at least 80%, at least 85%, at least 90%, at least 95%, or at least 99% identical to the amino acid sequence shown in SEQ ID NO: 22.

[SEQ ID NO: 22]
MALPVTALLLPLALLLHAARPQVQLVQSGAEVKKPGSSVKVSCKASGYTFTDYNMHWVRQAPGQGLEWIGYIYPY
NGGTGYNQKFKSKATITADESTNTAYMELSSLRSEDTAVYYCARGRPAMDYWGQGTLVTVSSGGGGSGGGGSGGG

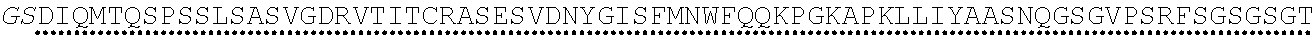

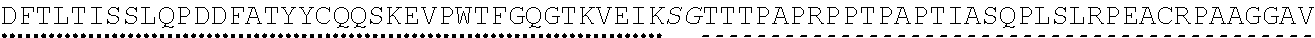

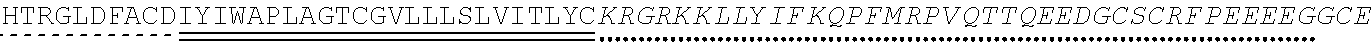

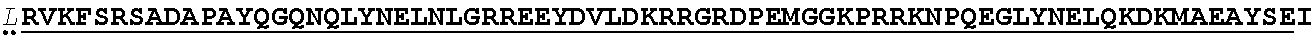

GMKGERRRGKGHDGLYQGLSTATKDTYDALHMQALPPR

In some embodiments, a CAR construct as shown in SEQ ID NO: 21 is included in a recombinant expression vector. In some embodiments the recombinant expression vector includes a promoter (e.g., an SFFV promoter or and EF1α promoter). In some embodiments, a recombinant expression vector including the CAR of SEQ ID NO: 21 comprises the sequence that is shown in SEQ ID NO: 23, or in a nucleic acid sequence that is at least 70%, at least 75%, at least 80%, at least 85%, at least 90%, at least 95%, or at least 99% identical to the nucleic acid sequence shown in SEQ ID NO: 23.

In some embodiments, a recombinant expression vector including the CAR of SEQ ID NO: 21 comprises the sequence that is shown in SEQ ID NO: 23, but does not include the SFFV promoter sequence (as shown in SEQ ID NO: 8). In some embodiments, a recombinant expression vector comprises the nucleic acid sequence shown in SEQ ID NO: 23, but does not include the SFFV sequence (as shown in SEQ ID NO: 8), and instead includes an EF1α promoter.

[SEQ ID NO: 23]
Tgggtctctctggttagaccagatctgagcctgggagctctctggctaactagggaacccactgcttaagcctca
ataaagcttgccttgagtgcttcaagtagtgtgtgcccgtctgttgtgtgactctggtaactagagatccctcag
acccttttagtcagtgtggaaaatctctagcagtggcgcccgaacagggacttgaaagcgaaagggaaaccagag
gagctctctcgacgcaggactcggcttgctgaagcgcgcacggcaagaggcgaggggcggcgactggtgagtacg
ccaaaaattttgactagcggaggctagaaggagagagatgggtgcgagagcgtcagtattaagcgggggagaatt
agatcgcgatgggaaaaaattcggttaaggccagggggaaagaaaaaatataaattaaaacatatagtatgggca
agcagggagctagaacgattcgcagttaatcctggcctgttagaaacatcagaaggctgtagacaaatactggga
cagctacaaccatcccttcagacaggatcagaagaacttagatcattatataatacagtagcaaccctctattgt
gtgcatcaaaggatagagataaaagacaccaaggaagctttagacaagatagaggaagagcaaaacaaaagtaag
accaccgcacagcaagcggccactgatcttcagacctggaggaggagatatgagggacaattggagaagtgaatt
atataaatataaagtagtaaaaattgaaccattaggagtagcacccaccaaggcaaagagaagagtggtgcagag
agaaaaaagagcagtgggaataggagctttgttccttgggttcttgggagcagcaggaagcactatgggcgcagc
gtcaatgacgctgacggtacaggccagacaattattgtctggtatagtgcagcagcagaacaatttgctgagggc
tattgaggcgcaacagcatctgttgcaactcacagtctggggcatcaagcagctccaggcaagaatcctggctgt
ggaaagatacctaaaggatcaacagctcctggggatttggggttgctctggaaaactcatttgcaccactgctgt
gccttggaatgctagttggagtaataaatctctggaacagaattggaatcacacgacctggatggagtgggacag
agaaattaacaattacacaagcttaatacactccttaattgaagaatcgcaaaaccagcaagaaaagaatgaaca
agaattattggaattagataaatgggcaagtttgtggaattggtttaacataacaaattggctgtggtatataaa
attattcataatgatagtaggaggcttggtaggtttaagaatagtttttgctgtactttctatagtgaatagagt
taggcagggatattcaccattatcgtttcagacccacctcccaaccccgaggggacccgacaggcccgaaggaat
agaagaagaaggtggagagagagacagagacagatccattcgattagtgaacggatctcgacggtatcggttaac
ttttaaaagaaaaggggggattggggggtacagtgcaggggaaagaatagtagacataatagcaacagacataca
aactaaagaattacaaaaacaaattacaaaattcaaaattttatcgatactagtggatctGCGATCGCGTAACGC
CATTTTGCAAGGCATGGAAAAATACCAAACCAAGAATAGAGAAGTTCAGATCAAGGGCGGGTACATGAAAATAGC
TAACGTTGGGCCAAACAGGATATCTGCGGTGAGCAGTTTCGGCCCCGGCCCGGGGCCAAGAACAGATGGTCACCG
CAGTTTCGGCCCCGGCCCGAGGCCAAGAACAGATGGTCCCCAGATATGGCCCAACCCTCAGCAGTTTCTTAAGAC
CCATCAGATGTTTCCAGGCTCCCCCAAGGACCTGAAATGACCCTGCGCCTTATTTGAATTAACCAATCAGCCTGC
TTCTCGCTTCTGTTCGCGCGCTTCTGCTTCCCGAGCTCTATAAAAGAGCTCACAACCCCTCACTCGGCGCGCCAG
TCCTCCGACAGACTGAGTCGTCTAGAgctagcGGATCCCCGGAATTCGACGCCACCATGGCTCTGCCCGTCACAG
CTCTGCTGCTGCCTCTGGCCCTGCTGCTGCACGCCGCCAGACCTCAGGTGCAGCTCGTGCAGAGCGGCGCTGAGG
TGAAGAAACCTGGCAGCAGCGTGAAGGTGAGCTGCAAGGCCTCCGGCTACACCTTCACCGACTACAACATGCACT
GGGTGAGGCAAGCCCCTGGCCAGGGACTGGAGTGGATCGGCTACATCTACCCTTACAACGGCGGCACAGGCTACA
ACCAGAAGTTCAAGTCCAAGGCCACCATCACCGCCGATGAGTCCACCAATACCGCCTACATGGAGCTCAGCAGCC
TGAGGTCCGAGGACACAGCCGTCTACTACTGCGCCAGGGGCAGGCCCGCTATGGACTACTGGGGCCAGGGCACCC
TGGTGACAGTGAGCTCTGGTGGCGGCGGATCCGGCGGCGGCGGCAGCGGCGGCGGCGGCTCCGACATTCAGATGA
CCCAGAGCCCTAGCAGCCTGAGCGCTTCCGTGGGAGACAGGGTGACCATCACATGCAGGGCCTCCGAGAGCGTGG
ACAATTACGGCATCAGCTTCATGAACTGGTTCCAGCAGAAGCCCGGCAAGGCCCCCAAACTGCTGATCTATGCCG
CCAGCAATCAGGGCTCCGGCGTGCCTAGCAGGTTTTCCGGCAGCGGCAGCGGCACCGACTTTACCCTGACCATCT
CCAGCCTGCAGCCTGACGATTTCGCCACCTACTACTGCCAGCAGAGCAAGGAGGTGCCTTGGACCTTTGGACAGG
GCACAAAGGTGGAGATCAAGTCCGGAACCACGACGCCAGCGCCGCGACCACCAACACCGGCGCCCACCATCGCGT
CGCAGCCCCTGTCCCTGCGCCCAGAGGCGTGCCGGCCAGCGGCGGGGGGCGCAGTGCACACGAGGGGGCTGGACT
TCGCCTGTGATATCTACATCTGGGCGCCCTTGGCCGGGACTTGTGGGGTCCTTCTCCTGTCACTGGTTATCACCC
TTTACTGCAAACGGGGCAGAAAGAAACTCCTGTATATATTCAAACAACCATTTATGAGACCAGTACAAACTACTC
AAGAGGAAGATGGCTGTAGCTGCCGATTTCCAGAAGAAGAAGAAGGAGGATGTGAACTGAGAGTGAAGTTCAGCA
GGAGCGCAGACGCCCCCGCGTACAAGCAGGGCCAGAACCAGCTCTATAACGAGCTCAATCTAGGACGAAGAGAGG
AGTACGATGTTTTGGACAAGAGACGTGGCCGGGACCCTGAGATGGGGGGAAAGCCGAGAAGGAAGAACCCTCAGG
AAGGCCTGTACAATGAACTGCAGAAAGATAAGATGGCGGAGGCCTACAGTGAGATTGGGATGAAAGGCGAGCGCC
GGAGGGGCAAGGGGCACGATGGCCTTTACCAGGGTCTCAGTACAGCCACCAAGGACACCTACGACGCCCTTCACA
TGCAGGCCCTGCCCCCTCGCgagggcagaggaagtcttctaacatgcggtgacgtggaggagaatcccggccctt
ccgggatgaccgagtacaagcccacggtgcgcctcgccacccgcgacgacgtccccagggccgtacgcaccctcg
ccgccgcgttcgccgactaccccgccacgcgccacaccgtcgatccggaccgccacatcgagcgggtcaccgagc
tgcaagaactcttcctcacgcgcgtcgggctcgacatcggcaaggtgtgggtcgcggacgacggcgccgcggtgg
cggtctggaccacgccggagagcgtcgaagcgggggcggtgttcgccgagatcggcccgcgcatggccgagttga
gcggttcccggctggccgcgcagcaacagatggaaggcctcctggcgccgcaccggcccaaggagcccgcgtggt
tcctggccaccgtcggcgtctcgcccgaccaccagggcaagggtctgggcagcgccgtcgtgctccccggagtgg
aggcggccgagcgcgccggggtgcccgccttcctggagacctccgcgccccgcaacctccccttctacgagcggc
tcggcttcaccgtcaccgccgacgtcgaggtgcccgaaggaccgcgcacctggtgcatgacccgcaagcccggtg
cctgaATTAATTAACCAATTGaatcaacctctggattacaaaatttgtgaaagattgactggtattcttaactat
gttgctccttttacgctatgtggatacgctgctttaatgcctttgtatcatgctattgcttcccgtatggctttc
attttctcctccttgtataaatcctggttgctgtctctttatgaggagttgtggcccgttgtcaggcaacgtggc
gtggtgtgcactgtgtttgctgacgcaacccccactggttggggcattgccaccacctgtcagctcctttccggg
actttcgctttccccctccctattgccacggcggaactcatcgccgcctgccttgcccgctgctggacaggggct
cggctgttgggcactgacaattccgtggtgttgtcggggaaatcatcgtcctttccttggctgctcgcctgtgtt
gccacctggattctgcgcgggacgtccttctgctacgtcccttcggccctcaatccagcggaccttccttcccgc
ggcctgctgccggctctgcggcctcttccgcgtcttcgccttcgccctcagacgagtcggatctccctttgggcc
gcctccccgcctggtacctttaagaccaatgacttacaaggcagctgtagatcttagccactttttaaaagaaaa
ggggggactggaagggctaattcactcccaacgaaaataagatctgctttttgcttgtactgggtctctctggtt
agaccagatctgagcctgggagctctctggctaactagggaacccactgcttaagcctcaataaagcttgccttg
agtgcttcaagtagtgtgtgcccgtctgttgtgtgactctggtaactagagatccctcagacccttttagtcagt
gtggaaaatctctagcaCGTATGTGTATGATACATAAGGTTATGTATTAATTGTAGCCGCGTTCTAACGACAATA
TGTACAAGCCTAATTGTGTAGCATCTGGCTTACTGAAGCAGACCCTATCATCTCTCTCGTAAACTGCCGTCAGAG
TCGGTTTGGTTGGACGAACCTTCTGAGTTTCTGGTAACGCCGTCCCGCACCCGGAAATGGTCAGCGAACCAATCA
GCAGGGTCATCGCTAGCCAGATCCTCTACGCCGGACGCATCGTGGCCGGCATCACCGGCGCCACAGGTGCGGTTG
CTGGCGCCTATATCGCCGACATCACCGATGGGGAAGATCGGGCTCGCCACTTCGGGCTCATGAGCGCTTGTTTCG
GCGTGGGTATGGTGGCAGGCCCCGTGGCCGGGGGACTGTTGGGCGCCATCTCCTTGCATGCACCATTCCTTGCGG
CGGCGGTGCTCAACGGCCTCAACCTACTACTGGGCTGCTTCCTAATGCAGGAGTCGCATAAGGGAGAGCGTCGAA
TGGTGCACTCTCAGTACAATCTGCTCTGATGCCGCATAGTTAAGCCAGCCCCGACACCCGCCAACACCCGCTGAC
GCGCCCTGACGGGCTTGTCTGCTCCCGGCATCCGCTTACAGACAAGCTGTGACCGTCTCCGGGAGCTGCATGTGT
CAGAGGTTTTCACCGTCATCACCGAAACGCGCGAGACGAAAGGGCCTCGTGATACGCCTATTTTTATAGGTTAAT
GTCATGATAATAATGGTTTCTTAGACGTCAGGTGGCACTTTTCGGGGAAATGTGCGCGGAACCCCTATTTGTTTA
TTTTTCTAAATACATTCAAATATGTATCCGCTCATGAGACAATAACCCTGATAAATGCTTCAATAATATTGAAAA
AGGAAGAGTATGAGTATTCAACATTTCCGTGTCGCCCTTATTCCCTTTTTTGCGGCATTTTGCCTTCCTGTTTTT
GCTCACCCAGAAACGCTGGTGAAAGTAAAAGATGCTGAAGATCAGTTGGGTGCACGAGTGGGTTACATCGAACTG
GATCTCAACAGCGGTAAGATCCTTGAGAGTTTTCGCCCCGAAGAACGTTTTCCAATGATGAGCACTTTTAAAGTT
CTGCTATGTGGCGCGGTATTATCCCGTATTGACGCCGGGCAAGAGCAACTCGGTCGCCGCATACACTATTCTCAG
AATGACTTGGTTGAGTACTCACCAGTCACAGAAAAGCATCTTACGGATGGCATGACAGTAAGAGAATTATGCAGT
GCTGCCATAACCATGAGTGATAACACTGCGGCCAACTTACTTCTGACAACGATCGGAGGACCGAAGGAGCTAACC
GCTTTTTTGCACAACATGGGGGATCATGTAACTCGCCTTGATCGTTGGGAACCGGAGCTGAATGAAGCCATACCA
AACGACGAGCGTGACACCACGATGCCTGTAGCAATGGCAACAACGTTGCGCAAACTATTAACTGGCGAACTACTT
ACTCTAGCTTCCCGGCAACAATTAATAGACTGGATGGAGGCGGATAAAGTTGCAGGACCACTTCTGCGCTCGGCC
CTTCCGGCTGGCTGGTTTATTGCTGATAAATCTGGAGCCGGTGAGCGTGGGTCTCGCGGTATCATTGCAGCACTG
GGGCCAGATGGTAAGCCCTCCCGTATCGTAGTTATCTACACGACGGGGAGTCAGGCAACTATGGATGAACGAAAT
AGACAGATCGCTGAGATAGGTGCCTCACTGATTAAGCATTGGTAACTGTCAGACCAAGTTTACTCATATATACTT
TAGATTGATTTAAAACTTCATTTTTAATTTAAAAGGATCTAGGTGAAGATCCTTTTTGATAATCTCATGACCAAA
ATCCCTTAACGTGAGTTTTCGTTCCACTGAGCGTCAGACCCCGTAGAAAAGATCAAAGGATCTTCTTGAGATCCT
TTTTTTCTGCGCGTAATCTGCTGCTTGCAAACAAAAAAACCACCGCTACCAGCGGTGGTTTGTTTGCCGGATCAA
GAGCTACCAACTCTTTTTCCGAAGGTAACTGGCTTCAGCAGAGCGCAGATACCAAATACTGTTCTTCTAGTGTAG
CCGTAGTTAGGCCACCACTTCAAGAACTCTGTAGCACCGCCTACATACCTCGCTCTGCTAATCCTGTTACCAGTG
GCTGCTGCCAGTGGCGATAAGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACACCGAACTGAGATACCTACAG
CGTGAGCTATGAGAAAGCGCCACGCTTCCCGAAGGGAGAAAGGCGGACAGGTATCCGGTAAGCGGCAGGGTCGGA
ACAGGAGAGCGCACGAGGGAGCTTCCAGGGGGAAACGCCTGGTATCTTTATAGTCCTGTCGGGTTTCGCCACCTC
TGACTTGAGCGTCGATTTTTGTGATGCTCGTCAGGGGGGCGGAGCCTATGGAAAAACGCCAGCAACGCGGCCTTT
TTACGGTTCCTGGCCTTTTGCTGGCCTTTTGCTCACATGTTCTTTCCTGCGTTATCCCCTGATTCTGTGGATAAC
CGTATTACCGCCTTTGAGTGAGCTGATACCGCTCGCCGCAGCCGAACGACCGAGCGCAGCGAGTCAGTGAGCGAG
GAAGCGGAAGAGCGCCCAATACGCAAACCGCCTCTCCCCGCGCGTTGGCCGATTCATTAATGCAGCTGTGGAATG
TGTGTCAGTTAGGGTGTGGAAAGTCCCCAGGCTCCCCAGCAGGCAGAAGTATGCAAAGCATGCATCTCAATTAGT
CAGCAACCAGGTGTGGAAAGTCCCCAGGCTCCCCAGCAGGCAGAAGTATGCAAAGCATGCATCTCAATTAGTCAG
CAACCATAGTCCCGCCCCTAACTCCGCCCATCCCGCCCCTAACTCCGCCCAGTTCCGCCCATTCTCCGCCCCATG
GCTGACTAATTTTTTTTATTTATGCAGAGGCCGAGGCCGCCTCGGCCTCTGAGCTATTCCAGAAGTAGTGAGGAG
GCTTTTTTGGAGGCCTAGGCTTTTGCAAAAAGCTTGGACACAAGACAGGCTTGCGAGATATGTTTGAGAATACCA
CTTTATCCCGCGTCAGGGAGAGGCAGTGCGTAAAAAGACGCGGACTCATGTGAAATACTGGTTTTTAGTGCGCCA
GATCTCTATAATCTCGCGCAACCTATTTTCCCCTCGAACACTTTTTAAGCCGTAGATAAACAGGCTGGGACACTT
CACATGAGCGAAAAATACATCGTCACCTGGGACATGTTGCAGATCCATGCACGTAAACTCGCAAGCCGACTGATG
CCTTCTGAACAATGGAAAGGCATTATTGCCGTAAGCCGTGGCGGTCTGTACCGGGTGCGTTACTGGCGCGTGAAC
TGGGTATTCGTCATGTCGATACCGTTTGTATTTCCAGCTACGATCACGACAACCAGCGCGAGCTTAAAGTGCTGA
AACGCGCAGAAGGCGATGGCGAAGGCTTCATCGTTATTGATGACCTGGTGGATACCGGTGGTACTGCGGTTGCGA
TTCGTGAAATGTATCCAAAAGCGCACTTTGTCACCATCTTCGCAAAACCGGCTGGTCGTCCGCTGGTTGATGACT
ATGTTGTTGATATCCCGCAAGATACCTGGATTGAACAGCCGTGGGATATGGGCGTCGTATTCGTCCCGCCAATCT
CCGGTCGCTAATCTTTTCAACGCCTGGCACTGCCGGGCGTTGTTCTTTTTAACTTCAGGCGGGTTACAATAGTTT
CCAGTAAGTATTCTGGAGGCTGCATCCATGACACAGGCAAACCTGAGCGAAACCCTGTTCAAACCCCGCTTTAAA
CATCCTGAAACCTCGACGCTAGTCCGCCGCTTTAATCACGGCGCACAACCGCCTGTGCAGTCGGCCCTTGATGGT
AAAACCATCCCTCACTGGTATCGCATGATTAACCGTCTGATGTGGATCTGGCGCGGCATTGACCCACGCGAAATC
CTCGACGTCCAGGCACGTATTGTGATGAGCGATGCCGAACGTACCGACGATGATTTATACGATACGGTGATTGGC
TACCGTGGCGGCAACTGGATTTATGAGTGGGCCCCGGATCTTTGTGAAGGAACCTTACTTCTGTGGTGTGACATA
ATTGGACAAACTACCTACAGAGATTTAAAGCTCTAAGGTAAATATAAAATTTTTAAGTGTATAATGTGTTAAACT
ACTGATTCTAATTGTTTGTGTATTTTAGATTCCAACCTATGGAACTGATGAATGGGAGCAGTGGTGGAATGCCTT
TAATGAGGAAAACCTGTTTTGCTCAGAAGAAATGCCATCTAGTGATGATGAGGCTACTGCTGACTCTCAACATTC
TACTCCTCCAAAAAAGAAGAGAAAGGTAGAAGACCCCAAGGACTTTCCTTCAGAATTGCTAAGTTTTTTGAGTCA
TGCTGTGTTTAGTAATAGAACTCTTGCTTGCTTTGCTATTTACACCACAAAGGAAAAAGCTGCACTGCTATACAA
GAAAATTATGGAAAAATATTCTGTAACCTTTATAAGTAGGCATAACAGTTATAATCATAACATACTGTTTTTTCT
TACTCCACACAGGCATAGAGTGTCTGCTATTAATAACTATGCTCAAAAATTGTGTACCTTTAGCTTTTTAATTTG
TAAAGGGGTTAATAAGGAATATTTGATGTATAGTGCCTTGACTAGAGATCATAATCAGCCATACCACATTTGTAG
AGGTTTTACTTGCTTTAAAAAACCTCCCACACCTCCCCCTGAACCTGAAACATAAAATGAATGCAATTGTTGTTG
TTAACTTGTTTATTGCAGCTTATAATGGTTACAAATAAAGCAATAGCATCACAAATTTCACAAATAAAGCATTTT
TTTCACTGCATTCTAGTTGTGGTTTGTCCAAACTCATCAATGTATCTTATCATGTCTGGATCAACTGGATAACTC
AAGCTAACCAAAATCATCCCAAACTTCCCACCCCATACCCTATTACCACTGCCAATTACCTGTGGTTTCATTTAC
TCTAAACCTGTGATTCCTCTGAATTATTTTCATTTTAAAGAAATTGTATTTGTTAAATATGTACTACAAACTTAG
TAG

6. M195-CD28-CD28-CD3z

An exemplary CAR construct, as described herein, comprises a CD33 binding domain (M195), a CD28 transmembrane domain, a CD28 hinge domain, a CD28 co-stimulatory domain, and a CD3ζ intracellular signaling domain.

In some embodiments, a CAR in encoded by a nucleic acid sequence that comprises the sequence that is shown in SEQ ID NO: 24, or in a nucleic acid sequence that is at least 70%, at least 75%, at least 80%, at least 85%, at least 90%, at least 95%, or at least 99% identical to the nucleic acid sequence shown in SEQ ID NO: 24.

[SEQ ID NO: 24]
ATGGCCCTGCCTGTGACAGCCCTGCTGCTGCCCCTGGCTCTGCTGCTGCATGCCGCCAGACCTATGGCTCTGCCC
GTGACCGCTCTCCTCCTGCCACTGGCACTGCTCCTCCACGCTGCTAGACCCCAGGTGCAGCTGGTGCAGTCTGGC
GCCGAAGTGAAGAAACCCGGCAGCAGCGTGAAGGTGTCCTGCAAGGCCAGCGGCTACACCTTCACCGACTACAAC
ATGCACTGGGTGCGCCAGGCTCCAGGCCAGGGACTGGAATGGATCGGCTACATCTACCCCTACAACGGCGGCACC
GGCTACAACCAGAAGTTCAAGAGCAAGGCCACCATCACCGCCGACGAGAGCACCAACACCGCCTACATGGAACTG
AGCAGCCTGCGGAGCGAGGACACCGCCGTGTACTACTGCGCCAGAGGCAGACCCGCCATGGACTACTGGGGCCAG
GGCACCCTGGTGACAGTGTCTAGCGGAGGCGGAGGCTCTGGCGGCGGAGGAAGTGGCGGAGGCGGCAGCGATATC
CAGATGACCCAGAGCCCCAGCAGCCTGAGCGCCAGCGTGGGCGACAGAGTGACCATCACCTGTCGGGCCAGCGAG
AGCGTGGACAACTACGGCATCAGCTTCATGAACTGGTTCCAGCAGAAGCCCGGCAAGGCCCCCAAGCTGCTGATC
TACGCCGCCAGCAATCAGGGCAGCGGCGTGCCCAGCAGATTCAGCGGCTCTGGCAGCGGCACCGACTTCACCCTG
AACATCAGCAGCCTGCAGCCCGACGACTTCGCCACCTACTACTGCCAGCAGAGCAAAGAGGTGCCCTGGACCTTC
GGACAGGGCACCAAGGTGGAAATCAAGACTAGTTCCGGAGCCGCCGCCATCGAAGTGATGTACCCCCCTCCCTAC
CTGGATAACGAGAAGAGCAACGGCACCATCATCCACGTGAAGGGAAAGCACCTGTGTCCCAGCCCCCTGTTTCCC
GGCCCTAGCAAGCCCTTCTGGGTGCTGGTGGTGGTCGGCGGAGTGCTGGCCTGCTACAGCCTCCTGGTGACCGTG
GCCTTCATCATCTTCTGGGTGAGGAGCAAGAGGTCCAGGCTGCTGCACAGCGACTACATGAATATGACCCCCAGA
AGGCCCGGCCCCACCAGAAAGCACTATCAGCCCTACGCCCCCCCCAGGGACTTTGCCGCCTACAGGAGCAGGGTG
AAGTTCAGCAGATCCGCCGATGCCCCTGCTTACCAGCAGGGCCAGAACCAGCTGTATAACGAGCTGAACCTGGGC
AGGAGGGAGGAATACGACGTGCTGGATAAGAGGAGGGGAAGGGACCCCGAGATGGGCGGAAAGCCCAGGAGGAAG
AACCCCCAGGAGGGCCTGTACAATGAGCTGCAGAAAGACAAGATGGCCGAGGCCTACAGCGAGATCGGCATGAAG
GGCGAGAGGAGGAGGGGCAAGGGCCATGACGGCCTGTACCAAGGCCTGTCCACCGCCACCAAGGATACCTACGAC
GCCCTGCACATGCAGGCCCTGCCTCCCAGGGGATCC

In some embodiments, a CAR comprises an amino acid sequence shown in SEQ ID NO: 25, or an amino acid sequence that is at least 70%, at least 75%, at least 80%, at least 85%, at least 90%, at least 95%, or at least 99% identical to the amino acid sequence shown in SEQ ID NO: 25.

[SEQ ID NO: 25]
MALPVTALLLPLALLLHAARPMALPVTALLLPLALLLHAARPQVQLVQSGAEVKKPGSSVKVSCKASGYTFTDYN
MHWVRQAPGQGLEWIGYIYPYNGGTGYNQKFKSKATITADESTNTAYMELSSLRSEDTAVYYCARGRPAMDYWGQ

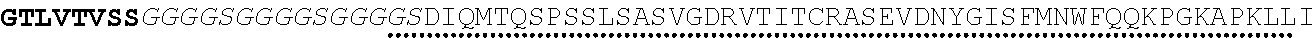

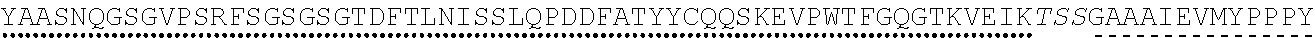

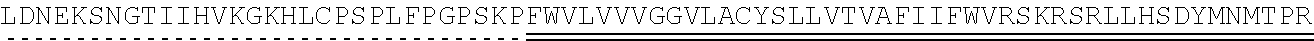

RPGPTRKHYQPYAPPRDFAAYRSRVKFSRSADAPAYQQGONQLYNELNLGRREEYDVLDKRRGRDPEMGGKPRRK
NPQEGLYNELQKDKMAEAYSEIGMKGERRRGKGHDGLYQGLSTATKDTYDALHMQALPPRGS

In some embodiments, a CAR construct as shown in SEQ ID NO: 24 is included in a recombinant expression vector. In some embodiments the recombinant expression vector includes a promoter (e.g., an SFFV promoter or and EF1α promoter). In some embodiments, a recombinant expression vector including the CAR of SEQ ID NO: 24 comprises the sequence that is shown in SEQ ID NO: 26, or in a nucleic acid sequence that is at least 70%, at least 75%, at least 80%, at least 85%, at least 90%, at least 95%, or at least 99% identical to the nucleic acid sequence shown in SEQ ID NO: 26.

In some embodiments, a recombinant expression vector including the CAR of SEQ ID NO: 24 comprises the sequence that is shown in SEQ ID NO: 26, but does not include the SFFV promoter sequence (as shown in SEQ ID NO: 8). In some embodiments, a recombinant expression vector comprises the nucleic acid sequence shown in SEQ ID NO: 26, but does not include the SFFV sequence (as shown in SEQ ID NO: 8), and instead includes an EF1α promoter sequence.

[SEQ ID NO: 26]
Tgggtctctctggttagaccagatctgagcctgggagctctctggctaactagggaacccactgcttaagcctca
ataaagcttgccttgagtgcttcaagtagtgtgtgcccgtctgttgtgtgactctggtaactagagatccctcag
acccttttagtcagtgtggaaaatctctagcagtggcgcccgaacagggacttgaaagcgaaagggaaaccagag
gagctctctcgacgcaggactcggcttgctgaagcgcgcacggcaagaggcgaggggcggcgactggtgagtacg
ccaaaaattttgactagcggaggctagaaggagagagatgggtgcgagagcgtcagtattaagcgggggagaatt
agatcgcgatgggaaaaaattcggttaaggccagggggaaagaaaaaatataaattaaaacatatagtatgggca
agcagggagctagaacgattcgcagttaatcctggcctgttagaaacatcagaaggctgtagacaaatactggga
cagctacaaccatcccttcagacaggatcagaagaacttagatcattatataatacagtagcaaccctctattgt
gtgcatcaaaggatagagataaaagacaccaaggaagctttagacaagatagaggaagagcaaaacaaaagtaag
accaccgcacagcaagcggccactgatcttcagacctggaggaggagatatgagggacaattggagaagtgaatt
atataaatataaagtagtaaaaattgaaccattaggagtagcacccaccaaggcaaagagaagagtggtgcagag
agaaaaaagagcagtgggaataggagctttgttccttgggttcttgggagcagcaggaagcactatgggcgcagc
gtcaatgacgctgacggtacaggccagacaattattgtctggtatagtgcagcagcagaacaatttgctgagggc
tattgaggcgcaacagcatctgttgcaactcacagtctggggcatcaagcagctccaggcaagaatcctggctgt
ggaaagatacctaaaggatcaacagctcctggggatttggggttgctctggaaaactcatttgcaccactgctgt
gccttggaatgctagttggagtaataaatctctggaacagaattggaatcacacgacctggatggagtgggacag
agaaattaacaattacacaagcttaatacactccttaattgaagaatcgcaaaaccagcaagaaaagaatgaaca
agaattattggaattagataaatgggcaagtttgtggaattggtttaacataacaaattggctgtggtatataaa
attattcataatgatagtaggaggcttggtaggtttaagaatagtttttgctgtactttctatagtgaatagagt
taggcagggatattcaccattatcgtttcagacccacctcccaaccccgaggggacccgacaggcccgaaggaat
agaagaagaaggtggagagagagacagagacagatccattcgattagtgaacggatctcgacggtatcggttaac
ttttaaaagaaaaggggggattggggggtacagtgcaggggaaagaatagtagacataatagcaacagacataca
aactaaagaattacaaaaacaaattacaaaattcaaaattttatcgatactagtggatctGCGATCGCGTAACGC
CATTTTGCAAGGCATGGAAAAATACCAAACCAAGAATAGAGAAGTTCAGATCAAGGGCGGGTACATGAAAATAGC
TAACGTTGGGCCAAACAGGATATCTGCGGTGAGCAGTTTCGGCCCCGGCCCGGGGCCAAGAACAGATGGTCACCG
CAGTTTCGGCCCCGGCCCGAGGCCAAGAACAGATGGTCCCCAGATATGGCCCAACCCTCAGCAGTTTCTTAAGAC
CCATCAGATGTTTCCAGGCTCCCCCAAGGACCTGAAATGACCCTGCGCCTTATTTGAATTAACCAATCAGCCTGC
TTCTCGCTTCTGTTCGCGCGCTTCTGCTTCCCGAGCTCTATAAAAGAGCTCACAACCCCTCACTCGGCGCGCCAG
TCCTCCGACAGACTGAGTCGTCTAGAgctagcGGATCCCCGGAATTCGACGCCACCATGGCCCTGCCTGTGACAG
CCCTGCTGCTGCCCCTGGCTCTGCTGCTGCATGCCGCCAGACCTATGGCTCTGCCCGTGACCGCTCTCCTCCTGC
CACTGGCACTGCTCCTCCACGCTGCTAGACCCCAGGTGCAGCTGGTGCAGTCTGGCGCCGAAGTGAAGAAACCCG
GCAGCAGCGTGAAGGTGTCCTGCAAGGCCAGCGGCTACACCTTCACCGACTACAACATGCACTGGGTGCGCCAGG
CTCCAGGCCAGGGACTGGAATGGATCGGCTACATCTACCCCTACAACGGCGGCACCGGCTACAACCAGAAGTTCA
AGAGCAAGGCCACCATCACCGCCGACGAGAGCACCAACACCGCCTACATGGAACTGAGCAGCCTGCGGAGCGAGG
ACACCGCCGTGTACTACTGCGCCAGAGGCAGACCCGCCATGGACTACTGGGGCCAGGGCACCCTGGTGACAGTGT
CTAGCGGAGGCGGAGGCTCTGGCGGCGGAGGAAGTGGCGGAGGCGGCAGCGATATCCAGATGACCCAGAGCCCCA
GCAGCCTGAGCGCCAGCGTGGGCGACAGAGTGACCATCACCTGTCGGGCCAGCGAGAGCGTGGACAACTACGGCA
TCAGCTTCATGAACTGGTTCCAGCAGAAGCCCGGCAAGGCCCCCAAGCTGCTGATCTACGCCGCCAGCAATCAGG
GCAGCGGCGTGCCCAGCAGATTCAGCGGCTCTGGCAGCGGCACCGACTTCACCCTGAACATCAGCAGCCTGCAGC
CCGACGACTTCGCCACCTACTACTGCCAGCAGAGCAAAGAGGTGCCCTGGACCTTCGGACAGGGCACCAAGGTGG
AAATCAAGACTAGTTCCGGAGCCGCCGCCATCGAAGTGATGTACCCCCCTCCCTACCTGGATAACGAGAAGAGCA
ACGGCACCATCATCCACGTGAAGGGAAAGCACCTGTGTCCCAGCCCCCTGTTTCCCGGCCCTAGCAAGCCCTTCT
GGGTGCTGGTGGTGGTCGGCGGAGTGCTGGCCTGCTACAGCCTCCTGGTGACCGTGGCCTTCATCATCTTCTGGG
TGAGGAGCAAGAGGTCCAGGCTGCTGCACAGCGACTACATGAATATGACCCCCAGAAGGCCCGGCCCCACCAGAA
AGCACTATCAGCCCTACGCCCCCCCCAGGGACTTTGCCGCCTACAGGAGCAGGGTGAAGTTCAGCAGATCCGCCG
ATGCCCCTGCTTACCAGCAGGGCCAGAACCAGCTGTATAACGAGCTGAACCTGGGCAGGAGGGAGGAATACGACG
TGCTGGATAAGAGGAGGGGAAGGGACCCCGAGATGGGCGGAAAGCCCAGGAGGAAGAACCCCCAGGAGGGCCTGT
ACAATGAGCTGCAGAAAGACAAGATGGCCGAGGCCTACAGCGAGATCGGCATGAAGGGCGAGAGGAGGAGGGGCA
AGGGCCATGACGGCCTGTACCAAGGCCTGTCCACCGCCACCAAGGATACCTACGACGCCCTGCACATGCAGGCCC
TGCCTCCCAGGGGATCCgagggcagaggaagtcttctaacatgcggtgacgtggaggagaatcccggcccttccg
ggatgaccgagtacaagcccacggtgcgcctcgccacccgcgacgacgtccccagggccgtacgcaccctcgccg
ccgcgttcgccgactaccccgccacgcgccacaccgtcgatccggaccgccacatcgagcgggtcaccgagctgc
aagaactcttcctcacgcgcgtcgggctcgacatcggcaaggtgtgggtcgcggacgacggcgccgcggtggcgg
tctggaccacgccggagagcgtcgaagcgggggcggtgttcgccgagatcggcccgcgcatggccgagttgagcg
gttcccggctggccgcgcagcaacagatggaaggcctcctggcgccgcaccggcccaaggagcccgcgtggttcc
tggccaccgtcggcgtctcgcccgaccaccagggcaagggtctgggcagcgccgtcgtgctccccggagtggagg
cggccgagcgcgccggggtgcccgccttcctggagacctccgcgccccgcaacctccccttctacgagcggctcg
gcttcaccgtcaccgccgacgtcgaggtgcccgaaggaccgcgcacctggtgcatgacccgcaagcccggtgcct
gaATTAATTAACCAATTGaatcaacctctggattacaaaatttgtgaaagattgactggtattcttaactatgtt
gctccttttacgctatgtggatacgctgctttaatgcctttgtatcatgctattgcttcccgtatggctttcatt
ttctcctccttgtataaatcctggttgctgtctctttatgaggagttgtggcccgttgtcaggcaacgtggcgtg
gtgtgcactgtgtttgctgacgcaacccccactggttggggcattgccaccacctgtcagctcctttccgggact
ttcgctttccccctccctattgccacggcggaactcatcgccgcctgccttgcccgctgctggacaggggctcgg
ctgttgggcactgacaattccgtggtgttgtcggggaaatcatcgtcctttccttggctgctcgcctgtgttgcc
acctggattctgcgcgggacgtccttctgctacgtcccttcggccctcaatccagcggaccttccttcccgcggc
ctgctgccggctctgcggcctcttccgcgtcttcgccttcgccctcagacgagtcggatctccctttgggccgcc
tccccgcctggtacctttaagaccaatgacttacaaggcagctgtagatcttagccactttttaaaagaaaaggg
gggactggaagggctaattcactcccaacgaaaataagatctgctttttgcttgtactgggtctctctggttaga
ccagatctgagcctgggagctctctggctaactagggaacccactgcttaagcctcaataaagcttgccttgagt
gcttcaagtagtgtgtgcccgtctgttgtgtgactctggtaactagagatccctcagacccttttagtcagtgtg
gaaaatctctagcaCGTATGTGTATGATACATAAGGTTATGTATTAATTGTAGCCGCGTTCTAACGACAATATGT
ACAAGCCTAATTGTGTAGCATCTGGCTTACTGAAGCAGACCCTATCATCTCTCTCGTAAACTGCCGTCAGAGTCG
GTTTGGTTGGACGAACCTTCTGAGTTTCTGGTAACGCCGTCCCGCACCCGGAAATGGTCAGCGAACCAATCAGCA
GGGTCATCGCTAGCCAGATCCTCTACGCCGGACGCATCGTGGCCGGCATCACCGGCGCCACAGGTGCGGTTGCTG
GCGCCTATATCGCCGACATCACCGATGGGGAAGATCGGGCTCGCCACTTCGGGCTCATGAGCGCTTGTTTCGGCG
TGGGTATGGTGGCAGGCCCCGTGGCCGGGGGACTGTTGGGCGCCATCTCCTTGCATGCACCATTCCTTGCGGCGG
CGGTGCTCAACGGCCTCAACCTACTACTGGGCTGCTTCCTAATGCAGGAGTCGCATAAGGGAGAGCGTCGAATGG
TGCACTCTCAGTACAATCTGCTCTGATGCCGCATAGTTAAGCCAGCCCCGACACCCGCCAACACCCGCTGACGCG
CCCTGACGGGCTTGTCTGCTCCCGGCATCCGCTTACAGACAAGCTGTGACCGTCTCCGGGAGCTGCATGTGTCAG
AGGTTTTCACCGTCATCACCGAAACGCGCGAGACGAAAGGGCCTCGTGATACGCCTATTTTTATAGGTTAATGTC
ATGATAATAATGGTTTCTTAGACGTCAGGTGGCACTTTTCGGGGAAATGTGCGCGGAACCCCTATTTGTTTATTT
TTCTAAATACATTCAAATATGTATCCGCTCATGAGACAATAACCCTGATAAATGCTTCAATAATATTGAAAAAGG
AAGAGTATGAGTATTCAACATTTCCGTGTCGCCCTTATTCCCTTTTTTGCGGCATTTTGCCTTCCTGTTTTTGCT
CACCCAGAAACGCTGGTGAAAGTAAAAGATGCTGAAGATCAGTTGGGTGCACGAGTGGGTTACATCGAACTGGAT
CTCAACAGCGGTAAGATCCTTGAGAGTTTTCGCCCCGAAGAACGTTTTCCAATGATGAGCACTTTTAAAGTTCTG
CTATGTGGCGCGGTATTATCCCGTATTGACGCCGGGCAAGAGCAACTCGGTCGCCGCATACACTATTCTCAGAAT
GACTTGGTTGAGTACTCACCAGTCACAGAAAAGCATCTTACGGATGGCATGACAGTAAGAGAATTATGCAGTGCT
GCCATAACCATGAGTGATAACACTGCGGCCAACTTACTTCTGACAACGATCGGAGGACCGAAGGAGCTAACCGCT
TTTTTGCACAACATGGGGGATCATGTAACTCGCCTTGATCGTTGGGAACCGGAGCTGAATGAAGCCATACCAAAC
GACGAGCGTGACACCACGATGCCTGTAGCAATGGCAACAACGTTGCGCAAACTATTAACTGGCGAACTACTTACT
CTAGCTTCCCGGCAACAATTAATAGACTGGATGGAGGCGGATAAAGTTGCAGGACCACTTCTGCGCTCGGCCCTT
CCGGCTGGCTGGTTTATTGCTGATAAATCTGGAGCCGGTGAGCGTGGGTCTCGCGGTATCATTGCAGCACTGGGG
CCAGATGGTAAGCCCTCCCGTATCGTAGTTATCTACACGACGGGGAGTCAGGCAACTATGGATGAACGAAATAGA
CAGATCGCTGAGATAGGTGCCTCACTGATTAAGCATTGGTAACTGTCAGACCAAGTTTACTCATATATACTTTAG
ATTGATTTAAAACTTCATTTTTAATTTAAAAGGATCTAGGTGAAGATCCTTTTTGATAATCTCATGACCAAAATC
CCTTAACGTGAGTTTTCGTTCCACTGAGCGTCAGACCCCGTAGAAAAGATCAAAGGATCTTCTTGAGATCCTTTT
TTTCTGCGCGTAATCTGCTGCTTGCAAACAAAAAAACCACCGCTACCAGCGGTGGTTTGTTTGCCGGATCAAGAG
CTACCAACTCTTTTTCCGAAGGTAACTGGCTTCAGCAGAGCGCAGATACCAAATACTGTTCTTCTAGTGTAGCCG
TAGTTAGGCCACCACTTCAAGAACTCTGTAGCACCGCCTACATACCTCGCTCTGCTAATCCTGTTACCAGTGGCT
GCTGCCAGTGGCGATAAGTCGTGTCTTACCGGGTTGGACTCAAGACGATAGTTACCGGATAAGGCGCAGCGGTCG
GGCTGAACGGGGGGTTCGTGCACACAGCCCAGCTTGGAGCGAACGACCTACACCGAACTGAGATACCTACAGCGT
GAGCTATGAGAAAGCGCCACGCTTCCCGAAGGGAGAAAGGCGGACAGGTATCCGGTAAGCGGCAGGGTCGGAACA
GGAGAGCGCACGAGGGAGCTTCCAGGGGGAAACGCCTGGTATCTTTATAGTCCTGTCGGGTTTCGCCACCTCTGA
CTTGAGCGTCGATTTTTGTGATGCTCGTCAGGGGGGCGGAGCCTATGGAAAAACGCCAGCAACGCGGCCTTTTTA
CGGTTCCTGGCCTTTTGCTGGCCTTTTGCTCACATGTTCTTTCCTGCGTTATCCCCTGATTCTGTGGATAACCGT
ATTACCGCCTTTGAGTGAGCTGATACCGCTCGCCGCAGCCGAACGACCGAGCGCAGCGAGTCAGTGAGCGAGGAA
GCGGAAGAGCGCCCAATACGCAAACCGCCTCTCCCCGCGCGTTGGCCGATTCATTAATGCAGCTGTGGAATGTGT
GTCAGTTAGGGTGTGGAAAGTCCCCAGGCTCCCCAGCAGGCAGAAGTATGCAAAGCATGCATCTCAATTAGTCAG
CAACCAGGTGTGGAAAGTCCCCAGGCTCCCCAGCAGGCAGAAGTATGCAAAGCATGCATCTCAATTAGTCAGCAA
CCATAGTCCCGCCCCTAACTCCGCCCATCCCGCCCCTAACTCCGCCCAGTTCCGCCCATTCTCCGCCCCATGGCT
GACTAATTTTTTTTATTTATGCAGAGGCCGAGGCCGCCTCGGCCTCTGAGCTATTCCAGAAGTAGTGAGGAGGCT
TTTTTGGAGGCCTAGGCTTTTGCAAAAAGCTTGGACACAAGACAGGCTTGCGAGATATGTTTGAGAATACCACTT
TATCCCGCGTCAGGGAGAGGCAGTGCGTAAAAAGACGCGGACTCATGTGAAATACTGGTTTTTAGTGCGCCAGAT
CTCTATAATCTCGCGCAACCTATTTTCCCCTCGAACACTTTTTAAGCCGTAGATAAACAGGCTGGGACACTTCAC
ATGAGCGAAAAATACATCGTCACCTGGGACATGTTGCAGATCCATGCACGTAAACTCGCAAGCCGACTGATGCCT
TCTGAACAATGGAAAGGCATTATTGCCGTAAGCCGTGGCGGTCTGTACCGGGTGCGTTACTGGCGCGTGAACTGG
GTATTCGTCATGTCGATACCGTTTGTATTTCCAGCTACGATCACGACAACCAGCGCGAGCTTAAAGTGCTGAAAC
GCGCAGAAGGCGATGGCGAAGGCTTCATCGTTATTGATGACCTGGTGGATACCGGTGGTACTGCGGTTGCGATTC
GTGAAATGTATCCAAAAGCGCACTTTGTCACCATCTTCGCAAAACCGGCTGGTCGTCCGCTGGTTGATGACTATG
TTGTTGATATCCCGCAAGATACCTGGATTGAACAGCCGTGGGATATGGGCGTCGTATTCGTCCCGCCAATCTCCG
GTCGCTAATCTTTTCAACGCCTGGCACTGCCGGGCGTTGTTCTTTTTAACTTCAGGCGGGTTACAATAGTTTCCA
GTAAGTATTCTGGAGGCTGCATCCATGACACAGGCAAACCTGAGCGAAACCCTGTTCAAACCCCGCTTTAAACAT
CCTGAAACCTCGACGCTAGTCCGCCGCTTTAATCACGGCGCACAACCGCCTGTGCAGTCGGCCCTTGATGGTAAA
ACCATCCCTCACTGGTATCGCATGATTAACCGTCTGATGTGGATCTGGCGCGGCATTGACCCACGCGAAATCCTC
GACGTCCAGGCACGTATTGTGATGAGCGATGCCGAACGTACCGACGATGATTTATACGATACGGTGATTGGCTAC
CGTGGCGGCAACTGGATTTATGAGTGGGCCCCGGATCTTTGTGAAGGAACCTTACTTCTGTGGTGTGACATAATT
GGACAAACTACCTACAGAGATTTAAAGCTCTAAGGTAAATATAAAATTTTTAAGTGTATAATGTGTTAAACTACT
GATTCTAATTGTTTGTGTATTTTAGATTCCAACCTATGGAACTGATGAATGGGAGCAGTGGTGGAATGCCTTTAA
TGAGGAAAACCTGTTTTGCTCAGAAGAAATGCCATCTAGTGATGATGAGGCTACTGCTGACTCTCAACATTCTAC
TCCTCCAAAAAAGAAGAGAAAGGTAGAAGACCCCAAGGACTTTCCTTCAGAATTGCTAAGTTTTTTGAGTCATGC
TGTGTTTAGTAATAGAACTCTTGCTTGCTTTGCTATTTACACCACAAAGGAAAAAGCTGCACTGCTATACAAGAA
AATTATGGAAAAATATTCTGTAACCTTTATAAGTAGGCATAACAGTTATAATCATAACATACTGTTTTTTCTTAC
TCCACACAGGCATAGAGTGTCTGCTATTAATAACTATGCTCAAAAATTGTGTACCTTTAGCTTTTTAATTTGTAA
TTTTACTTGCTTTAAAAAACCTCCCACACCTCCCCCTGAACCTGAAACATAAAATGAATGCAATTGTTGTTGTTA
ACTTGTTTATTGCAGCTTATAATGGTTACAAATAAAGCAATAGCATCACAAATTTCACAAATAAAGCATTTTTTT
CACTGCATTCTAGTTGTGGTTTGTCCAAACTCATCAATGTATCTTATCATGTCTGGATCAACTGGATAACTCAAG
CTAACCAAAATCATCCCAAACTTCCCACCCCATACCCTATTACCACTGCCAATTACCTGTGGTTTCATTTACTCT
AAACCTGTGATTCCTCTGAATTATTTTCATTTTAAAGAAATTGTATTTGTTAAATATGTACTACAAACTTAGTAG

7. My9.6-CD8-41BB-CD3z

An exemplary CAR construct, as described herein, comprises a CD33 binding domain (My9.6, wherein the VH-CDR3 comprises the amino acid sequence of LGGSLPDYGMDV [SEQ ID NO: 27]), a CD8a transmembrane domain, a CD8a hinge domain, a CD137 (4-1BB) co-stimulatory domain, and a CD3ζ intracellular signaling domain.

In some embodiments, a CAR in encoded by a nucleic acid sequence that comprises the sequence that is shown in SEQ ID NO: 28, or in a nucleic acid sequence that is at least 70%, at least 75%, at least 80%, at least 85%, at least 90%, at least 95%, or at least 99% identical to the nucleic acid sequence shown in SEQ ID NO: 28.

[SEQ ID NO: 28]
ATGGCCCTCCCTGTCACCGCCCTGCTGCTTCCGCTGGCTCTTCTGCTCCACGCCGCTCGGCCCCAAGTGCAACTC
GTCCAGTCCGGTGCAGAAGTCAAGAAGCCAGGAGAATCACTCAAGATTAGCTGCAAAGGCAGCGGCTACTCCTTC
ACTTCCTACTGGATCGGCTGGGTGCGCCAGATGCCCGGAAAGGGACTGGAGTGGATGGGAATCATCTACCCTGGC
GATAGCGACACCAGATACTCCCCGAGCTTTCAAGGCCAAGTGACCATTTCGGCCGACAAGTCGATCTCCACCGCG
TATCTGCAGTGGAGCTCACTGAAGGCTTCGGACACCGCCATGTACTACTGTGCCCGGCTGGGGGGAAGCCTGCCC
GATTACGGAATGGACGTGTGGGGCCAGGGAACCATGGTCACTGTGTCCTCCGCCTCCGGGGGTGGAGGCTCCGGT
GGAGGGGGGTCCGGTGGTGGAGGATCAGAAATTGTGCTGACCCAGTCTCCGCTGTCCTTGCCTGTGACCCCGGGC
GAACCCGCAAGCATCTCCTGCCGGTCGTCGCAGTCCCTGCTTCACTCCAACGGCTACAACTACCTCGATTGGTAC
CTCCAGAAGCCTGGACAGAGCCCACAGCTGTTGATCTACCTGGGCTCGAACCGGGCCTCAGGAGTGCCGGACAGG
TTCTCCGGCTCCGGGTCGGGAACCGACTTCACGCTGAAGATCTCCCGCGTGGAGGCCGAGGACGTGGGCGTGTAC
TATTGCATGCAGGCGCTGCAGACCCTTATTACATTCGGACAGGGGACTAAGGTCGATATCAAGACCACTACCCCA
GCACCGAGGCCACCCACCCCGGCTCCTACCATCGCCTCCCAGCCTCTGTCCCTGCGTCCGGAGGCATGTAGACCC
GCAGCTGGTGGGGCCGTGCATACCCGGGGTCTTGACTTCGCCTGCGATATCTACATTTGGGCCCCTCTGGCTGGT
ACTTGCGGGGTCCTGCTGCTTTCACTCGTGATCACTCTTTACTGTAAGCGCGGTCGGAAGAAGCTGCTGTACATC
TTTAAGCAACCCTTCATGAGGCCTGTGCAGACTACTCAAGAGGAGGACGGCTGTTCATGCCGGTTCCCAGAGGAG
GAGGAAGGCGGCTGCGAACTGCGCGTGAAATTCAGCCGCAGCGCAGATGCTCCAGCCTACAAGCAGGGGCAGAAC
CAGCTCTACAACGAACTCAATCTTGGTCGGAGAGAGGAGTACGACGTGCTGGACAAGCGGAGAGGACGGGACCCA
GAAATGGGCGGGAAGCCGCGCAGAAAGAATCCCCAAGAGGGCCTGTACAACGAGCTCCAAAAGGATAAGATGGCA
GAAGCCTATAGCGAGATTGGTATGAAAGGGGAACGCAGAAGAGGCAAAGGCCACGACGGACTGTACCAGGGACTC
AGCACCGCCACCAAGGACACCTATGACGCTCTTCACATGCAGGCCCTGCCGCCTCGG

In some embodiments, a CAR comprises an amino acid sequence shown in SEQ ID NO: 29, or an amino acid sequence that is at least 70%, at least 75%, at least 80%, at least 85%, at least 90%, at least 95%, or at least 99% identical to the amino acid sequence shown in SEQ ID NO: 29.

[SEQ ID NO: 29]
MALPVTALLLPLALLLHAARPQVQLVQSGAEVKKPGESLKISCKGSGYSFTSYWIGWVRQMPGKGLEWMGIIYPG
DSDTRYSPSFQGQVTISADKSISTAYLQWSSLKASDTAMYYCARLGGSLPDYGMDVWGQGTMVTVSSASGGGGSG

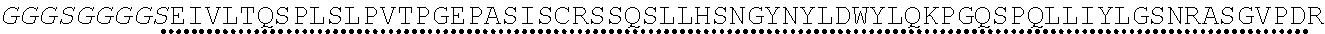

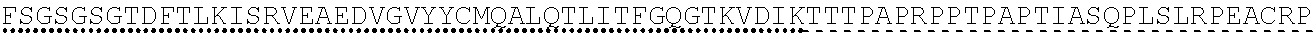

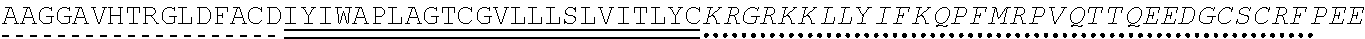

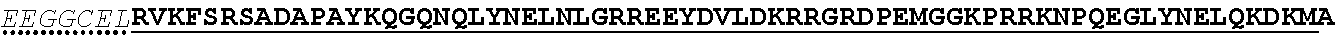

EAYSEIGMKGERRRGKGHDGLYQGLSTATKDTYDALHMQALPPR

In some embodiments, a CAR construct as shown in SEQ ID NO: 28 is included in a recombinant expression vector. In some embodiments the recombinant expression vector includes a promoter (e.g., an SFFV promoter or and EF1α promoter). In some embodiments, a recombinant expression vector including the CAR of SEQ ID NO: 28 comprises the sequence that is shown in SEQ ID NO: 30, or in a nucleic acid sequence that is at least 70%, at least 75%, at least 80%, at least 85%, at least 90%, at least 95%, or at least 99% identical to the nucleic acid sequence shown in SEQ ID NO: 30.

In some embodiments, a recombinant expression vector including the CAR of SEQ ID NO: 28 comprises the sequence that is shown in SEQ ID NO: 30, but does not include the SFFV promoter sequence (as shown in SEQ ID NO: 8). In some embodiments, a recombinant expression vector comprises the nucleic acid sequence shown in SEQ ID NO: 30, but does not include the SFFV sequence (as shown in SEQ ID NO: 8), and instead includes an EF1α promoter sequence.

[SEQ ID NO: 30]
Tgggtctctctggttagaccagatctgagcctgggagctctctggctaactagggaacccactgcttaagcctca
ataaagcttgccttgagtgcttcaagtagtgtgtgcccgtctgttgtgtgactctggtaactagagatccctcag
acccttttagtcagtgtggaaaatctctagcagtggcgcccgaacagggacttgaaagcgaaagggaaaccagag
gagctctctcgacgcaggactcggcttgctgaagcgcgcacggcaagaggcgaggggCggcgactggtgagtacg
ccaaaaattttgactagcggaggctagaaggagagagatgggtgcgagagcgtcagtattaagcgggggagaatt
agatcgcgatgggaaaaaattcggttaaggccagggggaaagaaaaaatataaattaaaacatatagtatgggca
agcagggagctagaacgattcgcagttaatcctggcctgttagaaacatcagaaggctgtagacaaatactggga
cagctacaaccatcccttcagacaggatcagaagaacttagatcattatataatacagtagcaaccctctattgt
gtgcatcaaaggatagagataaaagacaccaaggaagctttagacaagatagaggaagagcaaaacaaaagtaag
accaccgcacagcaagcggccactgatcttcagacctggaggaggagatatgagggacaattggagaagtgaatt
atataaatataaagtagtaaaaattgaaccattaggagtagcacccaccaaggcaaagagaagagtggtgcagag
agaaaaaagagcagtgggaataggagctttgttccttgggttcttgggagcagcaggaagcactatgggcgcagc
gtcaatgacgctgacggtacaggccagacaattattgtctggtatagtgcagcagcagaacaatttgctgagggc
tattgaggcgcaacagcatctgttgcaactcacagtctggggcatcaagcagctccaggcaagaatcctggctgt
ggaaagatacctaaaggatcaacagctcctggggatttggggttgctctggaaaactcatttgcaccactgctgt
gccttggaatgctagttggagtaataaatctctggaacagaattggaatcacacgacctggatggagtgggacag
agaaattaacaattacacaagcttaatacactccttaattgaagaatcgcaaaaccagcaagaaaagaatgaaca
agaattattggaattagataaatgggcaagtttgtggaattggtttaacataacaaattggctgtggtatataaa
attattcataatgatagtaggaggcttggtaggtttaagaatagtttttgctgtactttctatagtgaatagagt
taggcagggatattcaccattatcgtttcagacccacctcccaaccccgaggggacccgacaggcccgaaggaat
agaagaagaaggtggagagagagacagagacagatccattcgattagtgaacggatctcgacggtatcggttaac
ttttaaaagaaaaggggggattggggggtacagtgcaggggaaagaatagtagacataatagcaacagacataca
aactaaagaattacaaaaacaaattacaaaattcaaaattttatcgatactagtggatctGCGATCGCGTAACGC
CATTTTGCAAGGCATGGAAAAATACCAAACCAAGAATAGAGAAGTTCAGATCAAGGGCGGGTACATGAAAATAGC
TAACGTTGGGCCAAACAGGATATCTGCGGTGAGCAGTTTCGGCCCCGGCCCGGGGCCAAGAACAGATGGTCACCG
CAGTTTCGGCCCCGGCCCGAGGCCAAGAACAGATGGTCCCCAGATATGGCCCAACCCTCAGCAGTTTCTTAAGAC
CCATCAGATGTTTCCAGGCTCCCCCAAGGACCTGAAATGACCCTGCGCCTTATTTGAATTAACCAATCAGCCTGC
TTCTCGCTTCTGTTCGCGCGCTTCTGCTTCCCGAGCTCTATAAAAGAGCTCACAACCCCTCACTCGGCGCGCCAG
TCCTCCGACAGACTGAGTCGTCTAGAgctagcGGATCCCCGGAATTCGACGCCACCATGGCCCTCCCTGTCACCG
CCCTGCTGCTTCCGCTGGCTCTTCTGCTCCACGCCGCTCGGCCCCAAGTGCAACTCGTCCAGTCCGGTGCAGAAG
TCAAGAAGCCAGGAGAATCACTCAAGATTAGCTGCAAAGGCAGCGGCTACTCCTTCACTTCCTACTGGATCGGCT
GGGTGCGCCAGATGCCCGGAAAGGGACTGGAGTGGATGGGAATCATCTACCCTGGCGATAGCGACACCAGATACT
CCCCGAGCTTTCAAGGCCAAGTGACCATTTCGGCCGACAAGTCGATCTCCACCGCGTATCTGCAGTGGAGCTCAC
TGAAGGCTTCGGACACCGCCATGTACTACTGTGCCCGGCTGGGGGGAAGCCTGCCCGATTACGGAATGGACGTGT
GGGGCCAGGGAACCATGGTCACTGTGTCCTCCGCCTCCGGGGGTGGAGGCTCCGGTGGAGGGGGGTCCGGTGGTG
GAGGATCAGAAATTGTGCTGACCCAGTCTCCGCTGTCCTTGCCTGTGACCCCGGGCGAACCCGCAAGCATCTCCT
GCCGGTCGTCGCAGTCCCTGCTTCACTCCAACGGCTACAACTACCTCGATTGGTACCTCCAGAAGCCTGGACAGA
GCCCACAGCTGTTGATCTACCTGGGCTCGAACCGGGCCTCAGGAGTGCCGGACAGGTTCTCCGGCTCCGGGTCGG
GAACCGACTTCACGCTGAAGATCTCCCGCGTGGAGGCCGAGGACGTGGGCGTGTACTATTGCATGCAGGCGCTGC
AGACCCTTATTACATTCGGACAGGGGACTAAGGTCGATATCAAGACCACTACCCCAGCACCGAGGCCACCCACCC
CGGCTCCTACCATCGCCTCCCAGCCTCTGTCCCTGCGTCCGGAGGCATGTAGACCCGCAGCTGGTGGGGCCGTGC
ATACCCGGGGTCTTGACTTCGCCTGCGATATCTACATTTGGGCCCCTCTGGCTGGTACTTGCGGGGTCCTGCTGC
TTTCACTCGTGATCACTCTTTACTGTAAGCGCGGTCGGAAGAAGCTGCTGTACATCTTTAAGCAACCCTTCATGA
GGCCTGTGCAGACTACTCAAGAGGAGGACGGCTGTTCATGCCGGTTCCCAGAGGAGGAGGAAGGCGGCTGCGAAC
TGCGCGTGAAATTCAGCCGCAGCGCAGATGCTCCAGCCTACAAGCAGGGGCAGAACCAGCTCTACAACGAACTCA
ATCTTGGTCGGAGAGAGGAGTACGACGTGCTGGACAAGCGGAGAGGACGGGACCCAGAAATGGGCGGGAAGCCGC
GCAGAAAGAATCCCCAAGAGGGCCTGTACAACGAGCTCCAAAAGGATAAGATGGCAGAAGCCTATAGCGAGATTG
GTATGAAAGGGGAACGCAGAAGAGGCAAAGGCCACGACGGACTGTACCAGGGACTCAGCACCGCCACCAAGGACA
CCTATGACGCTCTTCACATGCAGGCCCTGCCGCCTCGGgagggcagaggaagtcttctaacatgcggtgacgtgg
aggagaatcccggcccttccgggatgaccgagtacaagcccacggtgcgcctcgccacccgcgacgacgtcccca
gggccgtacgcaccctcgccgccgcgttcgccgactaccccgccacgcgccacaccgtcgatccggaccgccaca
tcgagcgggtcaccgagctgcaagaactcttcctcacgcgcgtcgggctcgacatcggcaaggtgtgggtcgcgg
acgacggcgccgcggtggcggtctggaccacgccggagagcgtcgaagcgggggcggtgttcgccgagatcggcc
cgcgcatggccgagttgagcggttcccggctggccgcgcagcaacagatggaaggcctcctggcgccgcaccggc
ccaaggagcccgcgtggttcctggccaccgtcggcgtctcgcccgaccaccagggcaagggtctgggcagcgccg
tcgtgctccccggagtggaggcggccgagcgcgccggggtgcccgccttcctggagacctccgcgccccgcaacc
tccccttctacgagcggctcggcttcaccgtcaccgccgacgtcgaggtgcccgaaggaccgcgcacctggtgca
tgacccgcaagcccggtgcctgaATTAATTAACCAATTGaatcaacctctggattacaaaatttgtgaaagattg
actggtattcttaactatgttgctccttttacgctatgtggatacgctgctttaatgcctttgtatcatgctatt
gcttcccgtatggctttcattttctcctccttgtataaatcctggttgctgtctctttatgaggagttgtggccc
gttgtcaggcaacgtggcgtggtgtgcactgtgtttgctgacgcaacccccactggttggggcattgccaccacc
tgtcagctcctttccgggactttcgctttccccctccctattgccacggcggaactcatcgccgcctgccttgcc
cgctgctggacaggggctcggctgttgggcactgacaattccgtggtgttgtcggggaaatcatcgtcctttcct
tggctgctcgcctgtgttgccacctggattctgcgcgggacgtccttctgctacgtcccttcggccctcaatcca
gcggaccttccttcccgcggcctgctgccggctctgcggcctcttccgcgtcttcgccttcgccctcagacgagt
cggatctccctttgggccgcctccccgcctggtacctttaagaccaatgacttacaaggcagctgtagatcttag
ccactttttaaaagaaaaggggggactggaagggctaattcactcccaacgaaaataagatctgctttttgcttg
tactgggtctctctggttagaccagatctgagcctgggagctctctggctaactagggaacccactgcttaagcc
tcaataaagcttgccttgagtgcttcaagtagtgtgtgcccgtctgttgtgtgactctggtaactagagatccct
cagacccttttagtcagtgtggaaaatctctagcaCGTATGTGTATGATACATAAGGTTATGTATTAATTGTAGC
CGCGTTCTAACGACAATATGTACAAGCCTAATTGTGTAGCATCTGGCTTACTGAAGCAGACCCTATCATCTCTCT
CGTAAACTGCCGTCAGAGTCGGTTTGGTTGGACGAACCTTCTGAGTTTCTGGTAACGCCGTCCCGCACCCGGAAA
TGGTCAGCGAACCAATCAGCAGGGTCATCGCTAGCCAGATCCTCTACGCCGGACGCATCGTGGCCGGCATCACCG
GCGCCACAGGTGCGGTTGCTGGCGCCTATATCGCCGACATCACCGATGGGGAAGATCGGGCTCGCCACTTCGGGC
TCATGAGCGCTTGTTTCGGCGTGGGTATGGTGGCAGGCCCCGTGGCCGGGGGACTGTTGGGCGCCATCTCCTTGC
ATGCACCATTCCTTGCGGCGGCGGTGCTCAACGGCCTCAACCTACTACTGGGCTGCTTCCTAATGCAGGAGTCGC
ATAAGGGAGAGCGTCGAATGGTGCACTCTCAGTACAATCTGCTCTGATGCCGCATAGTTAAGCCAGCCCCGACAC
CCGCCAACACCCGCTGACGCGCCCTGACGGGCTTGTCTGCTCCCGGCATCCGCTTACAGACAAGCTGTGACCGTC
TCCGGGAGCTGCATGTGTCAGAGGTTTTCACCGTCATCACCGAAACGCGCGAGACGAAAGGGCCTCGTGATACGC
CTATTTTTATAGGTTAATGTCATGATAATAATGGTTTCTTAGACGTCAGGTGGCACTTTTCGGGGAAATGTGCGC
GGAACCCCTATTTGTTTATTTTTCTAAATACATTCAAATATGTATCCGCTCATGAGACAATAACCCTGATAAATG
CTTCAATAATATTGAAAAAGGAAGAGTATGAGTATTCAACATTTCCGTGTCGCCCTTATTCCCTTTTTTGCGGCA
TTTTGCCTTCCTGTTTTTGCTCACCCAGAAACGCTGGTGAAAGTAAAAGATGCTGAAGATCAGTTGGGTGCACGA
GTGGGTTACATCGAACTGGATCTCAACAGCGGTAAGATCCTTGAGAGTTTTCGCCCCGAAGAACGTTTTCCAATG
ATGAGCACTTTTAAAGTTCTGCTATGTGGCGCGGTATTATCCCGTATTGACGCCGGGCAAGAGCAACTCGGTCGC
CGCATACACTATTCTCAGAATGACTTGGTTGAGTACTCACCAGTCACAGAAAAGCATCTTACGGATGGCATGACA
GTAAGAGAATTATGCAGTGCTGCCATAACCATGAGTGATAACACTGCGGCCAACTTACTTCTGACAACGATCGGA
GGACCGAAGGAGCTAACCGCTTTTTTGCACAACATGGGGGATCATGTAACTCGCCTTGATCGTTGGGAACCGGAG
CTGAATGAAGCCATACCAAACGACGAGCGTGACACCACGATGCCTGTAGCAATGGCAACAACGTTGCGCAAACTA
TTAACTGGCGAACTACTTACTCTAGCTTCCCGGCAACAATTAATAGACTGGATGGAGGCGGATAAAGTTGCAGGA
CCACTTCTGCGCTCGGCCCTTCCGGCTGGCTGGTTTATTGCTGATAAATCTGGAGCCGGTGAGCGTGGGTCTCGC
GGTATCATTGCAGCACTGGGGCCAGATGGTAAGCCCTCCCGTATCGTAGTTATCTACACGACGGGGAGTCAGGCA
ACTATGGATGAACGAAATAGACAGATCGCTGAGATAGGTGCCTCACTGATTAAGCATTGGTAACTGTCAGACCAA
GTTTACTCATATATACTTTAGATTGATTTAAAACTTCATTTTTAATTTAAAAGGATCTAGGTGAAGATCCTTTTT
GATAATCTCATGACCAAAATCCCTTAACGTGAGTTTTCGTTCCACTGAGCGTCAGACCCCGTAGAAAAGATCAAA
GGATCTTCTTGAGATCCTTTTTTTCTGCGCGTAATCTGCTGCTTGCAAACAAAAAAACCACCGCTACCAGCGGTG
GTTTGTTTGCCGGATCAAGAGCTACCAACTCTTTTTCCGAAGGTAACTGGCTTCAGCAGAGCGCAGATACCAAAT
ACTGTTCTTCTAGTGTAGCCGTAGTTAGGCCACCACTTCAAGAACTCTGTAGCACCGCCTACATACCTCGCTCTG
CTAATCCTGTTACCAGTGGCTGCTGCCAGTGGCGATAAGTCGTGTCTTACCGGGTTGGACTCAAGACGATAGTTA
CCGGATAAGGCGCAGCGGTCGGGCTGAACGGGGGGTTCGTGCACACAGCCCAGCTTGGAGCGAACGACCTACACC
GAACTGAGATACCTACAGCGTGAGCTATGAGAAAGCGCCACGCTTCCCGAAGGGAGAAAGGCGGACAGGTATCCG
GTAAGCGGCAGGGTCGGAACAGGAGAGCGCACGAGGGAGCTTCCAGGGGGAAACGCCTGGTATCTTTATAGTCCT
GTCGGGTTTCGCCACCTCTGACTTGAGCGTCGATTTTTGTGATGCTCGTCAGGGGGGCGGAGCCTATGGAAAAAC
GCCAGCAACGCGGCCTTTTTACGGTTCCTGGCCTTTTGCTGGCCTTTTGCTCACATGTTCTTTCCTGCGTTATCC
CCTGATTCTGTGGATAACCGTATTACCGCCTTTGAGTGAGCTGATACCGCTCGCCGCAGCCGAACGACCGAGCGC
AGCGAGTCAGTGAGCGAGGAAGCGGAAGAGCGCCCAATACGCAAACCGCCTCTCCCCGCGCGTTGGCCGATTCAT
TAATGCAGCTGTGGAATGTGTGTCAGTTAGGGTGTGGAAAGTCCCCAGGCTCCCCAGCAGGCAGAAGTATGCAAA
GCATGCATCTCAATTAGTCAGCAACCAGGTGTGGAAAGTCCCCAGGCTCCCCAGCAGGCAGAAGTATGCAAAGCA
TGCATCTCAATTAGTCAGCAACCATAGTCCCGCCCCTAACTCCGCCCATCCCGCCCCTAACTCCGCCCAGTTCCG
CCCATTCTCCGCCCCATGGCTGACTAATTTTTTTTATTTATGCAGAGGCCGAGGCCGCCTCGGCCTCTGAGCTAT
TCCAGAAGTAGTGAGGAGGCTTTTTTGGAGGCCTAGGCTTTTGCAAAAAGCTTGGACACAAGACAGGCTTGCGAG
ATATGTTTGAGAATACCACTTTATCCCGCGTCAGGGAGAGGCAGTGCGTAAAAAGACGCGGACTCATGTGAAATA
CTGGTTTTTAGTGCGCCAGATCTCTATAATCTCGCGCAACCTATTTTCCCCTCGAACACTTTTTAAGCCGTAGAT
AAACAGGCTGGGACACTTCACATGAGCGAAAAATACATCGTCACCTGGGACATGTTGCAGATCCATGCACGTAAA
CTCGCAAGCCGACTGATGCCTTCTGAACAATGGAAAGGCATTATTGCCGTAAGCCGTGGCGGTCTGTACCGGGTG
CGTTACTGGCGCGTGAACTGGGTATTCGTCATGTCGATACCGTTTGTATTTCCAGCTACGATCACGACAACCAGC
GCGAGCTTAAAGTGCTGAAACGCGCAGAAGGCGATGGCGAAGGCTTCATCGTTATTGATGACCTGGTGGATACCG
GTGGTACTGCGGTTGCGATTCGTGAAATGTATCCAAAAGCGCACTTTGTCACCATCTTCGCAAAACCGGCTGGTC
GTCCGCTGGTTGATGACTATGTTGTTGATATCCCGCAAGATACCTGGATTGAACAGCCGTGGGATATGGGCGTCG
TATTCGTCCCGCCAATCTCCGGTCGCTAATCTTTTCAACGCCTGGCACTGCCGGGCGTTGTTCTTTTTAACTTCA
GGCGGGTTACAATAGTTTCCAGTAAGTATTCTGGAGGCTGCATCCATGACACAGGCAAACCTGAGCGAAACCCTG
TTCAAACCCCGCTTTAAACATCCTGAAACCTCGACGCTAGTCCGCCGCTTTAATCACGGCGCACAACCGCCTGTG
CAGTCGGCCCTTGATGGTAAAACCATCCCTCACTGGTATCGCATGATTAACCGTCTGATGTGGATCTGGCGCGGC
ATTGACCCACGCGAAATCCTCGACGTCCAGGCACGTATTGTGATGAGCGATGCCGAACGTACCGACGATGATTTA
TACGATACGGTGATTGGCTACCGTGGCGGCAACTGGATTTATGAGTGGGCCCCGGATCTTTGTGAAGGAACCTTA
CTTCTGTGGTGTGACATAATTGGACAAACTACCTACAGAGATTTAAAGCTCTAAGGTAAATATAAAATTTTTAAG
TGTATAATGTGTTAAACTACTGATTCTAATTGTTTGTGTATTTTAGATTCCAACCTATGGAACTGATGAATGGGA
GCAGTGGTGGAATGCCTTTAATGAGGAAAACCTGTTTTGCTCAGAAGAAATGCCATCTAGTGATGATGAGGCTAC
TGCTGACTCTCAACATTCTACTCCTCCAAAAAAGAAGAGAAAGGTAGAAGACCCCAAGGACTTTCCTTCAGAATT
GCTAAGTTTTTTGAGTCATGCTGTGTTTAGTAATAGAACTCTTGCTTGCTTTGCTATTTACACCACAAAGGAAAA
AGCTGCACTGCTATACAAGAAAATTATGGAAAAATATTCTGTAACCTTTATAAGTAGGCATAACAGTTATAATCA
TAACATACTGTTTTTTCTTACTCCACACAGGCATAGAGTGTCTGCTATTAATAACTATGCTCAAAAATTGTGTAC
CTTTAGCTTTTTAATTTGTAAAGGGGTTAATAAGGAATATTTGATGTATAGTGCCTTGACTAGAGATCATAATCA
GCCATACCACATTTGTAGAGGTTTTACTTGCTTTAAAAAACCTCCCACACCTCCCCCTGAACCTGAAACATAAAA
TGAATGCAATTGTTGTTGTTAACTTGTTTATTGCAGCTTATAATGGTTACAAATAAAGCAATAGCATCACAAATT
TCACAAATAAAGCATTTTTTTCACTGCATTCTAGTTGTGGTTTGTCCAAACTCATCAATGTATCTTATCATGTCT
GGATCAACTGGATAACTCAAGCTAACCAAAATCATCCCAAACTTCCCACCCCATACCCTATTACCACTGCCAATT
ACCTGTGGTTTCATTTACTCTAAACCTGTGATTCCTCTGAATTATTTTCATTTTAAAGAAATTGTATTTGTTAAA
TATGTACTACAAACTTAGTAG

8. My9.6-CD8-41BB-CD3z_2

An exemplary CAR construct, as described herein, comprises a CD33 binding domain (My9.6, wherein the VH-CDR3 sequence comprises the amino acid sequence RGGYSDYDYYFDF [SEQ ID NO: 31]), a CD8a transmembrane domain, a CD8a hinge domain, a CD137 (4-1BB) co-stimulatory domain, and a CD3ζ intracellular signaling domain.

In some embodiments, a CAR in encoded by a nucleic acid sequence that comprises the sequence that is shown in SEQ ID NO: 32, or in a nucleic acid sequence that is at least 70%, at least 75%, at least 80%, at least 85%, at least 90%, at least 95%, or at least 99% identical to the nucleic acid sequence shown in SEQ ID NO: 32.

[SEQ ID NO: 32]
ATGGCCCTCCCTGTCACCGCCCTGCTGCTTCCGCTGGCTCTTCTGCTCCACGCCGCTCGGCCCCAAGTGCAGCTC
GTCCAATCCGGTGCAGAAGTGAAGAAGCCTGGCGAATCCCTGAAGATCTCATGCAAAGGCTCGGGATACAGCTTC
ACCTCATATTGGATTGGATGGGTCAGACAGATGCCAGGAAAGGGTCTGGAGTGGATGGGAATCATCTACCCGGGA
GACAGCGATACCCGGTACTCCCCGAGCTTCCAGGGACAGGTCACCATCTCGGCCGACAAGTCCATTACTACTGCC
TACTTGCAATGGTCCTCGCTGCGCGCCTCCGATAGCGCCATGTACTACTGCGCGAGAGGCGGCTACTCCGACTAC
GACTACTACTTCGATTTCTGGGGACAGGGGACACTCGTGACTGTGTCCTCCGCGTCGGGTGGCGGCGGCTCGGGT
GGAGGAGGAAGCGGAGGGGGAGGCTCCGAAATTGTGATGACCCAGTCACCCCTGTCGCTCCCTGTGACTCCTGGG
GAACCGGCCTCCATCTCCTGCCGGAGCTCACAGAGCCTGCTGCACTCCAACGGATACAACTACCTCGATTGGTAC
CTTCAGAAGCCCGGCCAGTCGCCCCAGCTGCTGATCTACCTGGGGTCCAACCGGGCTAGCGGCGTGCCGGACCGC
TTCTCCGGTTCCGGGTCTGGAACCGACTTCACGCTGAAAATCTCCAGGGTGGAGGCCGAGGACGTGGGAGTGTAT
TACTGTATGCAGGCCCTGCAAACCCCCTTCACCTTTGGCGGGGGCACCAAGGTCGAGATTAAGACCACTACCCCA
GCACCGAGGCCACCCACCCCGGCTCCTACCATCGCCTCCCAGCCTCTGTCCCTGCGTCCGGAGGCATGTAGACCC
GCAGCTGGTGGGGCCGTGCATACCCGGGGTCTTGACTTCGCCTGCGATATCTACATTTGGGCCCCTCTGGCTGGT
ACTTGCGGGGTCCTGCTGCTTTCACTCGTGATCACTCTTTACTGTAAGCGCGGTCGGAAGAAGCTGCTGTACATC
TTTAAGCAACCCTTCATGAGGCCTGTGCAGACTACTCAAGAGGAGGACGGCTGTTCATGCCGGTTCCCAGAGGAG
GAGGAAGGCGGCTGCGAACTGCGCGTGAAATTCAGCCGCAGCGCAGATGCTCCAGCCTACAAGCAGGGGCAGAAC
CAGCTCTACAACGAACTCAATCTTGGTCGGAGAGAGGAGTACGACGTGCTGGACAAGCGGAGAGGACGGGACCCA
GAAATGGGCGGGAAGCCGCGCAGAAAGAATCCCCAAGAGGGCCTGTACAACGAGCTCCAAAAGGATAAGATGGCA
GAAGCCTATAGCGAGATTGGTATGAAAGGGGAACGCAGAAGAGGCAAAGGCCACGACGGACTGTACCAGGGACTC
AGCACCGCCACCAAGGACACCTATGACGCTCTTCACATGCAGGCCCTGCCGCCTCGG

In some embodiments, a CAR comprises an amino acid sequence shown in SEQ ID NO: 33, or an amino acid sequence that is at least 70%, at least 75%, at least 80%, at least 85%, at least 90%, at least 95%, or at least 99% identical to the amino acid sequence shown in SEQ ID NO: 33.

[SEQ ID NO: 33]
MALPVTALLLPLALLLHAARPQVQLVQSGAEVKKPGESLKISCKGSGYSFTSYWIGWVRQMPGKGLEWMGIIYPG
DSDTRYSPSFQGQVTISADKSITTAYLOWSSLRASDSAMYYCARGGYSDYDYYFDFWGQGTLVTVSSASGGGGSG

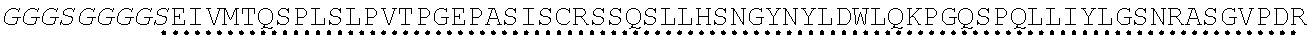

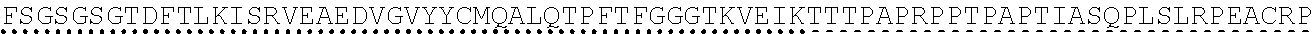

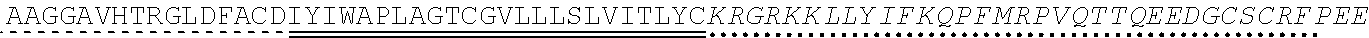

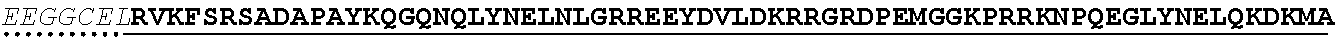

EAYSEIGMKGERRRGKGHDGLYQGLSTATKDTYDALHMQALPPR

In some embodiments, a CAR construct as shown in SEQ ID NO: 32 is included in a recombinant expression vector. In some embodiments the recombinant expression vector includes a promoter (e.g., an SFFV promoter or and EF1α promoter). In some embodiments, a recombinant expression vector including the CAR of SEQ ID NO: 32 comprises the sequence that is shown in SEQ ID NO: 34, or in a nucleic acid sequence that is at least 70%, at least 75%, at least 80%, at least 85%, at least 90%, at least 95%, or at least 99% identical to the nucleic acid sequence shown in SEQ ID NO: 34.

In some embodiments, a recombinant expression vector including the CAR of SEQ ID NO: 32 comprises the sequence that is shown in SEQ ID NO: 34, but does not include the SFFV promoter sequence (as shown in SEQ ID NO: 8). In some embodiments, a recombinant expression vector comprises the nucleic acid sequence shown in SEQ ID NO: 34, but does not include the SFFV sequence (as shown in SEQ ID NO: 8), and instead includes an EF1α promoter sequence.

[SEQ ID NO: 34]
Tgggtctctctggttagaccagatctgagcctgggagctctctggctaactagggaacccactgcttaagcctca
ataaagcttgccttgagtgcttcaagtagtgtgtgcccgtctgttgtgtgactctggtaactagagatccctcag
acccttttagtcagtgtggaaaatctctagcagtggcgcccgaacagggacttgaaagcgaaagggaaaccagag
gagctctctcgacgcaggactcggcttgctgaagcgcgcacggcaagaggcgaggggCggcgactggtgagtacg
ccaaaaattttgactagcggaggctagaaggagagagatgggtgcgagagcgtcagtattaagcgggggagaatt
agatcgcgatgggaaaaaattcggttaaggccagggggaaagaaaaaatataaattaaaacatatagtatgggca
agcagggagctagaacgattcgcagttaatcctggcctgttagaaacatcagaaggctgtagacaaatactggga
cagctacaaccatcccttcagacaggatcagaagaacttagatcattatataatacagtagcaaccctctattgt
gtgcatcaaaggatagagataaaagacaccaaggaagctttagacaagatagaggaagagcaaaacaaaagtaag
accaccgcacagcaagcggccactgatcttcagacctggaggaggagatatgagggacaattggagaagtgaatt
atataaatataaagtagtaaaaattgaaccattaggagtagcacccaccaaggcaaagagaagagtggtgcagag
agaaaaaagagcagtgggaataggagctttgttccttgggttcttgggagcagcaggaagcactatgggcgcagc
gtcaatgacgctgacggtacaggccagacaattattgtctggtatagtgcagcagcagaacaatttgctgagggc
tattgaggcgcaacagcatctgttgcaactcacagtctggggcatcaagcagctccaggcaagaatcctggctgt
ggaaagatacctaaaggatcaacagctcctggggatttggggttgctctggaaaactcatttgcaccactgctgt
gccttggaatgctagttggagtaataaatctctggaacagaattggaatcacacgacctggatggagtgggacag
agaaattaacaattacacaagcttaatacactccttaattgaagaatcgcaaaaccagcaagaaaagaatgaaca
agaattattggaattagataaatgggcaagtttgtggaattggtttaacataacaaattggctgtggtatataaa
attattcataatgatagtaggaggcttggtaggtttaagaatagtttttgctgtactttctatagtgaatagagt
taggcagggatattcaccattatcgtttcagacccacctcccaaccccgaggggacccgacaggcccgaaggaat
agaagaagaaggtggagagagagacagagacagatccattcgattagtgaacggatctcgacggtatcggttaac
ttttaaaagaaaaggggggattggggggtacagtgcaggggaaagaatagtagacataatagcaacagacataca
aactaaagaattacaaaaacaaattacaaaattcaaaattttatcgatactagtggatctGCGATCGCGTAACGC
CATTTTGCAAGGCATGGAAAAATACCAAACCAAGAATAGAGAAGTTCAGATCAAGGGCGGGTACATGAAAATAGC
TAACGTTGGGCCAAACAGGATATCTGCGGTGAGCAGTTTCGGCCCCGGCCCGGGGCCAAGAACAGATGGTCACCG
CAGTTTCGGCCCCGGCCCGAGGCCAAGAACAGATGGTCCCCAGATATGGCCCAACCCTCAGCAGTTTCTTAAGAC
CCATCAGATGTTTCCAGGCTCCCCCAAGGACCTGAAATGACCCTGCGCCTTATTTGAATTAACCAATCAGCCTGC
TTCTCGCTTCTGTTCGCGCGCTTCTGCTTCCCGAGCTCTATAAAAGAGCTCACAACCCCTCACTCGGCGCGCCAG
TCCTCCGACAGACTGAGTCGTCTAGAgctagcGGATCCCCGGAATTCGACGCCACCATGGCCCTCCCTGTCACCG
CCCTGCTGCTTCCGCTGGCTCTTCTGCTCCACGCCGCTCGGCCCCAAGTGCAGCTCGTCCAATCCGGTGCAGAAG
TGAAGAAGCCTGGCGAATCCCTGAAGATCTCATGCAAAGGCTCGGGATACAGCTTCACCTCATATTGGATTGGAT
GGGTCAGACAGATGCCAGGAAAGGGTCTGGAGTGGATGGGAATCATCTACCCGGGAGACAGCGATACCCGGTACT
CCCCGAGCTTCCAGGGACAGGTCACCATCTCGGCCGACAAGTCCATTACTACTGCCTACTTGCAATGGTCCTCGC
TGCGCGCCTCCGATAGCGCCATGTACTACTGCGCGAGAGGCGGCTACTCCGACTACGACTACTACTTCGATTTCT
GGGGACAGGGGACACTCGTGACTGTGTCCTCCGCGTCGGGTGGCGGCGGCTCGGGTGGAGGAGGAAGCGGAGGGG
GAGGCTCCGAAATTGTGATGACCCAGTCACCCCTGTCGCTCCCTGTGACTCCTGGGGAACCGGCCTCCATCTCCT
GCCGGAGCTCACAGAGCCTGCTGCACTCCAACGGATACAACTACCTCGATTGGTACCTTCAGAAGCCCGGCCAGT
CGCCCCAGCTGCTGATCTACCTGGGGTCCAACCGGGCTAGCGGCGTGCCGGACCGCTTCTCCGGTTCCGGGTCTG
GAACCGACTTCACGCTGAAAATCTCCAGGGTGGAGGCCGAGGACGTGGGAGTGTATTACTGTATGCAGGCCCTGC
AAACCCCCTTCACCTTTGGCGGGGGCACCAAGGTCGAGATTAAGACCACTACCCCAGCACCGAGGCCACCCACCC
CGGCTCCTACCATCGCCTCCCAGCCTCTGTCCCTGCGTCCGGAGGCATGTAGACCCGCAGCTGGTGGGGCCGTGC
ATACCCGGGGTCTTGACTTCGCCTGCGATATCTACATTTGGGCCCCTCTGGCTGGTACTTGCGGGGTCCTGCTGC
TTTCACTCGTGATCACTCTTTACTGTAAGCGCGGTCGGAAGAAGCTGCTGTACATCTTTAAGCAACCCTTCATGA
GGCCTGTGCAGACTACTCAAGAGGAGGACGGCTGTTCATGCCGGTTCCCAGAGGAGGAGGAAGGCGGCTGCGAAC
TGCGCGTGAAATTCAGCCGCAGCGCAGATGCTCCAGCCTACAAGCAGGGGCAGAACCAGCTCTACAACGAACTCA
ATCTTGGTCGGAGAGAGGAGTACGACGTGCTGGACAAGCGGAGAGGACGGGACCCAGAAATGGGCGGGAAGCCGC
GCAGAAAGAATCCCCAAGAGGGCCTGTACAACGAGCTCCAAAAGGATAAGATGGCAGAAGCCTATAGCGAGATTG
GTATGAAAGGGGAACGCAGAAGAGGCAAAGGCCACGACGGACTGTACCAGGGACTCAGCACCGCCACCAAGGACA
CCTATGACGCTCTTCACATGCAGGCCCTGCCGCCTCGGgagggcagaggaagtcttctaacatgcggtgacgtgg
aggagaatcccggcccttccgggatgaccgagtacaagcccacggtgcgcctcgccacccgcgacgacgtcccca
gggccgtacgcaccctcgccgccgcgttcgccgactaccccgccacgcgccacaccgtcgatccggaccgccaca
tcgagcgggtcaccgagctgcaagaactcttcctcacgcgcgtcgggctcgacatcggcaaggtgtgggtcgcgg
acgacggcgccgcggtggcggtctggaccacgccggagagcgtcgaagcgggggcggtgttcgccgagatcggcc
cgcgcatggccgagttgagcggttcccggctggccgcgcagcaacagatggaaggcctcctggcgccgcaccggc
ccaaggagcccgcgtggttcctggccaccgtcggcgtctcgcccgaccaccagggcaagggtctgggcagcgccg
tcgtgctccccggagtggaggcggccgagcgcgccggggtgcccgccttcctggagacctccgcgccccgcaacc
tccccttctacgagcggctcggcttcaccgtcaccgccgacgtcgaggtgcccgaaggaccgcgcacctggtgca
tgacccgcaagcccggtgcctgaATTAATTAACCAATTGaatcaacctctggattacaaaatttgtgaaagattg
actggtattcttaactatgttgctccttttacgctatgtggatacgctgctttaatgcctttgtatcatgctatt
gcttcccgtatggctttcattttctcctccttgtataaatcctggttgctgtctctttatgaggagttgtggccc
gttgtcaggcaacgtggcgtggtgtgcactgtgtttgctgacgcaacccccactggttggggcattgccaccacc
tgtcagctcctttccgggactttcgctttccccctccctattgccacggcggaactcategccgcctgccttgcc
cgctgctggacaggggctcggctgttgggcactgacaattccgtggtgttgtcggggaaatcatcgtcctttcct
tggctgctcgcctgtgttgccacctggattctgcgcgggacgtccttctgctacgtcccttcggccctcaatcca
gcggaccttccttcccgcggcctgctgccggctctgcggcctcttccgcgtcttcgccttcgccctcagacgagt
cggatctccctttgggccgcctccccgcctggtacctttaagaccaatgacttacaaggcagctgtagatcttag
ccactttttaaaagaaaaggggggactggaagggctaattcactcccaacgaaaataagatctgctttttgcttg
tactgggtctctctggttagaccagatctgagcctgggagctctctggctaactagggaacccactgcttaagcc
tcaataaagcttgccttgagtgcttcaagtagtgtgtgcccgtctgttgtgtgactctggtaactagagatccct
cagacccttttagtcagtgtggaaaatctctagcaCGTATGTGTATGATACATAAGGTTATGTATTAATTGTAGC
CGCGTTCTAACGACAATATGTACAAGCCTAATTGTGTAGCATCTGGCTTACTGAAGCAGACCCTATCATCTCTCT
CGTAAACTGCCGTCAGAGTCGGTTTGGTTGGACGAACCTTCTGAGTTTCTGGTAACGCCGTCCCGCACCCGGAAA
TGGTCAGCGAACCAATCAGCAGGGTCATCGCTAGCCAGATCCTCTACGCCGGACGCATCGTGGCCGGCATCACCG
GCGCCACAGGTGCGGTTGCTGGCGCCTATATCGCCGACATCACCGATGGGGAAGATCGGGCTCGCCACTTCGGGC
TCATGAGCGCTTGTTTCGGCGTGGGTATGGTGGCAGGCCCCGTGGCCGGGGGACTGTTGGGCGCCATCTCCTTGC
ATGCACCATTCCTTGCGGCGGCGGTGCTCAACGGCCTCAACCTACTACTGGGCTGCTTCCTAATGCAGGAGTCGC
ATAAGGGAGAGCGTCGAATGGTGCACTCTCAGTACAATCTGCTCTGATGCCGCATAGTTAAGCCAGCCCCGACAC
CCGCCAACACCCGCTGACGCGCCCTGACGGGCTTGTCTGCTCCCGGCATCCGCTTACAGACAAGCTGTGACCGTC
TCCGGGAGCTGCATGTGTCAGAGGTTTTCACCGTCATCACCGAAACGCGCGAGACGAAAGGGCCTCGTGATACGC
CTATTTTTATAGGTTAATGTCATGATAATAATGGTTTCTTAGACGTCAGGTGGCACTTTTCGGGGAAATGTGCGC
GGAACCCCTATTTGTTTATTTTTCTAAATACATTCAAATATGTATCCGCTCATGAGACAATAACCCTGATAAATG
CTTCAATAATATTGAAAAAGGAAGAGTATGAGTATTCAACATTTCCGTGTCGCCCTTATTCCCTTTTTTGCGGCA
TTTTGCCTTCCTGTTTTTGCTCACCCAGAAACGCTGGTGAAAGTAAAAGATGCTGAAGATCAGTTGGGTGCACGA
GTGGGTTACATCGAACTGGATCTCAACAGCGGTAAGATCCTTGAGAGTTTTCGCCCCGAAGAACGTTTTCCAATG
ATGAGCACTTTTAAAGTTCTGCTATGTGGCGCGGTATTATCCCGTATTGACGCCGGGCAAGAGCAACTCGGTCGC
CGCATACACTATTCTCAGAATGACTTGGTTGAGTACTCACCAGTCACAGAAAAGCATCTTACGGATGGCATGACA
GTAAGAGAATTATGCAGTGCTGCCATAACCATGAGTGATAACACTGCGGCCAACTTACTTCTGACAACGATCGGA
GGACCGAAGGAGCTAACCGCTTTTTTGCACAACATGGGGGATCATGTAACTCGCCTTGATCGTTGGGAACCGGAG
CTGAATGAAGCCATACCAAACGACGAGCGTGACACCACGATGCCTGTAGCAATGGCAACAACGTTGCGCAAACTA
TTAACTGGCGAACTACTTACTCTAGCTTCCCGGCAACAATTAATAGACTGGATGGAGGCGGATAAAGTTGCAGGA
CCACTTCTGCGCTCGGCCCTTCCGGCTGGCTGGTTTATTGCTGATAAATCTGGAGCCGGTGAGCGTGGGTCTCGC
GGTATCATTGCAGCACTGGGGCCAGATGGTAAGCCCTCCCGTATCGTAGTTATCTACACGACGGGGAGTCAGGCA
ACTATGGATGAACGAAATAGACAGATCGCTGAGATAGGTGCCTCACTGATTAAGCATTGGTAACTGTCAGACCAA
GTTTACTCATATATACTTTAGATTGATTTAAAACTTCATTTTTAATTTAAAAGGATCTAGGTGAAGATCCTTTTT
GATAATCTCATGACCAAAATCCCTTAACGTGAGTTTTCGTTCCACTGAGCGTCAGACCCCGTAGAAAAGATCAAA
GGATCTTCTTGAGATCCTTTTTTTCTGCGCGTAATCTGCTGCTTGCAAACAAAAAAACCACCGCTACCAGCGGTG
GTTTGTTTGCCGGATCAAGAGCTACCAACTCTTTTTCCGAAGGTAACTGGCTTCAGCAGAGCGCAGATACCAAAT
ACTGTTCTTCTAGTGTAGCCGTAGTTAGGCCACCACTTCAAGAACTCTGTAGCACCGCCTACATACCTCGCTCTG
CTAATCCTGTTACCAGTGGCTGCTGCCAGTGGCGATAAGTCGTGTCTTACCGGGTTGGACTCAAGACGATAGTTA
CCGGATAAGGCGCAGCGGTCGGGCTGAACGGGGGGTTCGTGCACACAGCCCAGCTTGGAGCGAACGACCTACACC
GAACTGAGATACCTACAGCGTGAGCTATGAGAAAGCGCCACGCTTCCCGAAGGGAGAAAGGCGGACAGGTATCCG
GTAAGCGGCAGGGTCGGAACAGGAGAGCGCACGAGGGAGCTTCCAGGGGGAAACGCCTGGTATCTTTATAGTCCT
GTCGGGTTTCGCCACCTCTGACTTGAGCGTCGATTTTTGTGATGCTCGTCAGGGGGGCGGAGCCTATGGAAAAAC
GCCAGCAACGCGGCCTTTTTACGGTTCCTGGCCTTTTGCTGGCCTTTTGCTCACATGTTCTTTCCTGCGTTATCC
CCTGATTCTGTGGATAACCGTATTACCGCCTTTGAGTGAGCTGATACCGCTCGCCGCAGCCGAACGACCGAGCGC
AGCGAGTCAGTGAGCGAGGAAGCGGAAGAGCGCCCAATACGCAAACCGCCTCTCCCCGCGCGTTGGCCGATTCAT
TAATGCAGCTGTGGAATGTGTGTCAGTTAGGGTGTGGAAAGTCCCCAGGCTCCCCAGCAGGCAGAAGTATGCAAA
GCATGCATCTCAATTAGTCAGCAACCAGGTGTGGAAAGTCCCCAGGCTCCCCAGCAGGCAGAAGTATGCAAAGCA
TGCATCTCAATTAGTCAGCAACCATAGTCCCGCCCCTAACTCCGCCCATCCCGCCCCTAACTCCGCCCAGTTCCG
CCCATTCTCCGCCCCATGGCTGACTAATTTTTTTTATTTATGCAGAGGCCGAGGCCGCCTCGGCCTCTGAGCTAT
TCCAGAAGTAGTGAGGAGGCTTTTTTGGAGGCCTAGGCTTTTGCAAAAAGCTTGGACACAAGACAGGCTTGCGAG
ATATGTTTGAGAATACCACTTTATCCCGCGTCAGGGAGAGGCAGTGCGTAAAAAGACGCGGACTCATGTGAAATA
CTGGTTTTTAGTGCGCCAGATCTCTATAATCTCGCGCAACCTATTTTCCCCTCGAACACTTTTTAAGCCGTAGAT
AAACAGGCTGGGACACTTCACATGAGCGAAAAATACATCGTCACCTGGGACATGTTGCAGATCCATGCACGTAAA
CTCGCAAGCCGACTGATGCCTTCTGAACAATGGAAAGGCATTATTGCCGTAAGCCGTGGCGGTCTGTACCGGGTG
CGTTACTGGCGCGTGAACTGGGTATTCGTCATGTCGATACCGTTTGTATTTCCAGCTACGATCACGACAACCAGC
GCGAGCTTAAAGTGCTGAAACGCGCAGAAGGCGATGGCGAAGGCTTCATCGTTATTGATGACCTGGTGGATACCG
GTGGTACTGCGGTTGCGATTCGTGAAATGTATCCAAAAGCGCACTTTGTCACCATCTTCGCAAAACCGGCTGGTC
GTCCGCTGGTTGATGACTATGTTGTTGATATCCCGCAAGATACCTGGATTGAACAGCCGTGGGATATGGGCGTCG
TATTCGTCCCGCCAATCTCCGGTCGCTAATCTTTTCAACGCCTGGCACTGCCGGGCGTTGTTCTTTTTAACTTCA
GGCGGGTTACAATAGTTTCCAGTAAGTATTCTGGAGGCTGCATCCATGACACAGGCAAACCTGAGCGAAACCCTG
TTCAAACCCCGCTTTAAACATCCTGAAACCTCGACGCTAGTCCGCCGCTTTAATCACGGCGCACAACCGCCTGTG
CAGTCGGCCCTTGATGGTAAAACCATCCCTCACTGGTATCGCATGATTAACCGTCTGATGTGGATCTGGCGCGGC
ATTGACCCACGCGAAATCCTCGACGTCCAGGCACGTATTGTGATGAGCGATGCCGAACGTACCGACGATGATTTA
TACGATACGGTGATTGGCTACCGTGGCGGCAACTGGATTTATGAGTGGGCCCCGGATCTTTGTGAAGGAACCTTA
CTTCTGTGGTGTGACATAATTGGACAAACTACCTACAGAGATTTAAAGCTCTAAGGTAAATATAAAATTTTTAAG
TGTATAATGTGTTAAACTACTGATTCTAATTGTTTGTGTATTTTAGATTCCAACCTATGGAACTGATGAATGGGA
GCAGTGGTGGAATGCCTTTAATGAGGAAAACCTGTTTTGCTCAGAAGAAATGCCATCTAGTGATGATGAGGCTAC
TGCTGACTCTCAACATTCTACTCCTCCAAAAAAGAAGAGAAAGGTAGAAGACCCCAAGGACTTTCCTTCAGAATT
GCTAAGTTTTTTGAGTCATGCTGTGTTTAGTAATAGAACTCTTGCTTGCTTTGCTATTTACACCACAAAGGAAAA
AGCTGCACTGCTATACAAGAAAATTATGGAAAAATATTCTGTAACCTTTATAAGTAGGCATAACAGTTATAATCA
TAACATACTGTTTTTTCTTACTCCACACAGGCATAGAGTGTCTGCTATTAATAACTATGCTCAAAAATTGTGTAC
CTTTAGCTTTTTAATTTGTAAAGGGGTTAATAAGGAATATTTGATGTATAGTGCCTTGACTAGAGATCATAATCA
GCCATACCACATTTGTAGAGGTTTTACTTGCTTTAAAAAACCTCCCACACCTCCCCCTGAACCTGAAACATAAAA
TGAATGCAATTGTTGTTGTTAACTTGTTTATTGCAGCTTATAATGGTTACAAATAAAGCAATAGCATCACAAATT
TCACAAATAAAGCATTTTTTTCACTGCATTCTAGTTGTGGTTTGTCCAAACTCATCAATGTATCTTATCATGTCT
GGATCAACTGGATAACTCAAGCTAACCAAAATCATCCCAAACTTCCCACCCCATACCCTATTACCACTGCCAATT
ACCTGTGGTTTCATTTACTCTAAACCTGTGATTCCTCTGAATTATTTTCATTTTAAAGAAATTGTATTTGTTAAA
TATGTACTACAAACTTAGTAG

9. My9.6-CD8-41BB-CD3z_3

An exemplary CAR construct, as described herein, comprises a CD33 binding domain (My9.6, wherein the CD33 binding domain comprises a VL-VH orientation), a CD8a transmembrane domain, a CD8a hinge domain, a CD137 (4-1BB) co-stimulatory domain, and a CD3 intracellular signaling domain.

In some embodiments, a CAR in encoded by a nucleic acid sequence that comprises the sequence that is shown in SEQ ID NO: 35, or in a nucleic acid sequence that is at least 70%, at least 75%, at least 80%, at least 85%, at least 90%, at least 95%, or at least 99% identical to the nucleic acid sequence shown in SEQ ID NO: 35.

[SEQ ID NO: 35]
ATGGCCCTGCCTGTGACAGCCCTGCTGCTGCCTCTGGCTCTGCTGCTGCATGCCGCTAGACCCGGATCCGAGATC
GTGCTGACACAGAGCCCTGGAAGCCTGGCCGTGTCTCCTGGCGAGCGCGTGACAATGAGCTGCAAGAGCAGCCAG
AGCGTGTTCTTCAGCAGCTCCCAGAAGAACTACCTGGCCTGGTATCAGCAGATCCCCGGCCAGAGCCCCAGACTG
CTGATCTACTGGGCCAGCACCAGAGAAAGCGGCGTGCCCGATAGATTCACCGGCAGCGGCTCTGGCACCGACTTC
ACCCTGACAATCAGCAGCGTGCAGCCCGAGGACCTGGCCATCTACTACTGCCACCAGTACCTGAGCAGCCGGACC
TTTGGCCAGGGCACCAAGCTGGAAATCAAGAGAGGCGGCGGAGGCTCTGGCGGAGGCGGATCTAGTGGCGGAGGA
TCTCAGGTGCAGCTGCAGCAGCCTGGCGCCGAGGTCGTGAAACCTGGCGCCTCTGTGAAGATGTCCTGCAAGGCC
AGCGGCTACACCTTCACCAGCTACTACATCCACTGGATCAAGCAGACCCCTGGACAGGGCCTGGAATGGGTGGGA
GTGATCTACCCCGGCAACGACGACATCAGCTACAACCAGAAGTTCCAGGGCAAGGCCACCCTGACCGCCGACAAG
TCTAGCACCACCGCCTACATGCAGCTGTCCAGCCTGACCAGCGAGGACAGCGCCGTGTACTACTGCGCCAGAGAA
GTGCGGCTGCGGTACTTCGATGTGTGGGGCCAGGGAACCACCGTGACCGTGTCATCTACCACTACCCCAGCACCG
AGGCCACCCACCCCGGCTCCTACCATCGCCTCCCAGCCTCTGTCCCTGCGTCCGGAGGCATGTAGACCCGCAGCT
GGTGGGGCCGTGCATACCCGGGGTCTTGACTTCGCCTGCGATATCTACATTTGGGCCCCTCTGGCTGGTACTTGC
GGGGTCCTGCTGCTTTCACTCGTGATCACTCTTTACTGTAAGCGCGGTCGGAAGAAGCTGCTGTACATCTTTAAG
CAACCCTTCATGAGGCCTGTGCAGACTACTCAAGAGGAGGACGGCTGTTCATGCCGGTTCCCAGAGGAGGAGGAA
GGCGGCTGCGAACTGCGCGTGAAATTCAGCCGCAGCGCAGATGCTCCAGCCTACAAGCAGGGGCAGAACCAGCTC
TACAACGAACTCAATCTTGGTCGGAGAGAGGAGTACGACGTGCTGGACAAGCGGAGAGGACGGGACCCAGAAATG
GGCGGGAAGCCGCGCAGAAAGAATCCCCAAGAGGGCCTGTACAACGAGCTCCAAAAGGATAAGATGGCAGAAGCC
TATAGCGAGATTGGTATGAAAGGGGAACGCAGAAGAGGCAAAGGCCACGACGGACTGTACCAGGGACTCAGCACC
GCCACCAAGGACACCTATGACGCTCTTCACATGCAGGCCCTGCCGCCTCGG

In some embodiments, a CAR comprises an amino acid sequence shown in SEQ ID NO: 36, or an amino acid sequence that is at least 70%, at least 75%, at least 80%, at least 85%, at least 90%, at least 95%, or at least 99% identical to the amino acid sequence shown in SEQ ID NO: 36.

[SEQ ID NO: 36]

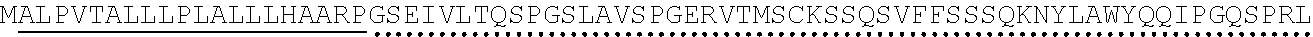

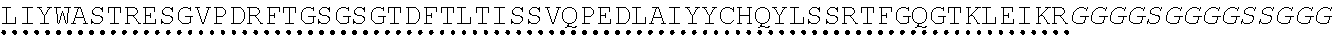

SQVQLQQPGAEVVKPGASVKMSCKASGYTFTSYYIHWIKQTPGQGLEWVGVIYPGNDDISYNQKFQGKATLTADK

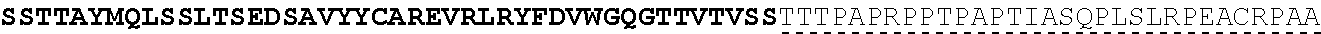

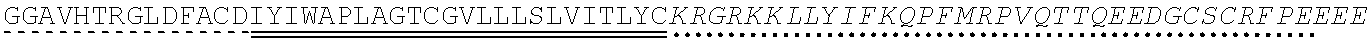

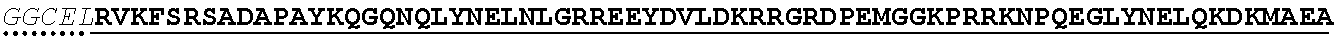

YSEIGMKGERRRGKGHDGLYQGLSTATKDTYDALHMQALPPR

In some embodiments, a CAR construct as shown in SEQ ID NO: 35 is included in a recombinant expression vector. In some embodiments the recombinant expression vector includes a promoter (e.g., an SFFV promoter or and EF1α promoter). In some embodiments, a recombinant expression vector including the CAR of SEQ ID NO: 35 comprises the sequence that is shown in SEQ ID NO: 37, or in a nucleic acid sequence that is at least 70%, at least 75%, at least 80%, at least 85%, at least 90%, at least 95%, or at least 99% identical to the nucleic acid sequence shown in SEQ ID NO: 37.

In some embodiments, a recombinant expression vector including the CAR of SEQ ID NO: 35 comprises the sequence that is shown in SEQ ID NO: 37, but does not include the SFFV promoter sequence (as shown in SEQ ID NO: 8). In some embodiments, a recombinant expression vector comprises the nucleic acid sequence shown in SEQ ID NO: 37, but does not include the SFFV sequence (as shown in SEQ ID NO: 8), and instead includes an EF1α promoter sequence.

[SEQ ID NO: 37]
Tgggtctctctggttagaccagatctgagcctgggagctctctggctaactagggaacccactgcttaagcctca
ataaagcttgccttgagtgcttcaagtagtgtgtgcccgtctgttgtgtgactctggtaactagagatccctcag
acccttttagtcagtgtggaaaatctctagcagtggcgcccgaacagggacttgaaagcgaaagggaaaccagag
gagctctctcgacgcaggactcggcttgctgaagcgcgcacggcaagaggcgaggggcggcgactggtgagtacg
ccaaaaattttgactagcggaggctagaaggagagagatgggtgcgagagcgtcagtattaagcgggggagaatt
agatcgcgatgggaaaaaattcggttaaggccagggggaaagaaaaaatataaattaaaacatatagtatgggca
agcagggagctagaacgattcgcagttaatcctggcctgttagaaacatcagaaggctgtagacaaatactggga
cagctacaaccatcccttcagacaggatcagaagaacttagatcattatataatacagtagcaaccctctattgt
gtgcatcaaaggatagagataaaagacaccaaggaagctttagacaagatagaggaagagcaaaacaaaagtaag
accaccgcacagcaagcggccactgatcttcagacctggaggaggagatatgagggacaattggagaagtgaatt
atataaatataaagtagtaaaaattgaaccattaggagtagcacccaccaaggcaaagagaagagtggtgcagag
agaaaaaagagcagtgggaataggagctttgttccttgggttcttgggagcagcaggaagcactatgggcgcagc
gtcaatgacgctgacggtacaggccagacaattattgtctggtatagtgcagcagcagaacaatttgctgagggc
tattgaggcgcaacagcatctgttgcaactcacagtctggggcatcaagcagctccaggcaagaatcctggctgt
ggaaagatacctaaaggatcaacagctcctggggatttggggttgctctggaaaactcatttgcaccactgctgt
gccttggaatgctagttggagtaataaatctctggaacagaattggaatcacacgacctggatggagtgggacag
agaaattaacaattacacaagcttaatacactccttaattgaagaatcgcaaaaccagcaagaaaagaatgaaca
agaattattggaattagataaatgggcaagtttgtggaattggtttaacataacaaattggctgtggtatataaa
attattcataatgatagtaggaggcttggtaggtttaagaatagtttttgctgtactttctatagtgaatagagt
taggcagggatattcaccattatcgtttcagacccacctcccaaccccgaggggacccgacaggcccgaaggaat
agaagaagaaggtggagagagagacagagacagatccattcgattagtgaacggatctcgacggtatcggttaac
ttttaaaagaaaaggggggattggggggtacagtgcaggggaaagaatagtagacataatagcaacagacataca
aactaaagaattacaaaaacaaattacaaaattcaaaattttatcgatactagtggatctGCGATCGCGTAACGC
CATTTTGCAAGGCATGGAAAAATACCAAACCAAGAATAGAGAAGTTCAGATCAAGGGCGGGTACATGAAAATAGC
TAACGTTGGGCCAAACAGGATATCTGCGGTGAGCAGTTTCGGCCCCGGCCCGGGGCCAAGAACAGATGGTCACCG
CAGTTTCGGCCCCGGCCCGAGGCCAAGAACAGATGGTCCCCAGATATGGCCCAACCCTCAGCAGTTTCTTAAGAC
CCATCAGATGTTTCCAGGCTCCCCCAAGGACCTGAAATGACCCTGCGCCTTATTTGAATTAACCAATCAGCCTGC
TTCTCGCTTCTGTTCGCGCGCTTCTGCTTCCCGAGCTCTATAAAAGAGCTCACAACCCCTCACTCGGCGCGCCAG
TCCTCCGACAGACTGAGTCGTCTAGAgctagcGGATCCCCGGAATTCGACGCCACCATGGCCCTGCCTGTGACAG
CCCTGCTGCTGCCTCTGGCTCTGCTGCTGCATGCCGCTAGACCCGGATCCGAGATCGTGCTGACACAGAGCCCTG
GAAGCCTGGCCGTGTCTCCTGGCGAGCGCGTGACAATGAGCTGCAAGAGCAGCCAGAGCGTGTTCTTCAGCAGCT
CCCAGAAGAACTACCTGGCCTGGTATCAGCAGATCCCCGGCCAGAGCCCCAGACTGCTGATCTACTGGGCCAGCA
CCAGAGAAAGCGGCGTGCCCGATAGATTCACCGGCAGCGGCTCTGGCACCGACTTCACCCTGACAATCAGCAGCG
TGCAGCCCGAGGACCTGGCCATCTACTACTGCCACCAGTACCTGAGCAGCCGGACCTTTGGCCAGGGCACCAAGC
TGGAAATCAAGAGAGGCGGCGGAGGCTCTGGCGGAGGCGGATCTAGTGGCGGAGGATCTCAGGTGCAGCTGCAGC
AGCCTGGCGCCGAGGTCGTGAAACCTGGCGCCTCTGTGAAGATGTCCTGCAAGGCCAGCGGCTACACCTTCACCA
GCTACTACATCCACTGGATCAAGCAGACCCCTGGACAGGGCCTGGAATGGGTGGGAGTGATCTACCCCGGCAACG
ACGACATCAGCTACAACCAGAAGTTCCAGGGCAAGGCCACCCTGACCGCCGACAAGTCTAGCACCACCGCCTACA
TGCAGCTGTCCAGCCTGACCAGCGAGGACAGCGCCGTGTACTACTGCGCCAGAGAAGTGCGGCTGCGGTACTTCG
ATGTGTGGGGCCAGGGAACCACCGTGACCGTGTCATCTACCACTACCCCAGCACCGAGGCCACCCACCCCGGCTC
CTACCATCGCCTCCCAGCCTCTGTCCCTGCGTCCGGAGGCATGTAGACCCGCAGCTGGTGGGGCCGTGCATACCC
GGGGTCTTGACTTCGCCTGCGATATCTACATTTGGGCCCCTCTGGCTGGTACTTGCGGGGTCCTGCTGCTTTCAC
TCGTGATCACTCTTTACTGTAAGCGCGGTCGGAAGAAGCTGCTGTACATCTTTAAGCAACCCTTCATGAGGCCTG
TGCAGACTACTCAAGAGGAGGACGGCTGTTCATGCCGGTTCCCAGAGGAGGAGGAAGGCGGCTGCGAACTGCGCG
TGAAATTCAGCCGCAGCGCAGATGCTCCAGCCTACAAGCAGGGGCAGAACCAGCTCTACAACGAACTCAATCTTG
GTCGGAGAGAGGAGTACGACGTGCTGGACAAGCGGAGAGGACGGGACCCAGAAATGGGCGGGAAGCCGCGCAGAA
AGAATCCCCAAGAGGGCCTGTACAACGAGCTCCAAAAGGATAAGATGGCAGAAGCCTATAGCGAGATTGGTATGA
AAGGGGAACGCAGAAGAGGCAAAGGCCACGACGGACTGTACCAGGGACTCAGCACCGCCACCAAGGACACCTATG
ACGCTCTTCACATGCAGGCCCTGCCGCCTCGGgagggcagaggaagtcttctaacatgcggtgacgtggaggaga
atcccggcccttccgggatgaccgagtacaagcccacggtgcgcctcgccacccgcgacgacgtccccagggccg
tacgcaccctcgccgccgcgttcgccgactaccccgccacgcgccacaccgtcgatccggaccgccacatcgagc
gggtcaccgagctgcaagaactcttcctcacgcgcgtcgggctcgacatcggcaaggtgtgggtcgcggacgacg
gcgccgcggtggcggtctggaccacgccggagagcgtcgaagcgggggcggtgttcgccgagatcggcccgcgca
tggccgagttgagcggttcccggctggccgcgcagcaacagatggaaggcctcctggcgccgcaccggcccaagg
agcccgcgtggttcctggccaccgtcggcgtctcgcccgaccaccagggcaagggtctgggcagcgccgtcgtgc
tccccggagtggaggcggccgagcgcgccggggtgcccgccttcctggagacctccgcgccccgcaacctcccct
tctacgagcggctcggcttcaccgtcaccgccgacgtcgaggtgcccgaaggaccgcgcacctggtgcatgaccc
gcaagcccggtgcctgaATTAATTAACCAATTGaatcaacctctggattacaaaatttgtgaaagattgactggt
attcttaactatgttgctccttttacgctatgtggatacgctgctttaatgcctttgtatcatgctattgcttcc
cgtatggctttcattttctcctccttgtataaatcctggttgctgtctctttatgaggagttgtggcccgttgtc
aggcaacgtggcgtggtgtgcactgtgtttgctgacgcaacccccactggttggggcattgccaccacctgtcag
ctcctttccgggactttcgctttccccctccctattgccacggcggaactcatcgccgcctgccttgcccgctgc
tggacaggggctcggctgttgggcactgacaattccgtggtgttgtcggggaaatcatcgtcctttccttggctg
ctcgcctgtgttgccacctggattctgcgcgggacgtccttctgctacgtcccttcggccctcaatccagcggac
cttccttcccgcggcctgctgccggctctgcggcctcttccgcgtcttcgccttcgccctcagacgagtcggatc
tccctttgggccgcctccccgcctggtacctttaagaccaatgacttacaaggcagctgtagatcttagccactt
tttaaaagaaaaggggggactggaagggctaattcactcccaacgaaaataagatctgctttttgcttgtactgg
gtctctctggttagaccagatctgagcctgggagctctctggctaactagggaacccactgcttaagcctcaata
aagcttgccttgagtgcttcaagtagtgtgtgcccgtctgttgtgtgactctggtaactagagatccctcagacc
cttttagtcagtgtggaaaatctctagcaCGTATGTGTATGATACATAAGGTTATGTATTAATTGTAGCCGCGTT
CTAACGACAATATGTACAAGCCTAATTGTGTAGCATCTGGCTTACTGAAGCAGACCCTATCATCTCTCTCGTAAA
CTGCCGTCAGAGTCGGTTTGGTTGGACGAACCTTCTGAGTTTCTGGTAACGCCGTCCCGCACCCGGAAATGGTCA
GCGAACCAATCAGCAGGGTCATCGCTAGCCAGATCCTCTACGCCGGACGCATCGTGGCCGGCATCACCGGCGCCA
CAGGTGCGGTTGCTGGCGCCTATATCGCCGACATCACCGATGGGGAAGATCGGGCTCGCCACTTCGGGCTCATGA
GCGCTTGTTTCGGCGTGGGTATGGTGGCAGGCCCCGTGGCCGGGGGACTGTTGGGCGCCATCTCCTTGCATGCAC
CATTCCTTGCGGCGGCGGTGCTCAACGGCCTCAACCTACTACTGGGCTGCTTCCTAATGCAGGAGTCGCATAAGG
GAGAGCGTCGAATGGTGCACTCTCAGTACAATCTGCTCTGATGCCGCATAGTTAAGCCAGCCCCGACACCCGCCA
ACACCCGCTGACGCGCCCTGACGGGCTTGTCTGCTCCCGGCATCCGCTTACAGACAAGCTGTGACCGTCTCCGGG
AGCTGCATGTGTCAGAGGTTTTCACCGTCATCACCGAAACGCGCGAGACGAAAGGGCCTCGTGATACGCCTATTT
TTATAGGTTAATGTCATGATAATAATGGTTTCTTAGACGTCAGGTGGCACTTTTCGGGGAAATGTGCGCGGAACC
CCTATTTGTTTATTTTTCTAAATACATTCAAATATGTATCCGCTCATGAGACAATAACCCTGATAAATGCTTCAA
TAATATTGAAAAAGGAAGAGTATGAGTATTCAACATTTCCGTGTCGCCCTTATTCCCTTTTTTGCGGCATTTTGC
CTTCCTGTTTTTGCTCACCCAGAAACGCTGGTGAAAGTAAAAGATGCTGAAGATCAGTTGGGTGCACGAGTGGGT
TACATCGAACTGGATCTCAACAGCGGTAAGATCCTTGAGAGTTTTCGCCCCGAAGAACGTTTTCCAATGATGAGC
ACTTTTAAAGTTCTGCTATGTGGCGCGGTATTATCCCGTATTGACGCCGGGCAAGAGCAACTCGGTCGCCGCATA
CACTATTCTCAGAATGACTTGGTTGAGTACTCACCAGTCACAGAAAAGCATCTTACGGATGGCATGACAGTAAGA
GAATTATGCAGTGCTGCCATAACCATGAGTGATAACACTGCGGCCAACTTACTTCTGACAACGATCGGAGGACCG
AAGGAGCTAACCGCTTTTTTGCACAACATGGGGGATCATGTAACTCGCCTTGATCGTTGGGAACCGGAGCTGAAT
GAAGCCATACCAAACGACGAGCGTGACACCACGATGCCTGTAGCAATGGCAACAACGTTGCGCAAACTATTAACT
GGCGAACTACTTACTCTAGCTTCCCGGCAACAATTAATAGACTGGATGGAGGCGGATAAAGTTGCAGGACCACTT
CTGCGCTCGGCCCTTCCGGCTGGCTGGTTTATTGCTGATAAATCTGGAGCCGGTGAGCGTGGGTCTCGCGGTATC
ATTGCAGCACTGGGGCCAGATGGTAAGCCCTCCCGTATCGTAGTTATCTACACGACGGGGAGTCAGGCAACTATG
GATGAACGAAATAGACAGATCGCTGAGATAGGTGCCTCACTGATTAAGCATTGGTAACTGTCAGACCAAGTTTAC
TCATATATACTTTAGATTGATTTAAAACTTCATTTTTAATTTAAAAGGATCTAGGTGAAGATCCTTTTTGATAAT
CTCATGACCAAAATCCCTTAACGTGAGTTTTCGTTCCACTGAGCGTCAGACCCCGTAGAAAAGATCAAAGGATCT
TCTTGAGATCCTTTTTTTCTGCGCGTAATCTGCTGCTTGCAAACAAAAAAACCACCGCTACCAGCGGTGGTTTGT
TTGCCGGATCAAGAGCTACCAACTCTTTTTCCGAAGGTAACTGGCTTCAGCAGAGCGCAGATACCAAATACTGTT
CTTCTAGTGTAGCCGTAGTTAGGCCACCACTTCAAGAACTCTGTAGCACCGCCTACATACCTCGCTCTGCTAATC
CTGTTACCAGTGGCTGCTGCCAGTGGCGATAAGTCGTGTCTTACCGGGTTGGACTCAAGACGATAGTTACCGGAT
AAGGCGCAGCGGTCGGGCTGAACGGGGGGTTCGTGCACACAGCCCAGCTTGGAGCGAACGACCTACACCGAACTG
AGATACCTACAGCGTGAGCTATGAGAAAGCGCCACGCTTCCCGAAGGGAGAAAGGCGGACAGGTATCCGGTAAGC
GGCAGGGTCGGAACAGGAGAGCGCACGAGGGAGCTTCCAGGGGGAAACGCCTGGTATCTTTATAGTCCTGTCGGG
TTTCGCCACCTCTGACTTGAGCGTCGATTTTTGTGATGCTCGTCAGGGGGGCGGAGCCTATGGAAAAACGCCAGC
AACGCGGCCTTTTTACGGTTCCTGGCCTTTTGCTGGCCTTTTGCTCACATGTTCTTTCCTGCGTTATCCCCTGAT
TCTGTGGATAACCGTATTACCGCCTTTGAGTGAGCTGATACCGCTCGCCGCAGCCGAACGACCGAGCGCAGCGAG
TCAGTGAGCGAGGAAGCGGAAGAGCGCCCAATACGCAAACCGCCTCTCCCCGCGCGTTGGCCGATTCATTAATGC
AGCTGTGGAATGTGTGTCAGTTAGGGTGTGGAAAGTCCCCAGGCTCCCCAGCAGGCAGAAGTATGCAAAGCATGC
ATCTCAATTAGTCAGCAACCAGGTGTGGAAAGTCCCCAGGCTCCCCAGCAGGCAGAAGTATGCAAAGCATGCATC
TCAATTAGTCAGCAACCATAGTCCCGCCCCTAACTCCGCCCATCCCGCCCCTAACTCCGCCCAGTTCCGCCCATT
CTCCGCCCCATGGCTGACTAATTTTTTTTATTTATGCAGAGGCCGAGGCCGCCTCGGCCTCTGAGCTATTCCAGA
AGTAGTGAGGAGGCTTTTTTGGAGGCCTAGGCTTTTGCAAAAAGCTTGGACACAAGACAGGCTTGCGAGATATGT
TTGAGAATACCACTTTATCCCGCGTCAGGGAGAGGCAGTGCGTAAAAAGACGCGGACTCATGTGAAATACTGGTT
TTTAGTGCGCCAGATCTCTATAATCTCGCGCAACCTATTTTCCCCTCGAACACTTTTTAAGCCGTAGATAAACAG
GCTGGGACACTTCACATGAGCGAAAAATACATCGTCACCTGGGACATGTTGCAGATCCATGCACGTAAACTCGCA
AGCCGACTGATGCCTTCTGAACAATGGAAAGGCATTATTGCCGTAAGCCGTGGCGGTCTGTACCGGGTGCGTTAC
TGGCGCGTGAACTGGGTATTCGTCATGTCGATACCGTTTGTATTTCCAGCTACGATCACGACAACCAGCGCGAGC
TTAAAGTGCTGAAACGCGCAGAAGGCGATGGCGAAGGCTTCATCGTTATTGATGACCTGGTGGATACCGGTGGTA
CTGCGGTTGCGATTCGTGAAATGTATCCAAAAGCGCACTTTGTCACCATCTTCGCAAAACCGGCTGGTCGTCCGC
TGGTTGATGACTATGTTGTTGATATCCCGCAAGATACCTGGATTGAACAGCCGTGGGATATGGGCGTCGTATTCG
TCCCGCCAATCTCCGGTCGCTAATCTTTTCAACGCCTGGCACTGCCGGGCGTTGTTCTTTTTAACTTCAGGCGGG
TTACAATAGTTTCCAGTAAGTATTCTGGAGGCTGCATCCATGACACAGGCAAACCTGAGCGAAACCCTGTTCAAA
CCCCGCTTTAAACATCCTGAAACCTCGACGCTAGTCCGCCGCTTTAATCACGGCGCACAACCGCCTGTGCAGTCG
GCCCTTGATGGTAAAACCATCCCTCACTGGTATCGCATGATTAACCGTCTGATGTGGATCTGGCGCGGCATTGAC
CCACGCGAAATCCTCGACGTCCAGGCACGTATTGTGATGAGCGATGCCGAACGTACCGACGATGATTTATACGAT
ACGGTGATTGGCTACCGTGGCGGCAACTGGATTTATGAGTGGGCCCCGGATCTTTGTGAAGGAACCTTACTTCTG
TGGTGTGACATAATTGGACAAACTACCTACAGAGATTTAAAGCTCTAAGGTAAATATAAAATTTTTAAGTGTATA
ATGTGTTAAACTACTGATTCTAATTGTTTGTGTATTTTAGATTCCAACCTATGGAACTGATGAATGGGAGCAGTG
GTGGAATGCCTTTAATGAGGAAAACCTGTTTTGCTCAGAAGAAATGCCATCTAGTGATGATGAGGCTACTGCTGA
CTCTCAACATTCTACTCCTCCAAAAAAGAAGAGAAAGGTAGAAGACCCCAAGGACTTTCCTTCAGAATTGCTAAG
TTTTTTGAGTCATGCTGTGTTTAGTAATAGAACTCTTGCTTGCTTTGCTATTTACACCACAAAGGAAAAAGCTGC
ACTGCTATACAAGAAAATTATGGAAAAATATTCTGTAACCTTTATAAGTAGGCATAACAGTTATAATCATAACAT
ACTGTTTTTTCTTACTCCACACAGGCATAGAGTGTCTGCTATTAATAACTATGCTCAAAAATTGTGTACCTTTAG
CTTTTTAATTTGTAAAGGGGTTAATAAGGAATATTTGATGTATAGTGCCTTGACTAGAGATCATAATCAGCCATA
CCACATTTGTAGAGGTTTTACTTGCTTTAAAAAACCTCCCACACCTCCCCCTGAACCTGAAACATAAAATGAATG
CAATTGTTGTTGTTAACTTGTTTATTGCAGCTTATAATGGTTACAAATAAAGCAATAGCATCACAAATTTCACAA
ATAAAGCATTTTTTTCACTGCATTCTAGTTGTGGTTTGTCCAAACTCATCAATGTATCTTATCATGTCTGGATCA
ACTGGATAACTCAAGCTAACCAAAATCATCCCAAACTTCCCACCCCATACCCTATTACCACTGCCAATTACCTGT
GGTTTCATTTACTCTAAACCTGTGATTCCTCTGAATTATTTTCATTTTAAAGAAATTGTATTTGTTAAATATGTA
CTACAAACTTAGTAG

10. My9.6-CD8-41BB-CD3z_4

An exemplary CAR construct, as described herein, comprises a CD33 binding domain (My9.6, wherein the CD33 binding domain comprises a VL-VH orientation), a CD8a transmembrane domain, an IgG4 hinge domain, a CD137 (4-1BB) co-stimulatory domain, and a CD3 t intracellular signaling domain.

In some embodiments, a CAR in encoded by a nucleic acid sequence that comprises the sequence that is shown in SEQ ID NO: 38, or in a nucleic acid sequence that is at least 70%, at least 75%, at least 80%, at least 85%, at least 90%, at least 95%, or at least 99% identical to the nucleic acid sequence shown in SEQ ID NO: 38.

[SEQ ID NO: 38]
ATGGCCCTGCCTGTGACAGCCCTGCTGCTGCCTCTGGCTCTGCTGCTGCATGCCGCTAGACCCGGATCCGAGATC
GTGCTGACACAGAGCCCTGGAAGCCTGGCCGTGTCTCCTGGCGAGCGCGTGACAATGAGCTGCAAGAGCAGCCAG
AGCGTGTTCTTCAGCAGCTCCCAGAAGAACTACCTGGCCTGGTATCAGCAGATCCCCGGCCAGAGCCCCAGACTG
CTGATCTACTGGGCCAGCACCAGAGAAAGCGGCGTGCCCGATAGATTCACCGGCAGCGGCTCTGGCACCGACTTC
ACCCTGACAATCAGCAGCGTGCAGCCCGAGGACCTGGCCATCTACTACTGCCACCAGTACCTGAGCAGCCGGACC
TTTGGCCAGGGCACCAAGCTGGAAATCAAGAGAGGCGGCGGAGGCTCTGGCGGAGGCGGATCTAGTGGCGGAGGA
TCTCAGGTGCAGCTGCAGCAGCCTGGCGCCGAGGTCGTGAAACCTGGCGCCTCTGTGAAGATGTCCTGCAAGGCC
AGCGGCTACACCTTCACCAGCTACTACATCCACTGGATCAAGCAGACCCCTGGACAGGGCCTGGAATGGGTGGGA
GTGATCTACCCCGGCAACGACGACATCAGCTACAACCAGAAGTTCCAGGGCAAGGCCACCCTGACCGCCGACAAG
TCTAGCACCACCGCCTACATGCAGCTGTCCAGCCTGACCAGCGAGGACAGCGCCGTGTACTACTGCGCCAGAGAA
GTGCGGCTGCGGTACTTCGATGTGTGGGGCCAGGGAACCACCGTGACCGTGTCATCTTCCGGAGAGAGCAAGTAC
GGCCCTCCCTGCCCCCCTTGCCCTGCCCCCGAGTTCCTGGGCGGACCCAGCGTGTTCCTGTTCCCCCCCAAGCCC
AAGGACACCCTGATGATCAGCCGGACCCCCGAGGTGACCTGTGTGGTGGTGGACGTGTCCCAGGAGGACCCCGAG
GTCCAGTTCAACTGGTACGTGGACGGCGTGGAGGTGCACAACGCCAAGACCAAGCCCCGGGAGGAGCAGTTCAAT
AGCACCTACCGGGTGGTGTCCGTGCTGACCGTGCTGCACCAGGACTGGCTGAACGGCAAGGAATACAAGTGTAAG
GTGTCCAACAAGGGCCTGCCCAGCAGCATCGAGAAAACCATCAGCAAGGCCAAGGGCCAGCCTCGGGAGCCCCAG
GTGTACACCCTGCCCCCTAGCCAAGAGGAGATGACCAAGAACCAGGTGTCCCTGACCTGCCTGGTGAAGGGCTTC
TACCCCAGCGACATCGCCGTGGAGTGGGAGAGCAACGGCCAGCCCGAGAACAACTACAAGACCACCCCCCCTGTG
CTGGACAGCGACGGCAGCTTCTTCCTGTACAGCCGGCTGACCGTGGACAAGAGCCGGTGGCAGGAGGGCAACGTC
TTTAGCTGCTCCGTGATGCACGAGGCCCTGCACAACCACTACACCCAGAAGAGCCTGAGCCTGTCCCTGGGCAAG
ATGATCTACATCTGGGCGCCCTTGGCCGGGACTTGTGGGGTCCTTCTCCTGTCACTGGTTATCACCCTTTACTGC
AAACGGGGCAGAAAGAAACTCCTGTATATATTCAAACAACCATTTATGAGACCAGTACAAACTACTCAAGAGGAA
GATGGCTGTAGCTGCCGATTTCCAGAAGAAGAAGAAGGAGGATGTGAACTGAGAGTGAAGTTCAGCAGGAGCGCA
GACGCCCCCGCGTACAAGCAGGGCCAGAACCAGCTCTATAACGAGCTCAATCTAGGACGAAGAGAGGAGTACGAT
GTTTTGGACAAGAGACGTGGCCGGGACCCTGAGATGGGGGGAAAGCCGAGAAGGAAGAACCCTCAGGAAGGCCTG
TACAATGAACTGCAGAAAGATAAGATGGCGGAGGCCTACAGTGAGATTGGGATGAAAGGCGAGCGCCGGAGGGGC
AAGGGGCACGATGGCCTTTACCAGGGTCTCAGTACAGCCACCAAGGACACCTACGACGCCCTTCACATGCAGGCC
CTGCCCCCTCGC

In some embodiments, a CAR comprises an amino acid sequence shown in SEQ ID NO: 39, or an amino acid sequence that is at least 70%, at least 75%, at least 80%, at least 85%, at least 90%, at least 95%, or at least 99% identical to the amino acid sequence shown in SEQ ID NO: 39.

[SEQ ID NO: 39]

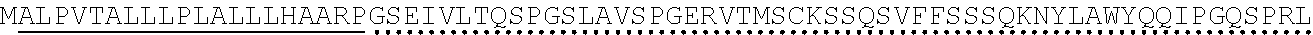

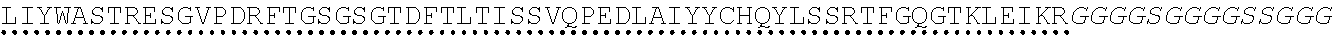

SQVQLQQPGAEVVKPGASVKMSCKASGYTFTSYYIHWIKQTPGQGLEWVGVIYPGNDDISYNQKFQGKATLTADK

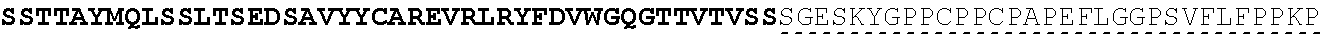

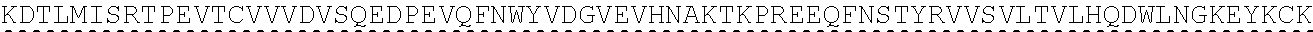

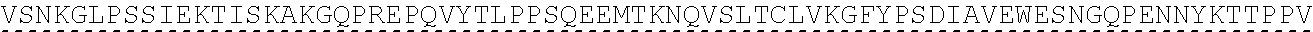

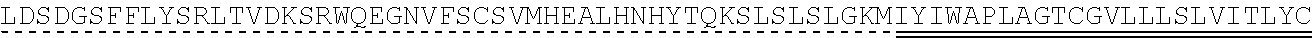

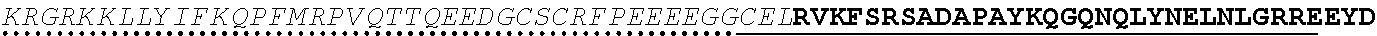

VLDKRRGRDPEMGGKPRRKNPQEGLYNELQKDKMAEAYSEIGMKGERRRGKGHDGLYQGLSTATKDTYDALHMQA
LPPR

In some embodiments, a CAR construct as shown in SEQ ID NO: 38 is included in a recombinant expression vector. In some embodiments the recombinant expression vector includes a promoter (e.g., an SFFV promoter or and EF1α promoter). In some embodiments, a recombinant expression vector including the CAR of SEQ ID NO: 38 comprises the sequence that is shown in SEQ ID NO: 40, or in a nucleic acid sequence that is at least 70%, at least 75%, at least 80%, at least 85%, at least 90%, at least 95%, or at least 99% identical to the nucleic acid sequence shown in SEQ ID NO: 40.

In some embodiments, a recombinant expression vector including the CAR of SEQ ID NO: 38 comprises the sequence that is shown in SEQ ID NO: 40, but does not include the SFFV promoter sequence (as shown in SEQ ID NO: 8). In some embodiments, a recombinant expression vector comprises the nucleic acid sequence shown in SEQ ID NO: 40, but does not include the SFFV sequence (as shown in SEQ ID NO: 8), and instead includes an EF1α promoter sequence.

[SEQ ID NO: 40]
Tgggtctctctggttagaccagatctgagcctgggagctctctggctaactagggaacccactgcttaagcctca
ataaagcttgccttgagtgcttcaagtagtgtgtgcccgtctgttgtgtgactctggtaactagagatccctcag
acccttttagtcagtgtggaaaatctctagcagtggcgcccgaacagggacttgaaagcgaaagggaaaccagag
gagctctctcgacgcaggactcggcttgctgaagcgcgcacggcaagaggcgaggggcggcgactggtgagtacg
ccaaaaattttgactagcggaggctagaaggagagagatgggtgcgagagcgtcagtattaagcgggggagaatt
agatcgcgatgggaaaaaattcggttaaggccagggggaaagaaaaaatataaattaaaacatatagtatgggca
agcagggagctagaacgattcgcagttaatcctggcctgttagaaacatcagaaggctgtagacaaatactggga
cagctacaaccatcccttcagacaggatcagaagaacttagatcattatataatacagtagcaaccctctattgt
gtgcatcaaaggatagagataaaagacaccaaggaagctttagacaagatagaggaagagcaaaacaaaagtaag
accaccgcacagcaagcggccactgatcttcagacctggaggaggagatatgagggacaattggagaagtgaatt
atataaatataaagtagtaaaaattgaaccattaggagtagcacccaccaaggcaaagagaagagtggtgcagag
agaaaaaagagcagtgggaataggagctttgttccttgggttcttgggagcagcaggaagcactatgggcgcagc
gtcaatgacgctgacggtacaggccagacaattattgtctggtatagtgcagcagcagaacaatttgctgagggc
tattgaggcgcaacagcatctgttgcaactcacagtctggggcatcaagcagctccaggcaagaatcctggctgt
ggaaagatacctaaaggatcaacagctcctggggatttggggttgctctggaaaactcatttgcaccactgcgt
gccttggaatgctagttggagtaataaatctctggaacagaattggaatcacacgacctggatggagtgggacag
agaaattaacaattacacaagcttaatacactccttaattgaagaatcgcaaaaccagcaagaaaagaatgaaca
agaattattggaattagataaatgggcaagtttgtggaattggtttaacataacaaattggctgtggtatataaa
attattcataatgatagtaggaggcttggtaggtttaagaatagtttttgctgtactttctatagtgaatagagt
taggcagggatattcaccattatcgtttcagacccacctcccaaccccgaggggacccgacaggcccgaaggaat
agaagaagaaggtggagagagagacagagacagatccattcgattagtgaacggatctcgacggtatcggttaac
ttttaaaagaaaaggggggattggggggtacagtgcaggggaaagaatagtagacataatagcaacagacataca
aactaaagaattacaaaaacaaattacaaaattcaaaattttatcgatactagtggatctGCGATCGCGTAACGC
CATTTTGCAAGGCATGGAAAAATACCAAACCAAGAATAGAGAAGTTCAGATCAAGGGCGGGTACATGAAAATAGC
TAACGTTGGGCCAAACAGGATATCTGCGGTGAGCAGTTTCGGCCCCGGCCCGGGGCCAAGAACAGATGGTCACCG
CAGTTTCGGCCCCGGCCCGAGGCCAAGAACAGATGGTCCCCAGATATGGCCCAACCCTCAGCAGTTTCTTAAGAC
CCATCAGATGTTTCCAGGCTCCCCCAAGGACCTGAAATGACCCTGCGCCTTATTTGAATTAACCAATCAGCCTGC
TTCTCGCTTCTGTTCGCGCGCTTCTGCTTCCCGAGCTCTATAAAAGAGCTCACAACCCCTCACTCGGCGCGCCAG
TCCTCCGACAGACTGAGTCGTCTAGAgctagcGGATCCCCGGAATTCGACGCCACCATGGCCCTGCCTGTGACAG
CCCTGCTGCTGCCTCTGGCTCTGCTGCTGCATGCCGCTAGACCCGGATCCGAGATCGTGCTGACACAGAGCCCTG
GAAGCCTGGCCGTGTCTCCTGGCGAGCGCGTGACAATGAGCTGCAAGAGCAGCCAGAGCGTGTTCTTCAGCAGCT
CCCAGAAGAACTACCTGGCCTGGTATCAGCAGATCCCCGGCCAGAGCCCCAGACTGCTGATCTACTGGGCCAGCA
CCAGAGAAAGCGGCGTGCCCGATAGATTCACCGGCAGCGGCTCTGGCACCGACTTCACCCTGACAATCAGCAGCG
TGCAGCCCGAGGACCTGGCCATCTACTACTGCCACCAGTACCTGAGCAGCCGGACCTTTGGCCAGGGCACCAAGC
TGGAAATCAAGAGAGGCGGCGGAGGCTCTGGCGGAGGCGGATCTAGTGGCGGAGGATCTCAGGTGCAGCTGCAGC
AGCCTGGCGCCGAGGTCGTGAAACCTGGCGCCTCTGTGAAGATGTCCTGCAAGGCCAGCGGCTACACCTTCACCA
GCTACTACATCCACTGGATCAAGCAGACCCCTGGACAGGGCCTGGAATGGGTGGGAGTGATCTACCCCGGCAACG
ACGACATCAGCTACAACCAGAAGTTCCAGGGCAAGGCCACCCTGACCGCCGACAAGTCTAGCACCACCGCCTACA
TGCAGCTGTCCAGCCTGACCAGCGAGGACAGCGCCGTGTACTACTGCGCCAGAGAAGTGCGGCTGCGGTACTTCG
ATGTGTGGGGCCAGGGAACCACCGTGACCGTGTCATCTTCCGGAGAGAGCAAGTACGGCCCTCCCTGCCCCCCTT
GCCCTGCCCCCGAGTTCCTGGGCGGACCCAGCGTGTTCCTGTTCCCCCCCAAGCCCAAGGACACCCTGATGATCA
GCCGGACCCCCGAGGTGACCTGTGTGGTGGTGGACGTGTCCCAGGAGGACCCCGAGGTCCAGTTCAACTGGTACG
TGGACGGCGTGGAGGTGCACAACGCCAAGACCAAGCCCCGGGAGGAGCAGTTCAATAGCACCTACCGGGTGGTGT
CCGTGCTGACCGTGCTGCACCAGGACTGGCTGAACGGCAAGGAATACAAGTGTAAGGTGTCCAACAAGGGCCTGC
CCAGCAGCATCGAGAAAACCATCAGCAAGGCCAAGGGCCAGCCTCGGGAGCCCCAGGTGTACACCCTGCCCCCTA
GCCAAGAGGAGATGACCAAGAACCAGGTGTCCCTGACCTGCCTGGTGAAGGGCTTCTACCCCAGCGACATCGCCG
TGGAGTGGGAGAGCAACGGCCAGCCCGAGAACAACTACAAGACCACCCCCCCTGTGCTGGACAGCGACGGCAGCT
TCTTCCTGTACAGCCGGCTGACCGTGGACAAGAGCCGGTGGCAGGAGGGCAACGTCTTTAGCTGCTCCGTGATGC
ACGAGGCCCTGCACAACCACTACACCCAGAAGAGCCTGAGCCTGTCCCTGGGCAAGATGATCTACATCTGGGCGC
CCTTGGCCGGGACTTGTGGGGTCCTTCTCCTGTCACTGGTTATCACCCTTTACTGCAAACGGGGCAGAAAGAAAC
TCCTGTATATATTCAAACAACCATTTATGAGACCAGTACAAACTACTCAAGAGGAAGATGGCTGTAGCTGCCGAT
TTCCAGAAGAAGAAGAAGGAGGATGTGAACTGAGAGTGAAGTTCAGCAGGAGCGCAGACGCCCCCGCGTACAAGC
AGGGCCAGAACCAGCTCTATAACGAGCTCAATCTAGGACGAAGAGAGGAGTACGATGTTTTGGACAAGAGACGTG
GCCGGGACCCTGAGATGGGGGGAAAGCCGAGAAGGAAGAACCCTCAGGAAGGCCTGTACAATGAACTGCAGAAAG
ATAAGATGGCGGAGGCCTACAGTGAGATTGGGATGAAAGGCGAGCGCCGGAGGGGCAAGGGGCACGATGGCCTTT
ACCAGGGTCTCAGTACAGCCACCAAGGACACCTACGACGCCCTTCACATGCAGGCCCTGCCCCCTCGCgagggca
gaggaagtcttctaacatgcggtgacgtggaggagaatcccggcccttccgggatgaccgagtacaagcccacgg
tgcgcctcgccacccgcgacgacgtccccagggccgtacgcaccctcgccgccgcgttcgccgactaccccgcca
cgcgccacaccgtcgatccggaccgccacatcgagcgggtcaccgagctgcaagaactcttcctcacgcgcgtcg
ggctcgacatcggcaaggtgtgggtcgcggacgacggcgccgcggtggcggtctggaccacgccggagagcgtcg
aagcgggggcggtgttcgccgagatcggcccgcgcatggccgagttgagcggttcccggctggccgcgcagcaac
agatggaaggcctcctggcgccgcaccggcccaaggagcccgcgtggttcctggccaccgtcggcgtctcgcccg
accaccagggcaagggtctgggcagcgccgtcgtgctccccggagtggaggcggccgagcgcgccggggtgcccg
ccttcctggagacctccgcgccccgcaacctccccttctacgagcggctcggcttcaccgtcaccgccgacgtcg
aggtgcccgaaggaccgcgcacctggtgcatgacccgcaagcccggtgcctgaATTAATTAACCAATTGaatcaa
cctctggattacaaaatttgtgaaagattgactggtattcttaactatgttgctccttttacgctatgtggatac
gctgctttaatgcctttgtatcatgctattgcttcccgtatggctttcattttctcctccttgtataaatcctgg
ttgctgtctctttatgaggagttgtggcccgttgtcaggcaacgtggcgtggtgtgcactgtgtttgctgacgca
acccccactggttggggcattgccaccacctgtcagctcctttccgggactttcgctttccccctccctattgcc
acggcggaactcatcgccgcctgccttgcccgctgctggacaggggctcggctgttgggcactgacaattccgtg
gtgttgtcggggaaatcatcgtcctttccttggctgctcgcctgtgttgccacctggattctgcgcgggacgtcc
ttctgctacgtcccttcggccctcaatccagcggaccttccttcccgcggcctgctgccggctctgcggcctctt
ccgcgtcttcgccttcgccctcagacgagtcggatctccctttgggccgcctccccgcctggtacctttaagacc
aatgacttacaaggcagctgtagatcttagccactttttaaaagaaaaggggggactggaagggctaattcactc
ccaacgaaaataagatctgctttttgcttgtactgggtctctctggttagaccagatctgagcctgggagctctc
tggctaactagggaacccactgcttaagcctcaataaagcttgccttgagtgcttcaagtagtgtgtgcccgtct
gttgtgtgactctggtaactagagatccctcagacccttttagtcagtgtggaaaatctctagcaCGTATGTGTA
TGATACATAAGGTTATGTATTAATTGTAGCCGCGTTCTAACGACAATATGTACAAGCCTAATTGTGTAGCATCTG
GCTTACTGAAGCAGACCCTATCATCTCTCTCGTAAACTGCCGTCAGAGTCGGTTTGGTTGGACGAACCTTCTGAG
TTTCTGGTAACGCCGTCCCGCACCCGGAAATGGTCAGCGAACCAATCAGCAGGGTCATCGCTAGCCAGATCCTCT
ACGCCGGACGCATCGTGGCCGGCATCACCGGCGCCACAGGTGCGGTTGCTGGCGCCTATATCGCCGACATCACCG
ATGGGGAAGATCGGGCTCGCCACTTCGGGCTCATGAGCGCTTGTTTCGGCGTGGGTATGGTGGCAGGCCCCGTGG
CCGGGGGACTGTTGGGCGCCATCTCCTTGCATGCACCATTCCTTGCGGCGGCGGTGCTCAACGGCCTCAACCTAC
TACTGGGCTGCTTCCTAATGCAGGAGTCGCATAAGGGAGAGCGTCGAATGGTGCACTCTCAGTACAATCTGCTCT
GATGCCGCATAGTTAAGCCAGCCCCGACACCCGCCAACACCCGCTGACGCGCCCTGACGGGCTTGTCTGCTCCCG
GCATCCGCTTACAGACAAGCTGTGACCGTCTCCGGGAGCTGCATGTGTCAGAGGTTTTCACCGTCATCACCGAAA
CGCGCGAGACGAAAGGGCCTCGTGATACGCCTATTTTTATAGGTTAATGTCATGATAATAATGGTTTCTTAGACG
TCAGGTGGCACTTTTCGGGGAAATGTGCGCGGAACCCCTATTTGTTTATTTTTCTAAATACATTCAAATATGTAT
CCGCTCATGAGACAATAACCCTGATAAATGCTTCAATAATATTGAAAAAGGAAGAGTATGAGTATTCAACATTTC
CGTGTCGCCCTTATTCCCTTTTTTGCGGCATTTTGCCTTCCTGTTTTTGCTCACCCAGAAACGCTGGTGAAAGTA
AAAGATGCTGAAGATCAGTTGGGTGCACGAGTGGGTTACATCGAACTGGATCTCAACAGCGGTAAGATCCTTGAG
AGTTTTCGCCCCGAAGAACGTTTTCCAATGATGAGCACTTTTAAAGTTCTGCTATGTGGCGCGGTATTATCCCGT
ATTGACGCCGGGCAAGAGCAACTCGGTCGCCGCATACACTATTCTCAGAATGACTTGGTTGAGTACTCACCAGTC
ACAGAAAAGCATCTTACGGATGGCATGACAGTAAGAGAATTATGCAGTGCTGCCATAACCATGAGTGATAACACT
GCGGCCAACTTACTTCTGACAACGATCGGAGGACCGAAGGAGCTAACCGCTTTTTTGCACAACATGGGGGATCAT
GTAACTCGCCTTGATCGTTGGGAACCGGAGCTGAATGAAGCCATACCAAACGACGAGCGTGACACCACGATGCCT
GTAGCAATGGCAACAACGTTGCGCAAACTATTAACTGGCGAACTACTTACTCTAGCTTCCCGGCAACAATTAATA
GACTGGATGGAGGCGGATAAAGTTGCAGGACCACTTCTGCGCTCGGCCCTTCCGGCTGGCTGGTTTATTGCTGAT
AAATCTGGAGCCGGTGAGCGTGGGTCTCGCGGTATCATTGCAGCACTGGGGCCAGATGGTAAGCCCTCCCGTATC
GTAGTTATCTACACGACGGGGAGTCAGGCAACTATGGATGAACGAAATAGACAGATCGCTGAGATAGGTGCCTCA
CTGATTAAGCATTGGTAACTGTCAGACCAAGTTTACTCATATATACTTTAGATTGATTTAAAACTTCATTTTTAA
TTTAAAAGGATCTAGGTGAAGATCCTTTTTGATAATCTCATGACCAAAATCCCTTAACGTGAGTTTTCGTTCCAC
TGAGCGTCAGACCCCGTAGAAAAGATCAAAGGATCTTCTTGAGATCCTTTTTTTCTGCGCGTAATCTGCTGCTTG
CAAACAAAAAAACCACCGCTACCAGCGGTGGTTTGTTTGCCGGATCAAGAGCTACCAACTCTTTTTCCGAAGGTA
ACTGGCTTCAGCAGAGCGCAGATACCAAATACTGTTCTTCTAGTGTAGCCGTAGTTAGGCCACCACTTCAAGAAC
TCTGTAGCACCGCCTACATACCTCGCTCTGCTAATCCTGTTACCAGTGGCTGCTGCCAGTGGCGATAAGTCGTGT
CTTACCGGGTTGGACTCAAGACGATAGTTACCGGATAAGGCGCAGCGGTCGGGCTGAACGGGGGGTTCGTGCACA
CAGCCCAGCTTGGAGCGAACGACCTACACCGAACTGAGATACCTACAGCGTGAGCTATGAGAAAGCGCCACGCTT
CCCGAAGGGAGAAAGGCGGACAGGTATCCGGTAAGCGGCAGGGTCGGAACAGGAGAGCGCACGAGGGAGCTTCCA
GGGGGAAACGCCTGGTATCTTTATAGTCCTGTCGGGTTTCGCCACCTCTGACTTGAGCGTCGATTTTTGTGATGC
TCGTCAGGGGGGCGGAGCCTATGGAAAAACGCCAGCAACGCGGCCTTTTTACGGTTCCTGGCCTTTTGCTGGCCT
TTTGCTCACATGTTCTTTCCTGCGTTATCCCCTGATTCTGTGGATAACCGTATTACCGCCTTTGAGTGAGCTGAT
ACCGCTCGCCGCAGCCGAACGACCGAGCGCAGCGAGTCAGTGAGCGAGGAAGCGGAAGAGCGCCCAATACGCAAA
CCGCCTCTCCCCGCGCGTTGGCCGATTCATTAATGCAGCTGTGGAATGTGTGTCAGTTAGGGTGTGGAAAGTCCC
CAGGCTCCCCAGCAGGCAGAAGTATGCAAAGCATGCATCTCAATTAGTCAGCAACCAGGTGTGGAAAGTCCCCAG
GCTCCCCAGCAGGCAGAAGTATGCAAAGCATGCATCTCAATTAGTCAGCAACCATAGTCCCGCCCCTAACTCCGC
CCATCCCGCCCCTAACTCCGCCCAGTTCCGCCCATTCTCCGCCCCATGGCTGACTAATTTTTTTTATTTATGCAG
AGGCCGAGGCCGCCTCGGCCTCTGAGCTATTCCAGAAGTAGTGAGGAGGCTTTTTTGGAGGCCTAGGCTTTTGCA
AAAAGCTTGGACACAAGACAGGCTTGCGAGATATGTTTGAGAATACCACTTTATCCCGCGTCAGGGAGAGGCAGT
GCGTAAAAAGACGCGGACTCATGTGAAATACTGGTTTTTAGTGCGCCAGATCTCTATAATCTCGCGCAACCTATT
TTCCCCTCGAACACTTTTTAAGCCGTAGATAAACAGGCTGGGACACTTCACATGAGCGAAAAATACATCGTCACC
TGGGACATGTTGCAGATCCATGCACGTAAACTCGCAAGCCGACTGATGCCTTCTGAACAATGGAAAGGCATTATT
GCCGTAAGCCGTGGCGGTCTGTACCGGGTGCGTTACTGGCGCGTGAACTGGGTATTCGTCATGTCGATACCGTTT
GTATTTCCAGCTACGATCACGACAACCAGCGCGAGCTTAAAGTGCTGAAACGCGCAGAAGGCGATGGCGAAGGCT
TCATCGTTATTGATGACCTGGTGGATACCGGTGGTACTGCGGTTGCGATTCGTGAAATGTATCCAAAAGCGCACT
TTGTCACCATCTTCGCAAAACCGGCTGGTCGTCCGCTGGTTGATGACTATGTTGTTGATATCCCGCAAGATACCT
GGATTGAACAGCCGTGGGATATGGGCGTCGTATTCGTCCCGCCAATCTCCGGTCGCTAATCTTTTCAACGCCTGG
CACTGCCGGGCGTTGTTCTTTTTAACTTCAGGCGGGTTACAATAGTTTCCAGTAAGTATTCTGGAGGCTGCATCC
ATGACACAGGCAAACCTGAGCGAAACCCTGTTCAAACCCCGCTTTAAACATCCTGAAACCTCGACGCTAGTCCGC
CGCTTTAATCACGGCGCACAACCGCCTGTGCAGTCGGCCCTTGATGGTAAAACCATCCCTCACTGGTATCGCATG
ATTAACCGTCTGATGTGGATCTGGCGCGGCATTGACCCACGCGAAATCCTCGACGTCCAGGCACGTATTGTGATG
AGCGATGCCGAACGTACCGACGATGATTTATACGATACGGTGATTGGCTACCGTGGCGGCAACTGGATTTATGAG
TGGGCCCCGGATCTTTGTGAAGGAACCTTACTTCTGTGGTGTGACATAATTGGACAAACTACCTACAGAGATTTA
AAGCTCTAAGGTAAATATAAAATTTTTAAGTGTATAATGTGTTAAACTACTGATTCTAATTGTTTGTGTATTTTA
GATTCCAACCTATGGAACTGATGAATGGGAGCAGTGGTGGAATGCCTTTAATGAGGAAAACCTGTTTTGCTCAGA
AGAAATGCCATCTAGTGATGATGAGGCTACTGCTGACTCTCAACATTCTACTCCTCCAAAAAAGAAGAGAAAGGT
AGAAGACCCCAAGGACTTTCCTTCAGAATTGCTAAGTTTTTTGAGTCATGCTGTGTTTAGTAATAGAACTCTTGC
TTGCTTTGCTATTTACACCACAAAGGAAAAAGCTGCACTGCTATACAAGAAAATTATGGAAAAATATTCTGTAAC
CTTTATAAGTAGGCATAACAGTTATAATCATAACATACTGTTTTTTCTTACTCCACACAGGCATAGAGTGTCTGC
TATTAATAACTATGCTCAAAAATTGTGTACCTTTAGCTTTTTAATTTGTAAAGGGGTTAATAAGGAATATTTGAT
GTATAGTGCCTTGACTAGAGATCATAATCAGCCATACCACATTTGTAGAGGTTTTACTTGCTTTAAAAAACCTCC
CACACCTCCCCCTGAACCTGAAACATAAAATGAATGCAATTGTTGTTGTTAACTTGTTTATTGCAGCTTATAATG
GTTACAAATAAAGCAATAGCATCACAAATTTCACAAATAAAGCATTTTTTTCACTGCATTCTAGTTGTGGTTTGT
CCAAACTCATCAATGTATCTTATCATGTCTGGATCAACTGGATAACTCAAGCTAACCAAAATCATCCCAAACTTC
CCACCCCATACCCTATTACCACTGCCAATTACCTGTGGTTTCATTTACTCTAAACCTGTGATTCCTCTGAATTAT
TTTCATTTTAAAGAAATTGTATTTGTTAAATATGTACTACAAACTTAGTAG

11. hM195-CD8-41BB-CD3z_2

An exemplary CAR construct, as described herein, comprises a CD33 binding domain (hM195 (Lintuzumab)), a CD8a transmembrane domain, a CD8a hinge domain, a CD137 (4-1BB) co-stimulatory domain, and a CD3ζ intracellular signaling domain.

In some embodiments, a CAR in encoded by a nucleic acid sequence that comprises the sequence that is shown in SEQ ID NO: 41, or in a nucleic acid sequence that is at least 70%, at least 75%, at least 80%, at least 85%, at least 90%, at least 95%, or at least 99% identical to the nucleic acid sequence shown in SEQ ID NO: 41.

[SEQ ID NO: 41]
ATGGCCCTGCCCGTGACCGCCCTGCTGCTGCCCCTGGCCCTGCTGCTGCACGCCGCCAGGCCCCAGGTGCAGCTG
GTGCAGAGCGGCGCCGAGGTGAAGAAGCCCGGCAGCAGCGTGAAGGTGAGCTGCAAGGCCAGCGGCTACACCTTC
ACCGACTACAACATGCACTGGGTGAGGCAGGCCCCCGGCCAGGGCCTGGAGTGGATCGGCTACATCTACCCCTAC
AACGGCGGCACCGGCTACAACCAGAAGTTCAAGAGCAAGGCCACCATCACCGCCGACGAGAGCACCAACACCGCC
TACATGGAGCTGAGCAGCCTGAGGAGCGAGGACACCGCCGTGTACTACTGCGCCAGGGGCAGGCCCGCCATGGAC
GTGTGGGGCCAGGGCACCCTGGTGACCGTGAGCAGCGGCGGCGGCGGCAGCGGCGGCGGCGGCAGCGGCGGCGGC
GGCAGCGACATCCAGATGACCCAGAGCCCCAGCAGCCTGAGCGCCAGCGTGGGCGACAGGGTGACCATCACCTGC
AGGGCCAGCGAGAGCGTGGACAACTACGGCATCAGCTTCATGAACTGGTTCCAGCAGAAGCCCGGCAAGGCCCCC
AAGCTGCTGATCTACGCCGCCAGCAACCAGGGCAGCGGCGTGCCCAGCAGGTTCAGCGGCAGCGGCAGCGGCACC
GACTTCACCCTGACCATCAGCAGCCTGCAGCCCGACGACTTCGCCACCTACTACTGCCAGCAGAGCAAGGAGGTG
CCCTGGACCTTCGGCCAGGGCACCAAGGTGGAGATCAAGGGCCTGGCCGTGAGCACCATCAGCAGCTTCTTCCCC
CCCGGCTACCAGATCTACATCTGGGCCCCCCTGGCCGGCACCTGCGGCGTGCTGCTGCTGAGCCTGGTGATCACC
CTGTACTGCAAGAGGGGCAGGAAGAAGCTGCTGTACATCTTCAAGCAGCCCTTCATGAGGCCCGTGCAGACCACC
CAGGAGGAGGACGGCTGCAGCTGCAGGTTCCCCGAGGAGGAGGAGGGCGGCTGCGAGCTGAGGGTGAAGTTCAGC
AGGAGCGCCGACGCCCCCGCCTACCAGCAGGGCCAGAACCAGCTGTACAACGAGCTGAACCTGGGCAGGAGGGAG
GAGTACGACGTGCTGGACAAGAGGAGGGGCAGGGACCCCGAGATGGGCGGCAAGCCCAGGAGGAAGAACCCCCAG
GAGGGCCTGTACAACGAGCTGCAGAAGGACAAGATGGCCGAGGCCTACAGCGAGATCGGCATGAAGGGCGAGAGG
AGGAGGGGCAAGGGCCACGACGGCCTGTACCAGGGCCTGAGCACCGCCACCAAGGACACCTACGACGCCCTGCAC
ATGCAGGCCCTGCCCCCCAGG

In some embodiments, a CAR comprises an amino acid sequence shown in SEQ ID NO: 42, or an amino acid sequence that is at least 70%, at least 75%, at least 80%, at least 85%, at least 90%, at least 95%, or at least 99% identical to the amino acid sequence shown in SEQ ID NO: 42.

[SEQ ID NO: 42]
MALPVTALLLPLALLLHAARPQVQLVQSGAEVKKPGSSVKVSCKASGYTFTDYNMHWVRQAPGQGLEWIGYIYPY
NGGTGYNQKFKSKATITADESTNTAYMELSSLRSEDTAVYYCARGRPAMDVWGQGTLVTVSSGGGGSGGGGSGGG

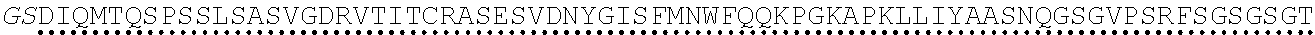

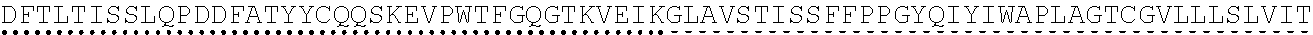

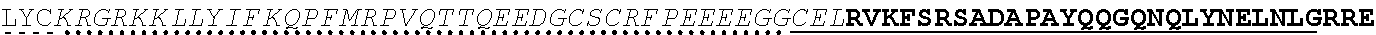

EYDVLDKRRGRDPEMGGKPRRKNPQEGLYNELQKDKMAEAYSEIGMKGERRRGKGHDGLYQGLSTATKDTYDALH
MQALPPR

In some embodiments, a CAR construct as shown in SEQ ID NO: 41 is included in a recombinant expression vector. In some embodiments the recombinant expression vector includes a promoter (e.g., an SFFV promoter or and EF1α promoter). In some embodiments, a recombinant expression vector including the CAR of SEQ ID NO: 41 comprises the sequence that is shown in SEQ ID NO: 43, or in a nucleic acid sequence that is at least 70%, at least 75%, at least 80%, at least 85%, at least 90%, at least 95%, or at least 99% identical to the nucleic acid sequence shown in SEQ ID NO: 43.

In some embodiments, a recombinant expression vector including the CAR of SEQ ID NO: 41 comprises the sequence that is shown in SEQ ID NO: 43, but does not include the SFFV promoter sequence (as shown in SEQ ID NO: 8). In some embodiments, a recombinant expression vector comprises the nucleic acid sequence shown in SEQ ID NO: 43, but does not include the SFFV sequence (as shown in SEQ ID NO: 8), and instead includes an EF1α promoter sequence.

[SEQ ID NO: 43]
Tgggtctctctggttagaccagatctgagcctgggagctctctggctaactagggaacccactgcttaagcctca
ataaagcttgccttgagtgcttcaagtagtgtgtgcccgtctgttgtgtgactctggtaactagagatccctcag
acccttttagtcagtgtggaaaatctctagcagtggcgcccgaacagggacttgaaagcgaaagggaaaccagag
gagctctctcgacgcaggactcggcttgctgaagcgcgcacggcaagaggcgaggggcggcgactggtgagtacg
ccaaaaattttgactagcggaggctagaaggagagagatgggtgcgagagcgtcagtattaagcgggggagaatt
agatcgcgatgggaaaaaattcggttaaggccagggggaaagaaaaaatataaattaaaacatatagtatgggca
agcagggagctagaacgattcgcagttaatcctggcctgttagaaacatcagaaggctgtagacaaatactggga
cagctacaaccatcccttcagacaggatcagaagaacttagatcattatataatacagtagcaaccctctattgt
gtgcatcaaaggatagagataaaagacaccaaggaagctttagacaagatagaggaagagcaaaacaaaagtaag
accaccgcacagcaagcggccactgatcttcagacctggaggaggagatatgagggacaattggagaagtgaatt
atataaatataaagtagtaaaaattgaaccattaggagtagcacccaccaaggcaaagagaagagtggtgcagag
agaaaaaagagcagtgggaataggagctttgttccttgggttcttgggagcagcaggaagcactatgggcgcagc
gtcaatgacgctgacggtacaggccagacaattattgtctggtatagtgcagcagcagaacaatttgctgagggc
tattgaggcgcaacagcatctgttgcaactcacagtctggggcatcaagcagctccaggcaagaatcctggctgt
ggaaagatacctaaaggatcaacagctcctggggatttggggttgctctggaaaactcatttgcaccactgctgt
gccttggaatgctagttggagtaataaatctctggaacagaattggaatcacacgacctggatggagtgggacag
agaaattaacaattacacaagcttaatacactccttaattgaagaatcgcaaaaccagcaagaaaagaatgaaca
agaattattggaattagataaatgggcaagtttgtggaattggtttaacataacaaattggctgtggtatataaa
attattcataatgatagtaggaggcttggtaggtttaagaatagtttttgctgtactttctatagtgaatagagt
taggcagggatattcaccattatcgtttcagacccacctcccaaccccgaggggacccgacaggcccgaaggaat
agaagaagaaggtggagagagagacagagacagatccattcgattagtgaacggatctcgacggtatcggttaac
ttttaaaagaaaaggggggattggggggtacagtgcaggggaaagaatagtagacataatagcaacagacataca
aactaaagaattacaaaaacaaattacaaaattcaaaattttatcgatactagtggatctGCGATCGCGTAACGC
CATTTTGCAAGGCATGGAAAAATACCAAACCAAGAATAGAGAAGTTCAGATCAAGGGCGGGTACATGAAAATAGC
TAACGTTGGGCCAAACAGGATATCTGCGGTGAGCAGTTTCGGCCCCGGCCCGGGGCCAAGAACAGATGGTCACCG
CAGTTTCGGCCCCGGCCCGAGGCCAAGAACAGATGGTCCCCAGATATGGCCCAACCCTCAGCAGTTTCTTAAGAC
CCATCAGATGTTTCCAGGCTCCCCCAAGGACCTGAAATGACCCTGCGCCTTATTTGAATTAACCAATCAGCCTGC
TTCTCGCTTCTGTTCGCGCGCTTCTGCTTCCCGAGCTCTATAAAAGAGCTCACAACCCCTCACTCGGCGCGCCAG
TCCTCCGACAGACTGAGTCGTCTAGAgctagcGGATCCCCGGAATTCGACGCCACCATGGCCCTGCCCGTGACCG
CCCTGCTGCTGCCCCTGGCCCTGCTGCTGCACGCCGCCAGGCCCCAGGTGCAGCTGGTGCAGAGCGGCGCCGAGG
TGAAGAAGCCCGGCAGCAGCGTGAAGGTGAGCTGCAAGGCCAGCGGCTACACCTTCACCGACTACAACATGCACT
GGGTGAGGCAGGCCCCCGGCCAGGGCCTGGAGTGGATCGGCTACATCTACCCCTACAACGGCGGCACCGGCTACA
ACCAGAAGTTCAAGAGCAAGGCCACCATCACCGCCGACGAGAGCACCAACACCGCCTACATGGAGCTGAGCAGCC
TGAGGAGCGAGGACACCGCCGTGTACTACTGCGCCAGGGGCAGGCCCGCCATGGACGTGTGGGGCCAGGGCACCC
TGGTGACCGTGAGCAGCGGCGGCGGCGGCAGCGGCGGCGGCGGCAGCGGCGGCGGCGGCAGCGACATCCAGATGA
CCCAGAGCCCCAGCAGCCTGAGCGCCAGCGTGGGCGACAGGGTGACCATCACCTGCAGGGCCAGCGAGAGCGTGG
ACAACTACGGCATCAGCTTCATGAACTGGTTCCAGCAGAAGCCCGGCAAGGCCCCCAAGCTGCTGATCTACGCCG
CCAGCAACCAGGGCAGCGGCGTGCCCAGCAGGTTCAGCGGCAGCGGCAGCGGCACCGACTTCACCCTGACCATCA
GCAGCCTGCAGCCCGACGACTTCGCCACCTACTACTGCCAGCAGAGCAAGGAGGTGCCCTGGACCTTCGGCCAGG
GCACCAAGGTGGAGATCAAGGGCCTGGCCGTGAGCACCATCAGCAGCTTCTTCCCCCCCGGCTACCAGATCTACA
TCTGGGCCCCCCTGGCCGGCACCTGCGGCGTGCTGCTGCTGAGCCTGGTGATCACCCTGTACTGCAAGAGGGGCA
GGAAGAAGCTGCTGTACATCTTCAAGCAGCCCTTCATGAGGCCCGTGCAGACCACCCAGGAGGAGGACGGCTGCA
GCTGCAGGTTCCCCGAGGAGGAGGAGGGCGGCTGCGAGCTGAGGGTGAAGTTCAGCAGGAGCGCCGACGCCCCCG
CCTACCAGCAGGGCCAGAACCAGCTGTACAACGAGCTGAACCTGGGCAGGAGGGAGGAGTACGACGTGCTGGACA
AGAGGAGGGGCAGGGACCCCGAGATGGGCGGCAAGCCCAGGAGGAAGAACCCCCAGGAGGGCCTGTACAACGAGC
TGCAGAAGGACAAGATGGCCGAGGCCTACAGCGAGATCGGCATGAAGGGCGAGAGGAGGAGGGGCAAGGGCCACG
ACGGCCTGTACCAGGGCCTGAGCACCGCCACCAAGGACACCTACGACGCCCTGCACATGCAGGCCCTGCCCCCCA
GGgagggcagaggaagtcttctaacatgcggtgacgtggaggagaatcccggcccttccgggatgaccgagtaca
agcccacggtgcgcctcgccacccgcgacgacgtccccagggccgtacgcaccctcgccgccgcgttcgccgact
accccgccacgcgccacaccgtcgatccggaccgccacatcgagcgggtcaccgagctgcaagaactcttcctca
cgcgcgtcgggctcgacatcggcaaggtgtgggtcgcggacgacggcgccgcggtggcggtctggaccacgccgg
agagcgtcgaagcgggggcggtgttcgccgagatcggcccgcgcatggccgagttgagcggttcccggctggccg
cgcagcaacagatggaaggcctcctggcgccgcaccggcccaaggagcccgcgtggttcctggccaccgtcggcg
tctcgcccgaccaccagggcaagggtctgggcagcgccgtcgtgctccccggagtggaggcggccgagcgcgccg
gggtgcccgccttcctggagacctccgcgccccgcaacctccccttctacgagcggctcggcttcaccgtcaccg
ccgacgtcgaggtgcccgaaggaccgcgcacctggtgcatgacccgcaagcccggtgcctgaATTAATTAACCAA
TTGaatcaacctctggattacaaaatttgtgaaagattgactggtattcttaactatgttgctccttttacgcta
tgtggatacgctgctttaatgcctttgtatcatgctattgcttcccgtatggctttcattttctcctccttgtat
aaatcctggttgctgtctctttatgaggagttgtggcccgttgtcaggcaacgtggcgtggtgtgcactgtgttt
gctgacgcaacccccactggttggggcattgccaccacctgtcagctcctttccgggactttcgctttccccctc
cctattgccacggcggaactcatcgccgcctgccttgcccgctgctggacaggggctcggctgttgggcactgac
aattccgtggtgttgtcggggaaatcatcgtcctttccttggctgctcgcctgtgttgccacctggattctgcgc
gggacgtccttctgctacgtcccttcggccctcaatccagcggaccttccttcccgcggcctgctgccggctctg
cggcctcttccgcgtcttcgccttcgccctcagacgagtcggatctccctttgggccgcctccccgcctggtacc
tttaagaccaatgacttacaaggcagctgtagatcttagccactttttaaaagaaaaggggggactggaagggct
aattcactcccaacgaaaataagatctgctttttgcttgtactgggtctctctggttagaccagatctgagcctg
ggagctctctggctaactagggaacccactgcttaagcctcaataaagcttgccttgagtgcttcaagtagtgtg
tgcccgtctgttgtgtgactctggtaactagagatccctcagacccttttagtcagtgtggaaaatctctagcac
GTATGTGTATGATACATAAGGTTATGTATTAATTGTAGCCGCGTTCTAACGACAATATGTACAAGCCTAATTGTG
TAGCATCTGGCTTACTGAAGCAGACCCTATCATCTCTCTCGTAAACTGCCGTCAGAGTCGGTTTGGTTGGACGAA
CCTTCTGAGTTTCTGGTAACGCCGTCCCGCACCCGGAAATGGTCAGCGAACCAATCAGCAGGGTCATCGCTAGCC
AGATCCTCTACGCCGGACGCATCGTGGCCGGCATCACCGGCGCCACAGGTGCGGTTGCTGGCGCCTATATCGCCG
ACATCACCGATGGGGAAGATCGGGCTCGCCACTTCGGGCTCATGAGCGCTTGTTTCGGCGTGGGTATGGTGGCAG
GCCCCGTGGCCGGGGGACTGTTGGGCGCCATCTCCTTGCATGCACCATTCCTTGCGGCGGCGGTGCTCAACGGCC
TCAACCTACTACTGGGCTGCTTCCTAATGCAGGAGTCGCATAAGGGAGAGCGTCGAATGGTGCACTCTCAGTACA
ATCTGCTCTGATGCCGCATAGTTAAGCCAGCCCCGACACCCGCCAACACCCGCTGACGCGCCCTGACGGGCTTGT
CTGCTCCCGGCATCCGCTTACAGACAAGCTGTGACCGTCTCCGGGAGCTGCATGTGTCAGAGGTTTTCACCGTCA
TCACCGAAACGCGCGAGACGAAAGGGCCTCGTGATACGCCTATTTTTATAGGTTAATGTCATGATAATAATGGTT
TCTTAGACGTCAGGTGGCACTTTTCGGGGAAATGTGCGCGGAACCCCTATTTGTTTATTTTTCTAAATACATTCA
AATATGTATCCGCTCATGAGACAATAACCCTGATAAATGCTTCAATAATATTGAAAAAGGAAGAGTATGAGTATT
CAACATTTCCGTGTCGCCCTTATTCCCTTTTTTGCGGCATTTTGCCTTCCTGTTTTTGCTCACCCAGAAACGCTG
GTGAAAGTAAAAGATGCTGAAGATCAGTTGGGTGCACGAGTGGGTTACATCGAACTGGATCTCAACAGCGGTAAG
ATCCTTGAGAGTTTTCGCCCCGAAGAACGTTTTCCAATGATGAGCACTTTTAAAGTTCTGCTATGTGGCGCGGTA
TTATCCCGTATTGACGCCGGGCAAGAGCAACTCGGTCGCCGCATACACTATTCTCAGAATGACTTGGTTGAGTAC
TCACCAGTCACAGAAAAGCATCTTACGGATGGCATGACAGTAAGAGAATTATGCAGTGCTGCCATAACCATGAGT
GATAACACTGCGGCCAACTTACTTCTGACAACGATCGGAGGACCGAAGGAGCTAACCGCTTTTTTGCACAACATG
GGGGATCATGTAACTCGCCTTGATCGTTGGGAACCGGAGCTGAATGAAGCCATACCAAACGACGAGCGTGACACC
ACGATGCCTGTAGCAATGGCAACAACGTTGCGCAAACTATTAACTGGCGAACTACTTACTCTAGCTTCCCGGCAA
CAATTAATAGACTGGATGGAGGCGGATAAAGTTGCAGGACCACTTCTGCGCTCGGCCCTTCCGGCTGGCTGGTTT
ATTGCTGATAAATCTGGAGCCGGTGAGCGTGGGTCTCGCGGTATCATTGCAGCACTGGGGCCAGATGGTAAGCCC
TCCCGTATCGTAGTTATCTACACGACGGGGAGTCAGGCAACTATGGATGAACGAAATAGACAGATCGCTGAGATA
GGTGCCTCACTGATTAAGCATTGGTAACTGTCAGACCAAGTTTACTCATATATACTTTAGATTGATTTAAAACTT
CATTTTTAATTTAAAAGGATCTAGGTGAAGATCCTTTTTGATAATCTCATGACCAAAATCCCTTAACGTGAGTTT
TCGTTCCACTGAGCGTCAGACCCCGTAGAAAAGATCAAAGGATCTTCTTGAGATCCTTTTTTTCTGCGCGTAATC
TGCTGCTTGCAAACAAAAAAACCACCGCTACCAGCGGTGGTTTGTTTGCCGGATCAAGAGCTACCAACTCTTTTT
CCGAAGGTAACTGGCTTCAGCAGAGCGCAGATACCAAATACTGTTCTTCTAGTGTAGCCGTAGTTAGGCCACCAC
TTCAAGAACTCTGTAGCACCGCCTACATACCTCGCTCTGCTAATCCTGTTACCAGTGGCTGCTGCCAGTGGCGAT
AAGTCGTGTCTTACCGGGTTGGACTCAAGACGATAGTTACCGGATAAGGCGCAGCGGTCGGGCTGAACGGGGGGT
TCGTGCACACAGCCCAGCTTGGAGCGAACGACCTACACCGAACTGAGATACCTACAGCGTGAGCTATGAGAAAGC
GCCACGCTTCCCGAAGGGAGAAAGGCGGACAGGTATCCGGTAAGCGGCAGGGTCGGAACAGGAGAGCGCACGAGG
GAGCTTCCAGGGGGAAACGCCTGGTATCTTTATAGTCCTGTCGGGTTTCGCCACCTCTGACTTGAGCGTCGATTT
TTGTGATGCTCGTCAGGGGGGCGGAGCCTATGGAAAAACGCCAGCAACGCGGCCTTTTTACGGTTCCTGGCCTTT
TGCTGGCCTTTTGCTCACATGTTCTTTCCTGCGTTATCCCCTGATTCTGTGGATAACCGTATTACCGCCTTTGAG
TGAGCTGATACCGCTCGCCGCAGCCGAACGACCGAGCGCAGCGAGTCAGTGAGCGAGGAAGCGGAAGAGCGCCCA
ATACGCAAACCGCCTCTCCCCGCGCGTTGGCCGATTCATTAATGCAGCTGTGGAATGTGTGTCAGTTAGGGTGTG
GAAAGTCCCCAGGCTCCCCAGCAGGCAGAAGTATGCAAAGCATGCATCTCAATTAGTCAGCAACCAGGTGTGGAA
AGTCCCCAGGCTCCCCAGCAGGCAGAAGTATGCAAAGCATGCATCTCAATTAGTCAGCAACCATAGTCCCGCCCC
TAACTCCGCCCATCCCGCCCCTAACTCCGCCCAGTTCCGCCCATTCTCCGCCCCATGGCTGACTAATTTTTTTTA
TTTATGCAGAGGCCGAGGCCGCCTCGGCCTCTGAGCTATTCCAGAAGTAGTGAGGAGGCTTTTTTGGAGGCCTAG
GCTTTTGCAAAAAGCTTGGACACAAGACAGGCTTGCGAGATATGTTTGAGAATACCACTTTATCCCGCGTCAGGG
AGAGGCAGTGCGTAAAAAGACGCGGACTCATGTGAAATACTGGTTTTTAGTGCGCCAGATCTCTATAATCTCGCG
CAACCTATTTTCCCCTCGAACACTTTTTAAGCCGTAGATAAACAGGCTGGGACACTTCACATGAGCGAAAAATAC
ATCGTCACCTGGGACATGTTGCAGATCCATGCACGTAAACTCGCAAGCCGACTGATGCCTTCTGAACAATGGAAA
GGCATTATTGCCGTAAGCCGTGGCGGTCTGTACCGGGTGCGTTACTGGCGCGTGAACTGGGTATTCGTCATGTCG
ATACCGTTTGTATTTCCAGCTACGATCACGACAACCAGCGCGAGCTTAAAGTGCTGAAACGCGCAGAAGGCGATG
GCGAAGGCTTCATCGTTATTGATGACCTGGTGGATACCGGTGGTACTGCGGTTGCGATTCGTGAAATGTATCCAA
AAGCGCACTTTGTCACCATCTTCGCAAAACCGGCTGGTCGTCCGCTGGTTGATGACTATGTTGTTGATATCCCGC
AAGATACCTGGATTGAACAGCCGTGGGATATGGGCGTCGTATTCGTCCCGCCAATCTCCGGTCGCTAATCTTTTC
AACGCCTGGCACTGCCGGGCGTTGTTCTTTTTAACTTCAGGCGGGTTACAATAGTTTCCAGTAAGTATTCTGGAG
GCTGCATCCATGACACAGGCAAACCTGAGCGAAACCCTGTTCAAACCCCGCTTTAAACATCCTGAAACCTCGACG
CTAGTCCGCCGCTTTAATCACGGCGCACAACCGCCTGTGCAGTCGGCCCTTGATGGTAAAACCATCCCTCACTGG
TATCGCATGATTAACCGTCTGATGTGGATCTGGCGCGGCATTGACCCACGCGAAATCCTCGACGTCCAGGCACGT
ATTGTGATGAGCGATGCCGAACGTACCGACGATGATTTATACGATACGGTGATTGGCTACCGTGGCGGCAACTGG
ATTTATGAGTGGGCCCCGGATCTTTGTGAAGGAACCTTACTTCTGTGGTGTGACATAATTGGACAAACTACCTAC
AGAGATTTAAAGCTCTAAGGTAAATATAAAATTTTTAAGTGTATAATGTGTTAAACTACTGATTCTAATTGTTTG
TGTATTTTAGATTCCAACCTATGGAACTGATGAATGGGAGCAGTGGTGGAATGCCTTTAATGAGGAAAACCTGTT
TTGCTCAGAAGAAATGCCATCTAGTGATGATGAGGCTACTGCTGACTCTCAACATTCTACTCCTCCAAAAAAGAA
GAGAAAGGTAGAAGACCCCAAGGACTTTCCTTCAGAATTGCTAAGTTTTTTGAGTCATGCTGTGTTTAGTAATAG
AACTCTTGCTTGCTTTGCTATTTACACCACAAAGGAAAAAGCTGCACTGCTATACAAGAAAATTATGGAAAAATA
TTCTGTAACCTTTATAAGTAGGCATAACAGTTATAATCATAACATACTGTTTTTTCTTACTCCACACAGGCATAG
AGTGTCTGCTATTAATAACTATGCTCAAAAATTGTGTACCTTTAGCTTTTTAATTTGTAAAGGGGTTAATAAGGA
ATATTTGATGTATAGTGCCTTGACTAGAGATCATAATCAGCCATACCACATTTGTAGAGGTTTTACTTGCTTTAA
AAAACCTCCCACACCTCCCCCTGAACCTGAAACATAAAATGAATGCAATTGTTGTTGTTAACTTGTTTATTGCAG
CTTATAATGGTTACAAATAAAGCAATAGCATCACAAATTTCACAAATAAAGCATTTTTTTCACTGCATTCTAGTT
GTGGTTTGTCCAAACTCATCAATGTATCTTATCATGTCTGGATCAACTGGATAACTCAAGCTAACCAAAATCATC
CCAAACTTCCCACCCCATACCCTATTACCACTGCCAATTACCTGTGGTTTCATTTACTCTAAACCTGTGATTCCT
CTGAATTATTTTCATTTTAAAGAAATTGTATTTGTTAAATATGTACTACAAACTTAGTAG

12. M195-CD8-41BB-CD3z

An exemplary CAR construct, as described herein, comprises a CD33 binding domain (M195) scFv, a CD8a transmembrane domain, a CD8a hinge domain, a CD137 (4-1BB) co-stimulatory domain, and a CD3ζ intracellular signaling domain.

In some embodiments, a CAR in encoded by a nucleic acid sequence that comprises the sequence that is shown in SEQ ID NO: 44, or in a nucleic acid sequence that is at least 70%, at least 75%, at least 80%, at least 85%, at least 90%, at least 95%, or at least 99% identical to the nucleic acid sequence shown in SEQ ID NO: 44.

[SEQ ID NO: 44]
ATGGCCCTGCCCGTGACCGCCCTGCTGCTGCCCCTGGCCCTGCTGCTGCACGCCGCCAGGCCCGAGGTGCAGCTG
CAGCAGAGCGGCCCCGAGCTGGTGAAGCCCGGCGCCAGCGTGAAGATCAGCTGCAAGGCCAGCGGCTACACCTTC
ACCGACTACAACATGCACTGGGTGAAGCAGAGCCACGGCAAGAGCCTGGAGTGGATCGGCTACATCTACCCCTAC
AACGGCGGCACCGGCTACAACCAGAAGTTCAAGAGCAAGGCCACCCTGACCGTGGACAACAGCAGCAGCACCGCC
TACATGGACGTGAGGAGCCTGACCAGCGAGGACAGCGCCGTGTACTACTGCGCCAGGGGCAGGCCCGCCATGGAC
TACTGGGGCCAGGGCACCAGCGTGACCGTGAGCAGCGGCGGCGGCGGCAGCGGCGGCGGCGGCAGCGGCGGCGGC
GGCAGCGACATCGTGCTGACCCAGAGCCCCGCCAGCCTGGCCGTGAGCCTGGGCCAGAGGGCCACCATCAGCTGC
AGGGCCAGCGAGAGCGTGGACAACTACGGCATCAGCTTCATGAACTGGTTCCAGCAGAAGCCCGGCCAGCCCCCC
AAGCTGCTGATCTACGCCGCCAGCAACCAGGGCAGCGGCGTGCCCGCCAGGTTCAGCGGCAGCGGCAGCGGCACC
GACTTCAGCCTGAACATCCACCCCATGGAGGAGGACGACACCGCCATGTACTTCTGCCAGCAGAGCAAGGAGGTG
CCCTGGACCTTCGGCGGCGGCACCAAGCTGGAGATCAAGGGCCTGGCCGTGAGCACCATCAGCAGCTTCTTCCCC
CCCGGCTACCAGATCTACATCTGGGCCCCCCTGGCCGGCACCTGCGGCGTGCTGCTGCTGAGCCTGGTGATCACC
CTGTACTGCAAGAGGGGCAGGAAGAAGCTGCTGTACATCTTCAAGCAGCCCTTCATGAGGCCCGTGCAGACCACC
CAGGAGGAGGACGGCTGCAGCTGCAGGTTCCCCGAGGAGGAGGAGGGCGGCTGCGAGCTGAGGGTGAAGTTCAGC
AGGAGCGCCGACGCCCCCGCCTACCAGCAGGGCCAGAACCAGCTGTACAACGAGCTGAACCTGGGCAGGAGGGAG
GAGTACGACGTGCTGGACAAGAGGAGGGGCAGGGACCCCGAGATGGGCGGCAAGCCCAGGAGGAAGAACCCCCAG
GAGGGCCTGTACAACGAGCTGCAGAAGGACAAGATGGCCGAGGCCTACAGCGAGATCGGCATGAAGGGCGAGAGG
AGGAGGGGCAAGGGCCACGACGGCCTGTACCAGGGCCTGAGCACCGCCACCAAGGACACCTACGACGCCCTGCAC
ATGCAGGCCCTGCCCCCCAGG

In some embodiments, a CAR comprises an amino acid sequence shown in SEQ ID NO: 45, or an amino acid sequence that is at least 70%, at least 75%, at least 80%, at least 85%, at least 90%, at least 95%, or at least 99% identical to the amino acid sequence shown in SEQ ID NO: 45.

[SEQ ID NO: 45]
MALPVTALLLPLALLLHAARPEVQLQQSGPELVKPGASVKISCKASGYTFTDYNMHWVKQSHGKSLEWIGYIYPY
NGGTGYNQKFKSKATLTVDNSSSTAYMDVRSLTSEDSAVYYCARGRPAMDYWGQGTSVTVSSGGGGSGGGGSGGG

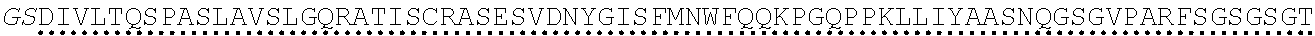

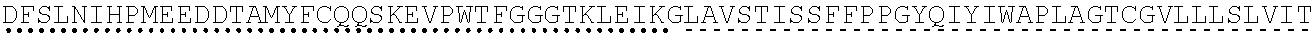

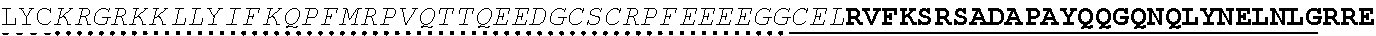

EYDVLDKRRGRDPEMGGKPRRKNPQEGLYNELQKDKMAEAYSEIGMKGERRRGKGHDGLYQGLSTATKDTYDALH
MQALPPR

In some embodiments, a CAR construct as shown in SEQ ID NO: 44 is included in a recombinant expression vector. In some embodiments the recombinant expression vector includes a promoter (e.g., an SFFV promoter or and EF1α promoter). In some embodiments, a recombinant expression vector including the CAR of SEQ ID NO: 44 comprises the sequence that is shown in SEQ ID NO: 46, or in a nucleic acid sequence that is at least 70%, at least 75%, at least 80%, at least 85%, at least 90%, at least 95%, or at least 99% identical to the nucleic acid sequence shown in SEQ ID NO: 46.

In some embodiments, a recombinant expression vector including the CAR of SEQ ID NO: 44 comprises the sequence that is shown in SEQ ID NO: 46, but does not include the SFFV promoter sequence (as shown in SEQ ID NO: 8). In some embodiments, a recombinant expression vector comprises the nucleic acid sequence shown in SEQ ID NO: 46, but does not include the SFFV sequence (as shown in SEQ ID NO: 8), and instead includes an EF1α promoter sequence.

[SEQ ID NO: 46]
Tgggtctctctggttagaccagatctgagcctgggagctctctggctaactagggaacccactgcttaagcctca
ataaagcttgccttgagtgcttcaagtagtgtgtgcccgtctgttgtgtgactctggtaactagagatccctcag
acccttttagtcagtgtggaaaatctctagcagtggcgcccgaacagggacttgaaagcgaaagggaaaccagag
gagctctctcgacgcaggactcggcttgctgaagcgcgcacggcaagaggcgaggggcggcgactggtgagtacg
ccaaaaattttgactagcggaggctagaaggagagagatgggtgcgagagcgtcagtattaagcgggggagaatt
agatcgcgatgggaaaaaattcggttaaggccagggggaaagaaaaaatataaattaaaacatatagtatgggca
agcagggagctagaacgattcgcagttaatcctggcctgttagaaacatcagaaggctgtagacaaatactggga
cagctacaaccatcccttcagacaggatcagaagaacttagatcattatataatacagtagcaaccctctattgt
gtgcatcaaaggatagagataaaagacaccaaggaagctttagacaagatagaggaagagcaaaacaaaagtaag
accaccgcacagcaagcggccactgatcttcagacctggaggaggagatatgagggacaattggagaagtgaatt
atataaatataaagtagtaaaaattgaaccattaggagtagcacccaccaaggcaaagagaagagtggtgcagag
agaaaaaagagcagtgggaataggagctttgttccttgggttcttgggagcagcaggaagcactatgggcgcagc
gtcaatgacgctgacggtacaggccagacaattattgtctggtatagtgcagcagcagaacaatttgctgagggc
tattgaggcgcaacagcatctgttgcaactcacagtctggggcatcaagcagctccaggcaagaatcctggctgt
ggaaagatacctaaaggatcaacagctcctggggatttggggttgctctggaaaactcatttgcaccactgctgt
gccttggaatgctagttggagtaataaatctctggaacagaattggaatcacacgacctggatggagtgggacag
agaaattaacaattacacaagcttaatacactccttaattgaagaatcgcaaaaccagcaagaaaagaatgaaca
agaattattggaattagataaatgggcaagtttgtggaattggtttaacataacaaattggctgtggtatataaa
attattcataatgatagtaggaggcttggtaggtttaagaatagtttttgctgtactttctatagtgaatagagt
taggcagggatattcaccattatcgtttcagacccacctcccaaccccgaggggacccgacaggcccgaaggaat
agaagaagaaggtggagagagagacagagacagatccattcgattagtgaacggatctcgacggtatcggttaac
ttttaaaagaaaaggggggattggggggtacagtgcaggggaaagaatagtagacataatagcaacagacataca
aactaaagaattacaaaaacaaattacaaaattcaaaattttatcgatactagtggatctGCGATCGCGTAACGC
CATTTTGCAAGGCATGGAAAAATACCAAACCAAGAATAGAGAAGTTCAGATCAAGGGCGGGTACATGAAAATAGC
TAACGTTGGGCCAAACAGGATATCTGCGGTGAGCAGTTTCGGCCCCGGCCCGGGGCCAAGAACAGATGGTCACCG
CAGTTTCGGCCCCGGCCCGAGGCCAAGAACAGATGGTCCCCAGATATGGCCCAACCCTCAGCAGTTTCTTAAGAC
CCATCAGATGTTTCCAGGCTCCCCCAAGGACCTGAAATGACCCTGCGCCTTATTTGAATTAACCAATCAGCCTGC
TTCTCGCTTCTGTTCGCGCGCTTCTGCTTCCCGAGCTCTATAAAAGAGCTCACAACCCCTCACTCGGCGCGCCAG
TCCTCCGACAGACTGAGTCGTCTAGAgctagcGGATCCCCGGAATTCGACGCCACCATGGCCCTGCCCGTGACCG
CCCTGCTGCTGCCCCTGGCCCTGCTGCTGCACGCCGCCAGGCCCGAGGTGCAGCTGCAGCAGAGCGGCCCCGAGC
TGGTGAAGCCCGGCGCCAGCGTGAAGATCAGCTGCAAGGCCAGCGGCTACACCTTCACCGACTACAACATGCACT
GGGTGAAGCAGAGCCACGGCAAGAGCCTGGAGTGGATCGGCTACATCTACCCCTACAACGGCGGCACCGGCTACA
ACCAGAAGTTCAAGAGCAAGGCCACCCTGACCGTGGACAACAGCAGCAGCACCGCCTACATGGACGTGAGGAGCC
TGACCAGCGAGGACAGCGCCGTGTACTACTGCGCCAGGGGCAGGCCCGCCATGGACTACTGGGGCCAGGGCACCA
GCGTGACCGTGAGCAGCGGCGGCGGCGGCAGCGGCGGCGGCGGCAGCGGCGGCGGCGGCAGCGACATCGTGCTGA
CCCAGAGCCCCGCCAGCCTGGCCGTGAGCCTGGGCCAGAGGGCCACCATCAGCTGCAGGGCCAGCGAGAGCGTGG
ACAACTACGGCATCAGCTTCATGAACTGGTTCCAGCAGAAGCCCGGCCAGCCCCCCAAGCTGCTGATCTACGCCG
CCAGCAACCAGGGCAGCGGCGTGCCCGCCAGGTTCAGCGGCAGCGGCAGCGGCACCGACTTCAGCCTGAACATCC
ACCCCATGGAGGAGGACGACACCGCCATGTACTTCTGCCAGCAGAGCAAGGAGGTGCCCTGGACCTTCGGCGGCG
GCACCAAGCTGGAGATCAAGGGCCTGGCCGTGAGCACCATCAGCAGCTTCTTCCCCCCCGGCTACCAGATCTACA
TCTGGGCCCCCCTGGCCGGCACCTGCGGCGTGCTGCTGCTGAGCCTGGTGATCACCCTGTACTGCAAGAGGGGCA
GGAAGAAGCTGCTGTACATCTTCAAGCAGCCCTTCATGAGGCCCGTGCAGACCACCCAGGAGGAGGACGGCTGCA
GCTGCAGGTTCCCCGAGGAGGAGGAGGGCGGCTGCGAGCTGAGGGTGAAGTTCAGCAGGAGCGCCGACGCCCCCG
CCTACCAGCAGGGCCAGAACCAGCTGTACAACGAGCTGAACCTGGGCAGGAGGGAGGAGTACGACGTGCTGGACA
AGAGGAGGGGCAGGGACCCCGAGATGGGCGGCAAGCCCAGGAGGAAGAACCCCCAGGAGGGCCTGTACAACGAGC
TGCAGAAGGACAAGATGGCCGAGGCCTACAGCGAGATCGGCATGAAGGGCGAGAGGAGGAGGGGCAAGGGCCACG
ACGGCCTGTACCAGGGCCTGAGCACCGCCACCAAGGACACCTACGACGCCCTGCACATGCAGGCCCTGCCCCCCA
GGgagggcagaggaagtcttctaacatgcggtgacgtggaggagaatcccggcccttccgggatgaccgagtaca
agcccacggtgcgcctcgccacccgcgacgacgtccccagggccgtacgcaccctcgccgccgcgttcgccgact
accccgccacgcgccacaccgtcgatccggaccgccacatcgagcgggtcaccgagctgcaagaactcttcctca
cgcgcgtcgggctcgacatcggcaaggtgtgggtcgcggacgacggcgccgcggtggcggtctggaccacgccgg
agagcgtcgaagcgggggcggtgttcgccgagatcggcccgcgcatggccgagttgagcggttcccggctggccg
cgcagcaacagatggaaggcctcctggcgccgcaccggcccaaggagcccgcgtggttcctggccaccgtcggcg
tctcgcccgaccaccagggcaagggtctgggcagcgccgtcgtgctccccggagtggaggcggccgagcgcgccg
gggtgcccgccttcctggagacctccgcgccccgcaacctccccttctacgagcggctcggcttcaccgtcaccg
ccgacgtcgaggtgcccgaaggaccgcgcacctggtgcatgacccgcaagcccggtgcctgaATTAATTAACCAA
TTGaatcaacctctggattacaaaatttgtgaaagattgactggtattcttaactatgttgctccttttacgcta
tgtggatacgctgctttaatgcctttgtatcatgctattgcttcccgtatggctttcattttctcctccttgtat
aaatcctggttgctgtctctttatgaggagttgtggcccgttgtcaggcaacgtggcgtggtgtgcactgtgttt
gctgacgcaacccccactggttggggcattgccaccacctgtcagctcctttccgggactttcgctttccccctc
cctattgccacggcggaactcatcgccgcctgccttgcccgctgctggacaggggctcggctgttgggcactgac
aattccgtggtgttgtcggggaaatcatcgtcctttccttggctgctcgcctgtgttgccacctggattctgcgc
gggacgtccttctgctacgtcccttcggccctcaatccagcggaccttccttcccgcggcctgctgccggctctg
cggcctcttccgcgtcttcgccttcgccctcagacgagtcggatctccctttgggccgcctccccgcctggtacc
tttaagaccaatgacttacaaggcagctgtagatcttagccactttttaaaagaaaaggggggactggaagggct
aattcactcccaacgaaaataagatctgctttttgcttgtactgggtctctctggttagaccagatctgagcctg
ggagctctctggctaactagggaacccactgcttaagcctcaataaagcttgccttgagtgcttcaagtagtgtg
tgcccgtctgttgtgtgactctggtaactagagatccctcagacccttttagtcagtgtggaaaatctctagcac
GTATGTGTATGATACATAAGGTTATGTATTAATTGTAGCCGCGTTCTAACGACAATATGTACAAGCCTAATTGTG
TAGCATCTGGCTTACTGAAGCAGACCCTATCATCTCTCTCGTAAACTGCCGTCAGAGTCGGTTTGGTTGGACGAA
CCTTCTGAGTTTCTGGTAACGCCGTCCCGCACCCGGAAATGGTCAGCGAACCAATCAGCAGGGTCATCGCTAGCC
AGATCCTCTACGCCGGACGCATCGTGGCCGGCATCACCGGCGCCACAGGTGCGGTTGCTGGCGCCTATATCGCCG
ACATCACCGATGGGGAAGATCGGGCTCGCCACTTCGGGCTCATGAGCGCTTGTTTCGGCGTGGGTATGGTGGCAG
GCCCCGTGGCCGGGGGACTGTTGGGCGCCATCTCCTTGCATGCACCATTCCTTGCGGCGGCGGTGCTCAACGGCC
TCAACCTACTACTGGGCTGCTTCCTAATGCAGGAGTCGCATAAGGGAGAGCGTCGAATGGTGCACTCTCAGTACA
ATCTGCTCTGATGCCGCATAGTTAAGCCAGCCCCGACACCCGCCAACACCCGCTGACGCGCCCTGACGGGCTTGT
CTGCTCCCGGCATCCGCTTACAGACAAGCTGTGACCGTCTCCGGGAGCTGCATGTGTCAGAGGTTTTCACCGTCA
TCACCGAAACGCGCGAGACGAAAGGGCCTCGTGATACGCCTATTTTTATAGGTTAATGTCATGATAATAATGGTT
TCTTAGACGTCAGGTGGCACTTTTCGGGGAAATGTGCGCGGAACCCCTATTTGTTTATTTTTCTAAATACATTCA
AATATGTATCCGCTCATGAGACAATAACCCTGATAAATGCTTCAATAATATTGAAAAAGGAAGAGTATGAGTATT
CAACATTTCCGTGTCGCCCTTATTCCCTTTTTTGCGGCATTTTGCCTTCCTGTTTTTGCTCACCCAGAAACGCTG
GTGAAAGTAAAAGATGCTGAAGATCAGTTGGGTGCACGAGTGGGTTACATCGAACTGGATCTCAACAGCGGTAAG
ATCCTTGAGAGTTTTCGCCCCGAAGAACGTTTTCCAATGATGAGCACTTTTAAAGTTCTGCTATGTGGCGCGGTA
TTATCCCGTATTGACGCCGGGCAAGAGCAACTCGGTCGCCGCATACACTATTCTCAGAATGACTTGGTTGAGTAC
TCACCAGTCACAGAAAAGCATCTTACGGATGGCATGACAGTAAGAGAATTATGCAGTGCTGCCATAACCATGAGT
GATAACACTGCGGCCAACTTACTTCTGACAACGATCGGAGGACCGAAGGAGCTAACCGCTTTTTTGCACAACATG
GGGGATCATGTAACTCGCCTTGATCGTTGGGAACCGGAGCTGAATGAAGCCATACCAAACGACGAGCGTGACACC
ACGATGCCTGTAGCAATGGCAACAACGTTGCGCAAACTATTAACTGGCGAACTACTTACTCTAGCTTCCCGGCAA
CAATTAATAGACTGGATGGAGGCGGATAAAGTTGCAGGACCACTTCTGCGCTCGGCCCTTCCGGCTGGCTGGTTT
ATTGCTGATAAATCTGGAGCCGGTGAGCGTGGGTCTCGCGGTATCATTGCAGCACTGGGGCCAGATGGTAAGCCC
TCCCGTATCGTAGTTATCTACACGACGGGGAGTCAGGCAACTATGGATGAACGAAATAGACAGATCGCTGAGATA
GGTGCCTCACTGATTAAGCATTGGTAACTGTCAGACCAAGTTTACTCATATATACTTTAGATTGATTTAAAACTT
CATTTTTAATTTAAAAGGATCTAGGTGAAGATCCTTTTTGATAATCTCATGACCAAAATCCCTTAACGTGAGTTT
TCGTTCCACTGAGCGTCAGACCCCGTAGAAAAGATCAAAGGATCTTCTTGAGATCCTTTTTTTCTGCGCGTAATC
TGCTGCTTGCAAACAAAAAAACCACCGCTACCAGCGGTGGTTTGTTTGCCGGATCAAGAGCTACCAACTCTTTTT
CCGAAGGTAACTGGCTTCAGCAGAGCGCAGATACCAAATACTGTTCTTCTAGTGTAGCCGTAGTTAGGCCACCAC
TTCAAGAACTCTGTAGCACCGCCTACATACCTCGCTCTGCTAATCCTGTTACCAGTGGCTGCTGCCAGTGGCGAT
AAGTCGTGTCTTACCGGGTTGGACTCAAGACGATAGTTACCGGATAAGGCGCAGCGGTCGGGCTGAACGGGGGGT
TCGTGCACACAGCCCAGCTTGGAGCGAACGACCTACACCGAACTGAGATACCTACAGCGTGAGCTATGAGAAAGC
GCCACGCTTCCCGAAGGGAGAAAGGCGGACAGGTATCCGGTAAGCGGCAGGGTCGGAACAGGAGAGCGCACGAGG
GAGCTTCCAGGGGGAAACGCCTGGTATCTTTATAGTCCTGTCGGGTTTCGCCACCTCTGACTTGAGCGTCGATTT
TTGTGATGCTCGTCAGGGGGGCGGAGCCTATGGAAAAACGCCAGCAACGCGGCCTTTTTACGGTTCCTGGCCTTT
TGCTGGCCTTTTGCTCACATGTTCTTTCCTGCGTTATCCCCTGATTCTGTGGATAACCGTATTACCGCCTTTGAG
TGAGCTGATACCGCTCGCCGCAGCCGAACGACCGAGCGCAGCGAGTCAGTGAGCGAGGAAGCGGAAGAGCGCCCA
ATACGCAAACCGCCTCTCCCCGCGCGTTGGCCGATTCATTAATGCAGCTGTGGAATGTGTGTCAGTTAGGGTGTG
GAAAGTCCCCAGGCTCCCCAGCAGGCAGAAGTATGCAAAGCATGCATCTCAATTAGTCAGCAACCAGGTGTGGAA
AGTCCCCAGGCTCCCCAGCAGGCAGAAGTATGCAAAGCATGCATCTCAATTAGTCAGCAACCATAGTCCCGCCCC
TAACTCCGCCCATCCCGCCCCTAACTCCGCCCAGTTCCGCCCATTCTCCGCCCCATGGCTGACTAATTTTTTTTA
TTTATGCAGAGGCCGAGGCCGCCTCGGCCTCTGAGCTATTCCAGAAGTAGTGAGGAGGCTTTTTTGGAGGCCTAG
GCTTTTGCAAAAAGCTTGGACACAAGACAGGCTTGCGAGATATGTTTGAGAATACCACTTTATCCCGCGTCAGGG
AGAGGCAGTGCGTAAAAAGACGCGGACTCATGTGAAATACTGGTTTTTAGTGCGCCAGATCTCTATAATCTCGCG
CAACCTATTTTCCCCTCGAACACTTTTTAAGCCGTAGATAAACAGGCTGGGACACTTCACATGAGCGAAAAATAC
ATCGTCACCTGGGACATGTTGCAGATCCATGCACGTAAACTCGCAAGCCGACTGATGCCTTCTGAACAATGGAAA
GGCATTATTGCCGTAAGCCGTGGCGGTCTGTACCGGGTGCGTTACTGGCGCGTGAACTGGGTATTCGTCATGTCG
ATACCGTTTGTATTTCCAGCTACGATCACGACAACCAGCGCGAGCTTAAAGTGCTGAAACGCGCAGAAGGCGATG
GCGAAGGCTTCATCGTTATTGATGACCTGGTGGATACCGGTGGTACTGCGGTTGCGATTCGTGAAATGTATCCAA
AAGCGCACTTTGTCACCATCTTCGCAAAACCGGCTGGTCGTCCGCTGGTTGATGACTATGTTGTTGATATCCCGC
AAGATACCTGGATTGAACAGCCGTGGGATATGGGCGTCGTATTCGTCCCGCCAATCTCCGGTCGCTAATCTTTTC
AACGCCTGGCACTGCCGGGCGTTGTTCTTTTTAACTTCAGGCGGGTTACAATAGTTTCCAGTAAGTATTCTGGAG
GCTGCATCCATGACACAGGCAAACCTGAGCGAAACCCTGTTCAAACCCCGCTTTAAACATCCTGAAACCTCGACG
CTAGTCCGCCGCTTTAATCACGGCGCACAACCGCCTGTGCAGTCGGCCCTTGATGGTAAAACCATCCCTCACTGG
TATCGCATGATTAACCGTCTGATGTGGATCTGGCGCGGCATTGACCCACGCGAAATCCTCGACGTCCAGGCACGT
ATTGTGATGAGCGATGCCGAACGTACCGACGATGATTTATACGATACGGTGATTGGCTACCGTGGCGGCAACTGG
ATTTATGAGTGGGCCCCGGATCTTTGTGAAGGAACCTTACTTCTGTGGTGTGACATAATTGGACAAACTACCTAC
AGAGATTTAAAGCTCTAAGGTAAATATAAAATTTTTAAGTGTATAATGTGTTAAACTACTGATTCTAATTGTTTG
TGTATTTTAGATTCCAACCTATGGAACTGATGAATGGGAGCAGTGGTGGAATGCCTTTAATGAGGAAAACCTGTT
TTGCTCAGAAGAAATGCCATCTAGTGATGATGAGGCTACTGCTGACTCTCAACATTCTACTCCTCCAAAAAAGAA
GAGAAAGGTAGAAGACCCCAAGGACTTTCCTTCAGAATTGCTAAGTTTTTTGAGTCATGCTGTGTTTAGTAATAG
AACTCTTGCTTGCTTTGCTATTTACACCACAAAGGAAAAAGCTGCACTGCTATACAAGAAAATTATGGAAAAATA
TTCTGTAACCTTTATAAGTAGGCATAACAGTTATAATCATAACATACTGTTTTTTCTTACTCCACACAGGCATAG
AGTGTCTGCTATTAATAACTATGCTCAAAAATTGTGTACCTTTAGCTTTTTAATTTGTAAAGGGGTTAATAAGGA
ATATTTGATGTATAGTGCCTTGACTAGAGATCATAATCAGCCATACCACATTTGTAGAGGTTTTACTTGCTTTAA
AAAACCTCCCACACCTCCCCCTGAACCTGAAACATAAAATGAATGCAATTGTTGTTGTTAACTTGTTTATTGCAG
CTTATAATGGTTACAAATAAAGCAATAGCATCACAAATTTCACAAATAAAGCATTTTTTTCACTGCATTCTAGTT
GTGGTTTGTCCAAACTCATCAATGTATCTTATCATGTCTGGATCAACTGGATAACTCAAGCTAACCAAAATCATC
CCAAACTTCCCACCCCATACCCTATTACCACTGCCAATTACCTGTGGTTTCATTTACTCTAAACCTGTGATTCCT
CTGAATTATTTTCATTTTAAAGAAATTGTATTTGTTAAATATGTACTACAAACTTAGTAG

13. My9.6-CD8-41BB-CD3z_5

An exemplary CAR construct, as described herein, comprises a CD33 binding domain (My9.6) scFv, a CD8a transmembrane domain, a CD8a hinge domain, a CD137 (4-1BB) co-stimulatory domain, and a CD3ζ intracellular signaling domain.

In some embodiments, a CAR in encoded by a nucleic acid sequence that comprises the sequence that is shown in SEQ ID NO: 47, or in a nucleic acid sequence that is at least 70%, at least 75%, at least 80%, at least 85%, at least 90%, at least 95%, or at least 99% identical to the nucleic acid sequence shown in SEQ ID NO: 47.

[SEQ ID NO: 47]
ATGGCCCTGCCCGTGACCGCCCTGCTGCTGCCCCTGGCCCTGCTGCTGCACGCCGCCAGGCCCCAGGTGCAGCTG
CAGCAGCCCGGCGCCGAGGTGGTGAAGCCCGGCGCCAGCGTGAAGATGAGCTGCAAGGCCAGCGGCTACACCTTC
ACCAGCTACTACATCCACTGGATCAAGCAGACCCCCGGCCAGGGCCTGGAGTGGGTGGGCGTGATCTACCCCGGC
AACGACGACATCAGCTACAACCAGAAGTTCAAGGGCAAGGCCACCCTGACCGCCGACAAGAGCAGCACCACCGCC
TACATGCAGCTGAGCAGCCTGACCAGCGAGGACAGCGCCGTGTACTACTGCGCCAGGGAGGTGAGGCTGAGGTAC
TTCGACGTGTGGGGCGCCGGCACCACCGTGACCGTGAGCAGCGGCGGCGGCGGCAGCGGCGGCGGCGGCAGCGGC
GGCGGCGGCAGCAACATCATGCTGACCCAGAGCCCCAGCAGCCTGGCCGTGAGCGCCGGCGAGAAGGTGACCATG
AGCTGCAAGAGCAGCCAGAGCGTGTTCTTCAGCAGCAGCCAGAAGAACTACCTGGCCTGGTACCAGCAGATCCCC
GGCCAGAGCCCCAAGCTGCTGATCTACTGGGCCAGCACCAGGGAGAGCGGCGTGCCCGACAGGTTCACCGGCAGC
GGCAGCGGCACCGACTTCACCCTGACCATCAGCAGCGTGCAGAGCGAGGACCTGGCCATCTACTACTGCCACCAG
TACCTGAGCAGCAGGACCTTCGGCGGCGGCACCAAGCTGGAGATCAAGAGGGGCCTGGCCGTGAGCACCATCAGC
AGCTTCTTCCCCCCCGGCTACCAGATCTACATCTGGGCCCCCCTGGCCGGCACCTGCGGCGTGCTGCTGCTGAGC
CTGGTGATCACCCTGTACTGCAAGAGGGGCAGGAAGAAGCTGCTGTACATCTTCAAGCAGCCCTTCATGAGGCCC
GTGCAGACCACCCAGGAGGAGGACGGCTGCAGCTGCAGGTTCCCCGAGGAGGAGGAGGGCGGCTGCGAGCTGAGG
GTGAAGTTCAGCAGGAGCGCCGACGCCCCCGCCTACCAGCAGGGCCAGAACCAGCTGTACAACGAGCTGAACCTG
GGCAGGAGGGAGGAGTACGACGTGCTGGACAAGAGGAGGGGCAGGGACCCCGAGATGGGCGGCAAGCCCAGGAGG
AAGAACCCCCAGGAGGGCCTGTACAACGAGCTGCAGAAGGACAAGATGGCCGAGGCCTACAGCGAGATCGGCATG
AAGGGCGAGAGGAGGAGGGGCAAGGGCCACGACGGCCTGTACCAGGGCCTGAGCACCGCCACCAAGGACACCTAC
GACGCCCTGCACATGCAGGCCCTGCCCCCCAGG

In some embodiments, a CAR comprises an amino acid sequence shown in SEQ ID NO: 48, or an amino acid sequence that is at least 70%, at least 75%, at least 80%, at least 85%, at least 90%, at least 95%, or at least 99% identical to the amino acid sequence shown in SEQ ID NO: 48.

[SEQ ID NO: 48]
MALPVTALLLPLALLLHAARPQVQLQQPGAEVVKPGASVKMSCKASGYTFTSYYIHWIKQTPGQGLEWVGVIYPG
NDDISYNQKFKGKATLTADKSSTTAYMQLSSLTSEDSAVYYCAREVRLRYFDVWGAGTTVTVSSGGGGSGGGGSG

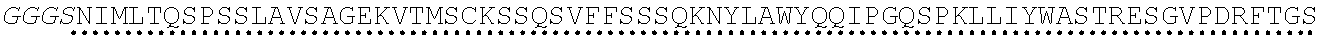

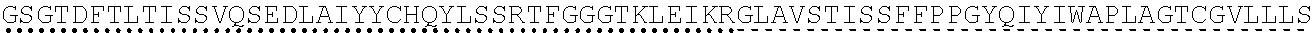

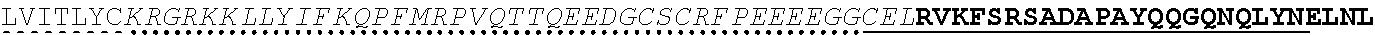

GRREEYDVLDKRRGRDPEMGGKPRRKNPQEGLYNELQKDKMAEAYSEIGMKGERRRGKGHDGLYQGLSTATKDTY
DALHMQALPPR

In some embodiments, a CAR construct as shown in SEQ ID NO: 47 is included in a recombinant expression vector. In some embodiments the recombinant expression vector includes a promoter (e.g., an SFFV promoter or and EF1α promoter). In some embodiments, a recombinant expression vector including the CAR of SEQ ID NO: 47 comprises the sequence that is shown in SEQ ID NO: 49, or in a nucleic acid sequence that is at least 70%, at least 75%, at least 80%, at least 85%, at least 90%, at least 95%, or at least 99% identical to the nucleic acid sequence shown in SEQ ID NO: 49.

In some embodiments, a recombinant expression vector including the CAR of SEQ ID NO: 47 comprises the sequence that is shown in SEQ ID NO: 49, but does not include the SFFV promoter sequence (as shown in SEQ ID NO: 8). In some embodiments, a recombinant expression vector comprises the nucleic acid sequence shown in SEQ ID NO: 49, but does not include the SFFV sequence (as shown in SEQ ID NO: 8), and instead includes an EF1α promoter sequence.

[SEQ ID NO: 49]
Tgggtctctctggttagaccagatctgagcctgggagctctctggctaactagggaacccactgcttaagcctca
ataaagcttgccttgagtgcttcaagtagtgtgtgcccgtctgttgtgtgactctggtaactagagatccctcag
acccttttagtcagtgtggaaaatctctagcagtggcgcccgaacagggacttgaaagcgaaagggaaaccagag
gagctctctcgacgcaggactcggcttgctgaagcgcgcacggcaagaggcgaggggcggcgactggtgagtacg
ccaaaaattttgactagcggaggctagaaggagagagatgggtgcgagagcgtcagtattaagcgggggagaatt
agatcgcgatgggaaaaaattcggttaaggccagggggaaagaaaaaatataaattaaaacatatagtatgggca
agcagggagctagaacgattcgcagttaatcctggcctgttagaaacatcagaaggctgtagacaaatactggga
cagctacaaccatcccttcagacaggatcagaagaacttagatcattatataatacagtagcaaccctctattgt
gtgcatcaaaggatagagataaaagacaccaaggaagctttagacaagatagaggaagagcaaaacaaaagtaag
accaccgcacagcaagcggccactgatcttcagacctggaggaggagatatgagggacaattggagaagtgaatt
atataaatataaagtagtaaaaattgaaccattaggagtagcacccaccaaggcaaagagaagagtggtgcagag
agaaaaaagagcagtgggaataggagctttgttccttgggttcttgggagcagcaggaagcactatgggcgcagc
gtcaatgacgctgacggtacaggccagacaattattgtctggtatagtgcagcagcagaacaatttgctgagggc
tattgaggcgcaacagcatctgttgcaactcacagtctggggcatcaagcagctccaggcaagaatcctggctgt
ggaaagatacctaaaggatcaacagctcctggggatttggggttgctctggaaaactcatttgcaccactgctgt
gccttggaatgctagttggagtaataaatctctggaacagaattggaatcacacgacctggatggagtgggacag
agaaattaacaattacacaagcttaatacactccttaattgaagaatcgcaaaaccagcaagaaaagaatgaaca
agaattattggaattagataaatgggcaagtttgtggaattggtttaacataacaaattggctgtggtatataaa
attattcataatgatagtaggaggcttggtaggtttaagaatagtttttgctgtactttctatagtgaatagagt
taggcagggatattcaccattatcgtttcagacccacctcccaaccccgaggggacccgacaggcccgaaggaat
agaagaagaaggtggagagagagacagagacagatccattcgattagtgaacggatctcgacggtatcggttaac
ttttaaaagaaaaggggggattggggggtacagtgcaggggaaagaatagtagacataatagcaacagacataca
aactaaagaattacaaaaacaaattacaaaattcaaaattttatcgatactagtggatctGCGATCGCGTAACGC
CATTTTGCAAGGCATGGAAAAATACCAAACCAAGAATAGAGAAGTTCAGATCAAGGGCGGGTACATGAAAATAGC
TAACGTTGGGCCAAACAGGATATCTGCGGTGAGCAGTTTCGGCCCCGGCCCGGGGCCAAGAACAGATGGTCACCG
CAGTTTCGGCCCCGGCCCGAGGCCAAGAACAGATGGTCCCCAGATATGGCCCAACCCTCAGCAGTTTCTTAAGAC
CCATCAGATGTTTCCAGGCTCCCCCAAGGACCTGAAATGACCCTGCGCCTTATTTGAATTAACCAATCAGCCTGC
TTCTCGCTTCTGTTCGCGCGCTTCTGCTTCCCGAGCTCTATAAAAGAGCTCACAACCCCTCACTCGGCGCGCCAG
TCCTCCGACAGACTGAGTCGTCTAGAgctagcGGATCCCCGGAATTCGACGCCACCATGGCCCTGCCCGTGACCG
CCCTGCTGCTGCCCCTGGCCCTGCTGCTGCACGCCGCCAGGCCCCAGGTGCAGCTGCAGCAGCCCGGCGCCGAGG
TGGTGAAGCCCGGCGCCAGCGTGAAGATGAGCTGCAAGGCCAGCGGCTACACCTTCACCAGCTACTACATCCACT
GGATCAAGCAGACCCCCGGCCAGGGCCTGGAGTGGGTGGGCGTGATCTACCCCGGCAACGACGACATCAGCTACA
ACCAGAAGTTCAAGGGCAAGGCCACCCTGACCGCCGACAAGAGCAGCACCACCGCCTACATGCAGCTGAGCAGCC
TGACCAGCGAGGACAGCGCCGTGTACTACTGCGCCAGGGAGGTGAGGCTGAGGTACTTCGACGTGTGGGGCGCCG
GCACCACCGTGACCGTGAGCAGCGGCGGCGGCGGCAGCGGCGGCGGCGGCAGCGGCGGCGGCGGCAGCAACATCA
TGCTGACCCAGAGCCCCAGCAGCCTGGCCGTGAGCGCCGGCGAGAAGGTGACCATGAGCTGCAAGAGCAGCCAGA
GCGTGTTCTTCAGCAGCAGCCAGAAGAACTACCTGGCCTGGTACCAGCAGATCCCCGGCCAGAGCCCCAAGCTGC
TGATCTACTGGGCCAGCACCAGGGAGAGCGGCGTGCCCGACAGGTTCACCGGCAGCGGCAGCGGCACCGACTTCA
CCCTGACCATCAGCAGCGTGCAGAGCGAGGACCTGGCCATCTACTACTGCCACCAGTACCTGAGCAGCAGGACCT
TCGGCGGCGGCACCAAGCTGGAGATCAAGAGGGGCCTGGCCGTGAGCACCATCAGCAGCTTCTTCCCCCCCGGCT
ACCAGATCTACATCTGGGCCCCCCTGGCCGGCACCTGCGGCGTGCTGCTGCTGAGCCTGGTGATCACCCTGTACT
GCAAGAGGGGCAGGAAGAAGCTGCTGTACATCTTCAAGCAGCCCTTCATGAGGCCCGTGCAGACCACCCAGGAGG
AGGACGGCTGCAGCTGCAGGTTCCCCGAGGAGGAGGAGGGCGGCTGCGAGCTGAGGGTGAAGTTCAGCAGGAGCG
CCGACGCCCCCGCCTACCAGCAGGGCCAGAACCAGCTGTACAACGAGCTGAACCTGGGCAGGAGGGAGGAGTACG
ACGTGCTGGACAAGAGGAGGGGCAGGGACCCCGAGATGGGCGGCAAGCCCAGGAGGAAGAACCCCCAGGAGGGCC
TGTACAACGAGCTGCAGAAGGACAAGATGGCCGAGGCCTACAGCGAGATCGGCATGAAGGGCGAGAGGAGGAGGG
GCAAGGGCCACGACGGCCTGTACCAGGGCCTGAGCACCGCCACCAAGGACACCTACGACGCCCTGCACATGCAGG
CCCTGCCCCCCAGGgagggcagaggaagtcttctaacatgcggtgacgtggaggagaatcccggcccttccggga
tgaccgagtacaagcccacggtgcgcctcgccacccgcgacgacgtccccagggccgtacgcaccctcgccgccg
cgttcgccgactaccccgccacgcgccacacegtcgatccggaccgccacatcgagcgggtcaccgagctgcaag
aactcttcctcacgcgcgtcgggctcgacatcggcaaggtgtgggtcgcggacgacggcgccgcggtggcggtct
ggaccacgccggagagcgtcgaagcgggggcggtgttcgccgagatcggcccgcgcatggccgagttgagcggtt
cccggctggccgcgcagcaacagatggaaggcctcctggcgccgcaccggcccaaggagcccgcgtggttcctgg
ccaccgtcggcgtctcgcccgaccaccagggcaagggtctgggcagcgccgtcgtgctccccggagtggaggcgg
ccgagcgcgccggggtgcccgccttcctggagacctccgcgccccgcaacctccccttctacgagcggctcggct
tcaccgtcaccgccgacgtcgaggtgcccgaaggaccgcgcacctggtgcatgacccgcaagcccggtgcctgaA
TTAATTAACCAATTGaatcaacctctggattacaaaatttgtgaaagattgactggtattcttaactatgttgct
ccttttacgctatgtggatacgctgctttaatgcctttgtatcatgctattgcttcccgtatggctttcattttc
tcctccttgtataaatcctggttgctgtctctttatgaggagttgtggcccgttgtcaggcaacgtggcgtggtg
tgcactgtgtttgctgacgcaacccccactggttggggcattgccaccacctgtcagctcctttccgggactttc
gctttccccctccctattgccacggcggaactcatcgccgcctgccttgcccgctgctggacaggggctcggctg
ttgggcactgacaattccgtggtgttgtcggggaaatcatcgtcctttccttggctgctcgcctgtgttgccacc
tggattctgcgcgggacgtccttctgctacgtcccttcggccctcaatccagcggaccttccttcccgcggcctg
ctgccggctctgcggcctcttccgcgtcttcgccttcgccctcagacgagtcggatctccctttgggccgcctcc
ccgcctggtacctttaagaccaatgacttacaaggcagctgtagatcttagccactttttaaaagaaaagggggg
actggaagggctaattcactcccaacgaaaataagatctgctttttgcttgtactgggtctctctggttagacca
gatctgagcctgggagctctctggctaactagggaacccactgcttaagcctcaataaagcttgccttgagtgct
tcaagtagtgtgtgcccgtctgttgtgtgactctggtaactagagatccctcagacccttttagtcagtgtggaa
aatctctagcaCGTATGTGTATGATACATAAGGTTATGTATTAATTGTAGCCGCGTTCTAACGACAATATGTACA
AGCCTAATTGTGTAGCATCTGGCTTACTGAAGCAGACCCTATCATCTCTCTCGTAAACTGCCGTCAGAGTCGGTT
TGGTTGGACGAACCTTCTGAGTTTCTGGTAACGCCGTCCCGCACCCGGAAATGGTCAGCGAACCAATCAGCAGGG
TCATCGCTAGCCAGATCCTCTACGCCGGACGCATCGTGGCCGGCATCACCGGCGCCACAGGTGCGGTTGCTGGCG
CCTATATCGCCGACATCACCGATGGGGAAGATCGGGCTCGCCACTTCGGGCTCATGAGCGCTTGTTTCGGCGTGG
GTATGGTGGCAGGCCCCGTGGCCGGGGGACTGTTGGGCGCCATCTCCTTGCATGCACCATTCCTTGCGGCGGCGG
TGCTCAACGGCCTCAACCTACTACTGGGCTGCTTCCTAATGCAGGAGTCGCATAAGGGAGAGCGTCGAATGGTGC
ACTCTCAGTACAATCTGCTCTGATGCCGCATAGTTAAGCCAGCCCCGACACCCGCCAACACCCGCTGACGCGCCC
TGACGGGCTTGTCTGCTCCCGGCATCCGCTTACAGACAAGCTGTGACCGTCTCCGGGAGCTGCATGTGTCAGAGG
TTTTCACCGTCATCACCGAAACGCGCGAGACGAAAGGGCCTCGTGATACGCCTATTTTTATAGGTTAATGTCATG
ATAATAATGGTTTCTTAGACGTCAGGTGGCACTTTTCGGGGAAATGTGCGCGGAACCCCTATTTGTTTATTTTTC
TAAATACATTCAAATATGTATCCGCTCATGAGACAATAACCCTGATAAATGCTTCAATAATATTGAAAAAGGAAG
AGTATGAGTATTCAACATTTCCGTGTCGCCCTTATTCCCTTTTTTGCGGCATTTTGCCTTCCTGTTTTTGCTCAC
CCAGAAACGCTGGTGAAAGTAAAAGATGCTGAAGATCAGTTGGGTGCACGAGTGGGTTACATCGAACTGGATCTC
AACAGCGGTAAGATCCTTGAGAGTTTTCGCCCCGAAGAACGTTTTCCAATGATGAGCACTTTTAAAGTTCTGCTA
TGTGGCGCGGTATTATCCCGTATTGACGCCGGGCAAGAGCAACTCGGTCGCCGCATACACTATTCTCAGAATGAC
TTGGTTGAGTACTCACCAGTCACAGAAAAGCATCTTACGGATGGCATGACAGTAAGAGAATTATGCAGTGCTGCC
ATAACCATGAGTGATAACACTGCGGCCAACTTACTTCTGACAACGATCGGAGGACCGAAGGAGCTAACCGCTTTT
TTGCACAACATGGGGGATCATGTAACTCGCCTTGATCGTTGGGAACCGGAGCTGAATGAAGCCATACCAAACGAC
GAGCGTGACACCACGATGCCTGTAGCAATGGCAACAACGTTGCGCAAACTATTAACTGGCGAACTACTTACTCTA
GCTTCCCGGCAACAATTAATAGACTGGATGGAGGCGGATAAAGTTGCAGGACCACTTCTGCGCTCGGCCCTTCCG
GCTGGCTGGTTTATTGCTGATAAATCTGGAGCCGGTGAGCGTGGGTCTCGCGGTATCATTGCAGCACTGGGGCCA
GATGGTAAGCCCTCCCGTATCGTAGTTATCTACACGACGGGGAGTCAGGCAACTATGGATGAACGAAATAGACAG
ATCGCTGAGATAGGTGCCTCACTGATTAAGCATTGGTAACTGTCAGACCAAGTTTACTCATATATACTTTAGATT
GATTTAAAACTTCATTTTTAATTTAAAAGGATCTAGGTGAAGATCCTTTTTGATAATCTCATGACCAAAATCCCT
TAACGTGAGTTTTCGTTCCACTGAGCGTCAGACCCCGTAGAAAAGATCAAAGGATCTTCTTGAGATCCTTTTTTT
CTGCGCGTAATCTGCTGCTTGCAAACAAAAAAACCACCGCTACCAGCGGTGGTTTGTTTGCCGGATCAAGAGCTA
CCAACTCTTTTTCCGAAGGTAACTGGCTTCAGCAGAGCGCAGATACCAAATACTGTTCTTCTAGTGTAGCCGTAG
TTAGGCCACCACTTCAAGAACTCTGTAGCACCGCCTACATACCTCGCTCTGCTAATCCTGTTACCAGTGGCTGCT
GCCAGTGGCGATAAGTCGTGTCTTACCGGGTTGGACTCAAGACGATAGTTACCGGATAAGGCGCAGCGGTCGGGC
TGAACGGGGGGTTCGTGCACACAGCCCAGCTTGGAGCGAACGACCTACACCGAACTGAGATACCTACAGCGTGAG
CTATGAGAAAGCGCCACGCTTCCCGAAGGGAGAAAGGCGGACAGGTATCCGGTAAGCGGCAGGGTCGGAACAGGA
GAGCGCACGAGGGAGCTTCCAGGGGGAAACGCCTGGTATCTTTATAGTCCTGTCGGGTTTCGCCACCTCTGACTT
GAGCGTCGATTTTTGTGATGCTCGTCAGGGGGGCGGAGCCTATGGAAAAACGCCAGCAACGCGGCCTTTTTACGG
TTCCTGGCCTTTTGCTGGCCTTTTGCTCACATGTTCTTTCCTGCGTTATCCCCTGATTCTGTGGATAACCGTATT
ACCGCCTTTGAGTGAGCTGATACCGCTCGCCGCAGCCGAACGACCGAGCGCAGCGAGTCAGTGAGCGAGGAAGCG
GAAGAGCGCCCAATACGCAAACCGCCTCTCCCCGCGCGTTGGCCGATTCATTAATGCAGCTGTGGAATGTGTGTC
AGTTAGGGTGTGGAAAGTCCCCAGGCTCCCCAGCAGGCAGAAGTATGCAAAGCATGCATCTCAATTAGTCAGCAA
CCAGGTGTGGAAAGTCCCCAGGCTCCCCAGCAGGCAGAAGTATGCAAAGCATGCATCTCAATTAGTCAGCAACCA
TAGTCCCGCCCCTAACTCCGCCCATCCCGCCCCTAACTCCGCCCAGTTCCGCCCATTCTCCGCCCCATGGCTGAC
TAATTTTTTTTATTTATGCAGAGGCCGAGGCCGCCTCGGCCTCTGAGCTATTCCAGAAGTAGTGAGGAGGCTTTT
TTGGAGGCCTAGGCTTTTGCAAAAAGCTTGGACACAAGACAGGCTTGCGAGATATGTTTGAGAATACCACTTTAT
CCCGCGTCAGGGAGAGGCAGTGCGTAAAAAGACGCGGACTCATGTGAAATACTGGTTTTTAGTGCGCCAGATCTC
TATAATCTCGCGCAACCTATTTTCCCCTCGAACACTTTTTAAGCCGTAGATAAACAGGCTGGGACACTTCACATG
AGCGAAAAATACATCGTCACCTGGGACATGTTGCAGATCCATGCACGTAAACTCGCAAGCCGACTGATGCCTTCT
GAACAATGGAAAGGCATTATTGCCGTAAGCCGTGGCGGTCTGTACCGGGTGCGTTACTGGCGCGTGAACTGGGTA
TTCGTCATGTCGATACCGTTTGTATTTCCAGCTACGATCACGACAACCAGCGCGAGCTTAAAGTGCTGAAACGCG
CAGAAGGCGATGGCGAAGGCTTCATCGTTATTGATGACCTGGTGGATACCGGTGGTACTGCGGTTGCGATTCGTG
AAATGTATCCAAAAGCGCACTTTGTCACCATCTTCGCAAAACCGGCTGGTCGTCCGCTGGTTGATGACTATGTTG
TTGATATCCCGCAAGATACCTGGATTGAACAGCCGTGGGATATGGGCGTCGTATTCGTCCCGCCAATCTCCGGTC
GCTAATCTTTTCAACGCCTGGCACTGCCGGGCGTTGTTCTTTTTAACTTCAGGCGGGTTACAATAGTTTCCAGTA
AGTATTCTGGAGGCTGCATCCATGACACAGGCAAACCTGAGCGAAACCCTGTTCAAACCCCGCTTTAAACATCCT
GAAACCTCGACGCTAGTCCGCCGCTTTAATCACGGCGCACAACCGCCTGTGCAGTCGGCCCTTGATGGTAAAACC
ATCCCTCACTGGTATCGCATGATTAACCGTCTGATGTGGATCTGGCGCGGCATTGACCCACGCGAAATCCTCGAC
GTCCAGGCACGTATTGTGATGAGCGATGCCGAACGTACCGACGATGATTTATACGATACGGTGATTGGCTACCGT
GGCGGCAACTGGATTTATGAGTGGGCCCCGGATCTTTGTGAAGGAACCTTACTTCTGTGGTGTGACATAATTGGA
CAAACTACCTACAGAGATTTAAAGCTCTAAGGTAAATATAAAATTTTTAAGTGTATAATGTGTTAAACTACTGAT
TCTAATTGTTTGTGTATTTTAGATTCCAACCTATGGAACTGATGAATGGGAGCAGTGGTGGAATGCCTTTAATGA
GGAAAACCTGTTTTGCTCAGAAGAAATGCCATCTAGTGATGATGAGGCTACTGCTGACTCTCAACATTCTACTCC
TCCAAAAAAGAAGAGAAAGGTAGAAGACCCCAAGGACTTTCCTTCAGAATTGCTAAGTTTTTTGAGTCATGCTGT
GTTTAGTAATAGAACTCTTGCTTGCTTTGCTATTTACACCACAAAGGAAAAAGCTGCACTGCTATACAAGAAAAT
TATGGAAAAATATTCTGTAACCTTTATAAGTAGGCATAACAGTTATAATCATAACATACTGTTTTTTCTTACTCC
ACACAGGCATAGAGTGTCTGCTATTAATAACTATGCTCAAAAATTGTGTACCTTTAGCTTTTTAATTTGTAAAGG
GGTTAATAAGGAATATTTGATGTATAGTGCCTTGACTAGAGATCATAATCAGCCATACCACATTTGTAGAGGTTT
TACTTGCTTTAAAAAACCTCCCACACCTCCCCCTGAACCTGAAACATAAAATGAATGCAATTGTTGTTGTTAACT
TGTTTATTGCAGCTTATAATGGTTACAAATAAAGCAATAGCATCACAAATTTCACAAATAAAGCATTTTTTTCAC
TGCATTCTAGTTGTGGTTTGTCCAAACTCATCAATGTATCTTATCATGTCTGGATCAACTGGATAACTCAAGCTA
ACCAAAATCATCCCAAACTTCCCACCCCATACCCTATTACCACTGCCAATTACCTGTGGTTTCATTTACTCTAAA
CCTGTGATTCCTCTGAATTATTTTCATTTTAAAGAAATTGTATTTGTTAAATATGTACTACAAACTTAGTAG

14. M2H12-CD8-41BB-CD3z

An exemplary CAR construct, as described herein, comprises a CD33 binding domain (M2H12) scFv, a CD8a transmembrane domain, a CD8a hinge domain, a CD137 (4-1BB) co-stimulatory domain, and a CD3ζ intracellular signaling domain.

In some embodiments, a CAR in encoded by a nucleic acid sequence that comprises the sequence that is shown in SEQ ID NO: 50, or in a nucleic acid sequence that is at least 70%, at least 75%, at least 80%, at least 85%, at least 90%, at least 95%, or at least 99% identical to the nucleic acid sequence shown in SEQ ID NO: 50.

[SEQ ID NO: 50]
ATGGCCCTGCCCGTGACCGCCCTGCTGCTGCCCCTGGCCCTGCTGCTGCACGCCGCCAGGCCCCAGGTGCAGCTG
CAGCAGAGCGGCCCCGAGCTGGTGAGGCCCGGCACCTTCGTGAAGATCAGCTGCAAGGCCAGCGGCTACACCTTC
ACCAACTACGACATCAACTGGGTGAACCAGAGGCCCGGCCAGGGCCTGGAGTGGATCGGCTGGATCTACCCCGGC
GACGGCAGCACCAAGTACAACGAGAAGTTCAAGGCCAAGGCCACCCTGACCGCCGACAAGAGCAGCAGCACCGCC
TACCTGCAGCTGAACAACCTGACCAGCGAGAACAGCGCCGTGTACTTCTGCGCCAGCGGCTACGAGGACGCCATG
GACTACTGGGGCCAGGGCACCAGCGTGACCGTGAGCAGCGGCGGCGGCGGCAGCGGCGGCGGCGGCAGCGGCGGC
GGCGGCAGCGACATCAAGATGACCCAGAGCCCCAGCAGCATGTACGCCAGCCTGGGCGAGAGGGTGATCATCAAC
TGCAAGGCCAGCCAGGACATCAACAGCTACCTGAGCTGGTTCCAGCAGAAGCCCGGCAAGAGCCCCAAGACCCTG
ATCTACAGGGCCAACAGGCTGGTGGACGGCGTGCCCAGCAGGTTCAGCGGCAGCGGCAGCGGCCAGGACTACAGC
CTGACCATCAGCAGCCTGGAGTACGAGGACATGGGCATCTACTACTGCCTGCAGTACGACGAGTTCCCCCTGACC
TTCGGCGCCGGCACCAAGCTGGAGCTGAAGAGGGGCCTGGCCGTGAGCACCATCAGCAGCTTCTTCCCCCCCGGC
TACCAGATCTACATCTGGGCCCCCCTGGCCGGCACCTGCGGCGTGCTGCTGCTGAGCCTGGTGATCACCCTGTAC
TGCAAGAGGGGCAGGAAGAAGCTGCTGTACATCTTCAAGCAGCCCTTCATGAGGCCCGTGCAGACCACCCAGGAG
GAGGACGGCTGCAGCTGCAGGTTCCCCGAGGAGGAGGAGGGCGGCTGCGAGCTGAGGGTGAAGTTCAGCAGGAGC
GCCGACGCCCCCGCCTACCAGCAGGGCCAGAACCAGCTGTACAACGAGCTGAACCTGGGCAGGAGGGAGGAGTAC
GACGTGCTGGACAAGAGGAGGGGCAGGGACCCCGAGATGGGCGGCAAGCCCAGGAGGAAGAACCCCCAGGAGGGC
CTGTACAACGAGCTGCAGAAGGACAAGATGGCCGAGGCCTACAGCGAGATCGGCATGAAGGGCGAGAGGAGGAGG
GGCAAGGGCCACGACGGCCTGTACCAGGGCCTGAGCACCGCCACCAAGGACACCTACGACGCCCTGCACATGCAG
GCCCTGCCCCCCAGG

In some embodiments, a CAR comprises an amino acid sequence shown in SEQ ID NO: 51, or an amino acid sequence that is at least 70%, at least 75%, at least 80%, at least 85%, at least 90%, at least 95%, or at least 99% identical to the amino acid sequence shown in SEQ ID NO: 51.

[SEQ ID NO: 51]
MALPVTALLLPLALLLHAARPQVQLQQSGPELVRPGTFVKISCKASGYTFTNYDINWVNQRPGQGLEWIGWIYPG
DGSTKYNEKFKAKATLTADKSSSTAYLQLNNLTSENSAVYFCASGYEDAMDYWGQGTSVTVSSGGGGSGGGGSGG

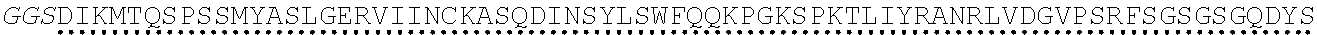

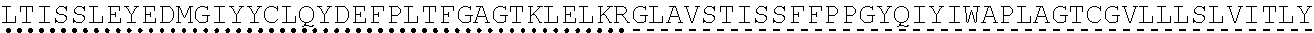

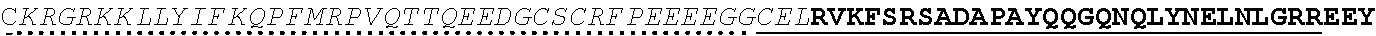

DVLDKRRGRDPEMGGKPRRKNPQEGLYNELQKDKMAEAYSEIGMKGERRRGKGHDGLYQGLSTATKDTYDALHMQ
ALPPR

In some embodiments, a CAR construct as shown in SEQ ID NO: 50 is included in a recombinant expression vector. In some embodiments the recombinant expression vector includes a promoter (e.g., an SFFV promoter or and EF1α promoter). In some embodiments, a recombinant expression vector including the CAR of SEQ ID NO: 50 comprises the sequence that is shown in SEQ ID NO: 52, or in a nucleic acid sequence that is at least 70%, at least 75%, at least 80%, at least 85%, at least 90%, at least 95%, or at least 99% identical to the nucleic acid sequence shown in SEQ ID NO: 52.

In some embodiments, a recombinant expression vector including the CAR of SEQ ID NO: 50 comprises the sequence that is shown in SEQ ID NO: 52, but does not include the SFFV promoter sequence (as shown in SEQ ID NO: 8). In some embodiments, a recombinant expression vector comprises the nucleic acid sequence shown in SEQ ID NO: 52, but does not include the SFFV sequence (as shown in SEQ ID NO: 8), and instead includes an EF1α promoter sequence.

[SEQ ID NO: 52]
Tgggtctctctggttagaccagatctgagcctgggagctctctggctaactagggaacccactgcttaagcctca
ataaagcttgccttgagtgcttcaagtagtgtgtgcccgtctgttgtgtgactctggtaactagagatccctcag
acccttttagtcagtgtggaaaatctctagcagtggcgcccgaacagggacttgaaagcgaaagggaaaccagag
gagctctctcgacgcaggactcggcttgctgaagcgcgcacggcaagaggcgaggggcggcgactggtgagtacg
ccaaaaattttgactagcggaggctagaaggagagagatgggtgcgagagcgtcagtattaagcgggggagaatt
agatcgcgatgggaaaaaattcggttaaggccagggggaaagaaaaaatataaattaaaacatatagtatgggca
agcagggagctagaacgattcgcagttaatcctggcctgttagaaacatcagaaggctgtagacaaatactggga
cagctacaaccatcccttcagacaggatcagaagaacttagatcattatataatacagtagcaaccctctattgt
gtgcatcaaaggatagagataaaagacaccaaggaagctttagacaagatagaggaagagcaaaacaaaagtaag
accaccgcacagcaagcggccactgatcttcagacctggaggaggagatatgagggacaattggagaagtgaatt
atataaatataaagtagtaaaaattgaaccattaggagtagcacccaccaaggcaaagagaagagtggtgcagag
agaaaaaagagcagtgggaataggagctttgttccttgggttcttgggagcagcaggaagcactatgggcgcagc
gtcaatgacgctgacggtacaggccagacaattattgtctggtatagtgcagcagcagaacaatttgctgagggc
tattgaggcgcaacagcatctgttgcaactcacagtctggggcatcaagcagctccaggcaagaatcctggctgt
ggaaagatacctaaaggatcaacagctcctggggatttggggttgctctggaaaactcatttgcaccactgctgt
gccttggaatgctagttggagtaataaatctctggaacagaattggaatcacacgacctggatggagtgggacag
agaaattaacaattacacaagcttaatacactccttaattgaagaatcgcaaaaccagcaagaaaagaatgaaca
agaattattggaattagataaatgggcaagtttgtggaattggtttaacataacaaattggctgtggtatataaa
attattcataatgatagtaggaggcttggtaggtttaagaatagtttttgctgtactttctatagtgaatagagt
taggcagggatattcaccattatcgtttcagacccacctcccaaccccgaggggacccgacaggcccgaaggaat
agaagaagaaggtggagagagagacagagacagatccattcgattagtgaacggatctcgacggtatcggttaac
ttttaaaagaaaaggggggattggggggtacagtgcaggggaaagaatagtagacataatagcaacagacataca
aactaaagaattacaaaaacaaattacaaaattcaaaattttatcgatactagtggatctGCGATCGCGTAACGC
CATTTTGCAAGGCATGGAAAAATACCAAACCAAGAATAGAGAAGTTCAGATCAAGGGCGGGTACATGAAAATAGC
TAACGTTGGGCCAAACAGGATATCTGCGGTGAGCAGTTTCGGCCCCGGCCCGGGGCCAAGAACAGATGGTCACCG
CAGTTTCGGCCCCGGCCCGAGGCCAAGAACAGATGGTCCCCAGATATGGCCCAACCCTCAGCAGTTTCTTAAGAC
CCATCAGATGTTTCCAGGCTCCCCCAAGGACCTGAAATGACCCTGCGCCTTATTTGAATTAACCAATCAGCCTGC
TTCTCGCTTCTGTTCGCGCGCTTCTGCTTCCCGAGCTCTATAAAAGAGCTCACAACCCCTCACTCGGCGCGCCAG
TCCTCCGACAGACTGAGTCGTCTAGAgctagcGGATCCCCGGAATTCGACGCCACCATGGCCCTGCCCGTGACCG
CCCTGCTGCTGCCCCTGGCCCTGCTGCTGCACGCCGCCAGGCCCCAGGTGCAGCTGCAGCAGAGCGGCCCCGAGC
TGGTGAGGCCCGGCACCTTCGTGAAGATCAGCTGCAAGGCCAGCGGCTACACCTTCACCAACTACGACATCAACT
GGGTGAACCAGAGGCCCGGCCAGGGCCTGGAGTGGATCGGCTGGATCTACCCCGGCGACGGCAGCACCAAGTACA
ACGAGAAGTTCAAGGCCAAGGCCACCCTGACCGCCGACAAGAGCAGCAGCACCGCCTACCTGCAGCTGAACAACC
TGACCAGCGAGAACAGCGCCGTGTACTTCTGCGCCAGCGGCTACGAGGACGCCATGGACTACTGGGGCCAGGGCA
CCAGCGTGACCGTGAGCAGCGGCGGCGGCGGCAGCGGCGGCGGCGGCAGCGGCGGCGGCGGCAGCGACATCAAGA
TGACCCAGAGCCCCAGCAGCATGTACGCCAGCCTGGGCGAGAGGGTGATCATCAACTGCAAGGCCAGCCAGGACA
TCAACAGCTACCTGAGCTGGTTCCAGCAGAAGCCCGGCAAGAGCCCCAAGACCCTGATCTACAGGGCCAACAGGC
TGGTGGACGGCGTGCCCAGCAGGTTCAGCGGCAGCGGCAGCGGCCAGGACTACAGCCTGACCATCAGCAGCCTGG
AGTACGAGGACATGGGCATCTACTACTGCCTGCAGTACGACGAGTTCCCCCTGACCTTCGGCGCCGGCACCAAGC
TGGAGCTGAAGAGGGGCCTGGCCGTGAGCACCATCAGCAGCTTCTTCCCCCCCGGCTACCAGATCTACATCTGGG
CCCCCCTGGCCGGCACCTGCGGCGTGCTGCTGCTGAGCCTGGTGATCACCCTGTACTGCAAGAGGGGCAGGAAGA
AGCTGCTGTACATCTTCAAGCAGCCCTTCATGAGGCCCGTGCAGACCACCCAGGAGGAGGACGGCTGCAGCTGCA
GGTTCCCCGAGGAGGAGGAGGGCGGCTGCGAGCTGAGGGTGAAGTTCAGCAGGAGCGCCGACGCCCCCGCCTACC
AGCAGGGCCAGAACCAGCTGTACAACGAGCTGAACCTGGGCAGGAGGGAGGAGTACGACGTGCTGGACAAGAGGA
GGGGCAGGGACCCCGAGATGGGCGGCAAGCCCAGGAGGAAGAACCCCCAGGAGGGCCTGTACAACGAGCTGCAGA
AGGACAAGATGGCCGAGGCCTACAGCGAGATCGGCATGAAGGGCGAGAGGAGGAGGGGCAAGGGCCACGACGGCC
TGTACCAGGGCCTGAGCACCGCCACCAAGGACACCTACGACGCCCTGCACATGCAGGCCCTGCCCCCCAGGgagg
gcagaggaagtcttctaacatgcggtgacgtggaggagaatcccggcccttccgggatgaccgagtacaagccca
cggtgcgcctcgccacccgcgacgacgtccccagggccgtacgcaccctcgccgccgcgttcgccgactaccccg
ccacgcgccacaccgtcgatccggaccgccacatcgagcgggtcaccgagctgcaagaactcttcctcacgcgcg
tcgggctcgacatcggcaaggtgtgggtcgcggacgacggcgccgcggtggcggtctggaccacgccggagagcg
tcgaagcgggggcggtgttcgccgagatcggcccgcgcatggccgagttgagcggttcccggctggccgcgcagc
aacagatggaaggcctcctggcgccgcaccggcccaaggagcccgcgtggttcctggccaccgtcggcgtctcgc
ccgaccaccagggcaagggtctgggcagcgccgtcgtgctccccggagtggaggcggccgagcgcgccggggtgc
ccgccttcctggagacctccgcgccccgcaacctccccttctacgagcggctcggcttcaccgtcaccgccgacg
tcgaggtgcccgaaggaccgcgcacctggtgcatgacccgcaagcccggtgcctgaATTAATTAACCAATTGaat
caacctctggattacaaaatttgtgaaagattgactggtattcttaactatgttgctccttttacgctatgtgga
tacgctgctttaatgcctttgtatcatgctattgcttcccgtatggctttcattttctcctccttgtataaatcc
tggttgctgtctctttatgaggagttgtggcccgttgtcaggcaacgtggcgtggtgtgcactgtgtttgctgac
gcaacccccactggttggggcattgccaccacctgtcagctcctttccgggactttcgctttccccctccctatt
gccacggcggaactcatcgccgcctgccttgcccgctgctggacaggggctcggctgttgggcactgacaattcc
gtggtgttgtcggggaaatcatcgtcctttccttggctgctcgcctgtgttgccacctggattctgcgcgggacg
tccttctgctacgtcccttcggccctcaatccagcggaccttccttcccgcggcctgctgccggctctgcggcct
cttccgcgtcttcgccttcgccctcagacgagtcggatctccctttgggccgcctccccgcctggtacctttaag
accaatgacttacaaggcagctgtagatcttagccactttttaaaagaaaaggggggactggaagggctaattca
ctcccaacgaaaataagatctgctttttgcttgtactgggtctctctggttagaccagatctgagcctgggagct
ctctggctaactagggaacccactgcttaagcctcaataaagcttgccttgagtgcttcaagtagtgtgtgcccg
tctgttgtgtgactctggtaactagagatccctcagacccttttagtcagtgtggaaaatctctagcaCGTATGT
GTATGATACATAAGGTTATGTATTAATTGTAGCCGCGTTCTAACGACAATATGTACAAGCCTAATTGTGTAGCAT
CTGGCTTACTGAAGCAGACCCTATCATCTCTCTCGTAAACTGCCGTCAGAGTCGGTTTGGTTGGACGAACCTTCT
GAGTTTCTGGTAACGCCGTCCCGCACCCGGAAATGGTCAGCGAACCAATCAGCAGGGTCATCGCTAGCCAGATCC
TCTACGCCGGACGCATCGTGGCCGGCATCACCGGCGCCACAGGTGCGGTTGCTGGCGCCTATATCGCCGACATCA
CCGATGGGGAAGATCGGGCTCGCCACTTCGGGCTCATGAGCGCTTGTTTCGGCGTGGGTATGGTGGCAGGCCCCG
TGGCCGGGGGACTGTTGGGCGCCATCTCCTTGCATGCACCATTCCTTGCGGCGGCGGTGCTCAACGGCCTCAACC
TACTACTGGGCTGCTTCCTAATGCAGGAGTCGCATAAGGGAGAGCGTCGAATGGTGCACTCTCAGTACAATCTGC
TCTGATGCCGCATAGTTAAGCCAGCCCCGACACCCGCCAACACCCGCTGACGCGCCCTGACGGGCTTGTCTGCTC
CCGGCATCCGCTTACAGACAAGCTGTGACCGTCTCCGGGAGCTGCATGTGTCAGAGGTTTTCACCGTCATCACCG
AAACGCGCGAGACGAAAGGGCCTCGTGATACGCCTATTTTTATAGGTTAATGTCATGATAATAATGGTTTCTTAG
ACGTCAGGTGGCACTTTTCGGGGAAATGTGCGCGGAACCCCTATTTGTTTATTTTTCTAAATACATTCAAATATG
TATCCGCTCATGAGACAATAACCCTGATAAATGCTTCAATAATATTGAAAAAGGAAGAGTATGAGTATTCAACAT
TTCCGTGTCGCCCTTATTCCCTTTTTTGCGGCATTTTGCCTTCCTGTTTTTGCTCACCCAGAAACGCTGGTGAAA
GTAAAAGATGCTGAAGATCAGTTGGGTGCACGAGTGGGTTACATCGAACTGGATCTCAACAGCGGTAAGATCCTT
GAGAGTTTTCGCCCCGAAGAACGTTTTCCAATGATGAGCACTTTTAAAGTTCTGCTATGTGGCGCGGTATTATCC
CGTATTGACGCCGGGCAAGAGCAACTCGGTCGCCGCATACACTATTCTCAGAATGACTTGGTTGAGTACTCACCA
GTCACAGAAAAGCATCTTACGGATGGCATGACAGTAAGAGAATTATGCAGTGCTGCCATAACCATGAGTGATAAC
ACTGCGGCCAACTTACTTCTGACAACGATCGGAGGACCGAAGGAGCTAACCGCTTTTTTGCACAACATGGGGGAT
CATGTAACTCGCCTTGATCGTTGGGAACCGGAGCTGAATGAAGCCATACCAAACGACGAGCGTGACACCACGATG
CCTGTAGCAATGGCAACAACGTTGCGCAAACTATTAACTGGCGAACTACTTACTCTAGCTTCCCGGCAACAATTA
ATAGACTGGATGGAGGCGGATAAAGTTGCAGGACCACTTCTGCGCTCGGCCCTTCCGGCTGGCTGGTTTATTGCT
GATAAATCTGGAGCCGGTGAGCGTGGGTCTCGCGGTATCATTGCAGCACTGGGGCCAGATGGTAAGCCCTCCCGT
ATCGTAGTTATCTACACGACGGGGAGTCAGGCAACTATGGATGAACGAAATAGACAGATCGCTGAGATAGGTGCC
TCACTGATTAAGCATTGGTAACTGTCAGACCAAGTTTACTCATATATACTTTAGATTGATTTAAAACTTCATTTT
TAATTTAAAAGGATCTAGGTGAAGATCCTTTTTGATAATCTCATGACCAAAATCCCTTAACGTGAGTTTTCGTTC
CACTGAGCGTCAGACCCCGTAGAAAAGATCAAAGGATCTTCTTGAGATCCTTTTTTTCTGCGCGTAATCTGCTGC
TTGCAAACAAAAAAACCACCGCTACCAGCGGTGGTTTGTTTGCCGGATCAAGAGCTACCAACTCTTTTTCCGAAG
GTAACTGGCTTCAGCAGAGCGCAGATACCAAATACTGTTCTTCTAGTGTAGCCGTAGTTAGGCCACCACTTCAAG
AACTCTGTAGCACCGCCTACATACCTCGCTCTGCTAATCCTGTTACCAGTGGCTGCTGCCAGTGGCGATAAGTCG
TGTCTTACCGGGTTGGACTCAAGACGATAGTTACCGGATAAGGCGCAGCGGTCGGGCTGAACGGGGGGTTCGTGC
ACACAGCCCAGCTTGGAGCGAACGACCTACACCGAACTGAGATACCTACAGCGTGAGCTATGAGAAAGCGCCACG
CTTCCCGAAGGGAGAAAGGCGGACAGGTATCCGGTAAGCGGCAGGGTCGGAACAGGAGAGCGCACGAGGGAGCTT
CCAGGGGGAAACGCCTGGTATCTTTATAGTCCTGTCGGGTTTCGCCACCTCTGACTTGAGCGTCGATTTTTGTGA
TGCTCGTCAGGGGGGCGGAGCCTATGGAAAAACGCCAGCAACGCGGCCTTTTTACGGTTCCTGGCCTTTTGCTGG
CCTTTTGCTCACATGTTCTTTCCTGCGTTATCCCCTGATTCTGTGGATAACCGTATTACCGCCTTTGAGTGAGCT
GATACCGCTCGCCGCAGCCGAACGACCGAGCGCAGCGAGTCAGTGAGCGAGGAAGCGGAAGAGCGCCCAATACGC
AAACCGCCTCTCCCCGCGCGTTGGCCGATTCATTAATGCAGCTGTGGAATGTGTGTCAGTTAGGGTGTGGAAAGT
CCCCAGGCTCCCCAGCAGGCAGAAGTATGCAAAGCATGCATCTCAATTAGTCAGCAACCAGGTGTGGAAAGTCCC
CAGGCTCCCCAGCAGGCAGAAGTATGCAAAGCATGCATCTCAATTAGTCAGCAACCATAGTCCCGCCCCTAACTC
CGCCCATCCCGCCCCTAACTCCGCCCAGTTCCGCCCATTCTCCGCCCCATGGCTGACTAATTTTTTTTATTTATG
CAGAGGCCGAGGCCGCCTCGGCCTCTGAGCTATTCCAGAAGTAGTGAGGAGGCTTTTTTGGAGGCCTAGGCTTTT
GCAAAAAGCTTGGACACAAGACAGGCTTGCGAGATATGTTTGAGAATACCACTTTATCCCGCGTCAGGGAGAGGC
AGTGCGTAAAAAGACGCGGACTCATGTGAAATACTGGTTTTTAGTGCGCCAGATCTCTATAATCTCGCGCAACCT
ATTTTCCCCTCGAACACTTTTTAAGCCGTAGATAAACAGGCTGGGACACTTCACATGAGCGAAAAATACATCGTC
ACCTGGGACATGTTGCAGATCCATGCACGTAAACTCGCAAGCCGACTGATGCCTTCTGAACAATGGAAAGGCATT
ATTGCCGTAAGCCGTGGCGGTCTGTACCGGGTGCGTTACTGGCGCGTGAACTGGGTATTCGTCATGTCGATACCG
TTTGTATTTCCAGCTACGATCACGACAACCAGCGCGAGCTTAAAGTGCTGAAACGCGCAGAAGGCGATGGCGAAG
GCTTCATCGTTATTGATGACCTGGTGGATACCGGTGGTACTGCGGTTGCGATTCGTGAAATGTATCCAAAAGCGC
ACTTTGTCACCATCTTCGCAAAACCGGCTGGTCGTCCGCTGGTTGATGACTATGTTGTTGATATCCCGCAAGATA
CCTGGATTGAACAGCCGTGGGATATGGGCGTCGTATTCGTCCCGCCAATCTCCGGTCGCTAATCTTTTCAACGCC
TGGCACTGCCGGGCGTTGTTCTTTTTAACTTCAGGCGGGTTACAATAGTTTCCAGTAAGTATTCTGGAGGCTGCA
TCCATGACACAGGCAAACCTGAGCGAAACCCTGTTCAAACCCCGCTTTAAACATCCTGAAACCTCGACGCTAGTC
CGCCGCTTTAATCACGGCGCACAACCGCCTGTGCAGTCGGCCCTTGATGGTAAAACCATCCCTCACTGGTATCGC
ATGATTAACCGTCTGATGTGGATCTGGCGCGGCATTGACCCACGCGAAATCCTCGACGTCCAGGCACGTATTGTG
ATGAGCGATGCCGAACGTACCGACGATGATTTATACGATACGGTGATTGGCTACCGTGGCGGCAACTGGATTTAT
GAGTGGGCCCCGGATCTTTGTGAAGGAACCTTACTTCTGTGGTGTGACATAATTGGACAAACTACCTACAGAGAT
TTAAAGCTCTAAGGTAAATATAAAATTTTTAAGTGTATAATGTGTTAAACTACTGATTCTAATTGTTTGTGTATT
TTAGATTCCAACCTATGGAACTGATGAATGGGAGCAGTGGTGGAATGCCTTTAATGAGGAAAACCTGTTTTGCTC
AGAAGAAATGCCATCTAGTGATGATGAGGCTACTGCTGACTCTCAACATTCTACTCCTCCAAAAAAGAAGAGAAA
GGTAGAAGACCCCAAGGACTTTCCTTCAGAATTGCTAAGTTTTTTGAGTCATGCTGTGTTTAGTAATAGAACTCT
TGCTTGCTTTGCTATTTACACCACAAAGGAAAAAGCTGCACTGCTATACAAGAAAATTATGGAAAAATATTCTGT
AACCTTTATAAGTAGGCATAACAGTTATAATCATAACATACTGTTTTTTCTTACTCCACACAGGCATAGAGTGTC
TGCTATTAATAACTATGCTCAAAAATTGTGTACCTTTAGCTTTTTAATTTGTAAAGGGGTTAATAAGGAATATTT
GATGTATAGTGCCTTGACTAGAGATCATAATCAGCCATACCACATTTGTAGAGGTTTTACTTGCTTTAAAAAACC
TCCCACACCTCCCCCTGAACCTGAAACATAAAATGAATGCAATTGTTGTTGTTAACTTGTTTATTGCAGCTTATA
ATGGTTACAAATAAAGCAATAGCATCACAAATTTCACAAATAAAGCATTTTTTTCACTGCATTCTAGTTGTGGTT
TGTCCAAACTCATCAATGTATCTTATCATGTCTGGATCAACTGGATAACTCAAGCTAACCAAAATCATCCCAAAC
TTCCCACCCCATACCCTATTACCACTGCCAATTACCTGTGGTTTCATTTACTCTAAACCTGTGATTCCTCTGAAT
TATTTTCATTTTAAAGAAATTGTATTTGTTAAATATGTACTACAAACTTAGTAG

15. DRB2-CD8-41BB-CD3z

An exemplary CAR construct, as described herein, comprises a CD33 binding domain (DRB2) scFv, a CD8a transmembrane domain, a CD8a hinge domain, a CD137 (4-1BB) co-stimulatory domain, and a CD3ζ intracellular signaling domain.

In some embodiments, a CAR in encoded by a nucleic acid sequence that comprises the sequence that is shown in SEQ ID NO: 53, or in a nucleic acid sequence that is at least 70%, at least 75%, at least 80%, at least 85%, at least 90%, at least 95%, or at least 99% identical to the nucleic acid sequence shown in SEQ ID NO: 53.

[SEQ ID NO: 53]
ATGGCCCTGCCCGTGACCGCCCTGCTGCTGCCCCTGGCCCTGCTGCTGCACGCCGCCAGGCCCGAGGTGAAGCTG
CAGGAGAGCGGCCCCGAGCTGGTGAAGCCCGGCGCCAGCGTGAAGATGAGCTGCAAGGCCAGCGGCTACAAGTTC
ACCGACTACGTGGTGCACTGGCTGAAGCAGAAGCCCGGCCAGGGCCTGGAGTGGATCGGCTACATCAACCCCTAC
AACGACGGCACCAAGTACAACGAGAAGTTCAAGGGCAAGGCCACCCTGACCAGCGACAAGAGCAGCAGCACCGCC
TACATGGAGGTGAGCAGCCTGACCAGCGAGGACAGCGCCGTGTACTACTGCGCCAGGGACTACAGGTACGAGGTG
TACGGCATGGACTACTGGGGCCAGGGCACCAGCGTGACCGTGAGCAGCGGCGGCGGCGGCAGCGGCGGCGGCGGC
AGCGGCGGCGGCGGCAGCGACATCGTGCTGACCCAGAGCCCCACCATCATGAGCGCCAGCCCCGGCGAGAGGGTG
ACCATGACCTGCACCGCCAGCAGCAGCGTGAACTACATCCACTGGTACCAGCAGAAGAGCGGCGACAGCCCCCTG
AGGTGGATCTTCGACACCAGCAAGGTGGCCAGCGGCGTGCCCGCCAGGTTCAGCGGCAGCGGCAGCGGCACCAGC
TACAGCCTGACCATCAGCACCATGGAGGCCGAGGACGCCGCCACCTACTACTGCCAGCAGTGGAGGAGCTACCCC
CTGACCTTCGGCGACGGCACCAGGCTGGAGCTGAAGAGGGCCGACGCCGCCCCCACCGTGAGCGGCCTGGCCGTG
AGCACCATCAGCAGCTTCTTCCCCCCCGGCTACCAGATCTACATCTGGGCCCCCCTGGCCGGCACCTGCGGCGTG
CTGCTGCTGAGCCTGGTGATCACCCTGTACTGCAAGAGGGGCAGGAAGAAGCTGCTGTACATCTTCAAGCAGCCC
TTCATGAGGCCCGTGCAGACCACCCAGGAGGAGGACGGCTGCAGCTGCAGGTTCCCCGAGGAGGAGGAGGGCGGC
TGCGAGCTGAGGGTGAAGTTCAGCAGGAGCGCCGACGCCCCCGCCTACCAGCAGGGCCAGAACCAGCTGTACAAC
GAGCTGAACCTGGGCAGGAGGGAGGAGTACGACGTGCTGGACAAGAGGAGGGGCAGGGACCCCGAGATGGGCGGC
AAGCCCAGGAGGAAGAACCCCCAGGAGGGCCTGTACAACGAGCTGCAGAAGGACAAGATGGCCGAGGCCTACAGC
GAGATCGGCATGAAGGGCGAGAGGAGGAGGGGCAAGGGCCACGACGGCCTGTACCAGGGCCTGAGCACCGCCACC
AAGGACACCTACGACGCCCTGCACATGCAGGCCCTGCCCCCCAGG

In some embodiments, a CAR comprises an amino acid sequence shown in SEQ ID NO: 54, or an amino acid sequence that is at least 70%, at least 75%, at least 80%, at least 85%, at least 90%, at least 95%, or at least 99% identical to the amino acid sequence shown in SEQ ID NO: 54.

[SEQ ID NO: 54]
MALPVTALLLPLALLLHAARPEVKLQESGPELVKPGASVKMSCKASGYKFTDYVVHWLKQKPGQGLEWIGYINPY
NDGTKYNEKFKGKATLTSDKSSSTAYMEVSSLTSEDSAVYYCARDYRYEVYGMDYWGQGTSVTVSSGGGGSGGGG

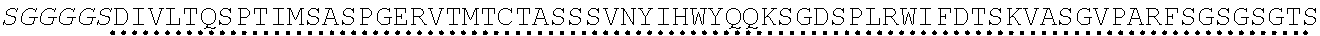

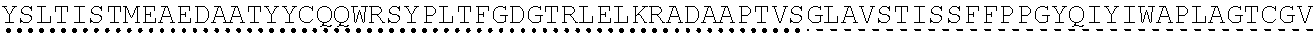

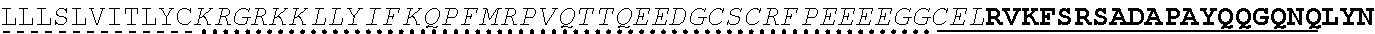

ELNLGRREEYDVLDKRRGRDPEMGGKPRRKNPQEGLYNELQKDKMAEAYSEIGMKGERRRGKGHDGLYQGLSTAT
KDTYDALHMQALPPR

In some embodiments, a CAR construct as shown in SEQ ID NO: 53 is included in a recombinant expression vector. In some embodiments the recombinant expression vector includes a promoter (e.g., an SFFV promoter or and EF1α promoter). In some embodiments, a recombinant expression vector including the CAR of SEQ ID NO: 53 comprises the sequence that is shown in SEQ ID NO: 55, or in a nucleic acid sequence that is at least 70%, at least 75%, at least 80%, at least 85%, at least 90%, at least 95%, or at least 99% identical to the nucleic acid sequence shown in SEQ ID NO: 55.

In some embodiments, a recombinant expression vector including the CAR of SEQ ID NO: 53 comprises the sequence that is shown in SEQ ID NO: 55, but does not include the SFFV promoter sequence (as shown in SEQ ID NO: 8). In some embodiments, a recombinant expression vector comprises the nucleic acid sequence shown in SEQ ID NO: 55, but does not include the SFFV sequence (as shown in SEQ ID NO: 8), and instead includes an EF1α promoter sequence.

[SEQ ID NO: 55]
Tgggtctctctggttagaccagatctgagcctgggagctctctggctaactagggaacccactgcttaagcctca
ataaagcttgccttgagtgcttcaagtagtgtgtgcccgtctgttgtgtgactctggtaactagagatccctcag
acccttttagtcagtgtggaaaatctctagcagtggcgcccgaacagggacttgaaagcgaaagggaaaccagag
gagctctctcgacgcaggactcggcttgctgaagcgcgcacggcaagaggcgaggggcggcgactggtgagtacg
ccaaaaattttgactagcggaggctagaaggagagagatgggtgcgagagcgtcagtattaagcgggggagaatt
agatcgcgatgggaaaaaattcggttaaggccagggggaaagaaaaaatataaattaaaacatatagtatgggca
agcagggagctagaacgattcgcagttaatcctggcctgttagaaacatcagaaggctgtagacaaatactggga
cagctacaaccatcccttcagacaggatcagaagaacttagatcattatataatacagtagcaaccctctattgt
gtgcatcaaaggatagagataaaagacaccaaggaagctttagacaagatagaggaagagcaaaacaaaagtaag
accaccgcacagcaagcggccactgatcttcagacctggaggaggagatatgagggacaattggagaagtgaatt
atataaatataaagtagtaaaaattgaaccattaggagtagcacccaccaaggcaaagagaagagtggtgcagag
agaaaaaagagcagtgggaataggagctttgttccttgggttcttgggagcagcaggaagcactatgggcgcagc
gtcaatgacgctgacggtacaggccagacaattattgtctggtatagtgcagcagcagaacaatttgctgagggc
tattgaggcgcaacagcatctgttgcaactcacagtctggggcatcaagcagctccaggcaagaatcctggctgt
ggaaagatacctaaaggatcaacagctcctggggatttggggttgctctggaaaactcatttgcaccactgctgt
gccttggaatgctagttggagtaataaatctctggaacagaattggaatcacacgacctggatggagtgggacag
agaaattaacaattacacaagcttaatacactccttaattgaagaatcgcaaaaccagcaagaaaagaatgaaca
agaattattggaattagataaatgggcaagtttgtggaattggtttaacataacaaattggctgtggtatataaa
attattcataatgatagtaggaggcttggtaggtttaagaatagtttttgctgtactttctatagtgaatagagt
taggcagggatattcaccattatcgtttcagacccacctcccaaccccgaggggacccgacaggcccgaaggaat
agaagaagaaggtggagagagagacagagacagatccattcgattagtgaacggatctcgacggtatcggttaac
ttttaaaagaaaaggggggattggggggtacagtgcaggggaaagaatagtagacataatagcaacagacataca
aactaaagaattacaaaaacaaattacaaaattcaaaattttatcgatactagtggatctGCGATCGCGTAACGC
CATTTTGCAAGGCATGGAAAAATACCAAACCAAGAATAGAGAAGTTCAGATCAAGGGCGGGTACATGAAAATAGC
TAACGTTGGGCCAAACAGGATATCTGCGGTGAGCAGTTTCGGCCCCGGCCCGGGGCCAAGAACAGATGGTCACCG
CAGTTTCGGCCCCGGCCCGAGGCCAAGAACAGATGGTCCCCAGATATGGCCCAACCCTCAGCAGTTTCTTAAGAC
CCATCAGATGTTTCCAGGCTCCCCCAAGGACCTGAAATGACCCTGCGCCTTATTTGAATTAACCAATCAGCCTGC
TTCTCGCTTCTGTTCGCGCGCTTCTGCTTCCCGAGCTCTATAAAAGAGCTCACAACCCCTCACTCGGCGCGCCAG
TCCTCCGACAGACTGAGTCGTCTAGAgctagcGGATCCCCGGAATTCGACGCCACCATGGCCCTGCCCGTGACCG
CCCTGCTGCTGCCCCTGGCCCTGCTGCTGCACGCCGCCAGGCCCGAGGTGAAGCTGCAGGAGAGCGGCCCCGAGC
TGGTGAAGCCCGGCGCCAGCGTGAAGATGAGCTGCAAGGCCAGCGGCTACAAGTTCACCGACTACGTGGTGCACT
GGCTGAAGCAGAAGCCCGGCCAGGGCCTGGAGTGGATCGGCTACATCAACCCCTACAACGACGGCACCAAGTACA
ACGAGAAGTTCAAGGGCAAGGCCACCCTGACCAGCGACAAGAGCAGCAGCACCGCCTACATGGAGGTGAGCAGCC
TGACCAGCGAGGACAGCGCCGTGTACTACTGCGCCAGGGACTACAGGTACGAGGTGTACGGCATGGACTACTGGG
GCCAGGGCACCAGCGTGACCGTGAGCAGCGGCGGCGGCGGCAGCGGCGGCGGCGGCAGCGGCGGCGGCGGCAGCG
ACATCGTGCTGACCCAGAGCCCCACCATCATGAGCGCCAGCCCCGGCGAGAGGGTGACCATGACCTGCACCGCCA
GCAGCAGCGTGAACTACATCCACTGGTACCAGCAGAAGAGCGGCGACAGCCCCCTGAGGTGGATCTTCGACACCA
GCAAGGTGGCCAGCGGCGTGCCCGCCAGGTTCAGCGGCAGCGGCAGCGGCACCAGCTACAGCCTGACCATCAGCA
CCATGGAGGCCGAGGACGCCGCCACCTACTACTGCCAGCAGTGGAGGAGCTACCCCCTGACCTTCGGCGACGGCA
CCAGGCTGGAGCTGAAGAGGGCCGACGCCGCCCCCACCGTGAGCGGCCTGGCCGTGAGCACCATCAGCAGCTTCT
TCCCCCCCGGCTACCAGATCTACATCTGGGCCCCCCTGGCCGGCACCTGCGGCGTGCTGCTGCTGAGCCTGGTGA
TCACCCTGTACTGCAAGAGGGGCAGGAAGAAGCTGCTGTACATCTTCAAGCAGCCCTTCATGAGGCCCGTGCAGA
CCACCCAGGAGGAGGACGGCTGCAGCTGCAGGTTCCCCGAGGAGGAGGAGGGCGGCTGCGAGCTGAGGGTGAAGT
TCAGCAGGAGCGCCGACGCCCCCGCCTACCAGCAGGGCCAGAACCAGCTGTACAACGAGCTGAACCTGGGCAGGA
GGGAGGAGTACGACGTGCTGGACAAGAGGAGGGGCAGGGACCCCGAGATGGGCGGCAAGCCCAGGAGGAAGAACC
CCCAGGAGGGCCTGTACAACGAGCTGCAGAAGGACAAGATGGCCGAGGCCTACAGCGAGATCGGCATGAAGGGCG
AGAGGAGGAGGGGCAAGGGCCACGACGGCCTGTACCAGGGCCTGAGCACCGCCACCAAGGACACCTACGACGCCC
TGCACATGCAGGCCCTGCCCCCCAGGgagggcagaggaagtcttctaacatgcggtgacgtggaggagaatcccg
gcccttccgggatgaccgagtacaagcccacggtgcgcctcgccacccgcgacgacgtccccagggccgtacgca
ccctcgccgccgcgttcgccgactaccccgccacgcgccacaccgtcgatccggaccgccacatcgagcgggtca
ccgagctgcaagaactcttcctcacgcgcgtcgggctcgacatcggcaaggtgtgggtcgcggacgacggcgccg
cggtggcggtctggaccacgccggagagcgtcgaagcgggggcggtgttcgccgagatcggcccgcgcatggccg
agttgagcggttcccggctggccgcgcagcaacagatggaaggcctcctggcgccgcaccggcccaaggagcccg
cgtggttcctggccaccgtcggcgtctcgcccgaccaccagggcaagggtctgggcagcgccgtcgtgctccccg
gagtggaggcggccgagcgcgccggggtgcccgccttcctggagacctccgcgccccgcaacctccccttctacg
agcggctcggcttcaccgtcaccgccgacgtcgaggtgcccgaaggaccgcgcacctggtgcatgacccgcaagc
ccggtgcctgaATTAATTAACCAATTGaatcaacctctggattacaaaatttgtgaaagattgactggtattctt
aactatgttgctccttttacgctatgtggatacgctgctttaatgcctttgtatcatgctattgcttcccgtatg
gctttcattttctcctccttgtataaatcctggttgctgtctctttatgaggagttgtggcccgttgtcaggcaa
cgtggcgtggtgtgcactgtgtttgctgacgcaacccccactggttggggcattgccaccacctgtcagctcctt
tccgggactttcgctttccccctccctattgccacggcggaactcatcgccgcctgccttgcccgctgctggaca
ggggctcggctgttgggcactgacaattccgtggtgttgtcggggaaatcatcgtcctttccttggctgctcgcc
tgtgttgccacctggattctgcgcgggacgtccttctgctacgtcccttcggccctcaatccagcggaccttcct
tcccgcggcctgctgccggctctgcggcctcttccgcgtcttcgccttcgccctcagacgagtcggatctccctt
tgggccgcctccccgcctggtacctttaagaccaatgacttacaaggcagctgtagatcttagccactttttaaa
agaaaaggggggactggaagggctaattcactcccaacgaaaataagatctgctttttgcttgtactgggtctct
ctggttagaccagatctgagcctgggagctctctggctaactagggaacccactgcttaagcctcaataaagctt
gccttgagtgcttcaagtagtgtgtgcccgtctgttgtgtgactctggtaactagagatccctcagaccctttta
gtcagtgtggaaaatctctagcaCGTATGTGTATGATACATAAGGTTATGTATTAATTGTAGCCGCGTTCTAACG
ACAATATGTACAAGCCTAATTGTGTAGCATCTGGCTTACTGAAGCAGACCCTATCATCTCTCTCGTAAACTGCCG
TCAGAGTCGGTTTGGTTGGACGAACCTTCTGAGTTTCTGGTAACGCCGTCCCGCACCCGGAAATGGTCAGCGAAC
CAATCAGCAGGGTCATCGCTAGCCAGATCCTCTACGCCGGACGCATCGTGGCCGGCATCACCGGCGCCACAGGTG
CGGTTGCTGGCGCCTATATCGCCGACATCACCGATGGGGAAGATCGGGCTCGCCACTTCGGGCTCATGAGCGCTT
GTTTCGGCGTGGGTATGGTGGCAGGCCCCGTGGCCGGGGGACTGTTGGGCGCCATCTCCTTGCATGCACCATTCC
TTGCGGCGGCGGTGCTCAACGGCCTCAACCTACTACTGGGCTGCTTCCTAATGCAGGAGTCGCATAAGGGAGAGC
GTCGAATGGTGCACTCTCAGTACAATCTGCTCTGATGCCGCATAGTTAAGCCAGCCCCGACACCCGCCAACACCC
GCTGACGCGCCCTGACGGGCTTGTCTGCTCCCGGCATCCGCTTACAGACAAGCTGTGACCGTCTCCGGGAGCTGC
ATGTGTCAGAGGTTTTCACCGTCATCACCGAAACGCGCGAGACGAAAGGGCCTCGTGATACGCCTATTTTTATAG
GTTAATGTCATGATAATAATGGTTTCTTAGACGTCAGGTGGCACTTTTCGGGGAAATGTGCGCGGAACCCCTATT
TGTTTATTTTTCTAAATACATTCAAATATGTATCCGCTCATGAGACAATAACCCTGATAAATGCTTCAATAATAT
TGAAAAAGGAAGAGTATGAGTATTCAACATTTCCGTGTCGCCCTTATTCCCTTTTTTGCGGCATTTTGCCTTCCT
GTTTTTGCTCACCCAGAAACGCTGGTGAAAGTAAAAGATGCTGAAGATCAGTTGGGTGCACGAGTGGGTTACATC
GAACTGGATCTCAACAGCGGTAAGATCCTTGAGAGTTTTCGCCCCGAAGAACGTTTTCCAATGATGAGCACTTTT
AAAGTTCTGCTATGTGGCGCGGTATTATCCCGTATTGACGCCGGGCAAGAGCAACTCGGTCGCCGCATACACTAT
TCTCAGAATGACTTGGTTGAGTACTCACCAGTCACAGAAAAGCATCTTACGGATGGCATGACAGTAAGAGAATTA
TGCAGTGCTGCCATAACCATGAGTGATAACACTGCGGCCAACTTACTTCTGACAACGATCGGAGGACCGAAGGAG
CTAACCGCTTTTTTGCACAACATGGGGGATCATGTAACTCGCCTTGATCGTTGGGAACCGGAGCTGAATGAAGCC
ATACCAAACGACGAGCGTGACACCACGATGCCTGTAGCAATGGCAACAACGTTGCGCAAACTATTAACTGGCGAA
CTACTTACTCTAGCTTCCCGGCAACAATTAATAGACTGGATGGAGGCGGATAAAGTTGCAGGACCACTTCTGCGC
TCGGCCCTTCCGGCTGGCTGGTTTATTGCTGATAAATCTGGAGCCGGTGAGCGTGGGTCTCGCGGTATCATTGCA
GCACTGGGGCCAGATGGTAAGCCCTCCCGTATCGTAGTTATCTACACGACGGGGAGTCAGGCAACTATGGATGAA
CGAAATAGACAGATCGCTGAGATAGGTGCCTCACTGATTAAGCATTGGTAACTGTCAGACCAAGTTTACTCATAT
ATACTTTAGATTGATTTAAAACTTCATTTTTAATTTAAAAGGATCTAGGTGAAGATCCTTTTTGATAATCTCATG
ACCAAAATCCCTTAACGTGAGTTTTCGTTCCACTGAGCGTCAGACCCCGTAGAAAAGATCAAAGGATCTTCTTGA
GATCCTTTTTTTCTGCGCGTAATCTGCTGCTTGCAAACAAAAAAACCACCGCTACCAGCGGTGGTTTGTTTGCCG
GATCAAGAGCTACCAACTCTTTTTCCGAAGGTAACTGGCTTCAGCAGAGCGCAGATACCAAATACTGTTCTTCTA
GTGTAGCCGTAGTTAGGCCACCACTTCAAGAACTCTGTAGCACCGCCTACATACCTCGCTCTGCTAATCCTGTTA
CCAGTGGCTGCTGCCAGTGGCGATAAGTCGTGTCTTACCGGGTTGGACTCAAGACGATAGTTACCGGATAAGGCG
CAGCGGTCGGGCTGAACGGGGGGTTCGTGCACACAGCCCAGCTTGGAGCGAACGACCTACACCGAACTGAGATAC
CTACAGCGTGAGCTATGAGAAAGCGCCACGCTTCCCGAAGGGAGAAAGGCGGACAGGTATCCGGTAAGCGGCAGG
GTCGGAACAGGAGAGCGCACGAGGGAGCTTCCAGGGGGAAACGCCTGGTATCTTTATAGTCCTGTCGGGTTTCGC
CACCTCTGACTTGAGCGTCGATTTTTGTGATGCTCGTCAGGGGGGCGGAGCCTATGGAAAAACGCCAGCAACGCG
GCCTTTTTACGGTTCCTGGCCTTTTGCTGGCCTTTTGCTCACATGTTCTTTCCTGCGTTATCCCCTGATTCTGTG
GATAACCGTATTACCGCCTTTGAGTGAGCTGATACCGCTCGCCGCAGCCGAACGACCGAGCGCAGCGAGTCAGTG
AGCGAGGAAGCGGAAGAGCGCCCAATACGCAAACCGCCTCTCCCCGCGCGTTGGCCGATTCATTAATGCAGCTGT
GGAATGTGTGTCAGTTAGGGTGTGGAAAGTCCCCAGGCTCCCCAGCAGGCAGAAGTATGCAAAGCATGCATCTCA
ATTAGTCAGCAACCAGGTGTGGAAAGTCCCCAGGCTCCCCAGCAGGCAGAAGTATGCAAAGCATGCATCTCAATT
AGTCAGCAACCATAGTCCCGCCCCTAACTCCGCCCATCCCGCCCCTAACTCCGCCCAGTTCCGCCCATTCTCCGC
CCCATGGCTGACTAATTTTTTTTATTTATGCAGAGGCCGAGGCCGCCTCGGCCTCTGAGCTATTCCAGAAGTAGT
GAGGAGGCTTTTTTGGAGGCCTAGGCTTTTGCAAAAAGCTTGGACACAAGACAGGCTTGCGAGATATGTTTGAGA
ATACCACTTTATCCCGCGTCAGGGAGAGGCAGTGCGTAAAAAGACGCGGACTCATGTGAAATACTGGTTTTTAGT
GCGCCAGATCTCTATAATCTCGCGCAACCTATTTTCCCCTCGAACACTTTTTAAGCCGTAGATAAACAGGCTGGG
ACACTTCACATGAGCGAAAAATACATCGTCACCTGGGACATGTTGCAGATCCATGCACGTAAACTCGCAAGCCGA
CTGATGCCTTCTGAACAATGGAAAGGCATTATTGCCGTAAGCCGTGGCGGTCTGTACCGGGTGCGTTACTGGCGC
GTGAACTGGGTATTCGTCATGTCGATACCGTTTGTATTTCCAGCTACGATCACGACAACCAGCGCGAGCTTAAAG
TGCTGAAACGCGCAGAAGGCGATGGCGAAGGCTTCATCGTTATTGATGACCTGGTGGATACCGGTGGTACTGCGG
TTGCGATTCGTGAAATGTATCCAAAAGCGCACTTTGTCACCATCTTCGCAAAACCGGCTGGTCGTCCGCTGGTTG
ATGACTATGTTGTTGATATCCCGCAAGATACCTGGATTGAACAGCCGTGGGATATGGGCGTCGTATTCGTCCCGC
CAATCTCCGGTCGCTAATCTTTTCAACGCCTGGCACTGCCGGGCGTTGTTCTTTTTAACTTCAGGCGGGTTACAA
TAGTTTCCAGTAAGTATTCTGGAGGCTGCATCCATGACACAGGCAAACCTGAGCGAAACCCTGTTCAAACCCCGC
TTTAAACATCCTGAAACCTCGACGCTAGTCCGCCGCTTTAATCACGGCGCACAACCGCCTGTGCAGTCGGCCCTT
GATGGTAAAACCATCCCTCACTGGTATCGCATGATTAACCGTCTGATGTGGATCTGGCGCGGCATTGACCCACGC
GAAATCCTCGACGTCCAGGCACGTATTGTGATGAGCGATGCCGAACGTACCGACGATGATTTATACGATACGGTG
ATTGGCTACCGTGGCGGCAACTGGATTTATGAGTGGGCCCCGGATCTTTGTGAAGGAACCTTACTTCTGTGGTGT
GACATAATTGGACAAACTACCTACAGAGATTTAAAGCTCTAAGGTAAATATAAAATTTTTAAGTGTATAATGTGT
TAAACTACTGATTCTAATTGTTTGTGTATTTTAGATTCCAACCTATGGAACTGATGAATGGGAGCAGTGGTGGAA
TGCCTTTAATGAGGAAAACCTGTTTTGCTCAGAAGAAATGCCATCTAGTGATGATGAGGCTACTGCTGACTCTCA
ACATTCTACTCCTCCAAAAAAGAAGAGAAAGGTAGAAGACCCCAAGGACTTTCCTTCAGAATTGCTAAGTTTTTT
GAGTCATGCTGTGTTTAGTAATAGAACTCTTGCTTGCTTTGCTATTTACACCACAAAGGAAAAAGCTGCACTGCT
ATACAAGAAAATTATGGAAAAATATTCTGTAACCTTTATAAGTAGGCATAACAGTTATAATCATAACATACTGTT
TTTTCTTACTCCACACAGGCATAGAGTGTCTGCTATTAATAACTATGCTCAAAAATTGTGTACCTTTAGCTTTTT
AATTTGTAAAGGGGTTAATAAGGAATATTTGATGTATAGTGCCTTGACTAGAGATCATAATCAGCCATACCACAT
TTGTAGAGGTTTTACTTGCTTTAAAAAACCTCCCACACCTCCCCCTGAACCTGAAACATAAAATGAATGCAATTG
TTGTTGTTAACTTGTTTATTGCAGCTTATAATGGTTACAAATAAAGCAATAGCATCACAAATTTCACAAATAAAG
CATTTTTTTCACTGCATTCTAGTTGTGGTTTGTCCAAACTCATCAATGTATCTTATCATGTCTGGATCAACTGGA
TAACTCAAGCTAACCAAAATCATCCCAAACTTCCCACCCCATACCCTATTACCACTGCCAATTACCTGTGGTTTC
ATTTACTCTAAACCTGTGATTCCTCTGAATTATTTTCATTTTAAAGAAATTGTATTTGTTAAATATGTACTACAA
ACTTAGTAG

16. CAR33VH-CD8-41BB-CD3z

An exemplary CAR construct, as described herein, comprises a CD33 binding domain (CAR33VH), a CD8a transmembrane domain, a CD8a hinge domain, a CD137 (4-1BB) co-stimulatory domain, and a CD3ζ intracellular signaling domain.

In some embodiments, a CAR in encoded by a nucleic acid sequence that comprises the sequence that is shown in SEQ ID NO: 56, or in a nucleic acid sequence that is at least 70%, at least 75%, at least 80%, at least 85%, at least 90%, at least 95%, or at least 99% identical to the nucleic acid sequence shown in SEQ ID NO: 56.

[SEQ ID NO: 56]
ATGCTGCTGCTGGTGACCAGCCTGCTGCTGTGCGAACTGCCGCATCCGGCGTTTCTGCTGATTCCGGAGGTGCAG
CTGGTGGAGTCTGGGGGAGGCTTGGTACAGCCTGGAGGGTCCCTGAGACTCTCCTGTGCAGCCTCTGGATTCACC
TTCAGTAGCTATGGCATGAGCTGGGTCCGCCAGGCTCCAAGACAAGGGCTTGAGTGGGTGGCCAACATAAAGCAA
GATGGAAGTGAGAAATACTATGCGGACTCAGTGAAGGGCCGATTCACCATCTCCAGAGACAATTCCAAGAACACG
CTGTATCTGCAAATGAACAGCCTGAGAGCCGAGGACACAGCCACGTATTACTGTGCGAAAGAAAATGTGGACTGG
GGCCAGGGCACCCTGGTCACCGTCTCCTCAGCGGCCGCAACTACCACCCCTGCCCCTCGGCCGCCGACTCCGGCC
CCAACCATCGCAAGCCAACCCCTCTCCTTGCGCCCCGAAGCTTGCCGCCCGGCCGCGGGTGGAGCCGTGCATACC
CGGGGGCTGGACTTTGCCTGCGATATCTACATTTGGGCCCCGCTGGCCGGCACTTGCGGCGTGCTCCTGCTGTCG
CTGGTCATCACCCTTTACTGCAAGAGGGGCCGGAAGAAGCTGCTTTACATCTTCAAGCAGCCGTTCATGCGGCCC
GTGCAGACGACTCAGGAAGAGGACGGATGCTCGTGCAGATTCCCTGAGGAGGAAGAGGGGGGATGCGAACTGCGC
GTCAAGTTCTCACGGTCCGCCGACGCCCCCGCATATCAACAGGGCCAGAATCAGCTCTACAACGAGCTGAACCTG
GGAAGGAGAGAGGAGTACGACGTGCTGGACAAGCGACGCGGACGCGACCCGGAGATGGGGGGGAAACCACGGCGG
AAAAACCCTCAGGAAGGACTGTACAACGAACTCCAGAAAGACAAGATGGCGGAAGCCTACTCAGAAATCGGGATG
AAGGGAGAGCGGAGGAGGGGAAAGGGCCACGACGGCCTGTACCAGGGCCTGAGCACCGCCACCAAGGACACCTAC
GACGCCCTGCACATGCAGGCCCTGCCCCCCAGG

In some embodiments, a CAR comprises an amino acid sequence shown in SEQ ID NO: 57, or an amino acid sequence that is at least 70%, at least 75%, at least 80%, at least 85%, at least 90%, at least 95%, or at least 99% identical to the amino acid sequence shown in SEQ ID NO: 57.

[SEQ ID NO: 57]
MLLLVTSLLLCELPHPAFLLIPEVQLVESGGGLVQPGGSLRLSCAASGFTFSSYGMSWVRQAPRQGLEWVANIKQ

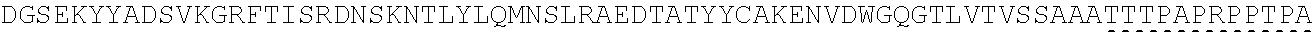

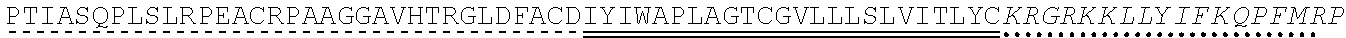

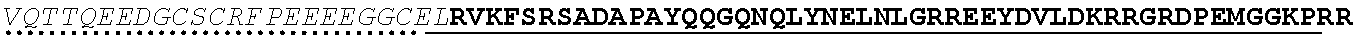

KNPQEGLYNELQKDKMAEAYSEIGMKGERRRGKGHDGLYQGLSTATKDTYDALHMQALPPR

In some embodiments, a CAR construct as shown in SEQ ID NO: 56 is included in a recombinant expression vector. In some embodiments the recombinant expression vector includes a promoter (e.g., an SFFV promoter or and EF1α promoter). In some embodiments, a recombinant expression vector including the CAR of SEQ ID NO: 56 comprises the sequence that is shown in SEQ ID NO: 58, or in a nucleic acid sequence that is at least 70%, at least 75%, at least 80%, at least 85%, at least 90%, at least 95%, or at least 99% identical to the nucleic acid sequence shown in SEQ ID NO: 58.

In some embodiments, a recombinant expression vector including the CAR of SEQ ID NO: 56 comprises the sequence that is shown in SEQ ID NO: 58, but does not include the SFFV promoter sequence (as shown in SEQ ID NO: 8). In some embodiments, a recombinant expression vector comprises the nucleic acid sequence shown in SEQ ID NO: 58, but does not include the SFFV sequence (as shown in SEQ ID NO: 8), and instead includes an EF1α promoter sequence.

[SEQ ID NO: 58]
Tgggtctctctggttagaccagatctgagcctgggagctctctggctaactagggaacccactgcttaagcctca
ataaagcttgccttgagtgcttcaagtagtgtgtgcccgtctgttgtgtgactctggtaactagagatccctcag
acccttttagtcagtgtggaaaatctctagcagtggcgcccgaacagggacttgaaagcgaaagggaaaccagag
gagctctctcgacgcaggactcggcttgctgaagcgcgcacggcaagaggcgaggggcggcgactggtgagtacg
ccaaaaattttgactagcggaggctagaaggagagagatgggtgcgagagcgtcagtattaagcgggggagaatt
agatcgcgatgggaaaaaattcggttaaggccagggggaaagaaaaaatataaattaaaacatatagtatgggca
agcagggagctagaacgattcgcagttaatcctggcctgttagaaacatcagaaggctgtagacaaatactggga
cagctacaaccatcccttcagacaggatcagaagaacttagatcattatataatacagtagcaaccctctattgt
gtgcatcaaaggatagagataaaagacaccaaggaagctttagacaagatagaggaagagcaaaacaaaagtaag
accaccgcacagcaagcggccactgatcttcagacctggaggaggagatatgagggacaattggagaagtgaatt
atataaatataaagtagtaaaaattgaaccattaggagtagcacccaccaaggcaaagagaagagtggtgcagag
agaaaaaagagcagtgggaataggagctttgttccttgggttcttgggagcagcaggaagcactatgggcgcagc
gtcaatgacgctgacggtacaggccagacaattattgtctggtatagtgcagcagcagaacaatttgctgagggc
tattgaggcgcaacagcatctgttgcaactcacagtctggggcatcaagcagctccaggcaagaatcctggctgt
ggaaagatacctaaaggatcaacagctcctggggatttggggttgctctggaaaactcatttgcaccactgctgt
gccttggaatgctagttggagtaataaatctctggaacagaattggaatcacacgacctggatggagtgggacag
agaaattaacaattacacaagcttaatacactccttaattgaagaatcgcaaaaccagcaagaaaagaatgaaca
agaattattggaattagataaatgggcaagtttgtggaattggtttaacataacaaattggctgtggtatataaa
attattcataatgatagtaggaggcttggtaggtttaagaatagtttttgctgtactttctatagtgaatagagt
taggcagggatattcaccattatcgtttcagacccacctcccaaccccgaggggacccgacaggcccgaaggaat
agaagaagaaggtggagagagagacagagacagatccattcgattagtgaacggatctcgacggtatcggttaac
ttttaaaagaaaaggggggattggggggtacagtgcaggggaaagaatagtagacataatagcaacagacataca
aactaaagaattacaaaaacaaattacaaaattcaaaattttatcgatactagtggatctGCGATCGCGTAACGC
CATTTTGCAAGGCATGGAAAAATACCAAACCAAGAATAGAGAAGTTCAGATCAAGGGCGGGTACATGAAAATAGC
TAACGTTGGGCCAAACAGGATATCTGCGGTGAGCAGTTTCGGCCCCGGCCCGGGGCCAAGAACAGATGGTCACCG
CAGTTTCGGCCCCGGCCCGAGGCCAAGAACAGATGGTCCCCAGATATGGCCCAACCCTCAGCAGTTTCTTAAGAC
CCATCAGATGTTTCCAGGCTCCCCCAAGGACCTGAAATGACCCTGCGCCTTATTTGAATTAACCAATCAGCCTGC
TTCTCGCTTCTGTTCGCGCGCTTCTGCTTCCCGAGCTCTATAAAAGAGCTCACAACCCCTCACTCGGCGCGCCAG
TCCTCCGACAGACTGAGTCGTCTAGAgctagcGGATCCCCGGAATTCGACGCCACCATGCTGCTGCTGGTGACCA
GCCTGCTGCTGTGCGAACTGCCGCATCCGGCGTTTCTGCTGATTCCGGAGGTGCAGCTGGTGGAGTCTGGGGGAG
GCTTGGTACAGCCTGGAGGGTCCCTGAGACTCTCCTGTGCAGCCTCTGGATTCACCTTCAGTAGCTATGGCATGA
GCTGGGTCCGCCAGGCTCCAAGACAAGGGCTTGAGTGGGTGGCCAACATAAAGCAAGATGGAAGTGAGAAATACT
ATGCGGACTCAGTGAAGGGCCGATTCACCATCTCCAGAGACAATTCCAAGAACACGCTGTATCTGCAAATGAACA
GCCTGAGAGCCGAGGACACAGCCACGTATTACTGTGCGAAAGAAAATGTGGACTGGGGCCAGGGCACCCTGGTCA
CCGTCTCCTCAGCGGCCGCAACTACCACCCCTGCCCCTCGGCCGCCGACTCCGGCCCCAACCATCGCAAGCCAAC
CCCTCTCCTTGCGCCCCGAAGCTTGCCGCCCGGCCGCGGGTGGAGCCGTGCATACCCGGGGGCTGGACTTTGCCT
GCGATATCTACATTTGGGCCCCGCTGGCCGGCACTTGCGGCGTGCTCCTGCTGTCGCTGGTCATCACCCTTTACT
GCAAGAGGGGCCGGAAGAAGCTGCTTTACATCTTCAAGCAGCCGTTCATGCGGCCCGTGCAGACGACTCAGGAAG
AGGACGGATGCTCGTGCAGATTCCCTGAGGAGGAAGAGGGGGGATGCGAACTGCGCGTCAAGTTCTCACGGTCCG
CCGACGCCCCCGCATATCAACAGGGCCAGAATCAGCTCTACAACGAGCTGAACCTGGGAAGGAGAGAGGAGTACG
ACGTGCTGGACAAGCGACGCGGACGCGACCCGGAGATGGGGGGGAAACCACGGCGGAAAAACCCTCAGGAAGGAC
TGTACAACGAACTCCAGAAAGACAAGATGGCGGAAGCCTACTCAGAAATCGGGATGAAGGGAGAGCGGAGGAGGG
GAAAGGGCCACGACGGCCTGTACCAGGGCCTGAGCACCGCCACCAAGGACACCTACGACGCCCTGCACATGCAGG
CCCTGCCCCCCAGGgagggcagaggaagtcttctaacatgcggtgacgtggaggagaatcccggcccttccggga
tgaccgagtacaagcccacggtgcgcctcgccacccgcgacgacgtccccagggccgtacgcaccctcgccgccg
cgttcgccgactaccccgccacgcgccacaccgtcgatccggaccgccacatcgagcgggtcaccgagctgcaag
aactcttcctcacgcgcgtcgggctcgacatcggcaaggtgtgggtcgcggacgacggcgccgcggtggcggtct
ggaccacgccggagagcgtcgaagcgggggcggtgttcgccgagatcggcccgcgcatggccgagttgagcggtt
cccggctggccgcgcagcaacagatggaaggcctcctggcgccgcaccggcccaaggagcccgcgtggttcctgg
ccaccgtcggcgtctcgcccgaccaccagggcaagggtctgggcagcgccgtcgtgctccccggagtggaggcgg
ccgagcgcgccggggtgcccgccttcctggagacctccgcgccccgcaacctccccttctacgagcggctcggct
tcaccgtcaccgccgacgtcgaggtgcccgaaggaccgcgcacctggtgcatgacccgcaagcccggtgcctgaA
TTAATTAACCAATTGaatcaacctctggattacaaaatttgtgaaagattgactggtattcttaactatgttgct
ccttttacgctatgtggatacgctgctttaatgcctttgtatcatgctattgcttcccgtatggctttcattttc
tcctccttgtataaatcctggttgctgtctctttatgaggagttgtggcccgttgtcaggcaacgtggcgtggtg
tgcactgtgtttgctgacgcaacccccactggttggggcattgccaccacctgtcagctcctttccgggactttc
gctttccccctccctattgccacggcggaactcatcgccgcctgccttgcccgctgctggacaggggctcggctg
ttgggcactgacaattccgtggtgttgtcggggaaatcatcgtcctttccttggctgctcgcctgtgttgccacc
tggattctgcgcgggacgtccttctgctacgtcccttcggccctcaatccagcggaccttccttcccgcggcctg
ctgccggctctgcggcctcttccgcgtcttcgccttcgccctcagacgagtcggatctccctttgggccgcctcc
ccgcctggtacctttaagaccaatgacttacaaggcagctgtagatcttagccactttttaaaagaaaagggggg
actggaagggctaattcactcccaacgaaaataagatctgctttttgcttgtactgggtctctcggttagacca
gatctgagcctgggagctctctggctaactagggaacccactgcttaagcctcaataaagcttgccttgagtgct
tcaagtagtgtgtgcccgtctgttgtgtgactctggtaactagagatccctcagacccttttagtcagtgtggaa
aatctctagcaCGTATGTGTATGATACATAAGGTTATGTATTAATTGTAGCCGCGTTCTAACGACAATATGTACA
AGCCTAATTGTGTAGCATCTGGCTTACTGAAGCAGACCCTATCATCTCTCTCGTAAACTGCCGTCAGAGTCGGTT
TGGTTGGACGAACCTTCTGAGTTTCTGGTAACGCCGTCCCGCACCCGGAAATGGTCAGCGAACCAATCAGCAGGG
TCATCGCTAGCCAGATCCTCTACGCCGGACGCATCGTGGCCGGCATCACCGGCGCCACAGGTGCGGTTGCTGGCG
CCTATATCGCCGACATCACCGATGGGGAAGATCGGGCTCGCCACTTCGGGCTCATGAGCGCTTGTTTCGGCGTGG
GTATGGTGGCAGGCCCCGTGGCCGGGGGACTGTTGGGCGCCATCTCCTTGCATGCACCATTCCTTGCGGCGGCGG
TGCTCAACGGCCTCAACCTACTACTGGGCTGCTTCCTAATGCAGGAGTCGCATAAGGGAGAGCGTCGAATGGTGC
ACTCTCAGTACAATCTGCTCTGATGCCGCATAGTTAAGCCAGCCCCGACACCCGCCAACACCCGCTGACGCGCCC
TGACGGGCTTGTCTGCTCCCGGCATCCGCTTACAGACAAGCTGTGACCGTCTCCGGGAGCTGCATGTGTCAGAGG
TTTTCACCGTCATCACCGAAACGCGCGAGACGAAAGGGCCTCGTGATACGCCTATTTTTATAGGTTAATGTCATG
ATAATAATGGTTTCTTAGACGTCAGGTGGCACTTTTCGGGGAAATGTGCGCGGAACCCCTATTTGTTTATTTTTC
TAAATACATTCAAATATGTATCCGCTCATGAGACAATAACCCTGATAAATGCTTCAATAATATTGAAAAAGGAAG
AGTATGAGTATTCAACATTTCCGTGTCGCCCTTATTCCCTTTTTTGCGGCATTTTGCCTTCCTGTTTTTGCTCAC
CCAGAAACGCTGGTGAAAGTAAAAGATGCTGAAGATCAGTTGGGTGCACGAGTGGGTTACATCGAACTGGATCTC
AACAGCGGTAAGATCCTTGAGAGTTTTCGCCCCGAAGAACGTTTTCCAATGATGAGCACTTTTAAAGTTCTGCTA
TGTGGCGCGGTATTATCCCGTATTGACGCCGGGCAAGAGCAACTCGGTCGCCGCATACACTATTCTCAGAATGAC
TTGGTTGAGTACTCACCAGTCACAGAAAAGCATCTTACGGATGGCATGACAGTAAGAGAATTATGCAGTGCTGCC
ATAACCATGAGTGATAACACTGCGGCCAACTTACTTCTGACAACGATCGGAGGACCGAAGGAGCTAACCGCTTTT
TTGCACAACATGGGGGATCATGTAACTCGCCTTGATCGTTGGGAACCGGAGCTGAATGAAGCCATACCAAACGAC
GAGCGTGACACCACGATGCCTGTAGCAATGGCAACAACGTTGCGCAAACTATTAACTGGCGAACTACTTACTCTA
GCTTCCCGGCAACAATTAATAGACTGGATGGAGGCGGATAAAGTTGCAGGACCACTTCTGCGCTCGGCCCTTCCG
GCTGGCTGGTTTATTGCTGATAAATCTGGAGCCGGTGAGCGTGGGTCTCGCGGTATCATTGCAGCACTGGGGCCA
GATGGTAAGCCCTCCCGTATCGTAGTTATCTACACGACGGGGAGTCAGGCAACTATGGATGAACGAAATAGACAG
ATCGCTGAGATAGGTGCCTCACTGATTAAGCATTGGTAACTGTCAGACCAAGTTTACTCATATATACTTTAGATT
GATTTAAAACTTCATTTTTAATTTAAAAGGATCTAGGTGAAGATCCTTTTTGATAATCTCATGACCAAAATCCCT
TAACGTGAGTTTTCGTTCCACTGAGCGTCAGACCCCGTAGAAAAGATCAAAGGATCTTCTTGAGATCCTTTTTTT
CTGCGCGTAATCTGCTGCTTGCAAACAAAAAAACCACCGCTACCAGCGGTGGTTTGTTTGCCGGATCAAGAGCTA
CCAACTCTTTTTCCGAAGGTAACTGGCTTCAGCAGAGCGCAGATACCAAATACTGTTCTTCTAGTGTAGCCGTAG
TTAGGCCACCACTTCAAGAACTCTGTAGCACCGCCTACATACCTCGCTCTGCTAATCCTGTTACCAGTGGCTGCT
GCCAGTGGCGATAAGTCGTGTCTTACCGGGTTGGACTCAAGACGATAGTTACCGGATAAGGCGCAGCGGTCGGGC
TGAACGGGGGGTTCGTGCACACAGCCCAGCTTGGAGCGAACGACCTACACCGAACTGAGATACCTACAGCGTGAG
CTATGAGAAAGCGCCACGCTTCCCGAAGGGAGAAAGGCGGACAGGTATCCGGTAAGCGGCAGGGTCGGAACAGGA
GAGCGCACGAGGGAGCTTCCAGGGGGAAACGCCTGGTATCTTTATAGTCCTGTCGGGTTTCGCCACCTCTGACTT
GAGCGTCGATTTTTGTGATGCTCGTCAGGGGGGCGGAGCCTATGGAAAAACGCCAGCAACGCGGCCTTTTTACGG
TTCCTGGCCTTTTGCTGGCCTTTTGCTCACATGTTCTTTCCTGCGTTATCCCCTGATTCTGTGGATAACCGTATT
ACCGCCTTTGAGTGAGCTGATACCGCTCGCCGCAGCCGAACGACCGAGCGCAGCGAGTCAGTGAGCGAGGAAGCG
GAAGAGCGCCCAATACGCAAACCGCCTCTCCCCGCGCGTTGGCCGATTCATTAATGCAGCTGTGGAATGTGTGTC
AGTTAGGGTGTGGAAAGTCCCCAGGCTCCCCAGCAGGCAGAAGTATGCAAAGCATGCATCTCAATTAGTCAGCAA
CCAGGTGTGGAAAGTCCCCAGGCTCCCCAGCAGGCAGAAGTATGCAAAGCATGCATCTCAATTAGTCAGCAACCA
TAGTCCCGCCCCTAACTCCGCCCATCCCGCCCCTAACTCCGCCCAGTTCCGCCCATTCTCCGCCCCATGGCTGAC
TAATTTTTTTTATTTATGCAGAGGCCGAGGCCGCCTCGGCCTCTGAGCTATTCCAGAAGTAGTGAGGAGGCTTTT
TTGGAGGCCTAGGCTTTTGCAAAAAGCTTGGACACAAGACAGGCTTGCGAGATATGTTTGAGAATACCACTTTAT
CCCGCGTCAGGGAGAGGCAGTGCGTAAAAAGACGCGGACTCATGTGAAATACTGGTTTTTAGTGCGCCAGATCTC
TATAATCTCGCGCAACCTATTTTCCCCTCGAACACTTTTTAAGCCGTAGATAAACAGGCTGGGACACTTCACATG
AGCGAAAAATACATCGTCACCTGGGACATGTTGCAGATCCATGCACGTAAACTCGCAAGCCGACTGATGCCTTCT
GAACAATGGAAAGGCATTATTGCCGTAAGCCGTGGCGGTCTGTACCGGGTGCGTTACTGGCGCGTGAACTGGGTA
TTCGTCATGTCGATACCGTTTGTATTTCCAGCTACGATCACGACAACCAGCGCGAGCTTAAAGTGCTGAAACGCG
CAGAAGGCGATGGCGAAGGCTTCATCGTTATTGATGACCTGGTGGATACCGGTGGTACTGCGGTTGCGATTCGTG
AAATGTATCCAAAAGCGCACTTTGTCACCATCTTCGCAAAACCGGCTGGTCGTCCGCTGGTTGATGACTATGTTG
TTGATATCCCGCAAGATACCTGGATTGAACAGCCGTGGGATATGGGCGTCGTATTCGTCCCGCCAATCTCCGGTC
GCTAATCTTTTCAACGCCTGGCACTGCCGGGCGTTGTTCTTTTTAACTTCAGGCGGGTTACAATAGTTTCCAGTA
AGTATTCTGGAGGCTGCATCCATGACACAGGCAAACCTGAGCGAAACCCTGTTCAAACCCCGCTTTAAACATCCT
GAAACCTCGACGCTAGTCCGCCGCTTTAATCACGGCGCACAACCGCCTGTGCAGTCGGCCCTTGATGGTAAAACC
ATCCCTCACTGGTATCGCATGATTAACCGTCTGATGTGGATCTGGCGCGGCATTGACCCACGCGAAATCCTCGAC
GTCCAGGCACGTATTGTGATGAGCGATGCCGAACGTACCGACGATGATTTATACGATACGGTGATTGGCTACCGT
GGCGGCAACTGGATTTATGAGTGGGCCCCGGATCTTTGTGAAGGAACCTTACTTCTGTGGTGTGACATAATTGGA
CAAACTACCTACAGAGATTTAAAGCTCTAAGGTAAATATAAAATTTTTAAGTGTATAATGTGTTAAACTACTGAT
TCTAATTGTTTGTGTATTTTAGATTCCAACCTATGGAACTGATGAATGGGAGCAGTGGTGGAATGCCTTTAATGA
GGAAAACCTGTTTTGCTCAGAAGAAATGCCATCTAGTGATGATGAGGCTACTGCTGACTCTCAACATTCTACTCC
TCCAAAAAAGAAGAGAAAGGTAGAAGACCCCAAGGACTTTCCTTCAGAATTGCTAAGTTTTTTGAGTCATGCTGT
GTTTAGTAATAGAACTCTTGCTTGCTTTGCTATTTACACCACAAAGGAAAAAGCTGCACTGCTATACAAGAAAAT
TATGGAAAAATATTCTGTAACCTTTATAAGTAGGCATAACAGTTATAATCATAACATACTGTTTTTTCTTACTCC
ACACAGGCATAGAGTGTCTGCTATTAATAACTATGCTCAAAAATTGTGTACCTTTAGCTTTTTAATTTGTAAAGG
GGTTAATAAGGAATATTTGATGTATAGTGCCTTGACTAGAGATCATAATCAGCCATACCACATTTGTAGAGGTTT
TACTTGCTTTAAAAAACCTCCCACACCTCCCCCTGAACCTGAAACATAAAATGAATGCAATTGTTGTTGTTAACT
TGTTTATTGCAGCTTATAATGGTTACAAATAAAGCAATAGCATCACAAATTTCACAAATAAAGCATTTTTTTCAC
TGCATTCTAGTTGTGGTTTGTCCAAACTCATCAATGTATCTTATCATGTCTGGATCAACTGGATAACTCAAGCTA
ACCAAAATCATCCCAAACTTCCCACCCCATACCCTATTACCACTGCCAATTACCTGTGGTTTCATTTACTCTAAA
CCTGTGATTCCTCTGAATTATTTTCATTTTAAAGAAATTGTATTTGTTAAATATGTACTACAAACTTAGTAG

17. CAR 5

An exemplary CAR construct, as described herein, comprises a CD33 binding domain as comprised in SEQ ID NO: 60, a CD8a transmembrane domain, a CD8a hinge domain, a CD137 (4-1BB) co-stimulatory domain, and a CD3ζ intracellular signaling domain.

In some embodiments, a CAR comprises an antibody V-D-J region. In some embodiments, the CAR (e.g., the V-D-J region) comprises an amino acid sequence shown in SEQ ID NO: 61, or an amino acid sequence that is at least 70%, at least 75%, at least 80%, at least 85%, at least 90%, at least 95%, or at least 99% identical to the amino acid sequence shown in SEQ ID NO: 61. In some embodiments, a CAR comprises an antibody comprising a first heavy chain CDR (CDR1), a second heavy chain CDR (CDR2), and a third heavy chain CDR (CDR3). In some embodiments, heavy chain CDR1 comprises an amino acid sequence shown in SEQ ID NO: 72, or an amino acid sequence having 1, 2, or 3 alterations (e.g., substitutions) relative thereto. In some embodiments, heavy chain CDR2 comprises an amino acid sequence shown in SEQ ID NO: 77, or an amino acid sequence having 1, 2, or 3 alterations (e.g., substitutions) relative thereto. In some embodiments, heavy chain CDR3 comprises an amino acid sequence shown in SEQ ID NO: 78, or an amino acid sequence having 1, 2, or 3 alterations (e.g., substitutions) relative thereto.

In some embodiments, a CAR comprises an antibody comprising 1, 2, 3, or 4 heavy chain framework regions (e.g., 4 heavy chain framework regions). In some embodiments, the CAR comprises a heavy chain framework region 1 (FR1). In some embodiments, the heavy chain FR1 comprises an amino acid sequence shown in SEQ ID NO: 79, or an amino acid sequence having no more than 1, 2, 3, 4, 5, 6, 7, or 8 alterations (e.g., substitutions) relative thereto. In some embodiments, the CAR comprises a heavy chain framework region 2 (FR2). In some embodiments, the heavy chain FR2 comprises an amino acid sequence shown in SEQ ID NO: 80, or an amino acid sequence having no more than 1, 2, 3, 4, 5, 6, 7, or 8 alterations (e.g., substitutions) relative thereto. In some embodiments, the CAR comprises a heavy chain framework region 3 (FR3). In some embodiments, the heavy chain FR3 comprises an amino acid sequence shown in SEQ ID NO: 81, or an amino acid sequence having no more than 1, 2, 3, 4, 5, 6, 7, or 8 alterations (e.g., substitutions) relative thereto. In some embodiments, the CAR comprises a heavy chain framework region 4 (FR4). In some embodiments, the heavy chain FR4 comprises an amino acid sequence shown in SEQ ID NO: 82, or an amino acid sequence having no more than 1, 2, 3, 4, 5, 6, 7, or 8 alterations (e.g., substitutions) relative thereto.

| | |
|---|---|
| V-D-J Region | DVQLVESGGGLVQPGGSLRLSCSVSGNIDRFYAMGWYRQAPG |
| | KQRELVAQLTNNEITTYGDSVEGQFSISGDFDANTVYLQMDSL |
| | KPEDTAVYYCHAHVTTTRWSQDYYWGQGTRVTVSS (SEQ ID |
| | NO: 61) |
| Heavy Chain FR1 | DVQLVESGGGLVQPGGSLRLSCSVS (SEQ ID NO: 79) |
| Heavy Chain CDR1 | GNIDRFYA (SEQ ID NO: 72) |
| Heavy Chain FR2 | MGWYRQAPGKQRELVAQ (SEQ ID NO: 80) |
| Heavy Chain CDR2 | LTNNEIT (SEQ ID NO: 77) |
| Heavy Chain FR3 | TYGDSVEGQFSISGDFDANTVYLQMDSLKPEDTAVYYC (SEQ |
| | ID NO: 81) |
| Heavy Chain CDR3 | HAHVTTTRWSQDYY (SEQ ID NO: 78) |
| Heavy Chain FR4 | WGQGTRVTVSS (SEQ ID NO: 82) |

In some embodiments, a CAR comprises an amino acid sequence shown in SEQ ID NO: 60, or an amino acid sequence that is at least 70%, at least 75%, at least 80%, at least 85%, at least 90%, at least 95%, or at least 99% identical to the amino acid sequence shown in SEQ ID NO: 60.

[SEQ ID NO: 60]
MELGLSWVVLAALLQGVQAQVKLEESGGGSVQAGESLRLSCTASGITFRDYDIDWYRQAPGKEREWLATITPSGT

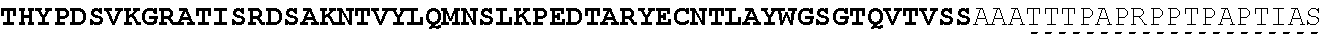

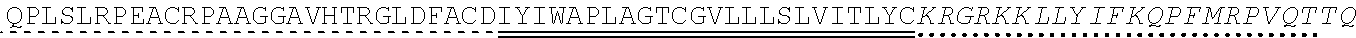

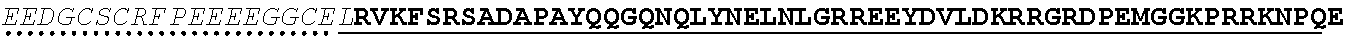

GLYNELQKDKMAEAYSEIGMKGERRRGKGHDGLYQGLSTATKDTYDALHMQALPPR

18. CAR 6

An exemplary CAR construct, as described herein, comprises a CD33 binding domain as comprised in SEQ ID NO: 90, a CD8a transmembrane domain, a CD8a hinge domain, a CD137 (4-1BB) co-stimulatory domain, and a CD3ζ intracellular signaling domain.

In some embodiments, a CAR comprises an antibody V-D-J region. In some embodiments, the CAR (e.g., the V-D-J region) comprises an amino acid sequence shown in SEQ ID NO: 83, or an amino acid sequence that is at least 70%, at least 75%, at least 80%, at least 85%, at least 90%, at least 95%, or at least 99% identical to the amino acid sequence shown in SEQ ID NO: 83. In some embodiments, a CAR comprises an antibody comprising a first heavy chain CDR (CDR1), a second heavy chain CDR (CDR2), and a third heavy chain CDR (CDR3). In some embodiments, heavy chain CDR1 comprises an amino acid sequence shown in SEQ ID NO: 62, or an amino acid sequence having 1, 2, or 3 alterations (e.g., substitutions) relative thereto. In some embodiments, heavy chain CDR2 comprises an amino acid sequence shown in SEQ ID NO: 63, or an amino acid sequence having 1, 2, or 3 alterations (e.g., substitutions) relative thereto. In some embodiments, heavy chain CDR3 comprises an amino acid sequence shown in SEQ ID NO: 64, or an amino acid sequence having 1, 2, or 3 alterations (e.g., substitutions) relative thereto.

In some embodiments, a CAR comprises an antibody comprising 1, 2, 3, or 4 heavy chain framework regions (e.g., 4 heavy chain framework regions). In some embodiments, the CAR comprises a heavy chain framework region 1 (FR1). In some embodiments, the heavy chain FR1 comprises an amino acid sequence shown in SEQ ID NO: 65, or an amino acid sequence having no more than 1, 2, 3, 4, 5, 6, 7, or 8 alterations (e.g., substitutions) relative thereto. In some embodiments, the CAR comprises a heavy chain framework region 2 (FR2). In some embodiments, the heavy chain FR2 comprises an amino acid sequence shown in SEQ ID NO: 66, or an amino acid sequence having no more than 1, 2, 3, 4, 5, 6, 7, or 8 alterations (e.g., substitutions) relative thereto. In some embodiments, the CAR comprises a heavy chain framework region 3 (FR3). In some embodiments, the heavy chain FR3 comprises an amino acid sequence shown in SEQ ID NO: 67, or an amino acid sequence having no more than 1, 2, 3, 4, 5, 6, 7, or 8 alterations (e.g., substitutions) relative thereto. In some embodiments, the CAR comprises a heavy chain framework region 4 (FR4). In some embodiments, the heavy chain FR4 comprises an amino acid sequence shown in SEQ ID NO: 68, or an amino acid sequence having no more than 1, 2, 3, 4, 5, 6, 7, or 8 alterations (e.g., substitutions) relative thereto.

| | |
|---|---|
| V-D-J Region | QVQLVETGGGLVRAGGSLRLSCAASGRTADIYNIGWFRQAPG |
| | KEREFVAAITWIGRTPYYADAVKGRFAFSTDSAKNTVSLQMD |
| | NLKPEDTGVYYCNAAHYLEGNTDYYWGQGTQVTVSS (SEQ |
| | ID NO: 83) |
| Heavy Chain FR1 | QVQLVETGGGLVRAGGSLRLSCAAS (SEQ ID NO: 65) |
| Heavy Chain CDR1 | GRTADIYN (SEQ ID NO: 62) |
| Heavy Chain FR2 | IGWFRQAPGKEREFVAA (SEQ ID NO: 66) |
| Heavy Chain CDR2 | ITWIGRTP (SEQ ID NO: 63) |
| Heavy Chain FR3 | YYADAVKGRFAFSTDSAKNTVSLQMDNLKPEDTGVYYC |
| | (SEQ ID NO: 67) |
| Heavy Chain CDR3 | NAAHYLEGNTDYY (SEQ ID NO: 64) |
| Heavy Chain FR4 | WGQGTQVTVSS (SEQ ID NO: 68) |

In some embodiments, a CAR comprises an amino acid sequence shown in SEQ ID NO: 90, or an amino acid sequence that is at least 70%, at least 75%, at least 80%, at least 85%, at least 90%, at least 95%, or at least 99% identical to the amino acid sequence shown in SEQ ID NO: 90.

[SEQ ID NO: 90]
MELGLSWVVLAALLQGVQAQVQLVETGGGLVRAGGSLRLSCAASGRTADIYNIGWFRQAPGKEREFVAAITWIGR

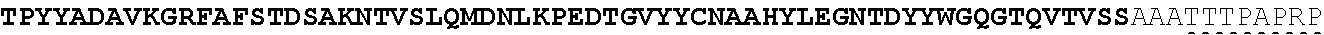

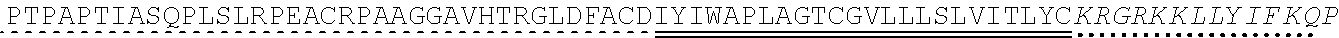

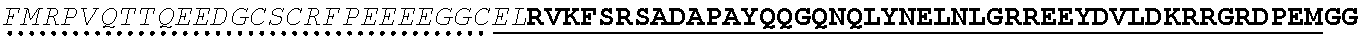

KPRRKNPQEGLYNELQKDKMAEAYSEIGMKGERRRGKGHDGLYQGLSTATKDTYDALHMQALPPR

19. CAR 7

An exemplary CAR construct, as described herein, comprises a CD33 binding domain as comprised in SEQ ID NO: 91, a CD8a transmembrane domain, a CD8a hinge domain, a CD137 (4-1BB) co-stimulatory domain, and a CD3ζ intracellular signaling domain.

In some embodiments, a CAR comprises an antibody V-D-J region. In some embodiments, the CAR (e.g., the V-D-J region) comprises an amino acid sequence shown in SEQ ID NO: 69, or an amino acid sequence that is at least 70%, at least 75%, at least 80%, at least 85%, at least 90%, at least 95%, or at least 99% identical to the amino acid sequence shown in SEQ ID NO: 69. In some embodiments, a CAR comprises an antibody comprising a first heavy chain CDR (CDR1), a second heavy chain CDR (CDR2), and a third heavy chain CDR (CDR3). In some embodiments, heavy chain CDR1 comprises an amino acid sequence shown in SEQ ID NO: 70, or an amino acid sequence having 1, 2, or 3 alterations (e.g., substitutions) relative thereto. In some embodiments, heavy chain CDR2 comprises an amino acid sequence shown in SEQ ID NO: 71, or an amino acid sequence having 1, 2, or 3 alterations (e.g., substitutions) relative thereto. In some embodiments, heavy chain CDR3 comprises an amino acid sequence shown in SEQ ID NO: 73, or an amino acid sequence having 1, 2, or 3 alterations (e.g., substitutions) relative thereto.

In some embodiments, a CAR comprises an antibody comprising 1, 2, 3, or 4 heavy chain framework regions (e.g., 4 heavy chain framework regions). In some embodiments, the CAR comprises a heavy chain framework region 1 (FR1). In some embodiments, the heavy chain FR1 comprises an amino acid sequence shown in SEQ ID NO: 74, or an amino acid sequence having no more than 1, 2, 3, 4, 5, 6, 7, or 8 alterations (e.g., substitutions) relative thereto. In some embodiments, the CAR comprises a heavy chain framework region 2 (FR2). In some embodiments, the heavy chain FR2 comprises an amino acid sequence shown in SEQ ID NO: 75, or an amino acid sequence having no more than 1, 2, 3, 4, 5, 6, 7, or 8 alterations (e.g., substitutions) relative thereto. In some embodiments, the CAR comprises a heavy chain framework region 3 (FR3). In some embodiments, the heavy chain FR3 comprises an amino acid sequence shown in SEQ ID NO: 76, or an amino acid sequence having no more than 1, 2, 3, 4, 5, 6, 7, or 8 alterations (e.g., substitutions) relative thereto. In some embodiments, the CAR comprises a heavy chain framework region 4 (FR4). In some embodiments, the heavy chain FR4 comprises an amino acid sequence shown in SEQ ID NO: 68, or an amino acid sequence having no more than 1, 2, 3, 4, 5, 6, 7, or 8 alterations (e.g., substitutions) relative thereto.

| | |
|---|---|
| V-D-J Region | QVQLVQPGGSLRLFCVASEEFFSIYAMGWYRQAPGKQHEMVA |
| | RFTRDGKITYADSAKGRFTITRDAKNTLNLQMNGLIPEDTAVY |
| | YCNINHYWGQGTQVTVSS (SEQ ID NO: 69) |
| Heavy Chain FR1 | QVQLVQPGGSLRLFCVAS (SEQ ID NO: 74) |
| Heavy Chain CDR1 | EEFFSIYA (SEQ ID NO: 70) |
| Heavy Chain FR2 | MGWYRQAPGKQHEMVAR (SEQ ID NO: 75) |
| Heavy Chain CDR2 | FTRDGKI (SEQ ID NO: 71) |
| Heavy Chain FR3 | TYADSAKGRFTITRDAKNTLNLQMNGLIPEDTAVYYC (SEQ |
| | ID NO: 76) |
| Heavy Chain CDR3 | NINHY (SEQ ID NO: 73) |
| Heavy Chain FR4 | WGQGTQVTVSS (SEQ ID NO: 68) |

In some embodiments, a CAR comprises an amino acid sequence shown in SEQ ID NO: 91, or an amino acid sequence that is at least 70%, at least 75%, at least 80%, at least 85%, at least 90%, at least 95%, or at least 99% identical to the amino acid sequence shown in SEQ ID NO: 91.

[SEQ ID NO: 91]
MELGLSWVVLAALLQGVQAQVQLVQPGGSLRLFCVASEEFFSIYAMGWYRQAPGKQHEMVARFTRDGKITYADSA

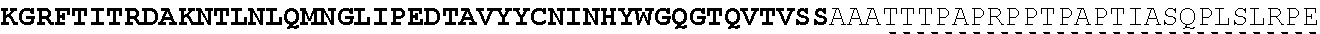

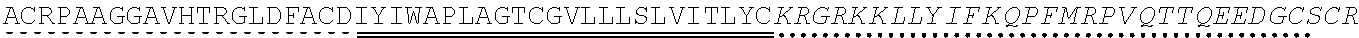

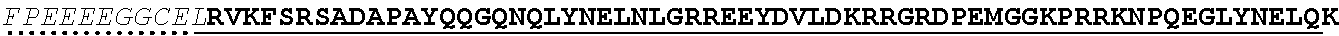

DKMAEAYSEIGMKGERRRGKGHDGLYQGLSTATKDTYDALHMQALPPR

20. CAR 8

An exemplary CAR construct, as described herein, comprises a CD33 binding domain as comprised in SEQ ID NO: 92, a CD8a transmembrane domain, a CD8a hinge domain, a CD137 (4-1BB) co-stimulatory domain, and a CD3ζ intracellular signaling domain.

In some embodiments, a CAR comprises an antibody V-D-J region. In some embodiments, the CAR (e.g., the V-D-J region) comprises an amino acid sequence shown in SEQ ID NO: 61, or an amino acid sequence that is at least 70%, at least 75%, at least 80%, at least 85%, at least 90%, at least 95%, or at least 99% identical to the amino acid sequence shown in SEQ ID NO: 61. In some embodiments, a CAR comprises an antibody comprising a first heavy chain CDR (CDR1), a second heavy chain CDR (CDR2), and a third heavy chain CDR (CDR3). In some embodiments, heavy chain CDR1 comprises an amino acid sequence shown in SEQ ID NO: 72, or an amino acid sequence having 1, 2, or 3 alterations (e.g., substitutions) relative thereto. In some embodiments, heavy chain CDR2 comprises an amino acid sequence shown in SEQ ID NO: 77, or an amino acid sequence having 1, 2, or 3 alterations (e.g., substitutions) relative thereto. In some embodiments, heavy chain CDR3 comprises an amino acid sequence shown in SEQ ID NO: 78, or an amino acid sequence having 1, 2, or 3 alterations (e.g., substitutions) relative thereto.

In some embodiments, a CAR comprises an antibody comprising 1, 2, 3, or 4 heavy chain framework regions (e.g., 4 heavy chain framework regions). In some embodiments, the CAR comprises a heavy chain framework region 1 (FR1). In some embodiments, the heavy chain FR1 comprises an amino acid sequence shown in SEQ ID NO: 79, or an amino acid sequence having no more than 1, 2, 3, 4, 5, 6, 7, or 8 alterations (e.g., substitutions) relative thereto. In some embodiments, the CAR comprises a heavy chain framework region 2 (FR2). In some embodiments, the heavy chain FR2 comprises an amino acid sequence shown in SEQ ID NO: 80, or an amino acid sequence having no more than 1, 2, 3, 4, 5, 6, 7, or 8 alterations (e.g., substitutions) relative thereto. In some embodiments, the CAR comprises a heavy chain framework region 3 (FR3). In some embodiments, the heavy chain FR3 comprises an amino acid sequence shown in SEQ ID NO: 81, or an amino acid sequence having no more than 1, 2, 3, 4, 5, 6, 7, or 8 alterations (e.g., substitutions) relative thereto. In some embodiments, the CAR comprises a heavy chain framework region 4 (FR4). In some embodiments, the heavy chain FR4 comprises an amino acid sequence shown in SEQ ID NO: 82, or an amino acid sequence having no more than 1, 2, 3, 4, 5, 6, 7, or 8 alterations (e.g., substitutions) relative thereto.

| | |
|---|---|
| V-D-J Region | DVQLVESGGGLVQPGGSLRLSCSVSGNIDRFYAMGWYRQAPG |
| | KQRELVAQLTNNEITTYGDSVEGQFSISGDFDANTVYLQMDSL |
| | KPEDTAVYYCHAHVTTTRWSQDYYWGQGTRVTVSS (SEQ ID |
| | NO: 61) |
| Heavy Chain FR1 | DVQLVESGGGLVQPGGSLRLSCSVS (SEQ ID NO: 79) |
| Heavy Chain CDR1 | GNIDRFYA (SEQ ID NO: 72) |
| Heavy Chain FR2 | MGWYRQAPGKQRELVAQ (SEQ ID NO: 80) |
| Heavy Chain CDR2 | LTNNEIT (SEQ ID NO: 77) |
| Heavy Chain FR3 | TYGDSVEGQFSISGDFDANTVYLQMDSLKPEDTAVYYC (SEQ |
| | ID NO: 81) |
| Heavy Chain CDR3 | HAHVTTTRWSQDYY (SEQ ID NO: 78) |
| Heavy Chain FR4 | WGQGTRVTVSS (SEQ ID NO: 82) |

In some embodiments, a CAR comprises an amino acid sequence shown in SEQ ID NO: 92, or an amino acid sequence that is at least 70%, at least 75%, at least 80%, at least 85%, at least 90%, at least 95%, or at least 99% identical to the amino acid sequence shown in SEQ ID NO: 92.

[SEQ ID NO: 92]
MELGLSWVVLAALLQGVQADVQLVESGGGLVQPGGSLRLSCSVSGNIDRFYAMGWYRQAPGKQRELVAQLTNNEI

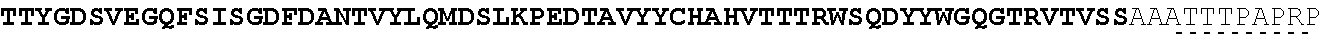

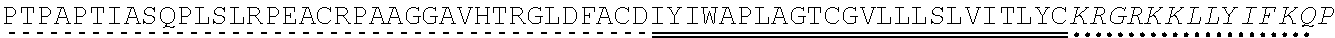

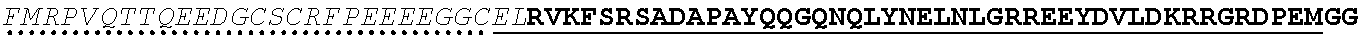

KPRRKNPQEGLYNELQKDKMAEAYSEIGMKGERRRGKGHDGLYQGLSTATKDTYDALHMQALPPR

In some embodiments, any nucleotide sequences described herein may be codon-optimized. Without being bound to a particular theory or mechanism, it is believed that codon optimization of the nucleotide sequence increases the translation efficiency of the mRNA transcripts. Codon optimization of the nucleotide sequence may involve substituting a native codon for another codon that encodes the same amino acid, but can be translated by tRNA that is more readily available within a cell, thus increasing translation efficiency. Optimization of the nucleotide sequence may also reduce secondary mRNA structures that would interfere with translation, thus increasing translation efficiency. In an embodiment of the invention, the codon-optimized nucleotide sequence may comprise, consist, or consist essentially of any one of the nucleic acid sequences described herein.

Any of the nucleic acids described herein may be recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

A recombinant nucleic acid may be one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques, such as those described in Green et al., supra. The nucleic acids can be constructed based on chemical synthesis and/or enzymatic ligation reactions using procedures known in the art. See, for example, Green et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methyl guanine, 1-methylinosine, 2,2-dimethylguanine, 2-methyladenine, 2-methylguanine, 3-methylcytosine, 5-methylcytosine, N6-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, CO) and Synthegen (Houston, TX).

The nucleic acids can comprise any isolated or purified nucleotide sequence which encodes any of the CAR constructs or functional portions or functional variants thereof. Alternatively, the nucleotide sequence can comprise a nucleotide sequence which is degenerate to any of the sequences or a combination of degenerate sequences.

Also provided herein are isolated or purified nucleic acids comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions may hybridize under high stringency conditions. The term “high stringency conditions” refers to a nucleotide sequence that specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the CARs constructs described herein. It is generally appreciated that conditions can be rendered more stringent by the addition of increasing amounts of formamide.

The present disclosure also provides nucleic acids comprising a nucleotide sequence that is at least about 70% or more, e.g., about 80%, about 90%, about 91%, about 92%, about 93%, about 94%, about 95%, about 96%, about 97%, about 98%, or about 99% identical to any of the nucleic acids described herein. Also with the scope of the present disclosure are functional portions of the CAR constructs described herein.

### Therapeutic Methods

Aspects of the present disclosure provide methods of treating a disease, disorder, or condition in a subject comprising administering to the subject a therapeutically effective amount of any of the CARs, nucleic acids, cells expressing any of the CARs, or pharmaceutical compositions described herein. In some embodiments, the methods involve administering a therapeutically effective amount of a pharmaceutical composition comprising cells (e.g., a population of cells) expressing any of the CARs described herein. In some aspects, the disclosure provides a method of treating cancer in a subject, comprising administering to the subject a therapeutically effective amount of any of the CARs, nucleic acids, cells expressing any of the CARs, or pharmaceutical compositions described herein. In some embodiments, the method is for treating a hematopoietic malignancy or pre-malignancy in a subject, comprising administering to the subject a therapeutically effective amount of a pharmaceutical composition comprising cells (e.g., a population of cells) expressing any of the CARs described herein.

In another aspect, the disclosure provides methods for stimulating an immune response to a target cell or tissue (e.g., a cancer cell, tumor cell, or tumor tissue) in a subject comprising administering to a subject a therapeutically effective amount of a pharmaceutical composition comprising cells (e.g., a population of cells) expressing any of the CARs described herein. In some embodiments, the disclosure includes use of the modified cells described herein in the manufacture of a medicament for the stimulating an immune response in a subject in need thereof. In some embodiments, the disclosure includes use of any of the CARs, nucleic acids, cells expressing any of the CARs, or pharmaceutical compositions described herein in the manufacture of a medicament for the treatment of a cancer in a subject in need thereof. In some embodiments, the method involves use of any of the CARs, nucleic acids, cell expressing any of the CARs, or pharmaceutical compositions described herein in the manufacture of a medicament for the treatment of a tumor or cancer in a subject in need thereof. In some embodiments, the method involves use of any of the CARs, nucleic acids, cell expressing any of the CARs, or pharmaceutical compositions described herein in the manufacture of a medicament for the treatment of a hematopoietic malignancy or pre-malignancy in a subject in need thereof.

The modified cells, (e.g., immune cells, such as T-lymphocytes, NK cells) generated as described herein possess targeted effector activity. In some embodiments, the modified cells have targeted effector activity directed against an antigen on a target cell, such as through specific binding to an antigen-binding domain of a CAR. In some embodiments, the targeted effector activity includes, but is not limited to, phagocytosis, targeted cellular cytotoxicity, antigen presentation, and cytokine secretion.

The CAR constructs described herein (including functional portions and variants thereof), nucleic acids, vectors, and host cells expressing any of the CARs described herein, such as, immune cells, T-lymphocytes, NK cells (including populations thereof), are collectively referred to as “CAR construct materials.”

Pharmaceutical Compositions

The CAR construct materials described herein can be formulated into a composition, such as a pharmaceutical composition. In some embodiments, the present disclosure provides a pharmaceutical composition comprising any of the CAR construct materials described herein and a pharmaceutically acceptable carrier. The pharmaceutical compositions containing any of the CAR construct materials can comprise more than one CAR construct material, e.g., a CAR construct and a nucleic acid, or two or more different CAR constructs. Alternatively, the pharmaceutical composition can comprise a CAR construct in combination with other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the pharmaceutical composition comprises a cell expressing any of the CAR constructs described herein or populations of such cells.

With respect to pharmaceutical compositions, the pharmaceutically acceptable carrier can be any of those conventionally used and is limited only by chemico-physical considerations, such as solubility and lack of reactivity with the active agent(s), and by the route of administration. Pharmaceutically acceptable carriers described herein, for example, vehicles, adjuvants, excipients, and diluents, are well known to those skilled in the art and are readily available to the public. It is preferred that the pharmaceutically acceptable carrier be one which has no detrimental side effects or toxicity under the conditions of use.

The choice of carrier will be determined in part by the particular CAR construct material, as well as by the particular methods used to administer the CAR construct material, for example to a subject. Accordingly, there are a variety of suitable formulations of the pharmaceutical composition of the invention. Methods for preparing administrable (e.g., parenterally administrable) compositions are known or apparent to those skilled in the art and are described in more detail in, for example, Remington: The Science and Practice of Pharmacy, Pharmaceutical Press; 22nd ed. (2012).

The CAR construct materials, including pharmaceutical compositions comprising any of the CAR materials, may be administered in any suitable manner. In some embodiments, CAR materials, including pharmaceutical compositions comprising any of the CAR materials, are administered by injection, (e.g., subcutaneously, intravenously, intratumorally, intraarterially, intramuscularly, intradermally, interperitoneally, or intrathecally). In some embodiments, CAR construct materials, including pharmaceutical compositions comprising any of the CAR materials, are administered intravenously. In some embodiments, CAR materials, including pharmaceutical compositions comprising any of the CAR materials, are administered by infusion. A suitable pharmaceutically acceptable carrier for the CAR construct materials described herein for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, IL), PLASMA-LYTE A (Baxter, Deerfield, IL), about 5% dextrose in water, or Ringer's lactate. In some embodiments, the pharmaceutically acceptable carrier is supplemented with human serum albumen.

Amounts effective for a therapeutic or prophylactic use will depend on, for example, the stage and severity of the disease or disorder being treated, the age, weight, and general state of health of the patient, and the judgment of the prescribing physician. The size of the dose will also be determined by the active selected, method of administration, timing and frequency of administration, the existence, nature, and extent of any adverse side-effects that might accompany the administration of a particular active, and the desired physiological effect. It will be appreciated by one of skill in the art that various diseases or disorders could require prolonged treatment involving multiple administrations, for example using the CAR construct materials described herein in each or various rounds of administration. By way of example and not intending to limit the invention, when the CAR construct material is a host cell expressing any of the CARs described herein, an exemplary dose of host cells may be a minimum of one million cells (1×10⁶ cells/dose).

For purposes of the invention, the amount or dose of the CAR construct material administered should be sufficient to effect a therapeutic or prophylactic response in the subject or animal over a reasonable time frame. For example, the dose of the CAR construct material should be sufficient to bind to antigen (i.e., CD33), or detect, treat or prevent cancer or hematopoietic malignancy or pre-malignancy, including reducing one or more symptoms and/or delaying the progression of the disease, in a period of from about 2 hours or longer, e.g., about 12 to about 24 or more hours, such as for about 1 day to 6 months or longer, from the time of administration. In some embodiments, the time period could be even longer. The dose will be determined by factors such as the efficacy of the particular CAR construct material, the condition of the animal (e.g., human), including the body weight of the animal (e.g., human) to be treated, and the severity of the disease in the subject.

An assay, which comprises, for example, comparing the extent to which target cells are lysed and/or IFN-gamma or IL-2 is secreted by cells expressing any o the CARs described herein upon administration of a given dose of such cells (e.g., T cells, NK cells) to a subject, among a set of subjects of which is each given a different dose of the cells, could be used to determine a starting dose to be administered to a subject. The extent to which target cells are lysed and/or IFN-gamma or IL-2 is secreted upon administration of a certain dose can be assayed by methods known in the art.

When the CAR construct materials are administered with one or more additional therapeutic agents, one or more additional therapeutic agents can be co-administered to a subject. The term “co-administering” refers to administering one or more additional therapeutic agents and the CAR construct materials sufficiently close in time such that the CAR construct materials can enhance the effect of one or more additional therapeutic agents, or vice versa. In this regard, CAR construct materials can be administered first and the one or more additional therapeutic agents can be administered second, or vice versa. Alternatively, CAR construct materials and the one or more additional therapeutic agents can be administered simultaneously. An exemplary therapeutic agent that may be co-administered with the CAR construct materials is IL-2.

It is contemplated that CAR construct materials described herein can be used in methods of treating or preventing a disease in a subject. Without being bound to a particular theory or mechanism, the CAR constructs have biological activity, e.g., CARs that recognize antigen, e.g., CD33, such that the CARs, when expressed by a cell, are able to mediate an immune response against the cell expressing the antigen, e.g., CD33. In this regard, in some embodiments, the methods of treating or preventing a disease, disorder, or condition (e.g., cancer, e.g., hematopoietic malignancy or pre-malignancy) in a subject (e.g., AML, MDS) comprising administering to the mammal any of the CAR constructs, the nucleic acids, the recombinant expression vectors, the host cells, the population of cells, and/or the pharmaceutical compositions described herein in an amount effective to treat or prevent the disease, disorder, or condition in a subject (e.g., cancer, hematopoietic malignancy or pre-malignancy) in the subject.

In some embodiments, the method further comprises lymphodepleting the subject (e.g., mammal) prior to administering any of the CAR construct materials described herein. Examples of lymphodepletion include, but may not be limited to, nonmyeloablative lymphodepleting chemotherapy, myeloablative lymphodepleting chemotherapy, total body irradiation, etc.

In some embodiments, the cells expressing the cells or populations of such cells are administered, the cells can be cells that are allogeneic or autologous to the subject. In some embodiments, the cells are autologous to the subject.

With respect to the methods of treatment, in some embodiments, the disease, disorder, or condition is cancer. The cancer can be any cancer, including any of acute lymphocytic cancer, acute myeloid leukemia (AML), alveola rhabdomyosarcoma, bladder cancer (e.g., bladder carcinoma), bone cancer, brain cancer (e.g., medulloblastoma), breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia (CLL), chronic myeloid cancer, colon cancer, esophageal cancer, cervical cancer, fibrosarcoma, gastrointestinal carcinoid tumor, head and neck cancer (e.g., head and neck squamous cell carcinoma), Hodgkin lymphoma, hypopharynx cancer, kidney cancer, larynx cancer, leukemia, liquid tumors, liver cancer, lung cancer (e.g., non-small cell lung carcinoma), lymphoma, malignant mesothelioma, mastocytoma, melanoma, multiple myeloma, nasopharynx cancer, non-Hodgkin lymphoma, B-chronic lymphocytic leukemia, B-precursor acute lymphoblastic leukemia (B-ALL), pre-B cell precursor acute lymphoblastic leukemia (BCP-ALL), B cell lymphoma, hairy cell leukemia, acute lymphocytic leukemia (ALL), and Burkitt's lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, stomach cancer, testicular cancer, thyroid cancer, and ureter cancer. Preferably, the cancer is a hematological malignancy (e.g., leukemia or lymphoma, including but not limited to Hodgkin lymphoma, non-Hodgkin lymphoma, CLL, acute lymphocytic cancer, acute myeloid leukemia (AML), B-chronic lymphocytic leukemia, hairy cell leukemia, acute lymphocytic leukemia (ALL) (also referred to as “acute lymphoblastic leukemia”), B-ALL, BCP-ALL, B cell lymphoma, and Burkitt's lymphoma). Preferably, the cancer is characterized by the expression of CD33.

In some embodiments, the disease, disorder, or condition is a hematologic malignancy, or a cancer of the blood. In some embodiments, the malignancy is a lymphoid malignancy or a myeloid malignancy. In some embodiments, the disease, disorder, or condition is a hematopoietic malignancy. In some embodiments, the disease, disorder, or condition is a leukemia, e.g., acute myeloid leukemia (AML). AML is characterized as a heterogeneous, clonal, neoplastic disease that originates from transformed cells that have progressively acquired critical genetic changes that disrupt key differentiation and growth-regulatory pathways. (Dohner et al., NEJM, (2015) 373:1136). Without wishing to be bound by theory, it is believed in some embodiments, that CD123 is expressed on myeloid leukemia cells as well as on normal myeloid and monocytic precursors and is an attractive target for AML therapy.

In some embodiments, the hematopoietic malignancy or hematological disorder associated with CD123 is a precancerous condition such as a myelodysplasia, a myelodysplastic syndrome or a preleukemia. Myelodysplastic syndromes (MDS) are hematological medical conditions characterized by disorderly and ineffective hematopoiesis, or blood production. Thus, the number and quality of blood-forming cells decline irreversibly. Some patients with MDS can develop severe anemia, while others are asymptomatic. The classification scheme for MDS is known in the art, with criteria designating the ratio or frequency of particular blood cell types, e.g., myeloblasts, monocytes, and red cell precursors. MDS includes refractory anemia, refractory anemia with ring sideroblasts, refractory anemia with excess blasts, refractory anemia with excess blasts in transformation, chronic myelomonocytic leukemia (CML). In some embodiments, MDS can progress to an acute myeloid leukemia (AML).

Furthermore, the treatment or prevention provided by the methods described herein can include treatment or prevention of one or more conditions or symptoms of the disease, e.g., cancer, being treated or prevented.

Aspects of the present disclosure also provide a method of detecting the presence of a disease, disorder, or condition (e.g., cancer) in a subject, comprising: (a) contacting a sample comprising one or more cells from the subject with any of the CAR constructs, the nucleic acids, the vectors, the host cells expressing any of the CARs, populations of such cells, or any of the pharmaceutical compositions described herein, thereby forming a complex, (b) and detecting the complex, wherein detection of the complex is indicative of the presence of the disease, disorder, or condition in the subject.

The sample may be obtained by any suitable method, e.g., biopsy or necropsy. A biopsy is the removal of tissue and/or cells from an individual. Such removal may be to collect tissue and/or cells from the individual in order to perform experimentation on the removed tissue and/or cells. This experimentation may include experiments to determine if the individual has and/or is suffering from a certain condition or disease-state. The condition or disease may be, e.g., cancer, e.g., a hematopoietic malignancy or pre-malignancy.

In some embodiments, the sample comprising cells of the subject can be a sample comprising whole cells, lysates thereof, or a fraction of the whole cell lysates, e.g., a nuclear or cytoplasmic fraction, a whole protein fraction, or a nucleic acid fraction. If the sample comprises whole cells, the cells can be any cells of the subject, e.g., the cells of any organ or tissue, including blood cells or endothelial cells.

In some embodiments, the contacting of the sample with any of the CARs, nucleic acids, vectors, cells expressing any of the CARs, populations of such cells, or any of the pharmaceutical compositions described herein can take place in vitro or in vivo with respect to the subject. Preferably, the contacting is in vitro.

Detection of the complex can occur through any number of ways known in the art. For instance, any of the CAR constructs, nucleic acids, vectors, host cells expressing any of the CARs, or populations of such cells, or any of the pharmaceutical compositions described herein, can be labeled with a detectable label such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

Methods of testing a CAR for the ability to recognize target cells and for antigen specificity are known in the art. For instance, Clay et al., J. Immunol. (1999) 163: 507-513, teaches methods of measuring the release of cytokines (e.g., interferon-g, granulocyte/monocyte colony stimulating factor (GM-CSF), tumor necrosis factor alpha (TNF-α) or interleukin 2 (IL-2)). In addition, CAR function can be evaluated by measurement of cellular cytotoxicity, as described in Zhao et al., J. Immunol. (2005) 174: 4415-4423.

Hematopoietic Cells Deficient in CD33

Aspects of the present disclosure also provide compositions and methods for the inhibition of a CD33 target antigen. Such treatment regimen can involve, for example, the following steps: (1) administering a therapeutically effective amount of a cell or population of cells, e.g., an immune cell (e.g., a T lymphocyte, NK cell) to the patient, where the cell comprises a nucleic acid sequence encoding any of the CARs targeting CD33 described herein; and (2) administering (e.g., infusing or reinfusing) the patient with hematopoietic stem cells, either autologous or allogeneic, where the hematopoietic cells have reduced expression of CD33. In some embodiments, the hematopoietic cells are genetically modified to have reduced or eliminated expression of CD33.

In some embodiments, the hematopoietic cells are hematopoietic stem cells HSCs). In some embodiments, the hematopoietic cells are hematopoietic progenitor cells (HPCs). Hematopoietic stem cells (HSCs) are capable of giving rise to both myeloid and lymphoid progenitor cells that further give rise to myeloid cells (e.g., monocytes, macrophages, neutrophils, basophils, dendritic cells, erythrocytes, platelets, etc) and lymphoid cells (e.g., T cells, B cells, NK cells), respectively. HSCs are characterized by the expression of the cell surface marker CD34 (e.g., CD34+), which can be used for the identification and/or isolation of HSCs, and absence of cell surface markers associated with commitment to a cell lineage. In some embodiments, the HSCs are peripheral blood HSCs.

In some embodiments, the hematopoietic cells (e.g., HSCs) are obtained from a subject, such as a mammalian subject. In some embodiments, the mammalian subject is a non-human primate, a rodent (e.g., mouse or rat), a bovine, a porcine, an equine, or a domestic animal. In some embodiments, hematopoietic cells (e.g., HSCs) are obtained from a human patient, such as a human patient having a hematopoietic malignancy or pre-malignancy. In some embodiments, the hematopoietic cells (e.g., HSCs) are obtained from a healthy donor. In some embodiments, the hematopoietic cells (e.g., HSCs) are obtained from the subject to whom the immune cells expressing the chimeric antigen receptors will be subsequently administered.

HSCs may be obtained from any suitable source using convention means known in the art. In some embodiments, HSCs are obtained from a sample from a subject, such as bone marrow sample or from a blood sample. Alternatively or in addition, HSCs may be obtained from an umbilical cord. In some embodiments, the HSCs are from bone marrow or peripheral blood mononuclear cells (PBMCs). In general, bone marrow cells may be obtained from iliac crest, femora, tibiae, spine, rib or other medullary spaces of a subject. Bone marrow may be taken out of the patient and isolated through various separations and washing procedures known in the art. An exemplary procedure for isolation of bone marrow cells comprises the following steps: a) extraction of a bone marrow sample; b) centrifugal separation of bone marrow suspension in three fractions and collecting the intermediate fraction, or buffycoat; c) the buffycoat fraction from step (b) is centrifuged one more time in a separation fluid, commonly Ficoll™, and an intermediate fraction which contains the bone marrow cells is collected; and d) washing of the collected fraction from step (c) for recovery of re-transfusable bone marrow cells. Methods of obtaining mammalian cells, such as hematopoietic stem cells, are described, e.g., in PCT/US2016/057339, which is herein incorporated by reference in its entirety.

HSCs typically reside in the bone marrow but can be mobilized into the circulating blood by administering a mobilizing agent in order to harvest HSCs from the peripheral blood. In some embodiments, the subject from which the HSCs are obtained is administered a mobilizing agent, such as granulocyte colony-stimulating factor (G-CSF). The number of the HSCs collected following mobilization using a mobilizing agent is typically greater than the number of cells obtained without use of a mobilizing agent.

In some embodiments, a sample is obtained from a subject and is then enriched for a desired cell type (e.g. CD34+/CD33− cells). For example, PBMCs and/or CD34+ hematopoietic cells can be isolated from blood as described herein. Cells can also be isolated from other cells, for example by isolation and/or activation with an antibody binding to an epitope on the cell surface of the desired cell type. Another exemplary method that can be used includes negative selection using antibodies to cell surface markers to selectively enrich for a specific cell type without activating the cell by receptor engagement.

Populations of HSC can be expanded prior to or after genetically engineering the HSC to become deficient a target antigen (i.e., CD33). The cells may be cultured under conditions that comprise an expansion medium comprising one or more cytokines, such as stem cell factor (SCF), Flt-3 ligand (FLt3L), thrombopoietin (TPO), Interleukin 3 (IL-3), or Interleukin 6 (IL-6). The cell may be expanded for about 1, 2, 3, 4, 5, 6, 7, 8, 9, 10, 11, 12, 13, 14, 15, 16, 17, 18, 19, 20, 21, 22, 23, 23, 25 days or any range necessary. In some embodiments, HSCs are expanded after isolation of a desired cell population (e.g., CD34+/CD33−) from a sample obtained from a subject and prior to genetic engineering. In some embodiments, the HSC are expanded after genetic engineering, thereby selectively expanding cells that have undergone the genetic modification and are deficient in a lineage-specific cell-surface antigen. In some embodiments, a cell (“a clone”) or several cells having a desired characteristic (e.g., phenotype or genotype) following genetic modification may be selected and independently expanded.

In some embodiments, the hematopoietic cells are genetically engineered to be deficient in a target antigen, e.g., a cell-surface lineage-specific antigen. In some embodiments, the hematopoietic cells are genetically engineered to be deficient in the same target antigen (e.g., cell-surface lineage-specific antigen) that is targeted by the CARs described herein. In some embodiments, the hematopoietic cells are genetically engineered to be deficient in CD33. In some embodiments, the hematopoietic cells are genetically engineered to be deficient in a domain of CD33. In some embodiments, the hematopoietic cells are genetically engineered to be deficient in the IgV domain of CD33. In some embodiments, the hematopoietic cells are genetically engineered to be deficient in the IgC2 domain of CD33. In some embodiments, the hematopoietic cells are genetically engineered to be deficient in the immunoglobulin C domain of CD33.

As used herein, a hematopoietic cell is considered to be deficient in a target antigen (e.g., a cell-surface lineage-specific antigen) if the hematopoietic cell has substantially reduced expression of the target antigen (e.g., a cell-surface lineage-specific antigen) as compared to a naturally-occurring hematopoietic cell of the same type as the genetically engineered hematopoietic cell (e.g., is characterized by the presence of the same cell surface markers, such as CD34). In some embodiments, the hematopoietic cell has no detectable expression of the target antigen (e.g., a cell-surface lineage-specific antigen). The expression level of a target antigen (e.g., a cell-surface lineage-specific antigen) can be assessed by any means known in the art. For example, the expression level of a target antigen (e.g., a cell-surface lineage-specific antigen) can be assessed by detecting the antigen with an antigen-specific antibody (e.g., flow cytometry methods, Western blotting) and/or by measuring the level of a transcript encoding the antigen (e.g., RT-qPCR, microarray).

In some embodiments, the expression of the target antigen (e.g., a cell-surface lineage-specific antigen) on the genetically engineered hematopoietic cell is compared to the expression of the target antigen (e.g., a cell-surface lineage-specific antigen) on a naturally occurring hematopoietic cell. In some embodiments, the genetic engineering results in a reduction in the expression level of the target antigen (e.g., a cell-surface lineage-specific antigen) by at least about 50%, 60%, 70%, 80%, 90%, 91%, 92%, 93%, 94%, 95%, 96%, 97%, 98%, or 99% as compared to the expression of the target antigen (e.g., a cell-surface lineage-specific antigen) on a naturally occurring hematopoietic cell.

In some embodiments, the hematopoietic cell is deficient in the whole endogenous gene encoding the target antigen (e.g., a cell-surface lineage-specific antigen). In some embodiments, the whole endogenous gene encoding the target antigen (e.g., a cell-surface lineage-specific antigen) has been deleted. In some embodiments, the hematopoietic cell comprises a portion of endogenous gene encoding the target antigen (e.g., a cell-surface lineage-specific antigen). In some embodiments, the hematopoietic cell expressing a portion (e.g. a truncated protein) of the target antigen (e.g., a cell-surface lineage-specific antigen). In other embodiments, a portion of the endogenous gene encoding the target antigen (e.g., a cell-surface lineage-specific antigen) has been deleted. In some embodiments, at least 10%, 20%, 30%, 40%, 50%, 60%, 70% or more of the gene encoding the target antigen (e.g., a cell-surface lineage-specific antigen) has been deleted.

As will be appreciated by one of ordinary skill in the art, a portion of the nucleotide sequence encoding the target antigen (e.g., a cell-surface lineage-specific antigen) may be deleted or one or more non-coding sequences, such that the hematopoietic cell is deficient in the antigen (e.g., has substantially reduced expression of the antigen).

In some embodiments, the target antigen (e.g., a cell-surface lineage-specific antigen is CD33. The predicted structure of CD33 includes two immunoglobulin domains, an IgV domain and an IgC2 domain. In some embodiments, a portion of the immunoglobulin C domain of CD33 is deleted.

Any of the genetically engineering hematopoietic cells, such as HSCs, that are deficient in a a target antigen (e.g., a cell-surface lineage-specific antigen) can be prepared by a routine method or by a method described herein. In some embodiments, the genetic engineering is performed using genome editing. As used herein, “genome editing” refers to a method of modifying the genome, including any protein-coding or non-coding nucleotide sequence, of an organism to knock-out the expression of a target gene. In general, genome editing methods involve use of an endonuclease that is capable of cleaving the nucleic acid of the genome, for example at a targeted nucleotide sequence. Repair of the double-stranded breaks in the genome may be repaired introducing mutations and/or exogenous nucleic acid may be inserted into the targeted site.

Genome editing methods are generally classified based on the type of endonuclease that is involved in generating double stranded breaks in the target nucleic acid. These methods include use of zinc finger nucleases (ZFN), transcription activator-like effector-based nuclease (TALEN), meganucleases, and CRISPR/Cas systems. Methods of editing the genome of HSCs described herein can be found, e.g., in WO 2017/066760, incorporated by reference herein.

Combination Therapy

As described herein, any of the CARs comprising an antigen-binding domain that binds to a cell-surface lineage-specific antigen (e.g., CD33 CAR), nucleic acids, vectors, cells expressing any of the CARs, and/or pharmaceutical compositions described herein may be administered to a subject in combination with hematopoietic cells that are deficient for the antigen (e.g., cell-surface lineage-specific antigen (i.e., CD33)).

In some embodiments, the agents and/or the hematopoietic cells may be mixed with a pharmaceutically acceptable carrier to form a pharmaceutical composition, which is also within the scope of the present disclosure.

To perform the methods described herein, an effective amount of the any of the CARs that target CD33, nucleic acids, vectors, cells expressing any of the CARs, and/or pharmaceutical compositions described herein and an effective amount of hematopoietic cells can be co-administered to a subject in need of the treatment.

As described herein, the hematopoietic cells and/or cells expressing chimeric antigen receptors (e.g., immune cells) may be autologous to the subject. i.e., the cells are obtained from the subject in need of the treatment, genetically engineered to be deficient for expression of the target antigen (e.g., cell-surface lineage-specific antigen) or for expression of the chimeric antigen receptor constructs, and then administered to the same subject. Administration of autologous cells to a subject may result in reduced rejection of the host cells as compared to administration of non-autologous cells. Alternatively, the hematopoietic cells and/or cells expressing chimeric antigen receptors (e.g., immune cells) are allogeneic cells, i.e., the cells are obtained from a first subject, genetically engineered to be deficient for expression of the target antigen (e.g., cell-surface lineage-specific antigen) or for expression of the chimeric antigen receptor constructs, and administered to a second subject that is different from the first subject but of the same species. For example, allogeneic immune cells may be derived from a human donor (e.g., a healthy donor) and administered to a human recipient who is different from the donor.

In some embodiments, the cells (e.g., immune cells) expressing any of the CARs described herein and/or hematopoietic cells are allogeneic cells and have been further genetically engineered to reduced graft-versus-host disease. For example, as described herein, the hematopoietic stem cells may be genetically engineered (e.g., using genome editing) to have reduced expression of CD45RA.

In some embodiments, the cells (e.g., immune cells) expressing any of the chimeric antigen receptors described herein are administered to a subject in an amount effective in to reduce the number of target cells (e.g., cancer cells, malignant cells) by least 20%, e.g., 50%, 80%, 100%, 2-fold, 5-fold, 10-fold, 20-fold, 50-fold, 100 fold or more.

A typical amount of cells, i.e., cells (e.g., immune cells) expressing any of the CARs described herein or hematopoietic cells, administered to a mammal (e.g., a human) can be, for example, in the range of one million to 100 billion cells; however, amounts below or above this exemplary range are also within the scope of the present disclosure. For example, the daily dose of cells can be about 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), preferably about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), more preferably about 100 million cells to about 50 billion cells (e.g., about 120 million cells, about 350 million cells, about 350 million cells, about 450 million cells, about 650 million cells, about 800 million cells, about 900 million cells, about 3 billion cells, about 30 billion cells, about 45 billion cells, or a range defined by any two of the foregoing values).

In some embodiments, the chimeric receptor (e.g., a nucleic acid encoding the chimeric receptor) is introduced into a cell (e.g., an immune cell), and the subject (e.g., human patient) receives an initial administration or dose of the cells expressing the chimeric antigen receptor. One or more subsequent administrations of the cells expressing the chimeric antigen receptor may be provided to the patient at intervals of 15 days, 14, 13, 12, 11, 10, 9, 8, 7, 6, 5, 4, 3, or 2 days after the previous administration. More than one dose of the cells expressing the chimeric antigen receptor can be administered to the subject per week, e.g., 2, 3, 4, or more administrations of the cells. The subject may receive more than one doses of the cells (e.g., an immune cell expressing a chimeric receptor) per week, followed by a week of no administration of the cells, and finally followed by one or more additional doses of the cells (e.g., more than one administration of immune cells expressing a chimeric receptor per week). The cells (e.g., immune cells) expressing a chimeric antigen receptor may be administered every other day for 3 administrations per week for two, three, four, five, six, seven, eight or more weeks.

In some embodiments, the methods involve administration of cells (e.g., immune cells) expressing the CAR targeting CD33 and a population of hematopoietic cells deficient in the antigen (e.g., CD33). Accordingly, in such therapeutic methods, the CAR recognizes (binds) a target cell expressing the target antigen for targeting killing. The hematopoietic cells that are deficient in the target antigen allow for repopulation of a cell type that is targeted by the cells/CARs. In some embodiments, the treatment of the patient can involve the following steps: (1) administering a therapeutically effective amount of cells (e.g., immune cells) expressing the CAR targeting CD33 to the patient and (2) infusing or reinfusing the patient with hematopoietic stem cells, either autologous or allogenic, where the hematopoietic cells have reduced expression of the target antigen. In some embodiments, the treatment of the patient can involve the following steps: (1) administering a therapeutically effective amount of cells (e.g., an immune cell) expressing a chimeric antigen receptor to the patient, wherein the cell comprises a nucleic acid sequence encoding a chimeric antigen receptor that binds a cell-surface lineage-specific, disease-associated antigen (i.e., CD33); and (2) infusing or reinfusing the patient with hematopoietic cells (e.g., hematopoietic stem cells), either autologous or allogenic, where the hematopoietic cells have reduced expression of a lineage specific disease-associated antigen (i.e., CD33).

The efficacy of the therapeutic methods using any of the CARs, nucleic acids, vectors, cells expressing any of the CARs, or pharmaceutical compositions comprising antigen any of the foregoing described herein and a population of hematopoietic cells deficient in the target antigen may be assessed by any method known in the art and would be evident to a skilled medical professional. For example, the efficacy of the therapy may be assessed by survival of the subject or cancer burden in the subject or tissue or sample thereof. In some embodiments, the efficacy of the therapy is assessed by quantifying the number of cells belonging to a particular population or lineage of cells. In some embodiments, the efficacy of the therapy is assessed by quantifying the number of cells presenting the target antigen.

In some embodiments, the CARs, nucleic acids, vectors, cells expressing any of the CARs, or pharmaceutical compositions comprising any of the foregoing described herein and the population of hematopoietic cells is administered concomitantly.

In some embodiments, any of the CARs, nucleic acids, vectors, cells expressing any of the CARs, or pharmaceutical compositions comprising is any of the foregoing described herein are is administered prior to administration of the hematopoietic cells. In some embodiments, any of the CARs, nucleic acids, vectors, cells expressing any of the CARs, or pharmaceutical compositions comprising any of the foregoing described herein are administered at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more prior to administration of the hematopoietic cells.

In some embodiments, the hematopoietic cells are administered prior to the any of the CARs, nucleic acids, vectors cells expressing any of the CARs, or pharmaceutical compositions comprising any of the foregoing described herein. In some embodiments, the population of hematopoietic cells is administered at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more prior to administration of any of the CARs, nucleic acids, vectors, cells expressing any of the CARs, or pharmaceutical compositions comprising any of the foregoing described herein.

In some embodiments, any of the CARs, nucleic acids, vectors, cells expressing any of the CARs, or pharmaceutical compositions comprising any of the foregoing described herein and the population of hematopoietic cells are administered at substantially the same time. In some embodiments, any of the CARs, nucleic acids, vectors, cells expressing any of the CARs, or pharmaceutical compositions comprising any of the foregoing described herein is administered and the patient is assessed for a period of time, the population of hematopoietic cells is administered and the patient is assessed for a period of time, after which any of the CARs, nucleic acids, vectors, cells expressing any of the CARs, or pharmaceutical compositions comprising any of the foregoing described herein is administered.

Also within the scope of the present disclosure are multiple administrations (e.g., doses) of any of the CARs, nucleic acids, vectors, cells expressing any of the CARs, or pharmaceutical compositions comprising any of the foregoing described herein and/or populations of hematopoietic cells. In some embodiments, any of the CARs, nucleic acids, vectors, cells expressing any of the CARs, or pharmaceutical compositions comprising any of the foregoing described herein and/or populations of hematopoietic cells are administered to the subject once. In some embodiments, any of the CARs, nucleic acids, vectors, cells expressing any of the CARs, or pharmaceutical compositions comprising any of the foregoing described herein and/or populations of hematopoietic cells are administered to the subject more than once (e.g., at least 2, 3, 4, 5, or more times). In some embodiments, any of the CARs, nucleic acids, vectors, cells expressing any of the CARs, or pharmaceutical compositions comprising any of the foregoing described herein and/or populations of hematopoietic cells are administered to the subject at a regular interval, e.g., every six months.

In some embodiments, the subject is a human subject having a hematopoietic malignancy or pre-malignancy. In some embodiments, the subject is a human subject that has been diagnosed with a hematopoietic malignancy or pre-malignancy. As used herein a hematopoietic malignancy refers to a malignant abnormality involving hematopoietic cells (e.g., blood cells, including progenitor and stem cells). Examples of hematopoietic malignancies and or pre-malignancies include, without limitation, Hodgkin's lymphoma, non-Hodgkin's lymphoma, leukemia, or multiple myeloma. Leukemias include acute myeloid leukemia (AML), myelodysplastic syndrome (MDS), chronic myelogenous leukemia, chronic lymphoblastic leukemia, and chronic lymphoid leukemia. In some embodiments, the hematopoietic malignancy is acute myeloid leukemia (AML). In some embodiments, the hematopoietic malignancy is myelodysplastic syndrome (MDS).

Kits for Therapeutic Uses

Also within the scope of the present disclosure are kits for use any of the CARs, nucleic acids, vectors, and/or cells expressing any of the CARs described herein in combination with populations of hematopoietic cells that are deficient in a target antigen (e.g., the cell-surface lineage-specific antigen (e.g., CD33)). Such kits may include one or more containers comprising a first pharmaceutical composition that comprises any of the CARs, nucleic acids, vectors, and/or cells expressing any of the CARs described herein, and a pharmaceutically acceptable carrier, and a second pharmaceutical composition that comprises a population of hematopoietic cells that are deficient in a target antigen (i.e., CD33), or a portion thereof, and a pharmaceutically acceptable carrier.

In some embodiments, the kit can comprise instructions for use in any of the methods described herein. The included instructions can comprise a description of administration of the first and second pharmaceutical compositions to a subject to achieve the intended activity in a subject. The kit may further comprise a description of selecting a subject suitable for treatment based on identifying whether the subject is in need of the treatment. In some embodiments, the instructions comprise a description of administering the first and second pharmaceutical compositions to a subject who is in need of the treatment.

The instructions relating to the use of the CARs, nucleic acids, vectors, and/or cells expressing any of the CARs described herein and the first and second pharmaceutical compositions described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port. At least one active agent in the pharmaceutical composition is a chimeric receptor variants as described herein.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### NFAT-Responsive Reporter Systems

Aspects of the present disclosure relate to nucleic acid constructs comprising a minimal nuclear factor of activated T cells (NFAT)-responsive promoter, which may be used, for example, to assess chimeric antigen receptors (CARs) and activation of a cell (e.g., T cells) expressing the CARs. CAR activation sets in motion an intracellular pathway leading to T-cell activation and effector function of the T cell, which involves NFAT signaling and gene expression (see, e.g., Hogan, Cell Calcium. (2017)63:66-9). As used herein, the term “NFAT-responsive promoter” refers to a promoter region that is activated by NFAT signaling and promotes expression of a gene that is operably linked to the NFAT-responsive promoter upon activation. In some embodiments, the gene that is operably linked (under control of) the NFAT-responsive promoter encodes a reporter molecule.

Nuclear factor of activated T-cells (NFAT) is a family of transcription factors, include NFAT1-NFAT-5, that are involved regulating immune responses, including regulating interleukin-2 (IL-2 expression) as well as T cell differentiation and self-tolerance. See, e.g., Macian Nat. Rev. Immunol. (2005) 5: 472-484. NFAT transcription factors comprise two components: a cytoplasmic Rel domain protein (NFAT family member) and a nuclear component comprising various transcription factors (Chow, Molecular and Cellular Biology, 1999; 19(3):2300-7). NFAT1 and NFAT2 are predominantly expressed in peripheral T cells that produce IL-2 and NFAT binding sites are generally found upstream (5′) of NFAT-regulated genes, such as IL-2. See, e.g., Chow, Molecular and Cellular Biology, (1999) 19(3):2300-7; Rooney et al., Molecular and Cellular Biology, (1995) 15(11):6299-310; and Shaw et al., Journal of Immunology, (2010) 185(9):4972-5, the entire contents of which are incorporated herein by reference.

As will be understood by one of ordinary skill in the art, in eukaryotic cells, a promoter operably linked to a gene typically includes a core promoter adjacent and 5′ to the transcription start site of the gene (coding sequence). Further upstream (5′) of the core promoter may be cis-regulatory regions, such as transcription factor binding site(s).

In some embodiments, the NFAT-responsive promoter comprises a plurality of NFAT-binding sites. In some embodiments, the NFAT-responsive promoter comprises least 1, 2, 3, 4, 5, 6, 7, 8, 9, 10, 11, 12, 13, 14, 15, 16, 17, 18, 19, 20 or more NFAT binding sites. In some embodiments, the NFAT-responsive promoter comprises six NFAT binding sites. In some embodiments, each of the NFAT binding sites of a NFAT-responsive promoter may be the same NFAT binding site (e.g., bind the same type of NFAT transcription factor) or be different NFAT binding sites (e.g., bind different types of NFAT transcription factors). In some embodiments, each of the NFAT binding site comprises the same nucleotide sequence. In some embodiments, the NFAT binding sites comprise different nucleotide sequence.

An example of a NFAT binding site is provided by the nucleotide sequence provided by SEQ ID NO: 84:

(SEQ ID NO: 84)
5′-GGAGGAAAAACTGTTTCATACAGAAGGCGT-3′.

In some embodiments, at least one of the NFAT binding site comprises the nucleotide sequence of SEQ ID NO: 84. In some embodiments, each of the NFAT binding site comprises the nucleotide sequence of SEQ ID NO: 84.

Each of the NFAT binding sites are located immediately adjacent to one another (e.g., in tandem without any additional nucleotides between the NFAT binding sites). Alternatively, one or more additional nucleotides may be present between two or more of the NFAT binding sites.

In some embodiments, the NFAT-responsive promoter comprises an IL-2 promoter, or portion thereof. In some embodiments, the NFAT-responsive promoter comprises a minimal IL-2 promoter. In some embodiments, the NFAT-responsive promoter comprises the core IL-2 promoter. In general, the naturally occurring IL-2 promoter is relative compact and includes a core promoter containing a TATA box and an upstream regulatory region. The core promoter is considered the region within approximately −40 and +40 nucleotides (e.g., 40 nucleotides upstream (5′) to 40 nucleotides downstream (3′)) of the transcription start site. See, e.g., Weaver et al. Mol. Immunol. (2007) 44(11) 2813-2819.

As used herein, the term “minimal IL-2 promoter” refers to the minimal portion of the IL-2 promoter requires for transcription. In some embodiments, the minimal IL-2 promoter is the IL-2 core promoter. In some embodiments, the NFAT-responsive promoter comprises the core IL-2 promoter comprising a TATA box. A TATA box (also referred to as a “Goldberg-Hogness box”) is a T/A rich sequence found upstream of a transcriptional start site (Shi & Zhou, BMC Bioinformatics (2006) 7, Article number S2). In some embodiments, the TATA box comprises the consensus sequence 5′-TATA(A/T)A(A/T)-3′. The TATA box is thought to be involved in formation of the preinitiation complex for gene transcription and bind a TATA-binding protein (TBP).

In some embodiments, the minimal IL-2 promoter comprises the nucleotide sequence of SEQ ID NO: 85.

An example of a minimal IL-2 promoter is provided by the nucleotide sequence provided by SEQ ID NO: 85:

(SEQ ID NO: 85)
5′-TAGAGGGTATATAATGGAAGCTCGAATTCCA-3′.

In some embodiments, the NFAT binding sites are located 5′ (upstream) of the minimal IL-2 promoter. In some embodiments, the NFAT binding sites are located at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 41, 42, 43, 44, 45, 46, 47, 48, 49, 50, or more nucleotides 5′ (upstream) of the minimal IL-2 promoter. In some embodiments, the NFAT responsive promoter comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 41, 42, 43, 44, 45, 46, 47, 48, 49, 50, or more nucleotides between the last NFAT binding site and the minimal IL-2 promoter.

An exemplary nucleotide sequence of a minimal NFAT-responsive promoter is provided by SEQ ID NO: 86. In some embodiments, the nucleotide sequence of the minimal NFAT-responsive promoter comprises, consists of, or consists essentially of the nucleotide sequence of SEQ ID NO: 86, or a sequence that is at least 70%, at least 75%, at least 80%, at least 85%, at least 90%, at least 95%, or at least 99% identical the nucleotide sequence of SEQ ID NO: 86.

Exemplary nucleotide sequence of a minimal NFAT-responsive promoter comprising 6 NFAT binding sites (SEQ ID NO: 86):

(SEQ ID NO: 86)
5′-
GGAGGAAAAACTGTTTCATACAGAAGGCGTGGAGGAAAAACTGTTTCATACAGAAGGCGTGGAGGAAAAACTGTT
TCATACAGAAGGCGTGGAGGAAAAACTGTTTCATACAGAAGGCGTGGAGGAAAAACTGTTTCATACAGAAGGCGT
GGAGGAAAAACTGTTTCATACAGAAGGCGTGATCTAGACTTAGAGGGTATATAATGGAAGCTCGAATTCCA-3′.

Any of the nucleic acid constructs encoding an IL-2 reporter system described herein may further comprise a nucleotide sequence encoding a second reporter molecule operably linked (under the control of) a constitutive promoter (also referred to as a constitutively active promoter). Preferably, the reporter molecule that is operably linked to the minimal NFAT-responsive promoter is different than the second reporter molecule operably linked to the constitutively active promoter, such that detection of the reporter molecule that is operably linked to the minimal NFAT-responsive promoter is indicative of activity of the NFAT-responsive promoter and detection of the reporter molecule that is operably linked to the constitutively active promoter is indicative of activity of the constitutively active promoter.

In some embodiments, the constitutive promoter controlling expression of the second reporter molecule is referred to as a “reference promoter.” Examples of constitutively active promoter include, without limitation, EF-1alpha (EF1a), CMV promoter, SV40 promoter, PGK1 promoter, Ubc promoter, beta actin promoter, CAG promoter, TRE promoter, UAS promoter, Ac5 promoter, polyhedrin promoter, and U6 promoter. In some embodiments, the constitutively active promoter is an EF1a promoter.

The nucleotide sequence of an elongation factor 1 alpha (EF-1alpha) promoter is provided by the nucleotide sequence of SEQ ID NO: 87.

EF1 alpha promoter
(SEQ ID NO: 87)
GGCTCCGGTGCCCGTCAGTGGGCAGAGCGCACATCGCCCACAGTCCCCGAGAAGTTGGGGGGAGGGGTCGGCAAT
TGATCCGGTGCCTAGAGAAGGTGGCGCGGGGTAAACTGGGAAAGTGATGTCGTGTACTGGCTCCGCCTTTTTCCC
GAGGGTGGGGGAGAACCGTATATAAGTGCAGTAGTCGCCGTGAACGTTCTTTTTCGCAACGGGTTTGCCGCCAGA
ACACAGGTAAGTGCCGTGTGTGGTTCCCGCGGGCCTGGCCTCTTTACGGGTTATGGCCCTTGCGTGCCTTGAATT
ACTTCCACCTGGCTGCAGTACGTGATTCTTGATCCCGAGCTTCGGGTTGGAAGTGGGTGGGAGAGTTCGAGGCCT
TGCGCTTAAGGAGCCCCTTCGCCTCGTGCTTGAGTTGAGGCCTGGCCTGGGCGCTGGGGCCGCCGCGTGCGAATC
TGGTGGCACCTTCGCGCCTGTCTCGCTGCTTTCGATAAGTCTCTAGCCATTTAAAATTTTTGATGACCTGCTGCG
ACGCTTTTTTTCTGGCAAGATAGTCTTGTAAATGCGGGCCAAGATCTGCACACTGGTATTTCGGTTTTTGGGGCC
GCGGGCGGCGACGGGGCCCGTGCGTCCCAGCGCACATGTTCGGCGAGGCGGGGCCTGCGAGCGCGGCCACCGAGA
ATCGGACGGGGGTAGTCTCAAGCTGGCCGGCCTGCTCTGGTGCCTGGCCTCGCGCCGCCGTGTATCGCCCCGCCC
TGGGCGGCAAGGCTGGCCCGGTCGGCACCAGTTGCGTGAGCGGAAAGATGGCCGCTTCCCGGCCCTGCTGCAGGG
AGCTCAAAATGGAGGACGCGGCGCTCGGGAGAGCGGGCGGGTGAGTCACCCACACAAAGGAAAAGGGCCTTTCCG
TCCTCAGCCGTCGCTTCATGTGACTCCACGGAGTACCGGGCGCCGTCCAGGCACCTCGATTAGTTCTCGAGCTTT
TGGAGTACGTCGTCTTTAGGTTGGGGGGAGGGGTTTTATGCGATGGAGTTTCCCCACACTGAGTGGGTGGAGACT
GAAGTTAGGCCAGCTTGGCACTTGATGTAATTCTCCTTGGAATTTGCCCTTTTTGAGTTTGGATCTTGGTTCATT
CTCAAGCCTCAGACAGTGGTTCAAAGTTTTTTTCTTCCATTTCAGGTGTCGTGA

The nucleic acid constructs described herein comprise a reporter molecule operably linked (under control of) a minimal NFAT-responsive promoter. In some embodiments, the nucleic acid construct comprises a second reporter molecule operably linked (under control of) a constitutively active promoter. Any suitable reporter molecule(s) may be used in the nucleic acid constructs described herein. Preferably a reporter molecule (a reporter protein) is readily detectable (directly or indirectly) upon expression. In some embodiments, the reporter molecule may be referred to as a screenable marker. Examples of reporter molecules include, without limitation, enzymes, such as β-glucuronidase, α-galactosidase, β-lactamase, and tyrosinase; luciferase; fluorescent markers/proteins. Fluorescent proteins include, but are not limited to, green fluorescent protein (GFP), red fluorescent protein (RFP), blue fluorescent protein (BFP), EBFP, cyan fluorescent protein, ECFP, EG fluorescent protein-yellow fluorescent protein, mWasabi, ZsGreen, yellow fluorescent protein (YFP), ZsYellow, mHoneydew, mApple, mRuby, mBanana, mOrange, mCherry, mCerulean, mTurquoise, mTangerine, mStrawberry, mGrape, mRaspberry, and mPlum. Selection of a suitable reporter molecule, such as a fluorescent protein, may depend on factors such as the means for detecting and/or quantifying the reporter molecule.

The nucleic acid constructs described herein comprise a reporter molecule operably linked (under control of) a minimal NFAT-responsive promoter. In some embodiments, the nucleic acid construct comprises a second reporter molecule operably linked (under control of) a constitutively active promoter. Any suitable reporter molecule(s) may be used in the nucleic acid constructs described herein. Preferably a reporter molecule (a reporter protein) is readily detectable (directly or indirectly) upon expression. In some embodiments, the reporter molecule may be referred to as a screenable marker. Examples of reporter molecules include, without limitation, enzymes, such as β-glucuronidase, α-galactosidase, β-lactamase, and tyrosinase; luciferase; fluorescent markers/proteins. Fluorescent proteins include, but are not limited to, green fluorescent protein (GFP), red fluorescent protein (RFP), blue fluorescent protein (BFP), EBFP, cyan fluorescent protein, ECFP, EG fluorescent protein-yellow fluorescent protein, mWasabi, ZsGreen, yellow fluorescent protein (YFP), ZsYellow, mHoneydew, mApple, mRuby, mBanana, mOrange, mCherry, mCerulean, mTurquoise, mTanerine, mStrawberry, mGrape, mRaspberry, and mPlum. Selection of a suitable reporter molecule, such as a fluorescent protein, may depend on factors such as the means for detecting and/or quantifying the reporter molecule.

In some embodiments, the reporter molecule is a fluorescent protein. In some embodiments, the reporter molecule operably linked to the NFAT-responsive promoter is a fluorescent protein. In some embodiments, fluorescent protein is mTurquoise or mOrange.

A nucleotide sequence encoding mTurquoise is provided by SEQ ID NO: 88.

mTurquoise.
(SEQ ID NO: 88)
ATGGTGAGCAAGGGCGAGGAGCTGTTCACCGGGGTGGTGCCCATCCTGGTCGAGCTGGACGGCGACGTAAACGGC
CACAAGTTCAGCGTGTCCGGCGAGGGCGAGGGCGATGCCACCTACGGCAAGCTGACCCTGAAGTTCATCTGCACC
ACCGGCAAGCTGCCCGTGCCCTGGCCCACCCTCGTGACCACCCTGTCCTGGGGCGTGCAGTGCTTCGCCCGCTAC
CCCGACCACATGAAGCAGCACGACTTCTTCAAGTCCGCCATGCCCGAAGGCTACGTCCAGGAGCGCACCATCTTC
TTCAAGGACGACGGCAACTACAAGACCCGCGCCGAGGTGAAGTTCGAGGGCGACACCCTGGTGAACCGCATCGAG
CTGAAGGGCATCGACTTCAAGGAGGACGGCAACATCCTGGGGCACAAGCTGGAGTACAACTACTTTAGCGACAAC
GTCTATATCACCGCCGACAAGCAGAAGAACGGCATCAAGGCCAACTTCAAGATCCGCCACAACATCGAGGACGGC
GGCGTGCAGCTCGCCGACCACTACCAGCAGAACACCCCCATCGGCGACGGCCCCGTGCTGCTGCCCGACAACCAC
TACCTGAGCACCCAGTCCAAGCTGAGCAAAGACCCCAACGAGAAGCGCGATCACATGGTCCTGCTGGAGTTCGTG
ACCGCCGCCGGGATCACTCTCGGCATGGACGAGCTGTACAAG

A nucleotide sequence encoding mOrange is provided by SEQ ID NO: 89.

mOrange
(SEQ ID NO: 89)
ATGGTGAGCAAGGGCGAGGAGAATAACATGGCCATCATCAAGGAGTTCATGCGCTTCAAGGTGCGCATGGAGGGC
TCCGTGAACGGCCACGAGTTCGAGATCGAGGGCGAGGGCGAGGGCCGCCCCTACGAGGGCTTTCAGACCGCTAAG
CTGAAGGTGACCAAGGGTGGCCCCCTGCCCTTCGCCTGGGACATCCTGTCCCCTCATTTCACCTACGGCTCCAAG
GCCTACGTGAAGCACCCCGCCGACATCCCCGACTACTTCAAGCTGTCCTTCCCCGAGGGCTTCAAGTGGGAGCGC
GTGATGAACTACGAGGACGGCGGCGTGGTGACCGTGACCCAGGACTCCTCCCTGCAGGACGGCGAGTTCATCTAC
AAGGTGAAGCTGCGCGGCACCAACTTCCCCTCCGACGGCCCCGTGATGCAGAAGAAGACCATGGGCTGGGAGGCC
TCCTCCGAGCGGATGTACCCCGAGGACGGTGCCCTGAAGGGCAAGATCAAGATGAGGCTGAAGCTGAAGGACGGC
GGCCACTACACCTCCGAGGTCAAGACCACCTACAAGGCCAAGAAGCCCGTGCAGCTGCCCGGCGCCTACATCGTC
GACATCAAGTTGGACATCACCTCCCACAACGAGGACTACACCATCGTGGAACAGTACGAACGCGCCGAGGGCCGC
CACTCCACCGGCGGCATGGACGAGCTGTACAAG

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.): Handbook of Experimental Immunology (D. M. Weir and C. C. Blackwell, eds.): Gene Transfer Vectors for Mammalian Cells (J. M. Miller and M. P. Calos, eds., 1987; Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: Generation and Evaluation of CAR Constructs

### CAR Constructs

CAR constructs are developed with CD33 specific single chain fragment variable sequences (scFv) or single domain antibody fragments (sdAb), linked with either a CD8a or CD28 transmembrane domain, paired with either a 4-1BB or CD28 co-stimulatory domain, and a CD3 (zeta) signaling domain. The CAR sequences were cloned in a third-generation lentiviral plasmid. The scFv or sdAb of the CD33 CAR constructs were derived from the following:
Lintuzumab (Hu195, SGN-33) (Co et al., J. Immunol. (1992), 148: 1149-54 (1992)) M195, which is the non-humanized version of Lintuzumab.
   CD33Mylo (gemtuzumab ozogamicin, Trade name: Mylotarg, Company: Wyeth, humanized mAb/calicheamicin, CD33; U.S. Pat. No. 5,739,116; Cowan et al., Front Biosci (Landmark Ed) (2013), 18: 1311-34). Also noted, in some cases, as “hP67.6”.

M9.6 Binding domain. Three configurations using the antigen binding domain of M9.6 were evaluated and included the following:

(i) VH-CDR3:
(SEQ ID NO: 27)
LGGSLPDYGMDV
(ii) VH-CDR3:
(SEQ ID NO: 31)
RGGYSDYDYYFDF

(iii) VL-VH orientation (swapped)

M2H12 Binding domain (scFv)

DRB2 Binding domain (scFv)

CAR33VH, which is a VH only binder

The following CAR constructs generated are shown in the amino acid sequences SEQ ID NOs: 10, 13, 16, 19, 22, 25, 29, 33, 36, 39, 42, 45, 48, 51, 54, and 57 and are encoded by the nucleic acid sequences shown in SEQ ID NOs: 9, 12, 15, 18, 21, 24, 28, 32, 35, 38, 41, 44, 47, 50, 53, and 56.

These CARs were subcloned into a lentiviral vector backbone. All restriction enzymes were purchased from New England Biolabs (Ipswich, MA, USA). The sequences of all CAR constructs was confirmed by sequencing at Macrogen (Rockville, MD, USA). The full vector sequences for each construct generated are shown in SEQ ID NOs: 11, 14, 17, 20, 23, 26, 30, 34, 37, 40, 43, 46, 49, 52, 55, and 58.

### Cell Lines

The GFP and luciferase expressing AML cells lines MV411, THP1, and MOLM14 contain varying levels of CD33 expression, and different genotypes for an exon 2 splice variance (Laszlo et al., Oncotarget, 7: 43281-94 (2016)) will be used to test the efficacy of the CAR constructs described above. Through DNA isolation, it was found that MOLM14 has a CC genotype and does not contain the SNP, while TE1P1 and MV411 are both heterozygous for the SNP with the CT genotype (Lamba et al., J. Clin. Oncol., 35: 2674-82 (2017)). This cell line does not express neither CD33 nor CD123. MV411 is an acute monocytic leukemia line established from a 10-year-old boy with acute monocytic leukemia (AML FAB M5). MOLM14 is an acute myeloid leukemia line established from the peripheral blood of a 20-year-old man with acute myeloid leukemia AML FAB M5a at relapse in 1995 after initial myelodysplastic syndrome (MDS, refractory anemia with excess of blasts, RAEB). THP-1 is a human monocytic cell line derived from an acute monocytic leukemia patient. K562 is a human erythroleukemia leukemia line established and derived from a 53-year-old female chronic myelogenous leukemia patient.

### CAR T-Cell Generation

The CD33 CAR-encoding lentiviral vectors are produced by transient transfection of the Lenti-X 293T lenti packaging cell line Lenti-X 293T cells and plated into poly-D lysine coated 15-cm plates (BD Biosciences, San Jose, CA, USA). The following day, Lenti-X 293T cells are transfected using lipofectamine 3000 (Thermo Fisher Scientific, Waltham, MA, USA) with plasmids encoding the CAR along with packaging and envelope vectors (pMDLg/pRRE, pMD-2G, and pRSV-Rev). Lentiviral supernatants are harvested at 24 and 48 hours post-transfection, centrifuged at 3000 RPM for 10 minutes to remove cell debris, and frozen on dry ice and stored at −80° C. Human PBMCs from normal donors are obtained with an NIH-approved protocol and activated with a 1:3 ratio of CD3/CD28 microbeads (Dynabeads Human T-Expander CD3/CD28, Thermo Fisher Scientific, Cat #11141D) in AIM-V media containing 40 IU/mL recombinant IL-2 and 5% FBS for 24 hours. Activated T cells are resuspended at 2 million cells per 2 mL of lentiviral supernatant plus 1 mL of fresh AIM-V media with 10 mcg/mL protamine sulfate and 100 IU/mL IL-2 in 6-well plates. Plates were centrifuged at 1000×g for 2 hours at 32° C. and incubated overnight at 37° C. A second transduction is performed on the following day by repeating the same transduction procedure described above. The CD3/CD28 beads were removed on the third day following transduction, and the cells were cultured at 300,000 cells/mL in AIM-V containing 100 IU/mL IL2 with fresh IL2-containing media added every 2-3 days until harvest on day 8 or 9.

### Flow Cytometry

Surface expression of CD33 CAR-transduced T cells is determined by flow cytometry using either protein-L (Themo Fisher) or a Biotinylated Human Siglec-3/CD33 Protein (Aero Biosystems, Newark, DE, USA) followed by incubation with Streptavidin-PE (BioLegend, San Diego, CA, USA).

### PDX

1 million cells of a PDX leukemia cell line JMM117 are injected into the NSG mice one week ahead of adoptive CAR T cell transfer. The mice are treated with CAR T cells on day 0. Two weeks later the mice are taken down and analysis is performed.

### Cytotoxicity Assay

5E4 of Target tumor cells in 100 μl of RPMI media are loaded into a 96-well plate (Corning® (Croning, NY) BioCoat™ Poly-L-Lysine 96-Well Clear TC-Treated Flat Bottom Assay Plate). An equal amount of CAR T cells are added into the designated well on the following day. The initial incucyte apoptosis marker (Essen BioScience, Ann Arbor, MI, USA) is diluted in 100 μl PBS and 1 μl of the diluent was added into each well. The plate is scanned for the GFP and or RFP fluorescent expression to monitor the cell apoptosis using an IncuCyte ZOOM® system every 30 minutes in a duration of 40 hours. The percentage of cell killing at each time point is baseline-corrected.

### Analysis of Cytokine Production

Target tumor cell and transduced CAR positive T cells are washed 3 times with 1×PBS and resuspended in RPMI at 1E6/ml. 100 μl of tumor cells with 100 μl of CAR positive T cells are loaded into each well of a 96-well plate. T cell only and tumor cell only controls are set up. All tests are performed in duplicate or triplicate. Cells are incubated for 18 hours at 37° C. and 120 ul of the culture supernatant was harvested for detection of cytokine production. Cytokine levels in supernatants were measured using either ELISA kits (R&D Systems, Minneapolis, MN, EISA) or a multiplex assay (Meso Scale Discovery, Rockville, MD, EISA).

### Bioenergetic Analyses

For the glycolysis stress test, the CAR-T cells are suspended in serum-free unbuffered DMEM medium (Sigma-Aldrich, St. Louis, MO, USA) supplemented with L-glutamine (200 mM) and NaCl (143 mM). 0.6 mL of a 0.5% Phenol Red solution (SigmaP0290) is added for a final concentration of 3 mg/L and adjust the pH to 7.35+/−0.05. CAR-T cells are plated onto Seahorse cell plates (3E5 cells per well), coated with Cell-Tak (Corning) to facilitate T cell attachment. Briefly, the cartridges are hydrated the day before the assay. On the day of the assay, the plates are coated with Cell-Tak and the cells are seeded in the Cell-Tak coated plates and placed on the XF24 Analyzer for the assay. The detailed procedure is as follows. The assay cartridge is initially hydrated with XF calibrant solution at 200 ul/well, hydro booster is added, and is wrapped in parafilm, and the sensor cartridge is placed on top of utility plate and incubated at 37° C. without CO₂ for overnight. The cell culture plate is then coated with Cell-Tak as follows: For 1 plate, 46 mi of Cell-Tak was diluted in 204 mi TC water and 1 ml of NaHCO₃. The mixer is dispensed 50 mi in each well and the plate is incubated at room temperature for at least 20 minutes. After removing the Cell-Tak solution, 250 mi of TC water is used to wash each well. CAR-T cells (3E5/well) are plated in 158 mi assay media. The cell culture plate is then spun at 450 rpm for 1 sec at slow acceleration and no deceleration, and then the plate was reversed in orientation and spun at 650 rpm for 1 sec at slow acceleration and no deceleration. The plate is then incubated at 37° C. 0% C02 for 25-30 minutes. After 25-30 minutes incubation, 158 ul of warm assay medium is added slowly and gently to the top of each well along the side of the wall using a manual P200 pipettor. The cell plates are incubated for 15-25 minutes. After 15-25 minutes, the plates are placed on XF24 Analyzer (after calibration finished). The XF assay is executed. Solution is injected sequentially through three ports: Port A: glucose 80 mM (96 mL of the stock solution in 3 ml assay media). Port B: oligomycin 18 mM (10.8 mi of the stock solution in 3 ml assay media). Port C: 2DG use stock solution. Glycolysis stress test is performed by measuring ECAR (mpH/min) at steady state after the cartridge ports are loaded with 75 mL of drug solution. For the mitochondrial stress test, CAR T cells are suspended in serum-free unbuffered DMEM medium with D-glucose (25 mM), and sodium pyruvate (1 mM). Mitochondrial stress test is performed similarly as the above by measuring OCR (pmol/min) at steady state and after sequential injection of oligomycin (0.5 mM), FCCP (0.5 mM), rotenone (1 mM) and antimycin A (1 mM) (Sigma-Aldrich). Experiments with the Seahorse system utilize the following assay conditions: 2 minutes mixture; 2 minutes wait; and 3 minutes measurement. All samples are tested in six replicates.

### Fluorescence Microscopy Imaging and Analysis

MOLM14 (4×10 s) tumor cells are plated in 1 ml of warm RPMI on the Cell-tak coated inner well of an ibidi m-Dish 35 mm and incubated overnight in a 37 C incubator. Tumor cells are then stained with Hoechst Dye (2.5 ug/ml). T cells are transduced to express CAR-mCherry fusion proteins. CAR-T positive cells are sorted and then 7.5 E5 of these CAR-T cells are incubated with the fixed MOLM14 cell in the dish for an hour. The cells are subsequently washed and fixed with freshly prepared 4% paraformaldehyde and mounted in a non-hardening mounting media in preparation for imaging.

To evaluate actin expression at the immune synapse, the above protocol is modified, and samples are permeabilized with 0.1% triton x after paraformaldehyde fixation. Cells are stained with Phalloidin 640 (165 nM) and then washed prior to mounting. Airyscan images are acquired using a Zeiss LSM 880. The exposure setting is the same for the entire experiment. Images are collected as a z stack to cover the entire volume of the immune synapse.

Some images will be acquired using a Nikon Eclipse Ti2 spinning disc confocal microscope with 63× objective. Z stacks of 0.5 uM thickness will be acquired in parallel over a range of 10 uM above and below the focal plane for the three channels (405, 488, 640 nm). Each channel is excited at 50% laser intensity with exposure times of 300 ms, 1 s, and 300 ms for 405, 488, and 640, respectively. ImageJ software is used for data analysis.

Quantitative analysis for n>10 immune synapses for each CAR is performed to evaluate CAR and actin accumulation. Specifically, the ratio of mean fluorescence intensity (MFI) at the synapse vs. ratio of the MFI at the rest of the T cell surface is determined. Additional parameters include ratio of MFPvolume at the IS vs. MFI* volume for the rest of the T cell surface, MF volume of IS vs. MFI*volume of T cell, and intracellular CAR signal vs. extracellular CAR signal is also evaluated. For actin, fluorescence intensity at the IS are normalized against the baseline actin T cell expression. MFPvolume of actin at the IS are determined and MFI* volume of unengaged T and tumor cells are subtracted to account for baseline actin expression.

An mCherry reporter sequence is included for measuring MFI.

The sequence for mCherry is:

[SEQ ID NO: 59]
ATGGTGAGCAAGGGCGAGGAGGATAACATGGCCATCATCAAGGAGTTCATGCGCTTCAAGGTGCACATGGAGGGC
TCCGTGAACGGCCACGAGTTCGAGATCGAGGGCGAGGGCGAGGGCCGCCCCTACGAGGGCACCCAGACCGCCAAG
CTGAAGGTGACCAAGGGTGGCCCCCTGCCCTTCGCCTGGGACATCCTGTCCCCTCAGTTCATGTACGGCTCCAAG
GCCTACGTGAAGCACCCCGCCGACATCCCCGACTACTTGAAGCTGTCCTTCCCCGAGGGCTTCAAGTGGGAGCGC
GTGATGAACTTCGAGGACGGCGGCGTGGTGACCGTGACCCAGGACTCCTCCCTGCAGGACGGCGAGTTCATCTAC
AAGGTGAAGCTGCGCGGCACCAACTTCCCCTCCGACGGCCCCGTAATGCAGAAGAAGACCATGGGCTGGGAGGCC
TCCTCCGAGCGGATGTACCCCGAGGACGGCGCCCTGAAGGGCGAGATCAAGCAGAGGCTGAAGCTGAAGGACGGC
GGCCACTACGACGCTGAGGTCAAGACCACCTACAAGGCCAAGAAGCCCGTGCAGCTGCCCGGCGCCTACAACGTC
AACATCAAGTTGGACATCACCTCCCACAACGAGGACTACACCATCGTGGAACAGTACGAACGCGCCGAGGGCCGC
CACTCCACCGGCGGCATGGACGAGCTGTACAAG

### In Vivo Experiments

Animal experiments are carried out under protocols approved by the NCI Bethesda Animal Care and Use Committee. AML cell lines and the xenografted human AML specimens are IV injected into NSG mice. For luciferase-expressing lines, leukemia is detected using the Xenogen IVIS Lumina (Caliper Life Sciences, Hopkinton, MA, USA). NSG are injected intraperitoneally with 3 mg D-luciferin (Caliper Life Sciences) and are imaged 4 minutes later with an exposure time of 1 min for AML cell lines. Living Image Version 4.1 software (Caliper Life Sciences) is used to analyze the total bioluminescent signal flux for each mouse as photons. At time of take down, bone marrow, spleen, and liver of mice are harvested assessed by flow cytometry.

### Statistical Analysis

Statistics analysis is performed using Prism 7.0 software. Plots are presented as mean+/−SD. Statistical significance of all data is calculated using an unpaired student t test. p<0.05 is considered as significant.

### Example 2: Establishing a T Cell Activation Reporter System

Exemplary nucleic acid constructs were designed to encode a reporter molecule operably linked to a minimal NFAT-responsive promoter and a second reporter molecule operably linked to a constitutive promoter (e.g., EF1a). The minimal NFAT-responsive promoter contained 6 NFAT binding sites upstream of a minimal IL-2 promoter comprising a TATA box and the coding sequence of the reporter molecule. The nucleic acids were produced using conventional methods known in the art.

The first nucleic acid construct (EF1a_mOrange_IL-2_mTurq) contained the mOrange reporter molecule under control of the constitutively active ElFalpha promoter and mTurquoise reporter molecule (mTurq) under control of the minimal NFAT-responsive promoter. The second nucleic acid construct (EF1a_mTurq_IL-2_mOrange) contained the mTurquoise reporter molecule under control of the constitutively active ElFalpha promoter and mOrange reporter molecule under control of the minimal NFAT-responsive promoter.

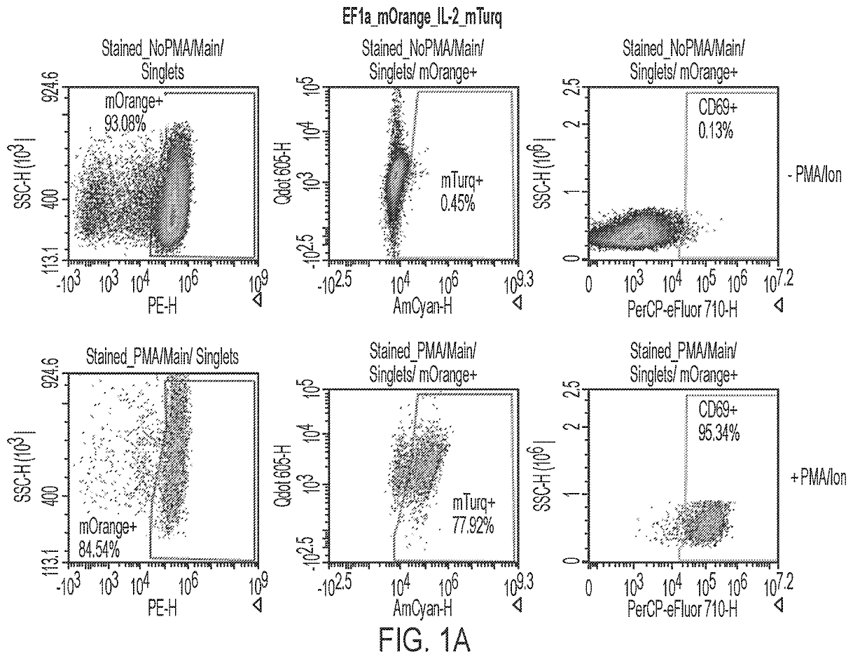

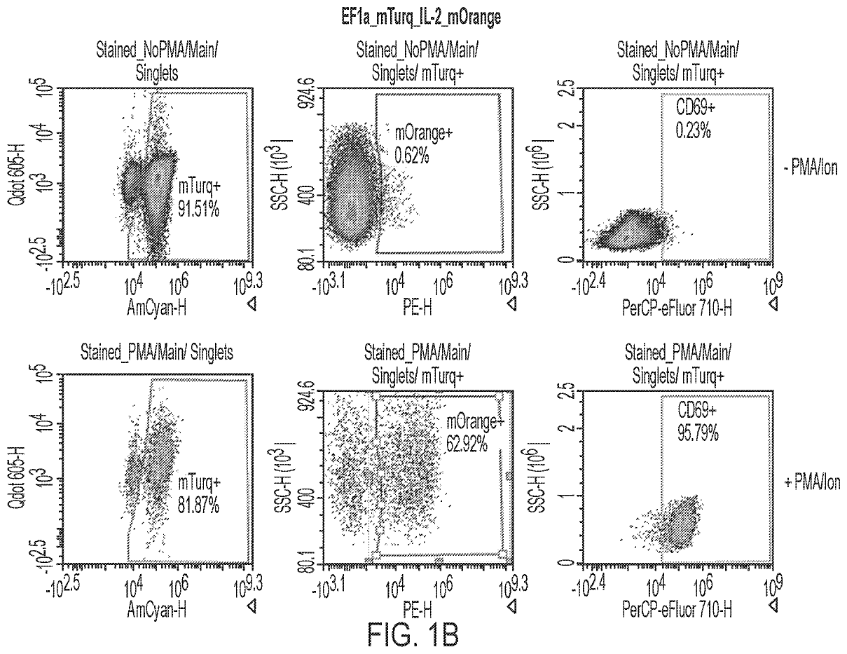

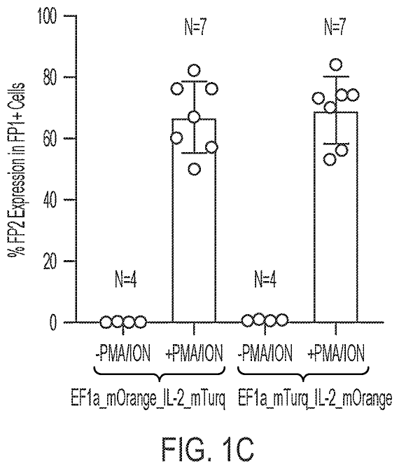

Two IL-2 reporter cell lines were generated by transducing the lentiviral vectors into Jurkat cells. 1×10⁶ cells/mL were activated using 2 μL phorbol myristate acetate (PMA) and ionomycin (a T-cell activation cocktail (see, e.g., BioLegend Activation Cocktail) for 24 hours and assessed for expression of each of the reporter molecules as well as CD69, an indicator of T cell activation, using flow cytometry. As shown in FIGS. 1A and 1B, expression of the reporter molecule under control of the minimal NFAT-responsive promoter was minimally detected when cells were not activated, which significantly increased when cells were activated with PMA/ionomycin. In contrast, expression of the reporter molecule under control of EF1a (the constitutive promoter) was detected in the presence and absence of cell activation. Expression of the reporter molecule under control of the minimal NFAT-responsive promoter was normalized to the expression of the reporter molecule under control of EF1a (the constitutive promoter). See, FIG. 1C.

These results indicate that the minimal NFAT-responsive promoter induces expression of the reporter molecule when activated. Expression of the reporter molecule under control of the minimal NFAT-responsive promoter relative to expression of reporter molecule under control of EF1a (the constitutive promoter) provides a means of normalizing expression to account for factors, such as any differing transduction efficiencies between the constructs.

### Example 3: Evaluating CAR Constructs Using a Reporter System

CAR constructs were designed to target CD33, as shown in Tables 1 and 5. CD33, also known as Siglec (Sialic-acid-binding immunoglobulin-like lectin) plays a role in mediating cell-cell interactions and in maintaining immune cells in a resting state. CD33 is expressed on the surface of the vast majority of AML blasts and chronic myeloid leukemia in blast crisis. It is also aberrantly expressed on a subset of T cell acute lymphoblastic leukemias. Normal tissue expression is restricted to normal myeloid cells. Currently, treating AML with a therapy that targets CD33 can be effective, but the therapy may be limited in utility due to toxicity to the normal blood and bone marrow. The methods described herein allow for comparison of CAR constructs, such as the activity and function of the CAR constructs, as well as high-throughput screening methods for identifying CAR constructs having desired properties (e.g., level of activation of T cells). Example CAR constructs are known in the art. See for example, PCT Publication No. WO 2019/178382 A1, as well as Kenderian, et al. Leukemia (2015) 29: 1637-1647.

Reporter cells containing the exemplary nucleic acid construct EF1a_mOrange_IL-2_mTurq or EF1a_mTurq_IL-2_mOrange were generated as described in Example 2. The cells were transduced with the 8 different CD33 CARs shown in Tables 1 and 5. Cells were co-cultured for 24 hours with either wild-type MOLM-13 cells (CD33+) or MOLM-13 cells that are deficient for CD33 (MOLM-13 CD33KO).

Following co-culture, expression of the reporter molecules was assessed by flow cytometry. Cells were pre-gated on Jurkat cells displaying the fluorescent marker linked to the EF1a promoter, which indicates cells that were transduced with the nucleic acid construct and are able to express the construct. Next, expression of the IL2 linked fluorescent reporter was determined in each co-culture for each of the CD33 CAR constructs as a percentage of constitutive-fluorescence-positive cells (e.g., in cells transduced with EF1a_mOrange_IL-2_mTurq, the expression of mTurq as a percentage of mOrange-positive cells). A ratio was determined for expression of the NFAT-inducible reporter when co-cultured in the presence of wild-type MOLM-13 cells relative to expression of the NFAT-inducible reporter when co-cultured in the presence to MOLM-13 CD33KO cells to determine activity of the CD33 CAR (CD33-specific activation). See, Table 2.

Results indicate that the IL-2 reporter system cells can be used as an objective and reliable reporter system for comparing activity of CAR constructs. Assessing expression of a reporter molecule that is constitutively expressed eliminates false outcomes, potentially due to altered transduction efficiencies, and verifies successful transduction of the reporter construct. Expression of the reporter molecule, driven only in activated cells, represents antigen recognition by and activity of the CAR construct.

TABLE 1
CD33 CAR Constructs Tested

| CD33 | CD33 CAR | | |
|---|---|---|---|
| CAR # | NAME | binder | costim |
| 1 | CD33-CAR1 | lintuzumab | 4-1BB |
| 2 | CD33-CAR2 | My96 | 4-1BB |
| 3 | CD33-CAR3 | mylotarg | CD28 |
| 4 | CD33-CAR4 | lintuzumab | CD28 |
| 5 | CD33-CAR5 | Binder 1 | 4-1BB |
| 6 | CD33-CAR6 | Binder 2 | 4-1BB |
| 7 | CD33-CAR7 | Binder 5 | 4-1BB |
| 8 | CD33-CAR8 | Binder 6 | 4-1BB |

TABLE 2
T Cell Activation Results for Table 1 CD33-CARs

| | Baseline FP2 | Testing FP2 | |
|---|---|---|---|
| | Expression | Expression | |
| CD33CAR | (CD33KO) | (CD33+) | Ratio |
| 1 | NA | NA | NA |
| 2 | 4.67 | 27.79 | 5.95 |
| 3 | 2.7 | 3.9 | 1.44 |
| 4 | 1.72 | 4.14 | 2.41 |
| 5 | 3.32 | 28.21 | 8.50 |
| 6 | 6.36 | 22.89 | 3.60 |
| 7 | 8.87 | 17.55 | 1.98 |
| 8 | 5.01 | 29.36 | 5.86 |

The results show that the anti-CD33 CARs of the disclosure (CD33 CARs 5-8) show T cell activating activity greater than at least one previously known anti-CD33 CAR. CD33-CAR5 showed the highest T cell activating activity of all CD33 CARs tested. These results demonstrate the potential of CD33 CARs of the present disclosure in the construction of CAR T therapeutics targeting CD33-expressing cancers.

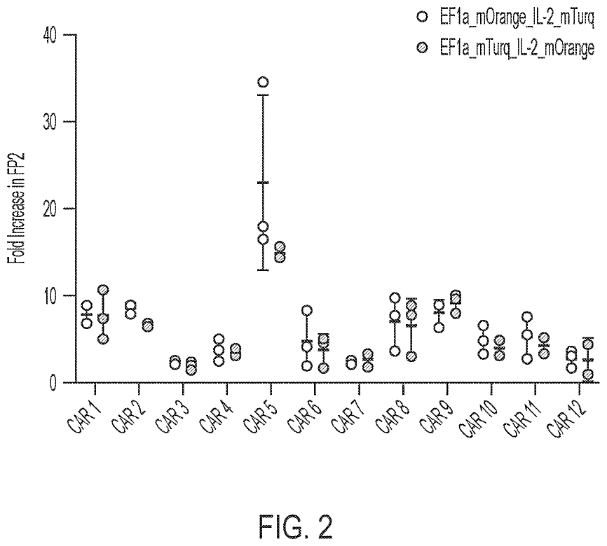

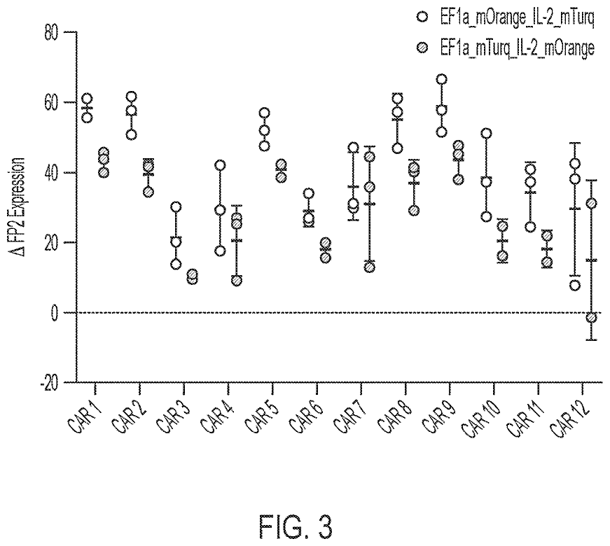

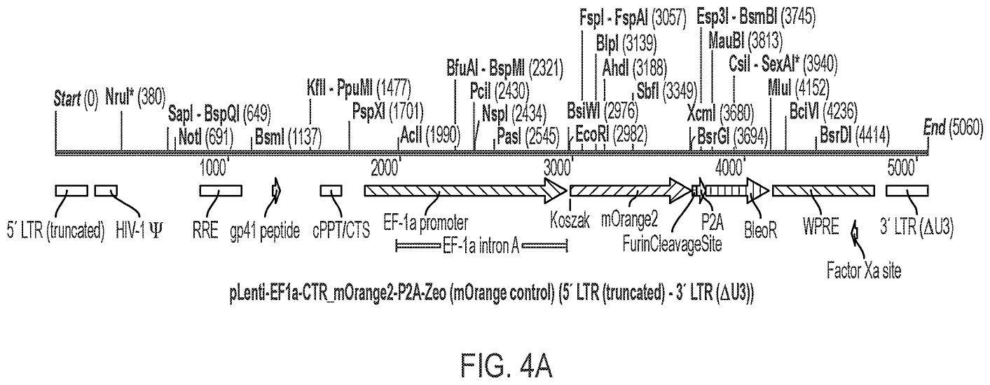

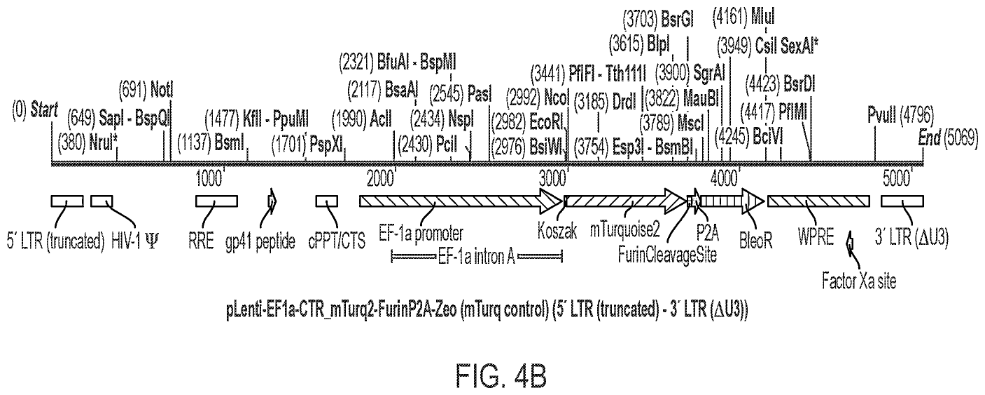

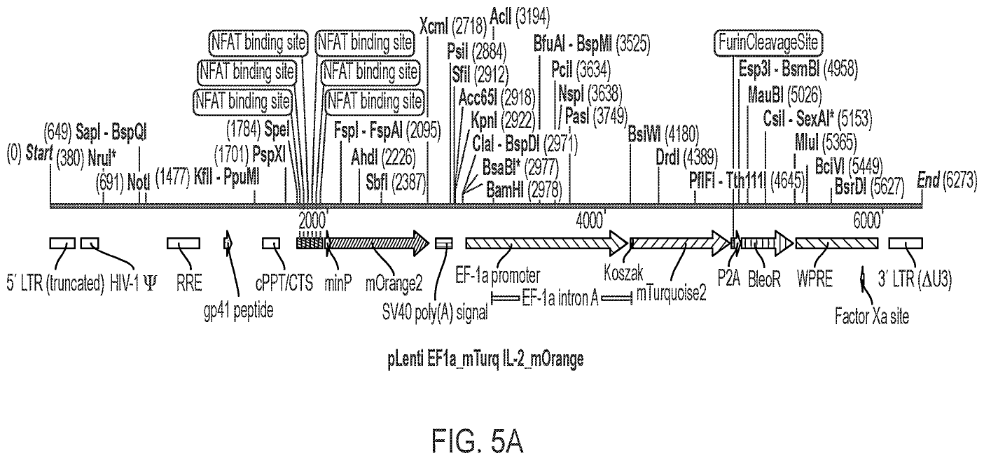

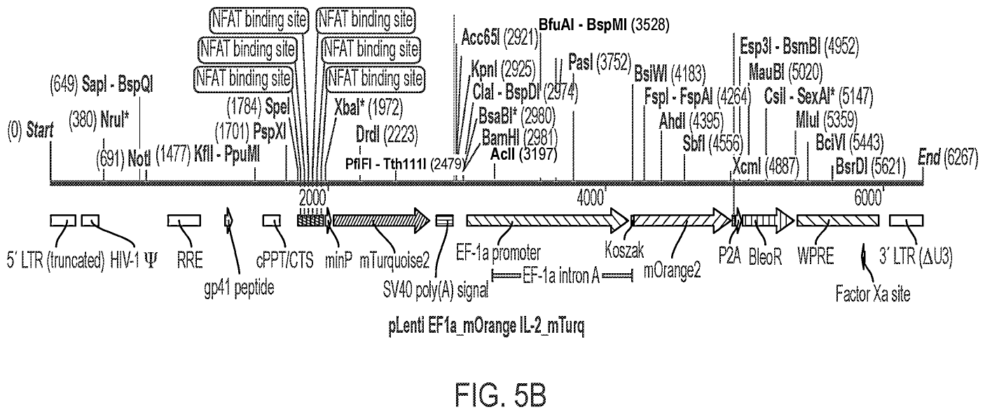

The extent to which the 8 CD33 CARs activate T cells was further evaluated by examining the fold increase in NFAT-inducible fluorescence (FIG. 2, data in Table 3) and the absolute change in NFAT-inducible fluorescence (ΔFP2) (FIG. 3). As controls, lentiviral vectors encoding known costimulatory or co-inhibitory agents (OX40, ICOS, TIM3, or a VH/VL against CD28) were transduced into Jurkat cells previously transduced with the EF1a_mOrange_IL-2 mTurq or EF1a_mTurq_IL-2 mOrange construct.

TABLE 3
Fold and Delta Increases in FP2 in Tested CARs

| | EF1a_ | EF1a_ | EF1a_ | EF1a_ |
|---|---|---|---|---|
| | mOrange_ | mTurq_ | mOrange_ | mTurq_ |
| | IL-2_ | IL-2_ | IL-2_ | IL-2_ |
| CAR | mTurq (Fold | mOrange (Fold | mTurq | mOrange |
| # | Increase) | Increase) | (Delta) | (Delta) |
| 1 | 0.182770672 | 0.36735279 | 0.06768948 | 0.06560674 |
| 2 | 0.060681329 | 0.029963018 | 0.09615783 | 0.11017492 |
| 3 | 0.064981794 | 0.224606151 | 0.3825048 | 0.07078721 |
| 4 | 0.337347505 | 0.131762658 | 0.41099215 | 0.48320023 |
| 5 | 0.43677822 | 0.046825787 | 0.09053537 | 0.04881308 |
| 6 | 0.675153525 | 0.482256984 | 0.15073833 | 0.12741785 |
| 7 | 0.097575115 | 0.270767959 | 0.26618319 | 0.52298681 |
| 8 | 0.437212356 | 0.475518004 | 0.13344737 | 0.18178983 |

The results in FIGS. 2 and 3 show that all CD33-CAR5-8 of the disclosure show T cell activating activity in the CAR-IRS assay to varying degrees. For example, CD33-CAR5 shows a high FP2 fold increase (FIG. 2), suggesting a higher T cell activating activity than other CARs tested.

TABLE 4
Sequences of CAR Constructs and Controls in Example 2

| | | Co- | |
|---|---|---|---|
| | | stimulatory | |
| Name | Binder | domain | Amino acid sequence |
| CD33- | huM195 | CD137 | MALPVTALLLPLALLLHAARPQVQLVQSGAEVKKP |
| CAR1 | | | GSSVKVSCKASGYTFTDYNMHWVRQAPGQGLEWIG |
| | | | YIYPYNGGTGYNQKFKSKATITADESTNTAYMELS |
| | | | SLRSEDTAVYYCARGRPAMDYWGQGTLVTVSSGGG |
| | | | GSGGGGSGGGGSDIQMTQSPSSLSASVGDRVTITC |
| | | | RASESVDNYGISFMNWFQQKPGKAPKLLIYAASNQ |
| | | | GSGVPSRFSGSGSGTDFTLTISSLQPDDFATYYCQ |
| | | | QSKEVPWTFGQGTKVEIKSGTTTPAPRPPTPAPTI |
| | | | ASQPLSLRPEACRPAAGGAVHTRGLDFACDIYIWA |
| | | | PLAGTCGVLLLSLVITLYCKRGRKKLLYIFKQPFM |
| | | | RPVQTTQEEDGCSCRFPEEEEGGCELRVKFSRSAD |
| | | | APAYKQGQNQLYNELNLGRREEYDVLDKRRGRDPE |
| | | | MGGKPRRKNPQEGLYNELQKDKMAEAYSEIGMKGE |
| | | | RRRGKGHDGLYQGLSTATKDTYDALHMQALPPR |
| | | | [SEQ ID NO: 22] |
| CD33- | CD33-1 | CD137 | MALPVTALLLPLALLLHAARPQVQLVQSGAEVKKP |
| CAR2 | | (4-1BB) | GESLKISCKGSGYSFTSYWIGWVRQMPGKGLEWMG |
| | | | IIYPGDSDTRYSPSFQGQVTISADKSISTAYLQWS |
| | | | SLKASDTAMYYCARLGGSLPDYGMDVWGQGTMVTV |
| | | | SSASGGGGSGGGGSGGGGSEIVLTQSPLSLPVTPG |
| | | | EPASISCRSSQSLLHSNGYNYLDWYLQKPGQSPQL |
| | | | LIYLGSNRASGVPDRFSGSGSGTDFTLKISRVEAE |
| | | | DVGVYYCMQALQTLITFGQGTKVDIKTTTPAPRPP |
| | | | TPAPTIASQPLSLRPEACRPAAGGAVHTRGLDFAC |
| | | | DIYIWAPLAGTCGVLLLSLVITLYCKRGRKKLLYI |
| | | | FKQPFMRPVQTTQEEDGCSCRFPEEEEGGCELRVK |
| | | | FSRSADAPAYKQGQNQLYNELNLGRREEYDVLDKR |
| | | | RGRDPEMGGKPRRKNPQEGLYNELQKDKMAEAYSE |
| | | | IGMKGERRRGKGHDGLYQGLSTATKDTYDALHMQA |
| | | | LPPR [SEQ ID NO: 29] |
| CD33- | Mylo | CD28 | MALPVTALLLPLALLLHAARPQVQLVQSGAEVKKP |
| CAR3 | | | GSSVKVSCKASGYTFTDYNMHWVRQAPGQGLEWIG |
| | | | YIYPYNGGTGYNQKFKSKATITADESTNTAYMELS |
| | | | SLRSEDTAVYYCARGRPAMDYWGQGTLVTVSSGGG |
| | | | GSGGGGSGGGGSDIQMTQSPSSLSASVGDRVTITC |
| | | | RASESVDNYGISFMNWFQQKPGKAPKLLIYAASNQ |
| | | | GSGVPSRFSGSGSGTDFTLTISSLQPDDFATYYCQ |
| | | | QSKEVPWTFGQGTKVEIKSGAAAIEVMYPPPYLDN |
| | | | EKSNGTIIHVKGKHLCPSPLFPGPSKPFWVLVVVG |
| | | | GVLACYSLLVTVAFIIFWVRSKRSRLLHSDYMNMT |
| | | | PRRPGPTRKHYQPYAPPRDFAAYRSRVKFSRSADA |
| | | | PAYQQGQNQLYNELNLGRREEYDVLDKRRGRDPEM |
| | | | GGKPRRKNPQEGLYNELQKDKMAEAYSEIGMKGER |
| | | | RRGKGHDGLYQGLSTATKDTYDALHMQALPPRGS |
| | | | [SEQ ID NO: 19] |
| CD33- | huM195 | CD28 | MALPVTALLLPLALLLHAARPEVQLVQSGAEVKKP |
| CAR4 | | | GSSVKVSCKASGYTITDSNIHWVRQAPGQSLEWIG |
| | | | YIYPYNGGTDYNQKFKNRATLTVDNPTNTAYMELS |
| | | | SLRSEDTAFYYCVNGNPWLAYWGQGTLVTVSSGGG |
| | | | GSGGGGSGGGGSDIQLTQSPSTLSASVGDRVTITC |
| | | | RASESLDNYGIRFLTWFQQKPGKAPKLLMYAASNQ |
| | | | GSGVPSRFSGSGSGTEFTLTISSLQPDDFATYYCQ |
| | | | QTKEVPWSFGQGTKVEVKRISSGAAAIEVMYPPPY |
| | | | LDNEKSNGTIIHVKGKHLCPSPLFPGPSKPFWVLV |
| | | | VVGGVLACYSLLVTVAFIIFWVRSKRSRLLHSDYM |
| | | | NMTPRRPGPTRKHYQPYAPPRDFAAYRSRVKFSRS |
| | | | ADAPAYQQGQNQLYNELNLGRREEYDVLDKRRGRD |
| | | | PEMGGKPRRKNPQEGLYNELQKDKMAEAYSEIGMK |
| | | | GERRRGKGHDGLYQGLSTATKDTYDALHMQALPPR |
| | | | GS [SEQ ID NO: 13] |
| CD33- | Binder 1 | CD137 | MELGLSWVVLAALLQGVQAQVKLEESGGGSVQAGE |
| CAR5 | | | SLRLSCTASGITFRDYDIDWYRQAPGKEREWLATI |
| | | | TPSGTTHYPDSVKGRATISRDSAKNTVYLQMNSLK |
| | | | PEDTARYECNTLAYWGSGTQVTVSSAAATTTPAPR |
| | | | PPTPAPTIASQPLSLRPEACRPAAGGAVHTRGLDF |
| | | | ACDIYIWAPLAGTCGVLLLSLVITLYCKRGRKKLL |
| | | | YIFKQPFMRPVQTTQEEDGCSCRFPEEEEGGCELR |
| | | | VKFSRSADAPAYQQGQNQLYNELNLGRREEYDVLD |
| | | | KRRGRDPEMGGKPRRKNPQEGLYNELQKDKMAEAY |
| | | | SEIGMKGERRRGKGHDGLYQGLSTATKDTYDALHM |
| | | | QALPPR [SEQ ID NO: 60] |
| CD33- | Binder 2 | CD137 | MELGLSWVVLAALLQGVQAQVQLVETGGGLVRAGG |
| CAR6 | | | SLRLSCAASGRTADIYNIGWFRQAPGKEREFVAAI |
| | | | TWIGRTPYYADAVKGRFAFSTDSAKNTVSLQMDNL |
| | | | KPEDTGVYYCNAAHYLEGNTDYYWGQGTQVTVSSA |
| | | | AATTTPAPRPPTPAPTIASQPLSLRPEACRPAAGG |
| | | | AVHTRGLDFACDIYIWAPLAGTCGVLLLSLVITLY |
| | | | CKRGRKKLLYIFKQPFMRPVQTTQEEDGCSCRFPE |
| | | | EEEGGCELRVKFSRSADAPAYQQGQNQLYNELNLG |
| | | | RREEYDVLDKRRGRDPEMGGKPRRKNPQEGLYNEL |
| | | | QKDKMAEAYSEIGMKGERRRGKGHDGLYQGLSTAT |
| | | | KDTYDALHMQALPPR [SEQ ID NO: 90] |
| CD33- | Binder 5 | CD137 | MELGLSWVVLAALLQGVQAQVQLVQPGGSLRLFCV |
| CAR7 | | | ASEEFFSIYAMGWYRQAPGKQHEMVARFTRDGKIT |
| | | | YADSAKGRFTITRDAKNTLNLQMNGLIPEDTAVYY |
| | | | CNINHYWGQGTQVTVSSAAATTTPAPRPPTPAPTI |
| | | | ASQPLSLRPEACRPAAGGAVHTRGLDFACDIYIWA |
| | | | PLAGTCGVLLLSLVITLYCKRGRKKLLYIFKQPFM |
| | | | RPVQTTQEEDGCSCRFPEEEEGGCELRVKFSRSAD |
| | | | APAYQQGQNQLYNELNLGRREEYDVLDKRRGRDPE |
| | | | MGGKPRRKNPQEGLYNELQKDKMAEAYSEIGMKGE |
| | | | RRRGKGHDGLYQGLSTATKDTYDALHMQALPPR |
| | | | [SEQ ID NO: 91] |
| CD33- | Binder 6 | CD137 | MELGLSWVVLAALLQGVQADVQLVESGGGLVQPGG |
| CAR8 | | | SLRLSCSVSGNIDRFYAMGWYRQAPGKQRELVAQL |
| | | | TNNEITTYGDSVEGQFSISGDFDANTVYLQMDSLK |
| | | | PEDTAVYYCHAHVTTTRWSQDYYWGQGTRVTVSSA |
| | | | AATTTPAPRPPTPAPTIASQPLSLRPEACRPAAGG |
| | | | AVHTRGLDFACDIYIWAPLAGTCGVLLLSLVITLY |
| | | | CKRGRKKLLYIFKQPFMRPVQTTQEEDGCSCRFPE |
| | | | EEEGGCELRVKFSRSADAPAYQQGQNQLYNELNLG |
| | | | RREEYDVLDKRRGRDPEMGGKPRRKNPQEGLYNEL |
| | | | QKDKMAEAYSEIGMKGERRRGKGHDGLYQGLSTAT |
| | | | KDTYDALHMQALPPR [SEQ ID NO: 92] |
| CD33- | huM195 | OX40 | MALPVTALLLPLALLLHAARPQVQLVQSGAEVKKP |
| CAR9 | | | GSSVKVSCKASGYTFTDYNMHWVRQAPGQGLEWIG |
| | | | YIYPYNGGTGYNQKFKSKATITADESTNTAYMELS |
| | | | SLRSEDTAVYYCARGRPAMDYWGQGTLVTVSSGGG |
| | | | GSGGGGSGGGGSDIQMTQSPSSLSASVGDRVTITC |
| | | | RASESVDNYGISFMNWFQQKPGKAPKLLIYAASNQ |
| | | | GSGVPSRFSGSGSGTDFTLTISSLQPDDFATYYCQ |
| | | | QSKEVPWTFGQGTKVEIKSGAAATTTPAPRPPTPA |
| | | | PTIASQPLSLRPEACRPAAGGAVHTRGLDFACDIY |
| | | | IWAPLAGTCGVLLLSLVITLYCRRDQRLPPDAHKP |
| | | | PGGGSFRTPIQEEQADAHSTLAKIRVKFSRSADAP |
| | | | AYQQGQNQLYNELNLGRREEYDVLDKRRGRDPEMG |
| | | | GKPRRKNPQEGLYNELQKDKMAEAYSEIGMKGERR |
| | | | RGKGHDGLYQGLSTATKDTYDALHMQALPPRGS |
| | | | [SEQ ID NO: 94] |
| CD33- | huM195 | ICOS | MALPVTALLLPLALLLHAARPQVQLVQSGAEVKKP |
| CAR10 | | | GSSVKVSCKASGYTFTDYNMHWVRQAPGQGLEWIG |
| | | | YIYPYNGGTGYNQKFKSKATITADESTNTAYMELS |
| | | | SLRSEDTAVYYCARGRPAMDYWGQGTLVTVSSGGG |
| | | | GSGGGGSGGGGSDIQMTQSPSSLSASVGDRVTITC |
| | | | RASESVDNYGISFMNWFQQKPGKAPKLLIYAASNQ |
| | | | GSGVPSRFSGSGSGTDFTLTISSLQPDDFATYYCQ |
| | | | QSKEVPWTFGQGTKVEIKSGAAATTTPAPRPPTPA |
| | | | PTIASQPLSLRPEACRPAAGGAVHTRGLDFACDIY |
| | | | IWAPLAGTCGVLLLSLVITLYCKKKYSSSVHDPNG |
| | | | EYMFMRAVNTAKKSRLTDVTLRVKFSRSADAPAYQ |
| | | | QGQNQLYNELNLGRREEYDVLDKRRGRDPEMGGKP |
| | | | RRKNPQEGLYNELQKDKMAEAYSEIGMKGERRRGK |
| | | | GHDGLYQGLSTATKDTYDALHMQALPPRGS [SEQ |
| | | | ID NO: 95] |
| CD33- | huM195 | TIM3 | MALPVTALLLPLALLLHAARPQVQLVQSGAEVKKP |
| CAR11 | | | GSSVKVSCKASGYTFTDYNMHWVRQAPGQGLEWIG |
| | | | YIYPYNGGTGYNQKFKSKATITADESINTAYMELS |
| | | | SLRSEDTAVYYCARGRPAMDYWGQGTLVTVSSGGG |
| | | | GSGGGGSGGGGSDIQMTQSPSSLSASVGDRVTITC |
| | | | RASESVDNYGISFMNWFQQKPGKAPKLLIYAASNQ |
| | | | GSGVPSRFSGSGSGTDFTLTISSLQPDDFATYYCQ |
| | | | QSKEVPWTFGQGTKVEIKSGAAATTTPAPRPPTPA |
| | | | PTIASQPLSLRPEACRPAAGGAVHTRGLDFACDIY |
| | | | IWAPLAGTCGVLLLSLVITLYCKWYSHSKEKIQNL |
| | | | SLISLANLPPSGLANAVAEGIRSEENIYTIEENVY |
| | | | EVEEPNEYYCYVSSRQQPSQPLGCRFAMPRVKFSR |
| | | | SADAPAYQQGQNQLYNELNLGRREEYDVLDKRRGR |
| | | | DPEMGGKPRRKNPQEGLYNELQKDKMAEAYSEIGM |
| | | | KGERRRGKGHDGLYQGLSTATKDTYDALHMQALPP |
| | | | RGS [SEQ ID NO: 96] |
| CD33- | huM195 | CD28 | MALPVTALLLPLALLLHAARPDIQMTQSPSSLSAS |
| CAR12 | (VL/VH) | | VGDRVTITCRASESVDNYGISFMNWFQQKPGKAPK |
| | | | LLIYAASNQGSGVPSRFSGSGSGTDFTLTISSLQP |
| | | | DDFATYYCQQSKEVPWTFGQGTKVEIKGGGGSGGG |
| | | | GSGGGGSQVQLVQSGAEVKKPGSSVKVSCKASGYT |
| | | | FTDYNMHWVRQAPGQGLEWIGYTYPYNGGTGYNQK |
| | | | FKSKATITADESTNTAYMELSSLRSEDTAVYYCAR |
| | | | GRPAMDYWGQGTLVTVSSSGAAAIEVMYPPPYLDN |
| | | | EKSNGTIIHVKGKHLCPSPLFPGPSKPFWVLVVVG |
| | | | GVLACYSLLVTVAFIIFWVRSKRSRLLHSDYMNMT |
| | | | PRRPGPTRKHYQPYAPPRDFAAYRSRVKFSRSADA |
| | | | PAYQQGQNQLYNELNLGRREEYDVLDKRRGRDPEM |
| | | | GGKPRRKNPQEGLYNELQKDKMAEAYSEIGMKGER |
| | | | RRGKGHDGLYQGLSTATKDTYDALHMQALPPRGS |
| | | | [SEQ ID NO: 97] |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

We claim:

1. An isolated nucleic acid molecule encoding a chimeric antigen receptor (CAR), wherein the CAR comprises a CD33 binding domain, a transmembrane domain, and an intracellular signaling domain,
wherein the encoded CD33 binding domain comprises a heavy chain variable region and/or a light chain variable region;
wherein the encoded transmembrane domain comprises a transmembrane domain of a protein selected from CD8a or CD28; and
wherein the encoded intracellular signaling domain comprises a functional signaling domain of CD3.

2. The isolated nucleic acid molecule of claim 1, wherein the heavy chain variable region and the light chain variable region are joined by a linker.

3. The isolated nucleic acid molecule of claim 1 or claim 2, wherein the encoded CD33 binding domain comprises a single-chain variable fragment (scFv), an Fab, an F(ab′)2, a dsFv, a diabody, or a tiabody.

4. The isolated nucleic acid molecule of any one of claims 1-3, wherein the encoded CD33 binding domain is connected to the transmembrane domain by a hinge region.

5. The isolated nucleic acid molecule of claim 4, wherein the encoded hinge region comprises a hinge region of a protein selected from CD8a, IgG4, or CD28.

6. The isolated nucleic acid molecule of any one of claims 1-5, wherein the encoded CAR further comprises one or more co-stimulatory domains.

7. The isolated nucleic acid molecule of claim 9, wherein the one more co-stimulatory domains comprises a functional signaling domain of 4-1BB and/or CD28.

8. The isolated nucleic acid molecule of any one of claims 1-7, wherein the isolated nucleic acid sequence further comprises a promoter sequence.

9. The isolated nucleic acid molecule of claim 8, wherein the promoter sequence is a SFFV (silencing-prone spleen focus forming virus) promoter sequence or a EF1α promoter sequence.

10. The isolated nucleic acid molecule of any one of claims 1-10b, wherein the encoded CAR comprises
(i) an amino acid sequence of any one of SEQ ID NOs: 10, 13, 16, 19, 22, 25, 29, 33, 36, 39, 42, 45, 48, 51, 54, 57, and 60-92; or
(ii) an amino acid sequence having 95-99% identity to any one of SEQ ID NOs: 10, 13, 16, 19, 22, 25, 29, 33, 36, 39, 42, 45, 48, 51, 54, 57, and 60-92.

11. The isolated nucleic acid molecule of any one of claims 1-10, wherein the nucleic acid molecule comprises
(i) a nucleotide sequence selected from any one of SEQ ID NOs: 9, 12, 15, 18, 21, 24, 28, 32, 35, 38, 41, 44, 47, 50, 53, and 56; or
(ii) a nucleotide sequence with 95-99% identity to any one of SEQ ID NOs: 9, 12, 15, 18, 21, 24, 28, 32, 35, 38, 41, 44, 47, 50, 53, and 56.

12. An expression vector comprising the nucleic acid molecule encoding a CAR of any one of claims 1-11.

13. The expression vector of claim 12, wherein the vector is a DNA vector, an RNA vector, a plasmid, a lentivirus vector, an adenoviral vector or a retrovirus vector.

14. The expression vector of claim 12 or claim 13, wherein the expression vector comprises
(i) a nucleotide sequence selected from any one of SEQ ID NOs: 11, 14, 17, 20, 23, 26, 30, 34, 37, 40, 43, 46, 49, 52, 55, and 58; or
(ii) a nucleotide sequence with 95-99% identity to any one of SEQ ID NOs: 11, 14, 17, 20, 23, 26, 30, 34, 37, 40, 43, 46, 49, 52, 55, and 58.

15. An immune effector cell comprising the nucleic acid molecule of any one of claims 1-14.

16. The immune effector cell of claim 15, wherein the cell is selected from the group consisting of a T cell, a Natural Killer (NK) cell, a cytotoxic T lymphocyte (CTL), a regulatory T cell, a human embryonic stem cell, and a pluripotent stem cell from which lymphoid cells may be differentiated.

17. A population of cells comprising at least one immune effector cell of claim 15 or claim 16.

18. A pharmaceutical composition comprising the population of cells of claim 17 and a pharmaceutically acceptable carrier.

19. A method of treating a hematopoietic malignancy, comprising administering to a subject in need thereof an effective amount of an agent targeting CD33, wherein the agent is an immune cell expressing a chimeric receptor (CAR), wherein the CAR comprises:
an antigen-binding domain that binds CD33 comprising a heavy chain variable region and/or a light chain variable region;
a transmembrane domain comprising a transmembrane domain of a protein selected from CD8a or CD28; and
an intracellular signaling domain comprising a functional signaling domain of CD3.

20. The method of claim 19, where the method further comprises administering a population of hematopoietic cells, wherein the hematopoietic cells are genetically-engineered such that the gene encoding CD33 that is targeted by the antigen-binding domain is engineered to reduce or eliminate the expression of CD33.

21. The method of claim 20, wherein the immune cells, the hematopoietic cells, or both, are allogeneic or autologous.

22. The method of any one of claim 20 or 21, wherein the hematopoietic cells are hematopoietic stem cells.

23. The method of claim 22, wherein the hematopoietic stem cells are from bone marrow cells or peripheral blood mononuclear cells (PBMCs).

24. The method of claim 22 or claim 23, wherein the hematopoietic stem cells are CD34+/CD33−.

25. The method of any one of claims 19-24, wherein the hematopoietic cells are prepared by editing the endogenous gene coding for CD33 to reduce or eliminate the expression of CD33.

26. The method of claim 25, wherein the endogenous gene is edited by CRISPR-Cas9.

27. The method of any one of claims 19-26, wherein the subject has or has been diagnosed with a hematopoietic malignancy or pre-malignancy characterized by the expression of CD33 on malignant cells or pre-malignant cells.

28. The method of any one of claims 19-27, wherein the subject has Hodgkin's lymphoma, non-Hodgkin's lymphoma, leukemia, or multiple myeloma.

29. The method of claim 28, wherein the leukemia is acute myeloid leukemia, myelodysplastic syndrome, chronic myelogenous leukemia, acute lymphoblastic leukemia, or chronic lymphoblastic leukemia.

30. The method of any one of claims 19-29, wherein the immune cells comprise one or more cell types selected from the group consisting of a T cell, a Natural Killer (NK) cell, a cytotoxic T lymphocyte (CTL), a regulatory T cell, a human embryonic stem cell, and a pluripotent stem cell from which lymphoid cells may be differentiated.

31. The method of any one of claims 19-30, wherein the antigen-binding domain in the CAR is a single-chain variable fragment (scFv), an Fab, an F(ab′)₂, a dsFv, a diabody, nanobody, or a triabody that specifically binds CD33.

32. The method of any one of claims 19-31, wherein the heavy chain variable region and the light chain variable region of the antigen-binding domain are joined by a linker.

33. The method of any one of claims 19-32, wherein the antigen-binding domain is connected to the transmembrane domain by a hinge region.

34. The method of claim 33, wherein the hinge region comprises a hinge region of a protein selected from CD8a, IgG4, or CD28.

35. The method of any one of claims 19-34, wherein the CAR further comprises one or more co-stimulatory domains.

36. The method of claim 35, wherein the one more co-stimulatory domains comprises a functional signaling domain of 4-1BB and/or CD28.

37. The method of any one of claims 19-36, wherein the encoded CAR comprises
(i) an amino acid sequence of any one of SEQ ID NOs: 10, 13, 16, 19, 22, 25, 29, 33, 36, 39, 42, 45, 48, 51, 54, 57, and 60-92; or
(ii) an amino acid sequence having 95-99% identity to any one of SEQ ID NOs: 10, 13, 16, 19, 22, 25, 29, 33, 36, 39, 42, 45, 48, 51, 54, 57, and 60-92.

38. The method of any one of claims 19-37, wherein the CAR is encoded by a nucleotide sequence that is
(i) selected from any one of SEQ ID NOs: 9, 12, 15, 18, 21, 24, 28, 32, 35, 38, 41, 44, 47, 50, 53, and 56; or
(ii) 95-99% identical to any one of SEQ ID NOs: 9, 12, 15, 18, 21, 24, 28, 32, 35, 38, 41, 44, 47, 50, 53, and 56.

39. The method of any one of claims 19-38, wherein the agent targeting CD33 further comprises a pharmaceutically acceptable carrier.
